SEQUENCE LISTING (1113 sequences)

SEQ ID NO: 1 - DNA, 50 bp, Homo sapiens
cgggaccgtttggccctctgtgacccaagtcagcctcggcgaagtgcttt

SEQ ID NO: 2 - DNA, 50 bp, Homo sapiens
cgcggccaggcagcgaaccccactaggaggcaccccaggcccctgggctt

SEQ ID NO: 3 - DNA, 50 bp, Homo sapiens
cgcgcctgggcccccaggacctcttgcagacgggagtccctcagacagtg

SEQ ID NO: 4 - DNA, 50 bp, Homo sapiens
ggttgcttccttttgtgccccccgggggtcaggaatcccaggtttctccg

SEQ ID NO: 5 - DNA, 50 bp, Homo sapiens
ctgaggccacctggggctggggatcccactcttcttgcagctgttgagcg

SEQ ID NO: 6 - DNA, 50 bp, Homo sapiens
agggccccctgggcctgtaccaaggtctgcgacagatgggtggggagccg

SEQ ID NO: 7 - DNA, 50 bp, Homo sapiens
gcctggcagaatcaggagccgggggagtagggctcccggaaagtttatcg

SEQ ID NO: 8 - DNA, 50 bp, Homo sapiens
gaggcagtgggcggggctgaagctgcaggtctggcagagggcgggctgcg

SEQ ID NO: 9 - DNA, 50 bp, Homo sapiens
gtgggcggggctgaagctgcaggtctggcagagggcgggctgcggcagcg

SEQ ID NO: 10 - DNA, 50 bp, Homo sapiens
cgcaagcatgaagcgtttaactgtgcaacacaaatcaacaacctgggctc

SEQ ID NO: 11 - DNA, 50 bp, Homo sapiens
cgcacccactgaatcagaagctgtaggagcgggagccagtggtttgcatt

SEQ ID NO: 12 - DNA, 50 bp, Homo sapiens
cgctgccaaacccctacacgaaagtttggttctgttccagaaactatgga

SEQ ID NO: 13 - DNA, 50 bp, Homo sapiens
ggacaaaaatcctcggtggcgggggcaaaaagtcgcggtgacaaaaagcg

SEQ ID NO: 14 - DNA, 50 bp, Homo sapiens
tttgaagcccttatgtctatggtaatctgttacagcccccgcaggaaacg

SEQ ID NO: 15 - DNA, 50 bp, Homo sapiens
cgggcaccttgctgctggccctcggccccacgcacctgcccttggctgcc

SEQ ID NO: 16 - DNA, 50 bp, Homo sapiens
cgtgaattgagatgtcatgccattgtaagatttttaaaaagggaaggaca

SEQ ID NO: 17 - DNA, 50 bp, Homo sapiens
cggcgcgcactgcacaatgaacatgcaaaacaccttcaactgcacttgct

SEQ ID NO: 18 - DNA, 50 bp, Homo sapiens
ccatagcggtgggcctttggcgacgtcagaggcgcgggtgttcgcctacg

SEQ ID NO: 19 - DNA, 50 bp, Homo sapiens
agctcctgccgtggtggtgaaatattgtggactgcttggctgactacacg

SEQ ID NO: 20 - DNA, 50 bp, Homo sapiens
cgtgctcagctgataaaatgctaaatagcatcaatgctgaccatgcttct

SEQ ID NO: 21 - DNA, 50 bp, Homo sapiens
cgtgtgtacacatgtatgtctttcaagcaaggctatgtattactcagaac

SEQ ID NO: 22 - DNA, 50 bp, Homo sapiens
gtcagggagataagtccctttgcggaatgtcaagacggtgagcaggcgcg

SEQ ID NO: 23 - DNA, 50 bp, Homo sapiens
cgcttcttaagggaaaggcatgaggtgggacaggcaaaccttggcaagtg

SEQ ID NO: 24 - DNA, 50 bp, Homo sapiens
cggcaggcatcccctctacctgaccggcttcttcggaggatacatcttgg

SEQ ID NO: 25 - DNA, 50 bp, Homo sapiens
attagcggagcctccgcctatgattggcttcgcccgggaagctggagacg

SEQ ID NO: 26 - DNA, 50 bp, Homo sapiens
cgggggctcttcctgctcaagctgggacttagcctgctcatgttgttggc

SEQ ID NO: 27 - DNA, 50 bp, Homo sapiens
ctttttttggcattggaattggcaagtggctgcccttgtctgttacaacg

SEQ ID NO: 28 - DNA, 50 bp, Homo sapiens
cggggtgcaggtcagggctccaggtgtcacctgcctgtccacacggggct

SEQ ID NO: 29 - DNA, 50 bp, Homo sapiens
cgccgtgtccctcacgctggctttcagaaggcttcacgcaagtgggctca

SEQ ID NO: 30 - DNA, 50 bp, Homo sapiens
cgggtgctgttatcaccccaatttcaccaatgaggaaagaggtgcagaga

SEQ ID NO: 31 - DNA, 50 bp, Homo sapiens
gtggttttctacctggacctctccttcatcttcctcctagaactgaagcg

SEQ ID NO: 32 - DNA, 50 bp, Homo sapiens
gctgtggcgtctccaactacactgactggttcgaggtgtacaacgccacg

SEQ ID NO: 33 - DNA, 50 bp, Homo sapiens
cgtggacttcccatcccctggaccccacggcggggacaaggaaacctaca

SEQ ID NO: 34 - DNA, 50 bp, Homo sapiens
agcctcactgaggaagtgactgtcccaggctctagaggtgcctgagggcg

SEQ ID NO: 35 - DNA, 50 bp, Homo sapiens
agacggggaggccgggcagagacgctcctcacctcccagacagggttgcg

SEQ ID NO: 36 - DNA, 50 bp, Homo sapiens
cgaccctttgtggagggcactggaggagctggcatttaagccctgtccag

SEQ ID NO: 37 - DNA, 50 bp, Homo sapiens
aatgcgtcgcagtcaagacagcgcaggggcctgggctgccgggcgctgcg

SEQ ID NO: 38 - DNA, 50 bp, Homo sapiens
caagggcacacagcgaggcagtttcagggcgggcagcctggggccccacg

SEQ ID NO: 39 - DNA, 50 bp, Homo sapiens
cggggcagtgctttcccagaaggattgctcggcatcctgcccttcccaga

SEQ ID NO: 40 - DNA, 50 bp, Homo sapiens
cgcacagcactgagtgctgtaaggaaatgcaaagcattggtggccctgcc

SEQ ID NO: 41 - DNA, 50 bp, Homo sapiens
cggtgcctccaaagaacagaaacctgtattcccatggcagggccaccaat

SEQ ID NO: 42 - DNA, 50 bp, Homo sapiens
tctcaaactcggggccagagaacagtgaagtaggagcagccgtaagtccg

SEQ ID NO: 43 - DNA, 50 bp, Homo sapiens
cgccccggcccgctccatctccaaagcatgcagagaatgtctcggcagcc

SEQ ID NO: 44 - DNA, 50 bp, Homo sapiens
gcacacacagcagcagcactgccacccagagcagggctaagatgaacacg

SEQ ID NO: 45 - DNA, 50 bp, Homo sapiens
cgggtattactgccatcgtcgtaccagcagggaaacaaactgagactccg

SEQ ID NO: 46 - DNA, 50 bp, Homo sapiens
cgcggctccatactattgcccagtgttttccgagcatctccatccttggc

SEQ ID NO: 47 - DNA, 50 bp, Homo sapiens
cagtccgtcatctcatctggggaaactgtttgcctttttctgatactacg

SEQ ID NO: 48 - DNA, 50 bp, Homo sapiens
cgtgtgcacatgcgcactctcatatgccaccccatgtgcctttagtccac

SEQ ID NO: 49 - DNA, 50 bp, Homo sapiens
caggagatgccgcggtagggggcggcggtgatgcagcagcggcttggccg

SEQ ID NO: 50 - DNA, 50 bp, Homo sapiens
cgggtgcccaggagcccacaaaacggccagaggagaaatgctttcaaagg

SEQ ID NO: 51 - DNA, 50 bp, Homo sapiens
cggccttctggctggcaagcccctgtgggccaggaagctcagacctaacc

SEQ ID NO: 52 - DNA, 50 bp, Homo sapiens
agtcactgaatcatgagtgacggtaacataaatgcccatcgggggaaacg

SEQ ID NO: 53 - DNA, 50 bp, Homo sapiens
actcgccttgatgttcattggggttcaaaataatcaagttctgcagaacg

SEQ ID NO: 54 - DNA, 50 bp, Homo sapiens
ggccaaggcaggcggctgggaggcgtaggttgtagcgagccgagatcacg

SEQ ID NO: 55 - DNA, 50 bp, Homo sapiens
acgatgttggctcactgcatcctccaccttctgggttcaagcgattctcg

SEQ ID NO: 56 - DNA, 50 bp, Homo sapiens
gtcatccccggggagaggctctcaatggggagtcccatctgcaggctgcg

SEQ ID NO: 57 - DNA, 50 bp, Homo sapiens
cgggttccttttaaagtgctgctagcgcgcactcgccctctcagcgttgc

SEQ ID NO: 58 - DNA, 50 bp, Homo sapiens
caaggccactgcaatttttgggatctagtgagtctgtaggaaatgtaacg

SEQ ID NO: 59 - DNA, 50 bp, Homo sapiens
tgaggtcaggcgtgcccttgcgtagcacaatcgcggggaggggtggctcg

SEQ ID NO: 60 - DNA, 50 bp, Homo sapiens
gcatgtggcatgggtggggtggccagcacgctacaggggcttcctatgcg

SEQ ID NO: 61 - DNA, 50 bp, Homo sapiens
cggaagaggctccagtgtcctcagcagacatgctttatttggttaaagca

SEQ ID NO: 62 - DNA, 50 bp, Homo sapiens
aagcttcagtgaatgtcccccgcttctaagatcctcttctttttgtcacg

SEQ ID NO: 63 - DNA, 50 bp, Homo sapiens
cgccaggctcgcgatgcctctacacaagtatcccgtgtggctctggaagc

SEQ ID NO: 64 - DNA, 50 bp, Homo sapiens
cgggagaaccttctgtgcaggcccggcgctgctccactccaggaaagaaa

SEQ ID NO: 65 - DNA, 50 bp, Homo sapiens
gaggcagggcaggcagacctgcagtcactcctctgcagagtcgcacaccg

SEQ ID NO: 66 - DNA, 50 bp, Homo sapiens
cgcagagaaggaaataacttgttcaaaagaacctactggggtgtgtgtac

SEQ ID NO: 67 - DNA, 50 bp, Homo sapiens
gggagtgggctggcagtgcgcgcacagcggcggcgagtgggtcgtgcacg

SEQ ID NO: 68 - DNA, 50 bp, Homo sapiens
tcgttcctcatgaaaacatcttgagtccaggatctccaattgcactcacg

SEQ ID NO: 69 - DNA, 50 bp, Homo sapiens
ttctctcaaaactcatcacccacttttctttaactgtttgttataagacg

SEQ ID NO: 70 - DNA, 50 bp, Homo sapiens
cggacctagactccaacagtgacagctctgagtaaagaagtcgccgaagc

SEQ ID NO: 71 - DNA, 50 bp, Homo sapiens
gaaggagaagcacccggagccggggggctcctgtgtctactgcaaaagcg

SEQ ID NO: 72 - DNA, 50 bp, Homo sapiens
tggccgggatcccgtgcttggcatacagccggtcttgttctcattgtccg

SEQ ID NO: 73 - DNA, 50 bp, Homo sapiens
ttcatgggtgtggtagcttgtgagaccggccggctgcgcccatgcccccg

SEQ ID NO: 74 - DNA, 50 bp, Homo sapiens
tttaacctttccaggcctcagtttcttcatctgttcagtggaaatgatcg

SEQ ID NO: 75 - DNA, 50 bp, Homo sapiens
tagttttcaccaccctgcctggagcagcacagccaatattggcagactcg

SEQ ID NO: 76 - DNA, 50 bp, Homo sapiens
cgcaagccggccctcacccagtatattgatagggccattgaggggttccg

SEQ ID NO: 77 - DNA, 50 bp, Homo sapiens
cggcctgctaaggaacctcaattaatagctcactgtagccttctgattct

SEQ ID NO: 78 - DNA, 50 bp, Homo sapiens
gacccagaggcactggggtagggctcggcagggcgggtccgggcggagcg

SEQ ID NO: 79 - DNA, 50 bp, Homo sapiens
ctgggctccatgtgtaatttcagggctcatggccacacccagcattgacg

SEQ ID NO: 80 - DNA, 50 bp, Homo sapiens
cgggtaagccccgctgtgttctcatacccgaacggcaggaacacaggcag

SEQ ID NO: 81 - DNA, 50 bp, Homo sapiens
cgcacagagccgcacgggactcagtttccacacacacagagccacacggg

SEQ ID NO: 82 - DNA, 50 bp, Homo sapiens
taagaataacttttgcctcctactgtgattctgaggcctccccaatcacg

SEQ ID NO: 83 - DNA, 50 bp, Homo sapiens
gtggtgacagagccacagagggctgtgagcttgcccggccccaggtaacg

SEQ ID NO: 84 - DNA, 50 bp, Homo sapiens
tggtggtggggctgccggccaatgggctggcgctgtgggtgctggccacg

SEQ ID NO: 85 - DNA, 50 bp, Homo sapiens
cggggcgctgagctcaggcccagaactggctgatttcagggatacccagg

SEQ ID NO: 86 - DNA, 50 bp, Homo sapiens
cggcgcagacaccccgttcggacgatcagagagtgccccaaccctacacc

SEQ ID NO: 87 - DNA, 50 bp, Homo sapiens
gatgaccgggatacagtttgctgcattgcaagggctcacccagtgttccg

SEQ ID NO: 88 - DNA, 50 bp, Homo sapiens
gtcaaaggacaagcccctgggacctgcccaatgctggttcaggggaagcg

SEQ ID NO: 89 - DNA, 50 bp, Homo sapiens
cggggcggaatatacgatttcatgctgatggcccacagaacataacagaa

SEQ ID NO: 90 - DNA, 50 bp, Homo sapiens
cgtgtggacgcgggaggaaaccagagtgcccagagacaacccacgcagac

SEQ ID NO: 91 - DNA, 50 bp, Homo sapiens
ccatgcagggctctagacagcggcgctgcagagcttcctgaggtcggccg

SEQ ID NO: 92 - DNA, 50 bp, Homo sapiens
ctagctttagctgcacattagagtcacctgcagctcttttcaaactgtcg

SEQ ID NO: 93 - DNA, 50 bp, Homo sapiens
ggggggtcaatactcacctggtcgtagacaggagccctggagtcggagcg

SEQ ID NO: 94 - DNA, 50 bp, Homo sapiens
cgtgtctcagtgacgctgattctgcttcacaacaaaagcaaaacaagaac

SEQ ID NO: 95 - DNA, 50 bp, Homo sapiens
cggaaaagggagtggacaggaggggtacagaaccaggctaatgagacaat

SEQ ID NO: 96 - DNA, 50 bp, Homo sapiens
gctggctttcaggatcactatccagcagcagctccgacttccagattccg

SEQ ID NO: 97 - DNA, 50 bp, Homo sapiens
cgctctctggcaaatgtagaggctatttctgctttttacaagaggtgctg

SEQ ID NO: 98 - DNA, 50 bp, Homo sapiens
cggccctccaaaattgcccccacattggctgccttataaatatgtctgtg

SEQ ID NO: 99 - DNA, 50 bp, Homo sapiens
cgggttcagtcattgaaagccttcttgtaattgctgaagggtatttgttt

SEQ ID NO: 100 - DNA, 50 bp, Homo sapiens
cctctttctgctcgccgaaggccgcgatggtgccataggtgagcttttcg

SEQ ID NO: 101 - DNA, 50 bp, Homo sapiens
cgcctcctgagcctgactctgctcttcccgcagctttggccccccggggc

SEQ ID NO: 102 - DNA, 50 bp, Homo sapiens
cgggcacgatgtcccaggaggacagagtttgcattgatgaaaacccttcc

SEQ ID NO: 103 - DNA, 50 bp, Homo sapiens
cgctgggtctgctcccattggctcaggtcatctgagcagctgtgctctgt

SEQ ID NO: 104 - DNA, 50 bp, Homo sapiens
ggtgctctggaccggcagccatgggaatgtcctagtcggcagcagtgtcg

SEQ ID NO: 105 - DNA, 50 bp, Homo sapiens
ctcacgtacagtctcctccctgtacacgaaggagactctgctctaacacg

SEQ ID NO: 106 - DNA, 50 bp, Homo sapiens
cgctgggatcgtggcttatggcgcagagacttccccatgaccaatcattc

SEQ ID NO: 107 - DNA, 50 bp, Homo sapiens
cgggcaccctgaggttgcccccattctttgaacagctatggacggggacc

SEQ ID NO: 108 - DNA, 50 bp, Homo sapiens
cgggcttcccggcagcaggttactctttaacgctcgactgcccacctcca

SEQ ID NO: 109 - DNA, 50 bp, Homo sapiens
cgccctgattggatagtagtgcaggcaccgcgccctcaggccttgcgtgt

SEQ ID NO: 110 - DNA, 50 bp, Homo sapiens
cgcaactgcagtttagcctggtgagttgcaattctggctgcacatgaaaa

SEQ ID NO: 111 - DNA, 50 bp, Homo sapiens
agttttacaagccctcacataaatctacacagctaacatgcacaagtccg

SEQ ID NO: 112 - DNA, 50 bp, Homo sapiens
cgccttgcgcgctcggcatgacgtcactcccaccaggcacctcgccttgt

SEQ ID NO: 113 - DNA, 50 bp, Homo sapiens
aggcgatccttgcgcttgggcagacagcacgtcacggcttttatctgccg

SEQ ID NO: 114 - DNA, 50 bp, Homo sapiens
agtcagacgtcaggacctgctgtggggatggacccacagctcatcccccg

SEQ ID NO: 115 - DNA, 50 bp, Homo sapiens
cccagtactttgggacgccaaggtgggtgggtcacaaggtcaggagatcg

SEQ ID NO: 116 - DNA, 50 bp, Homo sapiens
taccccagctaggccccttccccgcagaggatgtgtttctgttgtcctcg

SEQ ID NO: 117 - DNA, 50 bp, Homo sapiens
gaggtgggcgaggcagggcccacagacattggtgcaggctcagccgtgcg

SEQ ID NO: 118 - DNA, 50 bp, Homo sapiens
cgactggccacacaatggccaggtagcggtcaacactcatgcaagtgagc

SEQ ID NO: 119 - DNA, 50 bp, Homo sapiens
cttcccctcagccttggtttccatatctggaagatgggttaccaatagcg

SEQ ID NO: 120 - DNA, 50 bp, Homo sapiens
atgtcacttgcacaagagccagtcatgccacagggctagccttaatttcg

SEQ ID NO: 121 - DNA, 50 bp, Homo sapiens
aaataacttgtcctttacttaccctgcacatcaaatcttctcgccctgcg

SEQ ID NO: 122 - DNA, 50 bp, Homo sapiens
atttttattttattcctgccattgtttctccttcgagtcaatatcttccg

SEQ ID NO: 123 - DNA, 50 bp, Homo sapiens
gggcgccagccggcgaggagcgacgggagcctggggtgggtggcgaggcg

SEQ ID NO: 124 - DNA, 50 bp, Homo sapiens
cggctgtggtttcctgtgcgtcccaggtgagatcttgctctttacagggt

SEQ ID NO: 125 - DNA, 50 bp, Homo sapiens
aggacgacaaatggcaagatgtgagttgtggccaaggcgcgcgggcagcg

SEQ ID NO: 126 - DNA, 50 bp, Homo sapiens
cgggcttagtaagcccctggagaaaggaatcaaagatgtttctgaggcct

SEQ ID NO: 127 - DNA, 50 bp, Homo sapiens
cgctggccaccctcctaaaggatcactgtggtgccaggcaggaatgagct

SEQ ID NO: 128 - DNA, 50 bp, Homo sapiens
cggtgtgcaaacctgcaatgaattgcccagtgccttcttgtttgtaacgt

SEQ ID NO: 129 - DNA, 50 bp, Homo sapiens
attatagagaatattaagatgcttttggatcctgagcttcccaggaagcg

SEQ ID NO: 130 - DNA, 50 bp, Homo sapiens
cggctgggtctcatctgacacgcagccttccagcctctcattgcccgagg

SEQ ID NO: 131 - DNA, 50 bp, Homo sapiens
agggaagttgggagtattgtgctcccctgagagccgttgccacggttacg

SEQ ID NO: 132 - DNA, 50 bp, Homo sapiens
ccagcagcagcggcagcaaggtttacaatcctaaaccatgcaggcttccg

SEQ ID NO: 133 - DNA, 50 bp, Homo sapiens
tagccatttggcctcggcatcacggtgcgggagcgtggggcgcagactcg

SEQ ID NO: 134 - DNA, 50 bp, Homo sapiens
caagcaagactctccacccacaaactgcatattctttaaagtcactgtcg

SEQ ID NO: 135 - DNA, 50 bp, Homo sapiens
cgcttaaggaccttatagctagcaaatggcagagacaagcttggggcaga

SEQ ID NO: 136 - DNA, 50 bp, Homo sapiens
agatgttatcagctcagcgagactttccatgatctccctaaaatgaatcg

SEQ ID NO: 137 - DNA, 50 bp, Homo sapiens
cgcccagaactgggacttcccaggttctcatagtgattgtagatatcaat

SEQ ID NO: 138 - DNA, 50 bp, Homo sapiens
gctagtgagccgcgcgctgggccggcagcagggggttattttagtgcgcg

SEQ ID NO: 139 - DNA, 50 bp, Homo sapiens
ggctggaggcggctgggggcccggccagtgcgccaggagccgcaggaacg

SEQ ID NO: 140 - DNA, 50 bp, Homo sapiens
ggcaggcaggcggcaggcagagcgcgctctccgggcagtctgaaggaccg

SEQ ID NO: 141 - DNA, 50 bp, Homo sapiens
cgctccgccggtgctcgctctatttgaaaacgctgactgttggtccccca

SEQ ID NO: 142 - DNA, 50 bp, Homo sapiens
acagaaccccagaaaaactgtcccacaacccccagtgaatgaccaaatcg

SEQ ID NO: 143 - DNA, 50 bp, Homo sapiens
cggaagctgcaccctaaattccccgggacctgtggcaggccttcctggtg

SEQ ID NO: 144 - DNA, 50 bp, Homo sapiens
agccacgaagaaaacagttcaactcagaccaaaagataaagctgatctcg

SEQ ID NO: 145 - DNA, 50 bp, Homo sapiens
gcctggtccccgaaagcgctcagtcagaatccgagagcactgtggcggcg

SEQ ID NO: 146 - DNA, 50 bp, Homo sapiens
cgcctctcacagactgcttccccagcagggtgcagcttcttacagactgg

SEQ ID NO: 147 - DNA, 50 bp, Homo sapiens
tgattcagtccagtgattgggtttgtggctccaggcctcgcccacagacg

SEQ ID NO: 148 - DNA, 50 bp, Homo sapiens
cgtctcgccttgcgagcaagctcggaatccagttcctcaggaacccctcc

SEQ ID NO: 149 - DNA, 50 bp, Homo sapiens
cagcaaagaaatgactatagttggaacacaggacttttgcctcctgcccg

SEQ ID NO: 150 - DNA, 50 bp, Homo sapiens
cccatccatgacttcttatgtgtgctgtcggcagctgaccagcggccacg

SEQ ID NO: 151 - DNA, 50 bp, Homo sapiens
aggcagcagcccagtggcctcttgctgtggagcgcgggggcagcctcccg

SEQ ID NO: 152 - DNA, 50 bp, Homo sapiens
cgggtgttgtgtctctgcctatcttcacacatgcacccttgtgaacttgg

SEQ ID NO: 153 - DNA, 50 bp, Homo sapiens
cggactcattgccatgcatgcctagacattaatatgtgttcatggagggc

SEQ ID NO: 154 - DNA, 50 bp, Homo sapiens
tgtgctcagattcacaaagtgaaagtgcaaaaccccgtgttagctcatcg

SEQ ID NO: 155 - DNA, 50 bp, Homo sapiens
cgggctatagcgagaccccccatagtgagatctgctgcgggacctagagt

SEQ ID NO: 156 - DNA, 50 bp, Homo sapiens
cggtggcctgttgaggcccaactcatgcctctggcaccctttcaagaggc

SEQ ID NO: 157 - DNA, 50 bp, Homo sapiens
gcaaggtgaacccaccttgatcccggggctgcagagccagtgcgcctgcg

SEQ ID NO: 158 - DNA, 50 bp, Homo sapiens
cggttggcaagagcttggctagagagactgatgtagatggcagcttctta

SEQ ID NO: 159 - DNA, 50 bp, Homo sapiens
cgagaggcatttcagcagaattttgaaggtggagtgaaggaagtcactat

SEQ ID NO: 160 - DNA, 50 bp, Homo sapiens
aaacactctcccggctcagaaacggtgcaggtgcatttacatgtcaatcg

SEQ ID NO: 161 - DNA, 50 bp, Homo sapiens
cgcgggctctatctgcagcatccaccccctcagcagacgctggcataagc

SEQ ID NO: 162 - DNA, 50 bp, Homo sapiens
ggggccgcagtgtccttggctcaggcctcctacgcagggaagcagggtcg

SEQ ID NO: 163 - DNA, 50 bp, Homo sapiens
cgggaaagtatcacttgtgactgtttgcactaggtgcaaaagacagatag

SEQ ID NO: 164 - DNA, 50 bp, Homo sapiens
tctgtagagaagtcgaccgagaagtgagatgcggtggcgacctcggagcg

SEQ ID NO: 165 - DNA, 50 bp, Homo sapiens
cgctgcccatgcccagaccaaaaatggggtgtctttttagtctaaaagga

SEQ ID NO: 166 - DNA, 50 bp, Homo sapiens
cggccagacccgtcactcagatccccgcaccaggccagtgaagaggagcc

SEQ ID NO: 167 - DNA, 50 bp, Homo sapiens
ccagcccttctggcacagatgctgtgcatgtgaggtgcttctcagaagcg

SEQ ID NO: 168 - DNA, 50 bp, Homo sapiens
aggagatgagctatgtcttccaacctctccttttggaataaaacctttcg

SEQ ID NO: 169 - DNA, 50 bp, Homo sapiens
cgacgacaaagaagattttgatgaagttcaaattagagtcagcgtgctca

SEQ ID NO: 170 - DNA, 50 bp, Homo sapiens
gtaaagtgacacccatcccactcatcccattgctgccaatgacttcaacg

SEQ ID NO: 171 - DNA, 50 bp, Homo sapiens
caccttccactttaaagaacaccaaggtcggctgggtgtggtggctcacg

SEQ ID NO: 172 - DNA, 50 bp, Homo sapiens
cgagcccaggaaataagtgcgggtgaccgaattcaatcctgcatccaaca

SEQ ID NO: 173 - DNA, 50 bp, Homo sapiens
cggccccacttagcctgaaacggggccccatgcagggtagaggcctgcca

SEQ ID NO: 174 - DNA, 50 bp, Homo sapiens
cgcacaccgagtggcacgacctgcagaggttaccgccaggtttccatcct

SEQ ID NO: 175 - DNA, 50 bp, Homo sapiens
tggggtgagccactttcattttcccagcggggccaggcagtctttgctcg

SEQ ID NO: 176 - DNA, 50 bp, Homo sapiens
cgcaggtctgagagacatcacagcatcacagcgtttagtagtcttaaatg

SEQ ID NO: 177 - DNA, 50 bp, Homo sapiens
cgactaccactgctggtgcaaggaccaatcagcgacctattgaagtgcct

SEQ ID NO: 178 - DNA, 50 bp, Homo sapiens
cgggatggtgtgacaggcaagccaaagttgtatctgaaaactctgcaaca

SEQ ID NO: 179 - DNA, 50 bp, Homo sapiens
ggtccgctggggcagagtgcggagtgaaggggtgcactgggcactcagcg

SEQ ID NO: 180 - DNA, 50 bp, Homo sapiens
ctttaagatgtctgacagtgccctaagggcacgtgctccacaaacaaacg

SEQ ID NO: 181 - DNA, 50 bp, Homo sapiens
ctcaaagaaaataagccaggaccaggtgacccagtgggatccgcgaagcg

SEQ ID NO: 182 - DNA, 50 bp, Homo sapiens
tgcggccactgcaaagcgatgtaaatgtcccagagtggggcagctggtcg

SEQ ID NO: 183 - DNA, 50 bp, Homo sapiens
cgtgccacctggccaacctcgaaaggaggccctgctgggttcttcatctt

SEQ ID NO: 184 - DNA, 50 bp, Homo sapiens
cggcggacccagctgaccaaaggaactacagaagaggacgaagaaagcga

SEQ ID NO: 185 - DNA, 50 bp, Homo sapiens
tcgctttccagccttttgtttctggatcctcaaaactcagaacgtggccg

SEQ ID NO: 186 - DNA, 50 bp, Homo sapiens
tggagcaaatataggtctagacacgtagggcctgtgaggttttcctgacg

SEQ ID NO: 187 - DNA, 50 bp, Homo sapiens
gcgtgtgctcagcgtgtggttaccgtgtgcacagcacgagcttgaactcg

SEQ ID NO: 188 - DNA, 50 bp, Homo sapiens
cgtggccactcttcagcatctctgtcggaagaccgtcaacggccacctgg

SEQ ID NO: 189 - DNA, 50 bp, Homo sapiens
cgggaatggtgactcagccttccaggaacctgcgtggcgtctgttttttt

SEQ ID NO: 190 - DNA, 50 bp, Homo sapiens
cgggcgcagccccaggatgcaacagctctcatacaattggccaggaaggc

SEQ ID NO: 191 - DNA, 50 bp, Homo sapiens
cgggtttcccaagtgctggacgcgcaacaggtttccataaacccaaagtt

SEQ ID NO: 192 - DNA, 50 bp, Homo sapiens
cggcgagtgagtgaaaaattacattttcacttgggttctgaacattcagt

SEQ ID NO: 193 - DNA, 50 bp, Homo sapiens
cggggaaactctgaattggtggttgcttagaaaacacatagaagtgtgta

SEQ ID NO: 194 - DNA, 50 bp, Homo sapiens
tcagggttgaaaacgcaacagccaaaattaactcagtggatggctcaacg

SEQ ID NO: 195 - DNA, 50 bp, Homo sapiens
cgccggtggccgacggcttctgaggaattatcttttacttggcgccacac

SEQ ID NO: 196 - DNA, 50 bp, Homo sapiens
cgagattaaagattcagaaagtgagtaaagttaacactgagcccttgggc

SEQ ID NO: 197 - DNA, 50 bp, Homo sapiens
agttggtgagccagggaagtcttccctcgccctgagggtgcggggaagcg

SEQ ID NO: 198 - DNA, 50 bp, Homo sapiens
cggggccaaagaggagcgcaggtttaaaaagcaagtgtattagcaagttt

SEQ ID NO: 199 - DNA, 50 bp, Homo sapiens
cgcccacgcggacccctctccgtgtgcttgtctgtacttccctgatgggg

SEQ ID NO: 200 - DNA, 50 bp, Homo sapiens
cggccggagggccctgaaccctaggccaagactcatccacaacctagggg

SEQ ID NO: 201 - DNA, 50 bp, Homo sapiens
cgggagatcctcccgcggccggagggccctgaaccctaggccaagactca

SEQ ID NO: 202 - DNA, 50 bp, Homo sapiens
cgtgagcgggatcctgagtctgagttgccgcttaacccggcattttataa

SEQ ID NO: 203 - DNA, 50 bp, Homo sapiens
cggaggcaagcaagctccatgtggtggggacagcgcattgcgacctccac

SEQ ID NO: 204 - DNA, 50 bp, Homo sapiens
ccatgctataagtgaggtaaataaatatgctgagcaccatcgcacttacg

SEQ ID NO: 205 - DNA, 50 bp, Homo sapiens
cgcggcattcagcagaactcatgatgaaggaaacagttccatggcagggt

SEQ ID NO: 206 - DNA, 50 bp, Homo sapiens
cgacaaaatctgaccaggctagatctgaatctgttaatttctgagacggt

SEQ ID NO: 207 - DNA, 50 bp, Homo sapiens
cgcagaggagaatgtcagatgctcagctcggtcccctccgcctgacgctc

SEQ ID NO: 208 - DNA, 50 bp, Homo sapiens
cgcccgattcagacagctggactcagagggattctgctccacagagaaac

SEQ ID NO: 209 - DNA, 50 bp, Homo sapiens
cgtatgtgtttgagatagcagttgtttactatcacttgaaaattctgaat

SEQ ID NO: 210 - DNA, 50 bp, Homo sapiens
tcatgaactggagcctgaagtggctctgaggcctcccctggaaaggaacg

SEQ ID NO: 211 - DNA, 50 bp, Homo sapiens
cggtggattcctctcgggacacatctgccacccgctggactggcaaaaca

SEQ ID NO: 212 - DNA, 50 bp, Homo sapiens
cgaccatttgtatgtgtatctatgtcagaaagaatctttattaaaatatt

SEQ ID NO: 213 - DNA, 50 bp, Homo sapiens
taccatgtaatataccactaaacacacactatgtaatacactactaaacg

SEQ ID NO: 214 - DNA, 50 bp, Homo sapiens
ttgtctcagattacaggaaccaaatgcatgtctggccttgtgctgcttcg

SEQ ID NO: 215 - DNA, 50 bp, Homo sapiens
cgagcccgcccagcctctgaacctccgtgtcttcatctgtaaattgaggg

SEQ ID NO: 216 - DNA, 50 bp, Homo sapiens
gtgctctcagtccaattcacactgaatgaatgaatcagtcccagccatcg

SEQ ID NO: 217 - DNA, 50 bp, Homo sapiens
cgctgaggtcccgtttctgtagctgagtagcccggtgacactgccctccg

SEQ ID NO: 218 - DNA, 50 bp, Homo sapiens; identical to SEQ ID NO: 6

SEQ ID NO: 219 - DNA, 50 bp, Homo sapiens
cgccgcagtcccagtcgagccgcgacccttccggctgcccccaccccacc

SEQ ID NO: 220 - DNA, 50 bp, Homo sapiens
tgtgccttgacgggctgtcccaggcgcccctggaattgatgcagtgtccg

SEQ ID NO: 221 - DNA, 50 bp, Homo sapiens
cgcagaagaaagctgctcaagtgctggagtggccaaccccatgccgttcc

SEQ ID NO: 222 - DNA, 50 bp, Homo sapiens
cgcacatcctcagctagcagcatggagctgagttcagaggaggcacttag

SEQ ID NO: 223 - DNA, 50 bp, Homo sapiens
cgggagggaaaggtgttagatggtggttttaactcaccagggtttgaatt

SEQ ID NO: 224 - DNA, 50 bp, Homo sapiens
cggcctgattcgaagactggaatagaggatttctagaattctgtgactcc

SEQ ID NO: 225 - DNA, 50 bp, Homo sapiens
catgtcccagctcagctggtcctgagaagctgcctggtagacagtcaccg

SEQ ID NO: 226 - DNA, 50 bp, Homo sapiens
tagaaccaactggatactgcacccctcagtattggggcgtcacagcaccg

SEQ ID NO: 227 - DNA, 50 bp, Homo sapiens; identical to SEQ ID NO: 176

SEQ ID NO: 228 - DNA, 50 bp, Homo sapiens
caagtcctttctggttcccgtggtttttcaggtactgcggttcttattcg

SEQ ID NO: 229 - DNA, 50 bp, Homo sapiens
ctccctacgtgggggacgtgcaggatgatgcggggtcgggggggatttcg

SEQ ID NO: 230 - DNA, 50 bp, Homo sapiens
tccgcaaaaagcaggatccattcactttagagaagtgccctcaacaggcg

SEQ ID NO: 231 - DNA, 50 bp, Homo sapiens
tgtggtctccccagaccaccaggtgcttcacacacgcacaggggcactcg

SEQ ID NO: 232 - DNA, 50 bp, Homo sapiens
tgcagcctcgtaagcctcatggaagagcttggtttgtatcctttggaacg

SEQ ID NO: 233 - DNA, 50 bp, Homo sapiens
cagtgctgagccggatccagaaaattgaacaggtcctgaaggagcagccg

SEQ ID NO: 234 - DNA, 50 bp, Homo sapiens
cgtccccgaaatgaataatgcaggcaggagccaagatttctgcattagcg

SEQ ID NO: 235 - DNA, 50 bp, Homo sapiens
cggtgtggaggctttttttgtcagaagctctggggtttggctcttctctg

SEQ ID NO: 236 - DNA, 50 bp, Homo sapiens
cctctttgctgatctggactcagcctggtattcttgtcacaaggtcatcg

SEQ ID NO: 237 - DNA, 50 bp, Homo sapiens
tttttaaccagtttgaacattttactacttcctaacatcagaagcacccg

SEQ ID NO: 238 - DNA, 50 bp, Homo sapiens
gtgtggcaaggctgagctagttcgtctgccaggtaccagagcaagcctcg

SEQ ID NO: 239 - DNA, 50 bp, Homo sapiens
cactatgtttgctgcttgctattcacttatggtacatacacagtcagtcg

SEQ ID NO: 240 - DNA, 50 bp, Homo sapiens
gggacggtggtagtgcttctcaacattggcactggctccattgtcattcg

SEQ ID NO: 241 - DNA, 50 bp, Homo sapiens
tggggagccagctgacttaacaggcctggattacattcttgcttccaacg

SEQ ID NO: 242 - DNA, 50 bp, Homo sapiens
tgccatttaatctaacgccaccttctacggaccagagcaggtgctctgcg

SEQ ID NO: 243 - DNA, 50 bp, Homo sapiens
cggttaaattcgtgaggctgtttgctgtgtacacaatgctaagcagaaaa

SEQ ID NO: 244 - DNA, 50 bp, Homo sapiens; identical to SEQ ID NO: 182

SEQ ID NO: 245 - DNA, 50 bp, Homo sapiens
tctggtcctggcctgggggggtcgtggtctcgctggtgcagcctcctccg

SEQ ID NO: 246 - DNA, 50 bp, Homo sapiens
cgccaccatcgagtgtgagcagccccagcccgacctctacaagtaagcgg

SEQ ID NO: 247 - DNA, 50 bp, Homo sapiens
cgctgtgaaacctttgcctttgggtgtcatggtggaagcaaatcttagaa

SEQ ID NO: 248 - DNA, 50 bp, Homo sapiens
cgccagggcaaacattccttgggcgcctcgggcttttccctagggaagca

SEQ ID NO: 249 - DNA, 50 bp, Homo sapiens; identical to SEQ ID NO: 185

SEQ ID NO: 250 - DNA, 50 bp, Homo sapiens
cggagagacagccgctgtggttaggggtgtgcatccttctaccatgccca

SEQ ID NO: 251 - DNA, 50 bp, Homo sapiens
cgtggatctagccacagaaaggcaacagcgcttcaaagaggctctccgta

SEQ ID NO: 252 - DNA, 50 bp, Homo sapiens
cgcttttgcaaaacaggccgcacctagcccagattctggagctgattcag

SEQ ID NO: 253 - DNA, 50 bp, Homo sapiens
cgactctgagttctgacagggaaatacaatgagttttatgtggacagagt

SEQ ID NO: 254 - DNA, 50 bp, Homo sapiens
ctctctcccagacctcaggtaaaattaatgttctaggaacgtatgccacg

SEQ ID NO: 255 - DNA, 50 bp, Homo sapiens
tggggtggccgtctgctccgtctttggtggtgagaacccgcaccggtccg

SEQ ID NO: 256 - DNA, 50 bp, Homo sapiens
cggtgtctccgctccttgcccgtggccaggtggtggtgctcccaatgccc

SEQ ID NO: 257 - DNA, 50 bp, Homo sapiens
cgggctttaaggtggcccttgcagatcttgcagacattttcgtgggctcc

SEQ ID NO: 258 - DNA, 50 bp, Homo sapiens
agactccctctgaacacctgtggaacatgctgacctcccagtgtctttcg

SEQ ID NO: 259 - DNA, 50 bp, Homo sapiens
cgcacatgtgaggccaccattcataccacaccctgggtgggtcttgagtt

SEQ ID NO: 260 - DNA, 50 bp, Homo sapiens
tctgctcattaacaccctgttaataggaaccccagaaacctgagcttccg

SEQ ID NO: 261 - DNA, 50 bp, Homo sapiens
cggccttgcacaccatcttaagtgtgacgccacactgcccttcacagcct

SEQ ID NO: 262 - DNA, 50 bp, Homo sapiens; identical to SEQ ID NO: 188

SEQ ID NO: 263 - DNA, 50 bp, Homo sapiens
cgcgcctggtgcagccctcacctcaccaggtgttgatcatcttagtggtt

SEQ ID NO: 264 - DNA, 50 bp, Homo sapiens
cgcacttaatatgattattttttgatgccaggtccaccaagtgaagattg

SEQ ID NO: 265 - DNA, 50 bp, Homo sapiens
cgtgtaaaggtcagaggtccattcaccaatgcaatgatcctcactgcccc

SEQ ID NO: 266 - DNA, 50 bp, Homo sapiens; identical to SEQ ID NO: 189

SEQ ID NO: 267 - DNA, 50 bp, Homo sapiens
tattgcagtatcctaaagcgtgctgtatctctctgtccggcttttgcacg

SEQ ID NO: 268 - DNA, 50 bp, Homo sapiens
cggtcttgtaacaattggatggatgcctttgaagagcccctgtccctatt

SEQ ID NO: 269 - DNA, 50 bp, Homo sapiens
tctcctcccctgcactggctctgggtgcttatctctgcagaagcttctcg

SEQ ID NO: 270 - DNA, 50 bp, Homo sapiens
cggggtctagggaagagagatttcctaacctagactacaggcatcaggag

SEQ ID NO: 271 - DNA, 50 bp, Homo sapiens
tgattcctccccctttgaaagagcaggcagcatttttccaaggcaacccg

SEQ ID NO: 272 - DNA, 50 bp, Homo sapiens
cgctggtctcgggggggaacccaacagactcacatttcatgtgaaaggaa

SEQ ID NO: 273 - DNA, 50 bp, Homo sapiens
aaagatgtcattacacaaatacacaaatactagcatgcagacacacaacg

SEQ ID NO: 274 - DNA, 50 bp, Homo sapiens
gatgtatctcccacccaggagtgtttcttggccaggtgtggtggctcacg

SEQ ID NO: 275 - DNA, 50 bp, Homo sapiens
cgacaactctgtagtgatttagaagagaattgcaagacattaacacagag

SEQ ID NO: 276 - DNA, 50 bp, Homo sapiens; identical to SEQ ID NO: 193

SEQ ID NO: 277 - DNA, 50 bp, Homo sapiens
gtttcagcacctgggtcagcgcttcccagggtcagcaccagggatagacg

SEQ ID NO: 278 - DNA, 50 bp, Homo sapiens
cgcagctgtcgtctttgtacccggctttgcgcactccattgtggacgcga

SEQ ID NO: 279 - DNA, 50 bp, Homo sapiens; identical to SEQ ID NO: 124

SEQ ID NO: 280 - DNA, 50 bp, Homo sapiens
gcacctttgtgaggtgtttgtggggttgggggagcttcaggcgctactcg

SEQ ID NO: 281 - DNA, 50 bp, Homo sapiens
cggtacttccctgatggggcccacgcggacccctctccagtgtgcttgtc

SEQ ID NO: 282 - DNA, 50 bp, Homo sapiens
atcttcccagtacagagcttagcatacattgaataactggcatctgctcg

SEQ ID NO: 283 - DNA, 50 bp, Homo sapiens
actctgtcagatatcctcgatatttggggatagtagttcaaaaacagccg

SEQ ID NO: 284 - DNA, 50 bp, Homo sapiens
agctgtttccctcaggcggcctgttccacgccaggattagttttgcttcg

SEQ ID NO: 285 - DNA, 50 bp, Homo sapiens
cggtttgcaggttcaaagatgtctaaaagagaatggccagaatgtatata

SEQ ID NO: 286 - DNA, 50 bp, Homo sapiens
atccctcatctggactttgagtgattgtgttgctggcatggaccctttcg

SEQ ID NO: 287 - DNA, 50 bp, Homo sapiens
cagagcccacttagtgcgcgctagctgggcagggataggggtcctattcg

SEQ ID NO: 288 - DNA, 50 bp, Homo sapiens
acagtatttttgtcactccaagtatcccttatcttgcccttttatggtcg

SEQ ID NO: 289 - DNA, 50 bp, Homo sapiens
ctgaaaggcgcattgtcagcctggggaggcgacgcggcgcgaggacagcg

SEQ ID NO: 290 - DNA, 50 bp, Homo sapiens
cggagggcttattctatgtaaatagttgaaaggaactggataagactgta

SEQ ID NO: 291 - DNA, 50 bp, Homo sapiens; identical to SEQ ID NO: 205

SEQ ID NO: 292 - DNA, 50 bp, Homo sapiens
cgcccagctggaggctgagatgtcaggatttgtcttgcaaagttacttca

SEQ ID NO: 293 - DNA, 50 bp, Homo sapiens
cgcaaaacttaaggctatagaagatggggagagtatcttttgatgcagac

SEQ ID NO: 294 - DNA, 50 bp, Homo sapiens; identical to SEQ ID NO: 207

SEQ ID NO: 295 - DNA, 50 bp, Homo sapiens; identical to SEQ ID NO: 208

SEQ ID NO: 296 - DNA, 50 bp, Homo sapiens
cgctgcactcagccacgttacgcggagttcatctggggaaggagacaaat

SEQ ID NO: 297 - DNA, 50 bp, Homo sapiens
gaagggtcacacatgtcccagaatcccccaggagtgatgcaaatggagcg

SEQ ID NO: 298 - DNA, 50 bp, Homo sapiens
aaaccctaaacactgaagtctcctctggtcaggtgacccctatcagtccg

SEQ ID NO: 299 - DNA, 50 bp, Homo sapiens
gccaggagtcctcggtcctgggagggtttaaaagccagggggccgtctcg

SEQ ID NO: 300 - DNA, 50 bp, Homo sapiens
gaccctgggcttggggggatttttttggcctggagggctcctcttctccg

SEQ ID NO: 301 - DNA, 50 bp, Homo sapiens
ggccagacacccaggacccaaataacctaataatggctcatttgcctccg

SEQ ID NO: 302 - DNA, 50 bp, Homo sapiens
cggcattggtcctttattgaacatcctcccaaagctggggctagggttca

SEQ ID NO: 303 - DNA, 50 bp, Homo sapiens
ccagatgttttcactcgattttgcaaatctgctccaggtctaagacatcg

SEQ ID NO: 304 - DNA, 50 bp, Homo sapiens
cgggtcttcactctggttggctgccagatcctccaggggcggggtctcac

SEQ ID NO: 305 - DNA, 50 bp, Homo sapiens
tacagaacccctgaaacattcctgccaaatacagtcgcacgaatttgtcg

SEQ ID NO: 306 - DNA, 50 bp, Homo sapiens; identical to SEQ ID NO: 173

SEQ ID NO: 307 - DNA, 50 bp, Homo sapiens
cgcacagcaactggggtgtcctgaccagtgagctagagttccaccttact

SEQ ID NO: 308 - DNA, 50 bp, Homo sapiens
ttataatccaaaaatatttattaaccatctgctctgtgacaagcactgcg

SEQ ID NO: 309 - DNA, 50 bp, Homo sapiens
ctttggctcgagctccaggtgcagatttacgttcattttcttttgttccg

SEQ ID NO: 310 - DNA, 50 bp, Homo sapiens
aaaaacttttctgagccggagcctccagcgcgctgtgtattcgttttacg

SEQ ID NO: 311 - DNA, 50 bp, Homo sapiens; identical to SEQ ID NO: 221

SEQ ID NO: 312 - DNA, 50 bp, Homo sapiens
cgcagattccccatttgtgagcagagttaagctgaaggagcgtgggcatg

SEQ ID NO: 313 - DNA, 50 bp, Homo sapiens
cggccccagaaggcaaggcgattgttaggaggaattaaacaggctagtgc

SEQ ID NO: 314 - DNA, 50 bp, Homo sapiens
cgcccggcgaccactggtggttctatgctgctggggtgaaaagtctgaag

SEQ ID NO: 315 - DNA, 50 bp, Homo sapiens
aggacactggccagcctccagttggtttctgaagctagcagtcttagtcg

SEQ ID NO: 316 - DNA, 50 bp, Homo sapiens
cgccctgccctgggattctcagaccttgaaagccacaagtcttgaaagtc

SEQ ID NO: 317 - DNA, 50 bp, Homo sapiens; identical to SEQ ID NO: 176

SEQ ID NO: 318 - DNA, 50 bp, Homo sapiens; identical to SEQ ID NO: 229

SEQ ID NO: 319 - DNA, 50 bp, Homo sapiens
ctgaattctggtacctgttaatgacagcttctgagaggtttcttttatcg

SEQ ID NO: 320 - DNA, 50 bp, Homo sapiens
taaccttgaacaaagatcgtcgacgggcggccctcactggaaacctttcg

SEQ ID NO: 321 - DNA, 50 bp, Homo sapiens; identical to SEQ ID NO: 234

SEQ ID NO: 322 - DNA, 50 bp, Homo sapiens
cgagttgtaagtcaacctgctgggttgtgctgaagagaaattggagactg

SEQ ID NO: 323 - DNA, 50 bp, Homo sapiens
ttagggcctggagatgggcagacccaggctcacatcctagctctgacacg

SEQ ID NO: 324 - DNA, 50 bp, Homo sapiens
cggctctaagcggggcaagagctgcttttaatttgagacgtacacagttt

SEQ ID NO: 325 - DNA, 50 bp, Homo sapiens; identical to SEQ ID NO: 237

SEQ ID NO: 326 - DNA, 50 bp, Homo sapiens
acacataccccactaaagaagattcacaaggttgggtgcagtggttcacg

SEQ ID NO: 327 - DNA, 50 bp, Homo sapiens
actatgtacctggaaatagcaggggctcagggtttgatcctatgagaccg

SEQ ID NO: 328 - DNA, 50 bp, Homo sapiens
cggctacgagcacagagcatcatcatcatcagcctgccagccggccccgc

SEQ ID NO: 329 - DNA, 50 bp, Homo sapiens; identical to SEQ ID NO: 251

SEQ ID NO: 330 - DNA, 50 bp, Homo sapiens
cctctggacagtacttttgagacacacttatttctaccacgcaccttgcg

SEQ ID NO: 331 - DNA, 50 bp, Homo sapiens
cgagaagttcaagttcaagctgcagaacaccagtgtgcagaaggagcact

SEQ ID NO: 332 - DNA, 50 bp, Homo sapiens
cgcgcaggaaaagggaaacaaacagcagcttcgcagcctccctgggcctg

SEQ ID NO: 333 - DNA, 50 bp, Homo sapiens
ctatggtcaactgagtcagggctggatttctcacccaccctggccaatcg

SEQ ID NO: 334 - DNA, 50 bp, Homo sapiens
cgctcctattcaccgaacccctggctggctgacccgggaaaaacagtcta

SEQ ID NO: 335 - DNA, 50 bp, Homo sapiens
tccaccaggtctcttatcaatggacagtacaaacagtactaccacgaacg

SEQ ID NO: 336 - DNA, 50 bp, Homo sapiens
cgcgtgtttctctgcgctgtggtcgtcatggaccggaagcgtgtgcagcg

SEQ ID NO: 337 - DNA, 50 bp, Homo sapiens
ttttcttctttggcttttccatcactgctggctacaagtgtttgcaaccg

SEQ ID NO: 338 - DNA, 50 bp, Homo sapiens
ggggttgccggcaccttctctttctccccaggtcctttcatctgattccg

SEQ ID NO: 339 - DNA, 50 bp, Homo sapiens; identical to SEQ ID NO: 188

SEQ ID NO: 340 - DNA, 50 bp, Homo sapiens
cgcacggcctccagcagcaagctgatgctgtcctacaagaagccaaaagg

SEQ ID NO: 341 - DNA, 50 bp, Homo sapiens; identical to SEQ ID NO: 265

SEQ ID NO: 342 - DNA, 50 bp, Homo sapiens
gtcctaaagcaccaggatagatgacgagcagggccaagtccacacaagcg

SEQ ID NO: 343 - DNA, 50 bp, Homo sapiens
tttaagctatcaatgtggatgtgctggtgctgagacccacctccaacacg

SEQ ID NO: 344 - DNA, 50 bp, Homo sapiens; identical to SEQ ID NO: 189

SEQ ID NO: 345 - DNA, 50 bp, Homo sapiens
ctcctccgagtagccggcgccgtggatgatgcgcatctgcttgatgaacg

SEQ ID NO: 346 - DNA, 50 bp, Homo sapiens
actggaccctcggctcttccttggacttcttgtgtgttctgtgagcttcg

SEQ ID NO: 347 - DNA, 50 bp, Homo sapiens
tggggctttaaaaaccacagcccttgggcaggagggaccttcgatcctcg

SEQ ID NO: 348 - DNA, 50 bp, Homo sapiens
cgctccctcttaggtcacctgccatttcagacagggaagagaacagtgaa

SEQ ID NO: 349 - DNA, 50 bp, Homo sapiens
agcagctccaccggggatgcggctcgtcagggggtgtacgagaacttccg

SEQ ID NO: 350 - DNA, 50 bp, Homo sapiens; identical to SEQ ID NO: 274

SEQ ID NO: 351 - DNA, 50 bp, Homo sapiens
cgccacagagggcttccccaggtccaggccgggccagccttgacctctaa

SEQ ID NO: 352 - DNA, 50 bp, Homo sapiens
cgaacctgatgtggagacaaagtatccttcatggactttgaatttgatat

SEQ ID NO: 353 - DNA, 50 bp, Homo sapiens
aagggtagctgctgccactgctacattctacagaaagctcaatgagtccg

SEQ ID NO: 354 - DNA, 50 bp, Homo sapiens
cggctccctcacacccactgagagcatcatgtccctgggcacgcactccc

SEQ ID NO: 355 - DNA, 50 bp, Homo sapiens; identical to SEQ ID NO: 193

SEQ ID NO: 356 - DNA, 50 bp, Homo sapiens
cgctgcttcaactggagttaaccaagtgaacacagagctttacaaagaac

SEQ ID NO: 357 - DNA, 50 bp, Homo sapiens
ctcggtctcgtgtgtctgtgtgtgccagtcccttgtgctgacacaggacg

SEQ ID NO: 358 - DNA, 50 bp, Homo sapiens
tatgcagttgtaattctgacagtattgccaaattgttcacagtaaagacg

SEQ ID NO: 359 - DNA, 50 bp, Homo sapiens
cggcagagaggcctgagataaggtttttgcacatggctggtaagactgga

SEQ ID NO: 360 - DNA, 50 bp, Homo sapiens
cggttgtcagcaccatatttctaaaatgcaaatctggcaagtcattttct

SEQ ID NO: 361 - DNA, 50 bp, Homo sapiens
cgcggctgtaccacatagagaaagatatgcactagttcaaagagaatgcc

SEQ ID NO: 362 - DNA, 50 bp, Homo sapiens; identical to SEQ ID NO: 196

SEQ ID NO: 363 - DNA, 50 bp, Homo sapiens
aggccccaaggttcctgacagagctgctgttgggtgttggtgtaaagccg

SEQ ID NO: 364 - DNA, 50 bp, Homo sapiens
aaaaagactgcagctcccatcttaattgctctctcatgcttgtgtgtgcg

SEQ ID NO: 365 - DNA, 50 bp, Homo sapiens
gaattctgctggcgctgcagctgcagaatggtcggcggtggcgggaagcg

SEQ ID NO: 366 - DNA, 50 bp, Homo sapiens
agcttcagacacagtcaacgtgctgccaacgtggggagggccggaggacg

SEQ ID NO: 367 - DNA, 50 bp, Homo sapiens
cggagatgaaattaatgtgaaccactgaggcaaaggcatgaggccccctc

SEQ ID NO: 368 - DNA, 50 bp, Homo sapiens
cggctgccccctcaccagcaccctccgattccagcgacagcctcttggct

SEQ ID NO: 369 - DNA, 50 bp, Homo sapiens
gtccctggagccaggcgttaatcattgacctgattagcaagaacgctccg

SEQ ID NO: 370 - DNA, 50 bp, Homo sapiens
cgacatgacaagatttaagggaggcacatctcacagacaagcatgaaaac

SEQ ID NO: 371 - DNA, 50 bp, Homo sapiens
ctggcccggccgcccctcagctatttgttcacgtaatgcgattggaaacg

SEQ ID NO: 372 - DNA, 50 bp, Homo sapiens
taaatggacttactttgaggccatgatcaacatttaaatcttgctcttcg

SEQ ID NO: 373 - DNA, 50 bp, Homo sapiens; identical to SEQ ID NO: 287

SEQ ID NO: 374 - DNA, 50 bp, Homo sapiens
cggacacttccggtggaaggactgagcggcgctacacttcaagaattccg

SEQ ID NO: 375 - DNA, 50 bp, Homo sapiens
ttattgttttcctgtcttctacagcagaattggatattcccaaacaatcg

SEQ ID NO: 376 - DNA, 50 bp, Homo sapiens
cggggtctgggttttcttgtcccactgggagtttcaagccccaggtagaa

SEQ ID NO: 377 - DNA, 50 bp, Homo sapiens
tcaagggagggcgatattccagtgctgatcccattttcctcccctcctcg

SEQ ID NO: 378 - DNA, 50 bp, Homo sapiens
tcaggaaaactcatgccattctccattcaacggagggcgacattctagcg

SEQ ID NO: 379 - DNA, 50 bp, Homo sapiens
cgtccagcagacaaagactatggtttaatgaaaatagatgaaccaagcac

SEQ ID NO: 380 - DNA, 50 bp, Homo sapiens
actgatgatttccaggaactaccaaagccacggatcagctgagttacccg

SEQ ID NO: 381 - DNA, 50 bp, Homo sapiens; identical to SEQ ID NO: 289

SEQ ID NO: 382 - DNA, 50 bp, Homo sapiens
aacaacaaaaaacttaaatttcccttgacttttaagttcactcttgttcg

SEQ ID NO: 383 - DNA, 50 bp, Homo sapiens; identical to SEQ ID NO: 207

SEQ ID NO: 384 - DNA, 50 bp, Homo sapiens; identical to SEQ ID NO: 208

SEQ ID NO: 385 - DNA, 50 bp, Homo sapiens
tcccaccccttccattctgttgagctgagacgcgtgttttggtcgtttcg

SEQ ID NO: 386 - DNA, 50 bp, Homo sapiens
cctgatttacacttggctccacccataaagttcccactgattatcacacg

SEQ ID NO: 387 - DNA, 50 bp, Homo sapiens; identical to SEQ ID NO: 299

SEQ ID NO: 388 - DNA, 50 bp, Homo sapiens; identical to SEQ ID NO: 209

SEQ ID NO: 389 - DNA, 50 bp, Homo sapiens; identical to SEQ ID NO: 212

SEQ ID NO: 390 - DNA, 50 bp, Homo sapiens
cggtcacgtgataccccccagtgtacctttcctgcagcgtgttacgtggc

SEQ ID NO: 391 - DNA, 50 bp, Homo sapiens
ctggggctgagaccccatttctgggaggctcttcccagtgctggagaccg

SEQ ID NO: 392 - DNA, 50 bp, Homo sapiens
tctttgagccccagcttcctcatgtgtaaaacaggataatagcagtgtcg

SEQ ID NO: 393 - DNA, 50 bp, Homo sapiens
ctgctggggagcagtggacgccctccagggtctagggagcagataagacg

SEQ ID NO: 394 - DNA, 50 bp, Homo sapiens
gcacccccccatccacttcatgttcagaaaactcaaagagtcagaaaacg

SEQ ID NO: 395 - DNA, 50 bp, Homo sapiens
agctctggaatccaactgcctgagtggcaaacctatctgtaagacttacg

SEQ ID NO: 396 - DNA, 50 bp, Homo sapiens
gggcctttagtgctggctcttgagtacccagaccccagttaagaaatacg

SEQ ID NO: 397 - DNA, 50 bp, Homo sapiens
cgacaccctcaagggaggagtgcaggcactcaaagatttgagtcacaggc

SEQ ID NO: 398 - DNA, 50 bp, Homo sapiens; identical to SEQ ID NO: 221

SEQ ID NO: 399 - DNA, 50 bp, Homo sapiens
tggccttaggttctagtggctctggtgtgggcctgtgcccagcctttacg

SEQ ID NO: 400 - DNA, 50 bp, Homo sapiens
tttataaacctgctattcctttccttcggatccctggttttccatctccg

SEQ ID NO: 401 - DNA, 50 bp, Homo sapiens
ctgggcttgacagctcagagcagtgtccccagctgtggccctagaaggcg

SEQ ID NO: 402 - DNA, 50 bp, Homo sapiens
tttgcctctttttcagtcttgtaatcccaaacaaaatagaagagccatcg

SEQ ID NO: 403 - DNA, 50 bp, Homo sapiens
agaagcacgtggcacctgtcataggcccctctggctgctacaagttcccg

SEQ ID NO: 404 - DNA, 50 bp, Homo sapiens
cggcacctatcccagcaagatcccgggaacaaaatcctggaagaccctat

SEQ ID NO: 405 - DNA, 50 bp, Homo sapiens
cgcgtgagtgtcctaaggagtccaagcacaacagatggctgagtcacact

SEQ ID NO: 406 - DNA, 50 bp, Homo sapiens
cgagcctgttgtcaacattagccccgggtttcccagcaccaactccagcg

SEQ ID NO: 407 - DNA, 50 bp, Homo sapiens
cggagcgtgagcctagattctttggaaggctgtacagtttaaaatctaat

SEQ ID NO: 408 - DNA, 50 bp, Homo sapiens
cggcaggttggaaatccagtttgtggctgatgcaagcaaaccatgctgca

SEQ ID NO: 409 - DNA, 50 bp, Homo sapiens
cggttgtgaggtgctcacgtgttttggagatagcaaaagtctcaaataat

SEQ ID NO: 410 - DNA, 50 bp, Homo sapiens
tgtctcctcatctcctggatctttgcccagcaaaacctccaaagagaccg

SEQ ID NO: 411 - DNA, 50 bp, Homo sapiens
cgcacagccagcaggcgcctgggctccactctctacctggaatcaccagg

SEQ ID NO: 412 - DNA, 50 bp, Homo sapiens
cggcagtcttctccactgagggactgggctgggaagtcctgcgtttcagt

SEQ ID NO: 413 - DNA, 50 bp, Homo sapiens
cggccccacccggttcttgtgtcaagacaaaaagaaaacccaggtggcct

SEQ ID NO: 414 - DNA, 50 bp, Homo sapiens
cgctgatagacatcaggtgacaggaaatcagtagcttctgctaccttggg

SEQ ID NO: 415 - DNA, 50 bp, Homo sapiens
cggcaagcatcatcgccaggccccaggcctcctgtgggacgccagtcacc

SEQ ID NO: 416 - DNA, 50 bp, Homo sapiens
cacttcctgactaaggagctgcagcgatacatcgaagggctcaagaagcg

SEQ ID NO: 417 - DNA, 50 bp, Homo sapiens
tccaggaagcagcagaggtcgcgcagctcacggttctctgcctgcagacg

SEQ ID NO: 418 - DNA, 50 bp, Homo sapiens
ccagcttgaagcccggtgccagatgcataagccacttgcccgagagcacg

SEQ ID NO: 419 - DNA, 50 bp, Homo sapiens
cgtagccagtgcgaggttggctctactttgggtttatgggttgggcataa

SEQ ID NO: 420 - DNA, 50 bp, Homo sapiens
cgggcagctctactgcttggagaagagaaagctaggtaactgtacagttg

SEQ ID NO: 421 - DNA, 50 bp, Homo sapiens
cacagccagagcacttacagtgagccgagccacagtccagcaagctgccg

SEQ ID NO: 422 - DNA, 50 bp, Homo sapiens
cgacactgtgataatcatgaagttttgtgtgagaaagataagggagaaga

SEQ ID NO: 423 - DNA, 50 bp, Homo sapiens
atagctattcatccttagaccctgtccaaacagagcaacagtcatatacg

SEQ ID NO: 424 - DNA, 50 bp, Homo sapiens
tatttttgaaggagctggtcagcatttgcctagactctcgggacaacacg

SEQ ID NO: 425 - DNA, 50 bp, Homo sapiens
ccctctagtgagacccattagtgtataagctctgcctacaccgagtttcg

SEQ ID NO: 426 - DNA, 50 bp, Homo sapiens
cggccatgccctggccccagaagtcgttggtgaagatgccccagcagagc

SEQ ID NO: 427 - DNA, 50 bp, Homo sapiens
cggtgatccatagcaaaagagcctatgagtcaggtgatgatccattcatc

SEQ ID NO: 428 - DNA, 50 bp, Homo sapiens
actactttgggcctcggtttccctgctccttgtagatcagagaagggacg

SEQ ID NO: 429 - DNA, 50 bp, Homo sapiens
cggccatgtctggtcactgcgtatgaatgacatctgtctctgcaacgctt

SEQ ID NO: 430 - DNA, 50 bp, Homo sapiens; identical to SEQ ID NO: 242

SEQ ID NO: 431 - DNA, 50 bp, Homo sapiens
cgcagtgacaggaataatgccactgtcctgctgagtgggaaaggagaggg

SEQ ID NO: 432 - DNA, 50 bp, Homo sapiens
agaggtctcaggaaagtagcctttatttatgtggcaccgatcggaacccg

SEQ ID NO: 433 - DNA, 50 bp, Homo sapiens
cgcacagcacggaaagggaaggcagcagccacatggacctgggcccacgg

SEQ ID NO: 434 - DNA, 50 bp, Homo sapiens
cggcaaccccggccagacagggggtatccagagggcccaggacacccacc

SEQ ID NO: 435 - DNA, 50 bp, Homo sapiens
aagaacgactctccgcaatgcgcatgacagaaaagcggctgcttctaccg

SEQ ID NO: 436 - DNA, 50 bp, Homo sapiens
tcgaacgctggacccttgagacatgtgacgggacctgagggcagccaccg

SEQ ID NO: 437 - DNA, 50 bp, Homo sapiens
cggagtttctgttacctgcagggaatgctctcttaactaatccaggggtg

SEQ ID NO: 438 - DNA, 50 bp, Homo sapiens
cggaggctggggagatgacatcagaaaacctggtccaaactgctccaaaa

SEQ ID NO: 439 - DNA, 50 bp, Homo sapiens
ctttactcaatgccaagatccacattaacacaattccactgaatcctccg

SEQ ID NO: 440 - DNA, 50 bp, Homo sapiens; identical to SEQ ID NO: 184

SEQ ID NO: 441 - DNA, 50 bp, Homo sapiens; identical to SEQ ID NO: 186

SEQ ID NO: 442 - DNA, 50 bp, Homo sapiens
ctcttgatgacctcgttgtctgtgctggcaccatgggtctcatcctgacg

SEQ ID NO: 443 - DNA, 50 bp, Homo sapiens; identical to SEQ ID NO: 254

SEQ ID NO: 444 - DNA, 50 bp, Homo sapiens
aagtgtcaacgagatgacttgcgctacagcggcacactggtgaggaaccg

SEQ ID NO: 445 - DNA, 50 bp, Homo sapiens
cgcatctgcttcccatgagtctcttggggaagacaaagctcacattcttc

SEQ ID NO: 446 - DNA, 50 bp, Homo sapiens
ctcagggatctgtgagcagagaggcctggctgcaaagggtctggaggacg

SEQ ID NO: 447 - DNA, 50 bp, Homo sapiens
tgtgacagccctgggctgaaagactcccggtggccgtagtgtaatagccg

SEQ ID NO: 448 - DNA, 50 bp, Homo sapiens
caactgctggtctgttttctgcccctgtagttttgtatgaattctttacg

SEQ ID NO: 449 - DNA, 50 bp, Homo sapiens
cgcaatgtaaaattatgtccagttgtagatgtgtgtgttggggtttaact

SEQ ID NO: 450 - DNA, 50 bp, Homo sapiens
cgggctggaattgagctcagctaaacaaagtccttcagctttgaagggag

SEQ ID NO: 451 - DNA, 50 bp, Homo sapiens
cggacaagataattatgatataacaaaagcttgtgttatttaaggtgggg

SEQ ID NO: 452 - DNA, 50 bp, Homo sapiens
cgtccctggagcccacgaaaatgtctgcaagatctgcaagggccacctta

SEQ ID NO: 453 - DNA, 50 bp, Homo sapiens
cgtcccgacttcacgcgagggtttatacctgaacagacacagcagggaaa

SEQ ID NO: 454 - DNA, 50 bp, Homo sapiens
cgccctctctgtgtagctggaagagcctgaccactcacgtctctccaggc

SEQ ID NO: 455 - DNA, 50 bp, Homo sapiens
aacagccatgattcctaaatattaaacattttcctcatctgacaggatcg

SEQ ID NO: 456 - DNA, 50 bp, Homo sapiens
acagagacagagcccaagaatagaggcacacggggaagtagacaacatcg

SEQ ID NO: 457 - DNA, 50 bp, Homo sapiens
cgagggtagttggtcagagttgtgcgagctggttgcagggaaggctaccg

SEQ ID NO: 458 - DNA, 50 bp, Homo sapiens
caagctctgtgcaaaataatctgtagtgcttgctataacgagctataacg

SEQ ID NO: 459 - DNA, 50 bp, Homo sapiens
cggggataggcaggtaagtaggatcaaggagtgcaaacagccaagtgtga

SEQ ID NO: 460 - DNA, 50 bp, Homo sapiens; identical to SEQ ID NO: 188

SEQ ID NO: 461 - DNA, 50 bp, Homo sapiens
tcggggaagctcagggtcttggggtctcagggcggggaggcaggacgacg

SEQ ID NO: 462 - DNA, 50 bp, Homo sapiens
cgggcagatcccaaggtttcctgatattccctactgctgcagtctctgct

SEQ ID NO: 463 - DNA, 50 bp, Homo sapiens
gtcttcattgttactcagcggggacaccctcctggcgctccgggggctcg

SEQ ID NO: 464 - DNA, 50 bp, Homo sapiens
cgctggagaccaaaatcttgggctatgaccaccctgggcaacatagtgag

SEQ ID NO: 465 - DNA, 50 bp, Homo sapiens; identical to SEQ ID NO: 84

SEQ ID NO: 466 - DNA, 50 bp, Homo sapiens
cgcggcttgactagtaaagtgctcaactattctcgaccaccattgggtat

SEQ ID NO: 467 - DNA, 50 bp, Homo sapiens
atgtggcgggggctttggaccctggcggcccaagcggcacgtgggcctcg

SEQ ID NO: 468 - DNA, 50 bp, Homo sapiens
cggccgccgcccccacccgacccaaccgcccgcacaaaaggccggagccg

SEQ ID NO: 469 - DNA, 50 bp, Homo sapiens
cgggctcggtccagactgacaacaatgccctcctcgtacctgaagaactt

SEQ ID NO: 470 - DNA, 50 bp, Homo sapiens
acgaaggacagtgatccctaacagttcagcagtccctactgtggcaaccg

SEQ ID NO: 471 - DNA, 50 bp, Homo sapiens; identical to SEQ ID NO: 101

SEQ ID NO: 472 - DNA, 50 bp, Homo sapiens
gcacggggcctggcacgtagggggccttcattgcatctctcttcccttcg

SEQ ID NO: 473 - DNA, 50 bp, Homo sapiens
cgggtggggactaaacaaaaaggtcaaggagaaatgtcaacctcaagatg

SEQ ID NO: 474 - DNA, 50 bp, Homo sapiens
cgctgggctctagctctgtatctgtgctcctggcaggccagcaccccatc

SEQ ID NO: 475 - DNA, 50 bp, Homo sapiens
tcaaactgccgcgggcgtgcgggttgacgccgagtgtgtgtgtcggggcg

SEQ ID NO: 476 - DNA, 50 bp, Homo sapiens
ttccagttcgtgccatcaaggagggcgacctgagcaccaagtacgatgcg

SEQ ID NO: 477 - DNA, 50 bp, Homo sapiens
tgagatctctgtctctatcctatcctgtccctggccttctgaggcaagcg

SEQ ID NO: 478 - DNA, 50 bp, Homo sapiens; identical to SEQ ID NO: 190

SEQ ID NO: 479 - DNA, 50 bp, Homo sapiens
tgcatactgatttctgaacattaaatctgcatccaatcgtgtttcatccg

SEQ ID NO: 480 - DNA, 50 bp, Homo sapiens
aaaacaccaacaaaaaacgactcctaacatcactctgggtggtgaaagcg

SEQ ID NO: 481 - DNA, 50 bp, Homo sapiens
cgatccggaattcaggatgagccatgaagttatggatggaggttcgagat

SEQ ID NO: 482 - DNA, 50 bp, Homo sapiens
cgttgttgttgttttagttacgtttagctttgtcagcaacactttggccc

SEQ ID NO: 483 - DNA, 50 bp, Homo sapiens; identical to SEQ ID NO: 352

SEQ ID NO: 484 - DNA, 50 bp, Homo sapiens
cggccagagtcttatcccctggctctggctgatgaggaacacgtagaatg

SEQ ID NO: 485 - DNA, 50 bp, Homo sapiens
cgcggggggtccccctggcacctccaagcccatcccattgacgaagtaac

SEQ ID NO: 486 - DNA, 50 bp, Homo sapiens
cgagagggcagctttaagacattttagaattgtttcttaccattgtcttt

SEQ ID NO: 487 - DNA, 50 bp, Homo sapiens
caagtccagttttccacctaaatatcacttgaggccaacattctctttcg

SEQ ID NO: 488 - DNA, 50 bp, Homo sapiens
cacctgacagacaaagccctgggtccaaaatggctcatcggggtcaggcg

SEQ ID NO: 489 - DNA, 50 bp, Homo sapiens; identical to SEQ ID NO: 361

SEQ ID NO: 490 - DNA, 50 bp, Homo sapiens
tgtgcctccctggcagtcaaagttctaagggggactcctgaagccaggcg

SEQ ID NO: 491 - DNA, 50 bp, Homo sapiens
cggactcgttttattgactgacagtcagtgctagcagtgacttcagctgc

SEQ ID NO: 492 - DNA, 50 bp, Homo sapiens
gtcatagaaggctgtgttgcccccagcaagagcaaagccaccgctccacg

SEQ ID NO: 493 - DNA, 50 bp, Homo sapiens; identical to SEQ ID NO: 200

SEQ ID NO: 494 - DNA, 50 bp, Homo sapiens
gaaaacccagcagaacataaccgtgcaggtctccgacgtcaatgacaacg

SEQ ID NO: 495 - DNA, 50 bp, Homo sapiens
cgcaagagaagaaaaaaggtaagagtttagttctcaaaaagacaaagagt

SEQ ID NO: 496 - DNA, 50 bp, Homo sapiens
cgaaggaaaccttcgctggctccctagtgccctggaataacactgaaacc

SEQ ID NO: 497 - DNA, 50 bp, Homo sapiens
tttaggcaggtggccaattttggtccagctggtggcaagggcaggtgtcg

SEQ ID NO: 498 - DNA, 50 bp, Homo sapiens
cggcaggttttgggactcattcctgcaagcccaagccagaacttgttctg

SEQ ID NO: 499 - DNA, 50 bp, Homo sapiens
cgccattccccagcacccgcagcgggcactgtggctgcacgccgccctcg

SEQ ID NO: 500 - DNA, 50 bp, Homo sapiens
ctgtcagtagatctcagcgggagagcagtgctggtatgtgaatgtcctcg

SEQ ID NO: 501 - DNA, 50 bp, Homo sapiens
cagtcccagctttcactgccagggtcccagtcagattccaggaatttgcg

SEQ ID NO: 502 - DNA, 50 bp, Homo sapiens
cgcccctggagaggcctcttggggaatgaactgtccctcccaaccttgga

SEQ ID NO: 503 - DNA, 50 bp, Homo sapiens
cggcggcaggaactatcagtagacagctgctgcttccatgaaacggaaaa

SEQ ID NO: 504 - DNA, 50 bp, Homo sapiens
cgggccctgcagtccttaataaaaaattgcagatttcattctaggtgtca

SEQ ID NO: 505 - DNA, 50 bp, Homo sapiens; identical to SEQ ID NO: 380

SEQ ID NO: 506 - DNA, 50 bp, Homo sapiens
agatcctaggtgagggcaggttgactggcagggctggtttttcgggatcg

SEQ ID NO: 507 - DNA, 50 bp, Homo sapiens
aaggcctggctggctgcgagctcaggaggccgcctgaggactgcacaccg

SEQ ID NO: 508 - DNA, 50 bp, Homo sapiens; identical to SEQ ID NO: 205

SEQ ID NO: 509 - DNA, 50 bp, Homo sapiens
cggccccacaaattacgtgtgtgcaggattaagggaaaccagcaaggggt

SEQ ID NO: 510 - DNA, 50 bp, Homo sapiens
cgcgccttttattgacgcaaacctcccgttctcgcgggaacttgggaacg

SEQ ID NO: 511 - DNA, 50 bp, Homo sapiens
cgccatcttcacaccccgctccgtcgcgtacgcacctgggtcacgtgata

SEQ ID NO: 512 - DNA, 50 bp, Homo sapiens; identical to SEQ ID NO: 207

SEQ ID NO: 513 - DNA, 50 bp, Homo sapiens
cggccctgcacacgtgtccactgtgacgcgtctgtcatggtcccctcggc

SEQ ID NO: 514 - DNA, 50 bp, Homo sapiens
cggctggggaccgtagcaagagcaagagggctcctcagcagtccctgtgg

SEQ ID NO: 515 - DNA, 50 bp, Homo sapiens
cgccagtatggaaacacagtgtgtaaagcaagcttcgagagaggaaagag

SEQ ID NO: 516 - DNA, 50 bp, Homo sapiens
cgcctacaactctctcggtcaaacaaccgaaaacatcccgtgccggaagc

SEQ ID NO: 517 - DNA, 50 bp, Homo sapiens
ggtgcgccctgggcagctgggtggcctcaagccctactggcgagactccg

SEQ ID NO: 518 - DNA, 50 bp, Homo sapiens; identical to SEQ ID NO: 209

SEQ ID NO: 519 - DNA, 50 bp, Homo sapiens
ggatgcactgcgagcctgccaggaattccaggcctcgcagtgttctggcg

SEQ ID NO: 520 - DNA, 50 bp, Homo sapiens
cgcttccacatttggacgcccccacccagcggcctccgtggcacacaccg

SEQ ID NO: 521 - DNA, 50 bp, Homo sapiens
ggaggacttggggtgtggcaggggagcagccaagccggcagatgactacg

SEQ ID NO: 522 - DNA, 50 bp, Homo sapiens; identical to SEQ ID NO: 300

SEQ ID NO: 523 - DNA, 50 bp, Homo sapiens
acacagaaatgggggttctcacactacagattaaggagtgtcccaggccg

SEQ ID NO: 524 - DNA, 50 bp, Homo sapiens
cgggacaagaaacaacatgtgagcagcacagccattcagttgggatgctg

SEQ ID NO: 525 - DNA, 50 bp, Homo sapiens
ggcgggccttgcctccagcaggggtgtgaagagcccaggcttattttccg

SEQ ID NO: 526 - DNA, 50 bp, Homo sapiens; identical to SEQ ID NO: 301

SEQ ID NO: 527 - DNA, 50 bp, Homo sapiens
tcctgcaggcctcaccatagggcacctctttggcccaattcagggctccg

SEQ ID NO: 528 - DNA, 50 bp, Homo sapiens
ctctgaagcctgcacctctccattaaacacagaacagctgccagaggacg

SEQ ID NO: 529 - DNA, 50 bp, Homo sapiens
cggtgtgagtgctcactcaatgcaatgtgttggtgatggtaactgtcctt

SEQ ID NO: 530 - DNA, 50 bp, Homo sapiens; identical to SEQ ID NO: 302

SEQ ID NO: 531 - DNA, 50 bp, Homo sapiens
cgctgctgcctgggactcagtgattatcccattttgcagaccagaaagag

SEQ ID NO: 532 - DNA, 50 bp, Homo sapiens
agccggacaagtgagaccaggcggccccttgggtgggcagggtctgtccg

SEQ ID NO: 533 - DNA, 50 bp, Homo sapiens
cggctcagcttgctcacccaccccagaaggagcgatttgcatttatgaag

SEQ ID NO: 534 - DNA, 50 bp, Homo sapiens
cgggcaaggaaaggatacaggtatctgcctttcgcatggcactgagcaaa

SEQ ID NO: 535 - DNA, 50 bp, Homo sapiens
cgggacaagcttgacaaggtgggtgtcttcctggactatgaccaaggctt

SEQ ID NO: 536 - DNA, 50 bp, Homo sapiens
cggtagagaatattttgactccttgagtaatgggagttcttttgtggtct

SEQ ID NO: 537 - DNA, 50 bp, Homo sapiens
cgcgctgcagtcacgcctcccgctgccagcccggcaccgggatcttaatc

SEQ ID NO: 538 - DNA, 50 bp, Homo sapiens
ggaggaggatccccgagaggggcgcaagttgcgatgtccggaagccgacg

SEQ ID NO: 539 - DNA, 50 bp, Homo sapiens
cgccagcagatacaaaggcagacaaagtgagcgaggcagtttccaggacc

SEQ ID NO: 540 - DNA, 50 bp, Homo sapiens; identical to SEQ ID NO: 309

SEQ ID NO: 541 - DNA, 50 bp, Homo sapiens; identical to SEQ ID NO: 221

SEQ ID NO: 542 - DNA, 50 bp, Homo sapiens
cgggtggaaggagaatgaggaggcaaccaaaataagatcaaaaagtcagg

SEQ ID NO: 543 - DNA, 50 bp, Homo sapiens
cgatgagcaaaatcacacatcctcatgtccatctggtgctctgtattgtg

SEQ ID NO: 544 - DNA, 50 bp, Homo sapiens
gcggagccagcgctcttgggggcagggctgcgagtcccctgtgtgggacg

SEQ ID NO: 545 - DNA, 50 bp, Homo sapiens
cgtgacctgtctgtaccctagcgcggggcttctgtacgctgctaggaagc

SEQ ID NO: 546 - DNA, 50 bp, Homo sapiens
gaagcagttaagacaacaatacccaggactagccagatcccgaattaccg

SEQ ID NO: 547 - DNA, 50 bp, Homo sapiens
cgtcgggctcacagaaggctcaggtttcctccagtatagttcaggggatg

SEQ ID NO: 548 - DNA, 50 bp, Homo sapiens
cgcctcatacaagaacacagaacagggagcttgtctccttggggattaga

SEQ ID NO: 549 - DNA, 50 bp, Homo sapiens
cgctgcggcaaacctaggagggacccaaaagcagaaaatcctagaaaaga

SEQ ID NO: 550 - DNA, 50 bp, Homo sapiens
cggaaactgatctagtcagggaggtcagagaatatctccctgaggaaaag

SEQ ID NO: 551 - DNA, 50 bp, Homo sapiens
cgcactgactggtgagcgctttgcaccaattagtagcaggtgctacagcc

SEQ ID NO: 552 - DNA, 50 bp, Homo sapiens; identical to SEQ ID NO: 229

SEQ ID NO: 553 - DNA, 50 bp, Homo sapiens
cggggtgcattagctgtgacttttgctgcatccagtcatgcacatgtgcc

SEQ ID NO: 554 - DNA, 50 bp, Homo sapiens
gctttctgctttcaccgggaggccctttaattgttaccaaaaataggacg

SEQ ID NO: 555 - DNA, 50 bp, Homo sapiens
aggtactggatgcacggcaggccctgccactgcttctcagaaccctctcg

SEQ ID NO: 556 - DNA, 50 bp, Homo sapiens
aattctttcacagttctggagtcaagaagtccaaaatgaaggtgttcacg

SEQ ID NO: 557 - DNA, 50 bp, Homo sapiens
cggccctacacgggcaccgggcctcagtcctgccaggcccacgccatcct

SEQ ID NO: 558 - DNA, 50 bp, Homo sapiens; identical to SEQ ID NO: 235

SEQ ID NO: 559 - DNA, 50 bp, Homo sapiens
gaagctggcgacgaacccgtgtctcaagaatttctacggcttgtttcccg

SEQ ID NO: 560 - DNA, 50 bp, Homo sapiens
cggctgcaggccccagagtccacgagggtgggtacaaatcccctgggcac

SEQ ID NO: 561 - DNA, 50 bp, Homo sapiens
tgattggcccaaagcatcgtgaatattcatgacagcagccacaaaggccg

SEQ ID NO: 562 - DNA, 50 bp, Homo sapiens
ttctcgggtcttcgtgagcattaagtcaatgaacccagggcaccaaagcg

SEQ ID NO: 563 - DNA, 50 bp, Homo sapiens
aggccagcgaacatccagctgtcagttggtctggggactatcagcattcg

SEQ ID NO: 564 - DNA, 50 bp, Homo sapiens
gaagatggtgaacctgagatgacctgtcaatgggattcccagacagtccg

SEQ ID NO: 565 - DNA, 50 bp, Homo sapiens
tggataaaggggtttctaatacccagctcacatggctgtggtgacaaacg

SEQ ID NO: 566 - DNA, 50 bp, Homo sapiens
gtgaacacctgccgaagcctttgccttgggagaggctccagcaaaatccg

SEQ ID NO: 567 - DNA, 50 bp, Homo sapiens
cgggcactgactggggacccaggtgagtctagcaaatgacaaggcccaac

SEQ ID NO: 568 - DNA, 50 bp, Homo sapiens
cggcaaggtttataacgagaataaatgaaataaatacaaaatccagaata

SEQ ID NO: 569 - DNA, 50 bp, Homo sapiens
cggggacttgatatggaaagaattaatgtactggcttttttgtatagatg

SEQ ID NO: 570 - DNA, 50 bp, Homo sapiens
cgggtagggttaggctcagttcaggcagaacctgccattcgctcattcac

SEQ ID NO: 571 - DNA, 50 bp, Homo sapiens
attcatctctagtctctgtaaaatgccagccctcttctgggttaacagcg

SEQ ID NO: 572 - DNA, 50 bp, Homo sapiens
cgcaccgtctcacctgagttttctgccactggatcatgtgggtctggttg

SEQ ID NO: 573 - DNA, 50 bp, Homo sapiens
gagtgttccccatgctgacttctgccaggctgcagggaataaagctaacg

SEQ ID NO: 574 - DNA, 50 bp, Homo sapiens
cggaaaggttcattggctaatttgccacagttggttttcaatatgtccat

SEQ ID NO: 575 - DNA, 50 bp, Homo sapiens
ctgtgcagacgccatttgcccccagcttaattgagcatatctctccctcg

SEQ ID NO: 576 - DNA, 50 bp, Homo sapiens
cggtgggaacgggaagcttccccaatcagactgcatcctgagggacagga

SEQ ID NO: 577 - DNA, 50 bp, Homo sapiens; identical to SEQ ID NO: 251

SEQ ID NO: 578 - DNA, 50 bp, Homo sapiens; identical to SEQ ID NO: 253

SEQ ID NO: 579 - DNA, 50 bp, Homo sapiens
cggcctttgtggatgccaacctttgtcacagccaggagggggcctccctc

SEQ ID NO: 580 - DNA, 50 bp, Homo sapiens
cggcctcaggccaagcatgagaacaacgtcagacaaatccccattgattg

SEQ ID NO: 581 - DNA, 50 bp, Homo sapiens
cgccgacccacagggcacaatggtctacaaagttggaaatgaaggtctgc

SEQ ID NO: 582 - DNA, 50 bp, Homo sapiens
gcacgggcagctgagctgggggtacagggcacccccgtgtcccagggtcg

SEQ ID NO: 583 - DNA, 50 bp, Homo sapiens
caggaaactcctacctcatcacggcagctgcatgtgtgggtaagcccacg

SEQ ID NO: 584 - DNA, 50 bp, Homo sapiens
cggcagttcttagtgagggcaattcattcaatccagagccagaaacagga

SEQ ID NO: 585 - DNA, 50 bp, Homo sapiens
cggccacctcagacttctgtataggaggaggcctgactgggagatccctg

SEQ ID NO: 586 - DNA, 50 bp, Homo sapiens
tgtcccaggatgacatcaagggcattcaggagctctatggtaaacctccg

SEQ ID NO: 587 - DNA, 50 bp, Homo sapiens
tatggacagtcttttgcctggcgtgggggccaagtggacaataccacacg

SEQ ID NO: 588 - DNA, 50 bp, Homo sapiens
ctccctccacgggacccagcctgcagaagggtcctgcaggaagtggcacg

SEQ ID NO: 589 - DNA, 50 bp, Homo sapiens
gagtttctactctgcctacaggtctcgggtgaattcacacaaactcttcg

SEQ ID NO: 590 - DNA, 50 bp, Homo sapiens
caagatccagggacctagaggcctcggcgatgacactgcgctgaacgacg

SEQ ID NO: 591 - DNA, 50 bp, Homo sapiens
cgccaatggttccagcaaagccccaggactgctctgctcctggtcttgta

SEQ ID NO: 592 - DNA, 50 bp, Homo sapiens
tcagagctgttttcgtgggtttttaacttccagttgctcaagggtccacg

SEQ ID NO: 593 - DNA, 50 bp, Homo sapiens
cggctccacacttccccagcgtggcctgcatttctattgggcagctggcg

SEQ ID NO: 594 - DNA, 50 bp, Homo sapiens
tatgagaggccagaatattgcttccgttcccttttcatctctgcacaccg

SEQ ID NO: 595 - DNA, 50 bp, Homo sapiens
cgccctgatagcaccgcagaggttctgatctaattgatctgggatgcggc

SEQ ID NO: 596 - DNA, 50 bp, Homo sapiens
atatacagtggccatgaagcaggcaccacttagaaacctctgtattaacg

SEQ ID NO: 597 - DNA, 50 bp, Homo sapiens
cggcagaagcttcaccacaaaagcgaaatgggcacaccacaggtaagact

SEQ ID NO: 598 - DNA, 50 bp, Homo sapiens
cggctagagactcactgactcatgcgtgtgtggtaggaatcttccaggaa

SEQ ID NO: 599 - DNA, 50 bp, Homo sapiens
cgggacggggacttcctggaagattcctaccacacacgcatgagtcagtg

SEQ ID NO: 600 - DNA, 50 bp, Homo sapiens; identical to SEQ ID NO: 188

SEQ ID NO: 601 - DNA, 50 bp, Homo sapiens
ggaaacttgctgtgggtgaccatggcgcacggagccagcgtggatctgcg

SEQ ID NO: 602 - DNA, 50 bp, Homo sapiens
gggtggtacaaggggcgccccttcgctgttttaaaatctgatgcttcacg

SEQ ID NO: 603 - DNA, 50 bp, Homo sapiens
gatttctccttgaacaatccccgcaaagatagcagccaaaaaaggatgcg

SEQ ID NO: 604 - DNA, 50 bp, Homo sapiens
gaaagcactaaattatcctgcaaactcccggctgatgcctaaggaattcg

SEQ ID NO: 605 - DNA, 50 bp, Homo sapiens
cggaagcacagtttgcaacagtttggggttgtcttattttcactgctggc

SEQ ID NO: 606 - DNA, 50 bp, Homo sapiens
cgcacggatgggacctgcttctcactcctgcacagctgaacctccaggac

SEQ ID NO: 607 - DNA, 50 bp, Homo sapiens
ccaggagtgcgctgctctcgggaaggcatcccatggcctgagcagcagcg

SEQ ID NO: 608 - DNA, 50 bp, Homo sapiens
gctgggtccccgtgtagcacttagctaaagatgatcagcagctgagaacg

SEQ ID NO: 609 - DNA, 50 bp, Homo sapiens
acagtatacagtcggtgctcaggatgcatttgttttcctgtcccccagcg

SEQ ID NO: 610 - DNA, 50 bp, Homo sapiens
tgccggccacgtggttgctgggcaggtgtggggtcaggctgtggatcccg

SEQ ID NO: 611 - DNA, 50 bp, Homo sapiens
cggctcagccaaccccctagataatagcggtttctcaaactgtggtcccg

SEQ ID NO: 612 - DNA, 50 bp, Homo sapiens; identical to SEQ ID NO: 268

SEQ ID NO: 613 - DNA, 50 bp, Homo sapiens
cctattcttccagaaccttcctggagtattccgttttctctaggacaacg

SEQ ID NO: 614 - DNA, 50 bp, Homo sapiens
cgggggaattcattgataaggagagcaagcatcccaagaacatccccatc

SEQ ID NO: 615 - DNA, 50 bp, Homo sapiens
cggacacagcaccaggtccagattggcaagaaaggaccctgggcttgctc

SEQ ID NO: 616 - DNA, 50 bp, Homo sapiens
atcttcaagtctcactttggtcctcagtttgtagtatctattgcagaccg

SEQ ID NO: 617 - DNA, 50 bp, Homo sapiens
cggtaccagtaactgccaggaaaaggaagggaatatgtccagaaaaagca

SEQ ID NO: 618 - DNA, 50 bp, Homo sapiens
tggttattccccaggagccgcgcgaagcatgagctaattttcagtgagcg

SEQ ID NO: 619 - DNA, 50 bp, Homo sapiens
cgcacatgaggcacagagaaaggacaaaacgcagccacacctgagctgcg

SEQ ID NO: 620 - DNA, 50 bp, Homo sapiens
gtgcctcccttctgcactgtgggtgtgtggagctgtctttcttggagacg

SEQ ID NO: 621 - DNA, 50 bp, Homo sapiens
tgagcaggagcgagacagagcaccacggggcacgtgaggccagggcaccg

SEQ ID NO: 622 - DNA, 50 bp, Homo sapiens
tcccacagtgaacccctctaggtgcaattacaggatgattttgtgtctcg

SEQ ID NO: 623 - DNA, 50 bp, Homo sapiens
gccccaggaccccgggatgtgcagcctaggggtggggttgaggttctgcg

SEQ ID NO: 624 - DNA, 50 bp, Homo sapiens
acctcttgtggtattcctctatcaggcaatctgggtgggcagagacttcg

SEQ ID NO: 625 - DNA, 50 bp, Homo sapiens
agtcttagcttaaggaaatgtgccacctgcgtgcagggatgaggtcatcg

SEQ ID NO: 626 - DNA, 50 bp, Homo sapiens
ccatgctgcggatcctgtgcctggcactctgcagcctgctgactggcacg

SEQ ID NO: 627 - DNA, 50 bp, Homo sapiens
ttcatcgcaatgggctgtatagcttggcccgtctcattgtaacaaattcg

SEQ ID NO: 628 - DNA, 50 bp, Homo sapiens
tgggctagcccagcgccaccttcctggagactgtctggtggaagacaccg

SEQ ID NO: 629 - DNA, 50 bp, Homo sapiens
cgctgctgaatgcacagcactgtgaatgcacttccatcactgaatataca

SEQ ID NO: 630 - DNA, 50 bp, Homo sapiens
cggggctgagtacttggctgaccaccctagggccacaccagcccagggga

SEQ ID NO: 631 - DNA, 50 bp, Homo sapiens
cgcacaatacgtggaaggccatggaaggcatcttcatcaagcctagtgtg

SEQ ID NO: 632 - DNA, 50 bp, Homo sapiens
aagcacttgatgtttgccctcacactgttcctctcccattccagttgtcg

SEQ ID NO: 633 - DNA, 50 bp, Homo sapiens
ggccttacaccagtgcagagggcaggcttgaggctgatctggtacacacg

SEQ ID NO: 634 - DNA, 50 bp, Homo sapiens
tagaatagtgactggcacataataggtgttcaataaatgtcaacccaccg

SEQ ID NO: 635 - DNA, 50 bp, Homo sapiens
cgcctcagctggtgctccaagacctgctggccagactggtgaggccaggt

SEQ ID NO: 636 - DNA, 50 bp, Homo sapiens
cggggacagggctcctaatgtgtttgtcaagtaaataatgagtaataatc

SEQ ID NO: 637 - DNA, 50 bp, Homo sapiens
cggtgaggccagtgcactcaagtttgggaactaccaatatagagtaaaag

SEQ ID NO: 638 - DNA, 50 bp, Homo sapiens
acatacgtcacattggcagtgtttgtaggatatcgggctgtagggatccg

SEQ ID NO: 639 - DNA, 50 bp, Homo sapiens
gctagctgccgggcattgggagggcttgacgggccagaggtgactgagcg

SEQ ID NO: 640 - DNA, 50 bp, Homo sapiens
cggcacctcgcctcgaccttgccattttatactcaattggggcgtagggt

SEQ ID NO: 641 - DNA, 50 bp, Homo sapiens
gcaggtcattactatgggtaactggggctccatctctctgaaataccacg

SEQ ID NO: 642 - DNA, 50 bp, Homo sapiens
ccctggctggtgtttcatgtccaccagctctgcgttcagcacagcactcg

SEQ ID NO: 643 - DNA, 50 bp, Homo sapiens
cgtctgtcccaaagggcgacacaatcctgaggcagctgttcgcaaactcg

SEQ ID NO: 644 - DNA, 50 bp, Homo sapiens
cggcgcacctctccagtgggacccgcaggacgccgtctctcagccccacg

SEQ ID NO: 645 - DNA, 50 bp, Homo sapiens
taaggcccttcagaaataggatcgacagggctggagtggaggggcccccg

SEQ ID NO: 646 - DNA, 50 bp, Homo sapiens
ctagcatggtgcctaacagctcctaccaaatagtagatgctcagtaaacg

SEQ ID NO: 647 - DNA, 50 bp, Homo sapiens
cggtcagtctgcctacagagtgcttgccaggcaatgaaggaagagcatgc

SEQ ID NO: 648 - DNA, 50 bp, Homo sapiens
cgtgcgaacatgaactaataatccctttgggtgtctggaatgcctgccca

SEQ ID NO: 649 - DNA, 50 bp, Homo sapiens; identical to SEQ ID NO: 199

SEQ ID NO: 650 - DNA, 50 bp, Homo sapiens; identical to SEQ ID NO: 281

SEQ ID NO: 651 - DNA, 50 bp, Homo sapiens
caacactgtgaaaccctgtctctactaaaatacaaaatattagtcagtcg

SEQ ID NO: 652 - DNA, 50 bp, Homo sapiens
cggtagggtttggaggtattattcagtgtagaggaaacagtatgcataaa

SEQ ID NO: 653 - DNA, 50 bp, Homo sapiens
cgctggcaagatgaatcacaataacaactctgaaggcaagaggattggtg

SEQ ID NO: 654 - DNA, 50 bp, Homo sapiens
cggcagaattgtgcttaatagttggtgaggtttctatgggcgatgtggaa

SEQ ID NO: 655 - DNA, 50 bp, Homo sapiens
cttccttcagggagtcgtatatatgagagggctttagcttgctctcctcg

SEQ ID NO: 656 - DNA, 50 bp, Homo sapiens
cgggacacgtactatctgcctcagcctagtgctaagcactttacacattt

SEQ ID NO: 657 - DNA, 50 bp, Homo sapiens
cggggctcatggctaaagttaaggtactgatcaaagttttggatgtaaat

SEQ ID NO: 658 - DNA, 50 bp, Homo sapiens
cgctccaggcagcgctccacttccgcggcttcgcccttgacagcgccctg

SEQ ID NO: 659 - DNA, 50 bp, Homo sapiens
ggagggcatcttattttaccaggtaaacaagggtcactaagaggcatgcg

SEQ ID NO: 660 - DNA, 50 bp, Homo sapiens
ttcacacttagggctcaggacgcccctgtaggcagggtgctggtgggacg

SEQ ID NO: 661 - DNA, 50 bp, Homo sapiens
cgcaaaagcaagattcgctccaggagtttcatgccacaggatggtcgacg

SEQ ID NO: 662 - DNA, 50 bp, Homo sapiens
gctcatcacctgggggcagcttttgtacctgcaccctgtatgaggctccg

SEQ ID NO: 663 - DNA, 50 bp, Homo sapiens
ggtctctggcatctgtcccgtctccagcacaaacaacatctgggcaatcg

SEQ ID NO: 664 - DNA, 50 bp, Homo sapiens
gctgagggtgccaggttgtcctccaaggccatcagaaagaaggcaccacg

SEQ ID NO: 665 - DNA, 50 bp, Homo sapiens; identical to SEQ ID NO: 205

SEQ ID NO: 666 - DNA, 50 bp, Homo sapiens
cagcctcatgaactccatgggtgtcgtcacgctcctcatttggctcttcg

SEQ ID NO: 667 - DNA, 50 bp, Homo sapiens
cgttcagtgccactcaggcctggctgcgaagatacagatggtatatgtga

SEQ ID NO: 668 - DNA, 50 bp, Homo sapiens
ggcagtgttgactgcgttccataccgggacatccaacacaacatttgtcg

SEQ ID NO: 669 - DNA, 50 bp, Homo sapiens
tgattctggacctcagctcttcaacaaagctgaggtccagaatcaaaccg

SEQ ID NO: 670 - DNA, 50 bp, Homo sapiens
agctttctgggtctccttaatgagcctgaggcctgctgcctgcaagtgcg

SEQ ID NO: 671 - DNA, 50 bp, Homo sapiens
cctgaggctctgggagcagatgggagcacaggctctgttttggccctgcg

SEQ ID NO: 672 - DNA, 50 bp, Homo sapiens
tgcaatgataagtttgctgaactgcatctctgcccaatgccacttcctcg

SEQ ID NO: 673 - DNA, 50 bp, Homo sapiens
cccatgcaggagaaacccgtttggtgtttactactaagtcatgatctccg

SEQ ID NO: 674 - DNA, 50 bp, Homo sapiens
cgctgtgtgtgttgagacacacgcaacattcgtgcacatagaactcatga

SEQ ID NO: 675 - DNA, 50 bp, Homo sapiens
cggtgccccacaggccactgtgcaggatctccactcggccctgacacagg

SEQ ID NO: 676 - DNA, 50 bp, Homo sapiens
cgggtccccagtcctcaaggtcccagcacaatgccccaggaggaagactc

SEQ ID NO: 677 - DNA, 50 bp, Homo sapiens
cgccccaagtcatgagggaagcttccagactgactcaggctcaggctggc

SEQ ID NO: 678 - DNA, 50 bp, Homo sapiens
cggtgactgcaggctcctggatcccatggagcagaaacctgaacttgtct

SEQ ID NO: 679 - DNA, 50 bp, Homo sapiens
tggctaccgtgctgtggggtgcctcgaaagtgtgaatcaccaaatattcg

SEQ ID NO: 680 - DNA, 50 bp, Homo sapiens
tgttcaatgtaataaacaactggctctttgctgcttcagatctgtgctcg

SEQ ID NO: 681 - DNA, 50 bp, Homo sapiens
ttatcaaagactttatattttctgtaaaatactacacagtaaatcttccg

SEQ ID NO: 682 - DNA, 50 bp, Homo sapiens
ctaccctactggaaaatagtgttaattattattcagttctatttccatcg

SEQ ID NO: 683 - DNA, 50 bp, Homo sapiens
tcttctttccacaggttctcaggcaagagccacctgctattgccgaaccg

SEQ ID NO: 684 - DNA, 50 bp, Homo sapiens
cgtggctatgctgtggtctccctgagcctactctagctggaaaggtaatt

SEQ ID NO: 685 - DNA, 50 bp, Homo sapiens; identical to SEQ ID NO: 212

SEQ ID NO: 686 - DNA, 50 bp, Homo sapiens
cgacactgcacatcaccgagaccaggcacagtgccggttatgcctaagca

SEQ ID NO: 687 - DNA, 50 bp, Homo sapiens
ctcccgagttcgcccaacttactactctgcatgctggtgcctagtctgcg

SEQ ID NO: 688 - DNA, 50 bp, Homo sapiens
caacggaacgaagctgcatctaagtcttattctagaattctttgtgaccg

SEQ ID NO: 689 - DNA, 50 bp, Homo sapiens
cggacaattggggccaaacgaggaaggacacagacccaaaagccagaccc

SEQ ID NO: 690 - DNA, 50 bp, Homo sapiens; identical to SEQ ID NO: 391

SEQ ID NO: 691 - DNA, 50 bp, Homo sapiens
aacaattatgggcttaaagaattgatcattacagcccctgggatttagcg

SEQ ID NO: 692 - DNA, 50 bp, Homo sapiens
cgccataagtgcccccagaacttcagcgcccaccatggcgcacaaggccg

SEQ ID NO: 693 - DNA, 50 bp, Homo sapiens
cggtgagtcatgcttcaggtttgccagggcctgtgtcccgcaaaaaggcg

SEQ ID NO: 694 - DNA, 50 bp, Homo sapiens
cgctgggaggcaccacttgacatgacctgggacaggtggctcagtctctc

SEQ ID NO: 695 - DNA, 50 bp, Homo sapiens
ctctgcatcctccccaagcaaaacctcaccaaggtggtgagatcctgacg

SEQ ID NO: 696 - DNA, 50 bp, Homo sapiens
cgggggcgccgcctagaaacacacaatggcccctcctggccaaccccccg

SEQ ID NO: 697 - DNA, 50 bp, Homo sapiens
cgcgtcataaaggacaagcagacccaactgaaccgcggctttgccttcat

SEQ ID NO: 698 - DNA, 50 bp, Homo sapiens
caacctcaggttccttttaatggagtagtcacttattcattcaacatacg

SEQ ID NO: 699 - DNA, 50 bp, Homo sapiens
acagtcatactgtcctgagtaaggacagtcaacaaaggatcatttctccg

SEQ ID NO: 700 - DNA, 50 bp, Homo sapiens
cgcgtaacactgtgcctggctttgcaaatatattttacagatagtgtagt

SEQ ID NO: 701 - DNA, 50 bp, Homo sapiens
ctgaccaaggcatcacaataaatacagttagcagcacacagaagcctgcg

SEQ ID NO: 702 - DNA, 50 bp, Homo sapiens
cgacattaaaagaccaatggagaatgtatattagcaatctgtaaggcagt

SEQ ID NO: 703 - DNA, 50 bp, Homo sapiens
cgccgccatctcaggtctcttggctttgccagggcccaccggagaaaact

SEQ ID NO: 704 - DNA, 50 bp, Homo sapiens
gtcagttttctccggtgggccctggcaaagccaagagacctgagatggcg

SEQ ID NO: 705 - DNA, 50 bp, Homo sapiens
gagaggcgcaggcgagctggaggggagcggcgcgcggcggccagtgggcg

SEQ ID NO: 706 - DNA, 50 bp, Homo sapiens
gagagttcaaagtgagcttacttccagtctcacgtacagatgattctacg

SEQ ID NO: 707 - DNA, 50 bp, Homo sapiens
cgggatggaagttagcgaatttgggcaacttgcctctactgcccagtcga

SEQ ID NO: 708 - DNA, 50 bp, Homo sapiens
cgtgggcgagttggtggcaaaatctgaactggatactcacggagggcccg

SEQ ID NO: 709 - DNA, 50 bp, Homo sapiens
ctttggtgcagaaccaccgcatccactttctcaggcccaggggaggggcg

SEQ ID NO: 710 - DNA, 50 bp, Homo sapiens
aagcaggcatggttgatttcccgctagcgggaacgatctggcctcagacg

SEQ ID NO: 711 - DNA, 50 bp, Homo sapiens
cggcagagagttgccactgcaacgcctggctcccagccctcccacccagc

SEQ ID NO: 712 - DNA, 50 bp, Homo sapiens
cgccatctcagccctggtgagccccaaatctgagagggagggaaggtcct

SEQ ID NO: 713 - DNA, 50 bp, Homo sapiens
cgcccccagctgccagtccccagcagctcagtcctgcagtgagagtcttg

SEQ ID NO: 714 - DNA, 50 bp, Homo sapiens
cggaggccaggccacgcagaacctcacagaccaaagctgcttcgcagtcg

SEQ ID NO: 715 - DNA, 50 bp, Homo sapiens
cctgcgggactaccagaaggtgctagtgcagctggagagcctggagaccg

SEQ ID NO: 716 - DNA, 50 bp, Homo sapiens
cggccgggactctccatgtcctatggactctcgctgaagccactcggccc

SEQ ID NO: 717 - DNA, 50 bp, Homo sapiens; identical to SEQ ID NO: 302

SEQ ID NO: 718 - DNA, 50 bp, Homo sapiens
gcggtgtgcactgtgacccgccttcccgggaaagtcagagcacagaagcg

SEQ ID NO: 719 - DNA, 50 bp, Homo sapiens
cgcccctgcctgctcgctgcactccaggtcacagttggcacagcgcaccg

SEQ ID NO: 720 - DNA, 50 bp, Homo sapiens
gaggagtgggtgggagacacgcgtgtccatgccgcgagggtgtgtcgacg

SEQ ID NO: 721 - DNA, 50 bp, Homo sapiens
cgccctcggagcccctgcagtcccctcaccgggcgctcccaaacagcccg

SEQ ID NO: 722 - DNA, 50 bp, Homo sapiens
gccgaatttctgaggtatccgggctctggtggactgaggggggcgacgcg

SEQ ID NO: 723 - DNA, 50 bp, Homo sapiens
cggggagggggtagttgtactctgcttgtacagtccttgagcccagttta

SEQ ID NO: 724 - DNA, 50 bp, Homo sapiens
tcccacttttgtgaaccttgagtttttgaaactttcactgaaaaaaaccg

SEQ ID NO: 725 - DNA, 50 bp, Homo sapiens
cgcaggaggagttgggaggacaaaaggagaaaatgaactagttgggttac

SEQ ID NO: 726 - DNA, 50 bp, Homo sapiens; identical to SEQ ID NO: 545

SEQ ID NO: 727 - DNA, 50 bp, Homo sapiens; identical to SEQ ID NO: 547

SEQ ID NO: 728 - DNA, 50 bp, Homo sapiens
tcttttaacccagctaacacttctgttaatagtgtcttcagccagatgcg

SEQ ID NO: 729 - DNA, 50 bp, Homo sapiens
ggggcaggacatctcccagacagctggggacaccctttccagcgggagcg

SEQ ID NO: 730 - DNA, 50 bp, Homo sapiens
cggtggggacacagaaccaaaccatatcgtttggtattccagaatttctg

SEQ ID NO: 731 - DNA, 50 bp, Homo sapiens
tctttgcaacgtccctcaagcacttaaggagaggcggcagggcccaagcg

SEQ ID NO: 732 - DNA, 50 bp, Homo sapiens
agcaggacaggcacccgctggccgggaggtcattgactggcagaacgacg

SEQ ID NO: 733 - DNA, 50 bp, Homo sapiens
ggaggaggacttgaatacacgcgtcgctgactcaaagatggtcccctccg

SEQ ID NO: 734 - DNA, 50 bp, Homo sapiens
cccggagcagtgggttcctctgccgggccaggagggtgatggagggaccg

SEQ ID NO: 735 - DNA, 50 bp, Homo sapiens
cgcgccggctgacgtcaccctgtgtcgaccagccccctgagcccggaaag

SEQ ID NO: 736 - DNA, 50 bp, Homo sapiens
gaagcacaaaggcgacgtgactcctagaatctgtccagaacccatggtcg

SEQ ID NO: 737 - DNA, 50 bp, Homo sapiens
gtgacagctgagctgagacctcaagacacagtcacctgggccaccttccg

SEQ ID NO: 738 - DNA, 50 bp, Homo sapiens
cttatgcaaatcacttaggtacatgcaaaagtatcccttctcccggagcg

SEQ ID NO: 739 - DNA, 50 bp, Homo sapiens
cgcgccgcatctccgggaacaacagtctccctcccgagcatcacagccag

SEQ ID NO: 740 - DNA, 50 bp, Homo sapiens
cgctcctcctagctagcggccgccgcccgccgccgcctgcgcctccagct

SEQ ID NO: 741 - DNA, 50 bp, Homo sapiens
cggccccagcctgcccagggcccagctgctggagacccgcagctcgtccc

SEQ ID NO: 742 - DNA, 50 bp, Homo sapiens
cggagggctcaggttacaccccataggagggttcaggtgtccccacatct

SEQ ID NO: 743 - DNA, 50 bp, Homo sapiens
ctccagctggcgcactacaaggtaagaactgaccgcctagggttttgtcg

SEQ ID NO: 744 - DNA, 50 bp, Homo sapiens; identical to SEQ ID NO: 563

SEQ ID NO: 745 - DNA, 50 bp, Homo sapiens
cgcgcagttaaaaaagaactcttactttggttggttggaaaagagctgat

SEQ ID NO: 746 - DNA, 50 bp, Homo sapiens
gtcagagttcgggtgtctgtgggtctctgagcctctgctggcagcacccg

SEQ ID NO: 747 - DNA, 50 bp, Homo sapiens
cgcagctcaagaccctggcgcagttccccaacacgctgctgggcaaccct

SEQ ID NO: 748 - DNA, 50 bp, Homo sapiens
cggctggcaggggccctgtgcccccaggtaccccgtacccctgccaaagt

SEQ ID NO: 749 - DNA, 50 bp, Homo sapiens
cgcaagactaggcaacctccagccagtccctgggtcgggcggatcctccc

SEQ ID NO: 750 - DNA, 50 bp, Homo sapiens
aacatgagacaactgaagtgccctggtttatgtgccctgctcctcctccg

SEQ ID NO: 751 - DNA, 50 bp, Homo sapiens
atttcacagataaggaaactgaggcccaagatggcacagtggcctcgacg

SEQ ID NO: 752 - DNA, 50 bp, Homo sapiens
gagacatggcgacggcgaaggtggcagcctggagccttgggctgaggtcg

SEQ ID NO: 753 - DNA, 50 bp, Homo sapiens
cgcccagcccagattttggcagtgccgaagctccacactttcctaagcag

SEQ ID NO: 754 - DNA, 50 bp, Homo sapiens; identical to SEQ ID NO: 246

SEQ ID NO: 755 - DNA, 50 bp, Homo sapiens
attgtctcccaatttcttcgtctgtaaaatggggctggcagcagtgcccg

SEQ ID NO: 756 - DNA, 50 bp, Homo sapiens
cagccactactccagactttgttcctacaactgacgattgccagaatccg

SEQ ID NO: 757 - DNA, 50 bp, Homo sapiens
cggatccgtttgaaacctgtccggtccctgctttctatgccaggcaaaaa

SEQ ID NO: 758 - DNA, 50 bp, Homo sapiens
cgtggggtagggactttcatcaggacatagagtggcagtcatgtgcttcg

SEQ ID NO: 759 - DNA, 50 bp, Homo sapiens
agtgtccgagtaagcctaatgacacacattcacataactaagaagacacg

SEQ ID NO: 760 - DNA, 50 bp, Homo sapiens
agtccacgtgtcagaggtgtttgaaccagagcaaccctatcttaaatacg

SEQ ID NO: 761 - DNA, 50 bp, Homo sapiens
actttctccttcttgtttttattcggcatggctggagcgggccgccgccg

SEQ ID NO: 762 - DNA, 50 bp, Homo sapiens
cgcagagagatgtaccaacactgtgctcctgagtgacttataaagagcca

SEQ ID NO: 763 - DNA, 50 bp, Homo sapiens
tttaagttaaaattgcccagacgtggtattctgaatctgggtgattggcg

SEQ ID NO: 764 - DNA, 50 bp, Homo sapiens
cgctcctggttgtcacattctggctcttttatggggtgggaaatctttca

SEQ ID NO: 765 - DNA, 50 bp, Homo sapiens
cgctgccaggaggagccgaggcgagtgggcgtggctctggcgagtgcccg

SEQ ID NO: 766 - DNA, 50 bp, Homo sapiens
cgccagccccaagcaagccaggcagtgtcctggtgtggcagctcacaggc

SEQ ID NO: 767 - DNA, 50 bp, Homo sapiens
tcatccaggctgcagtgcaatggagcgatcttggcttaccataacttccg

SEQ ID NO: 768 - DNA, 50 bp, Homo sapiens
gtggtggagatgcctgacatggcttggggacctgctgccgggggagctcg

SEQ ID NO: 769 - DNA, 50 bp, Homo sapiens
gtgcagggggagctggcgcagctcaaggcctgggtgaggaagctgcagcg

SEQ ID NO: 770 - DNA, 50 bp, Homo sapiens
cggggctctcccagatgcccacgctgcctgctcccactggagacggggac

SEQ ID NO: 771 - DNA, 50 bp, Homo sapiens
aattggaatgtggcagggaaacgctgggagacccctccccatccttctcg

SEQ ID NO: 772 - DNA, 50 bp, Homo sapiens
cggcaacatcactccagagagcccagcagcttcctgggagaggcaggctg

SEQ ID NO: 773 - DNA, 50 bp, Homo sapiens
gagggtcaggtctgtgctgggggtgaggtccatgctgggggtcaggtccg

SEQ ID NO: 774 - DNA, 50 bp, Homo sapiens
gtctggtcaagagagatccagacaaaatcaaccttctgcgtcaacagccg

SEQ ID NO: 775 - DNA, 50 bp, Homo sapiens
cgcagggcactcggagcaggtctctggctgcctggcacaggaagggcctc

SEQ ID NO: 776 - DNA, 50 bp, Homo sapiens
cgctgatactgacatttacagatgaaaccgtctgagacccgcttcccagc

SEQ ID NO: 777 - DNA, 50 bp, Homo sapiens
agggtgtcggcagcaagatggctccgggggtttagacactgctggcttcg

SEQ ID NO: 778 - DNA, 50 bp, Homo sapiens
cgcaccccagccagagagggaatactgcacctcccagaatgtggcctggg

SEQ ID NO: 779 - DNA, 50 bp, Homo sapiens
aagtacccctgccgctattgtgagaaagtgtttgctctggcggagtaccg

SEQ ID NO: 780 - DNA, 50 bp, Homo sapiens
tcaggagaaatttctaactgtaaccaaagggagcaggcaggacccagacg

SEQ ID NO: 781 - DNA, 50 bp, Homo sapiens
cgcggctcaaccaagcctagaagtgggggcggggaatcttccgccctcct

SEQ ID NO: 782 - DNA, 50 bp, Homo sapiens
cggtattgtcatgttgcatcaaggttcctcattgcagacagaaatctgcc

SEQ ID NO: 783 - DNA, 50 bp, Homo sapiens; identical to SEQ ID NO: 597

SEQ ID NO: 784 - DNA, 50 bp, Homo sapiens; identical to SEQ ID NO: 188

SEQ ID NO: 785 - DNA, 50 bp, Homo sapiens
agctgaaggtcttgaggcgcaggctggtgtccagggggcggctcatcccg

SEQ ID NO: 786 - DNA, 50 bp, Homo sapiens; identical to SEQ ID NO: 601

SEQ ID NO: 787 - DNA, 50 bp, Homo sapiens
cgcacccagccctttacaagcagcacaaacgtatttggttgtctggggcc

SEQ ID NO: 788 - DNA, 50 bp, Homo sapiens
caggaggcatgtgggggcctctgccaggcactgcacaggagggcgcgtcg

SEQ ID NO: 789 - DNA, 50 bp, Homo sapiens
gcctcgcgggattaggggccggtagaggtgggcgggtcgggcggaagtcg

SEQ ID NO: 790 - DNA, 50 bp, Homo sapiens
cgccgaggcccgctgcccgcccccgcccgcccccgcccgcccgcctcacc

SEQ ID NO: 791 - DNA, 50 bp, Homo sapiens
cgcccggctggctagaccattgctaatggctcaagagttcagttctagca

SEQ ID NO: 792 - DNA, 50 bp, Homo sapiens
cgcctgcaattaaaggccagattggaggcatctgatccccagccctgatt

SEQ ID NO: 793 - DNA, 50 bp, Homo sapiens
tgttggacctctaaacggtaattacgggcgacaacgcagatccaggatcg

SEQ ID NO: 794 - DNA, 50 bp, Homo sapiens
gagcagcagcagcatgtacctggccatgctggacggagatgctttgaacg

SEQ ID NO: 795 - DNA, 50 bp, Homo sapiens
gcagccgcagagacccttgggaagtgtggtcttcgggtgccggcggttcg

SEQ ID NO: 796 - DNA, 50 bp, Homo sapiens
cggaagggatgttgttttgcttccgcagacacctgacctccaacttccta

SEQ ID NO: 797 - DNA, 50 bp, Homo sapiens
cgggaatggtgctgtctgcccagccccacccggccttgtaatgaacactt

SEQ ID NO: 798 - DNA, 50 bp, Homo sapiens
tgcctgcaaactaaggcagcattattcagacctgggctaccctggtcccg

SEQ ID NO: 799 - DNA, 50 bp, Homo sapiens
cggtctggttagaaaatatccagccccacagcaaggggaaagagtaacat

SEQ ID NO: 800 - DNA, 50 bp, Homo sapiens
cggatttctgaaaatgtggttattccagagaattccttcacgagatttaa

SEQ ID NO: 801 - DNA, 50 bp, Homo sapiens
cgggtgcttccaaccagtctgctgctcctgccccacccgtgcacgacagc

SEQ ID NO: 802 - DNA, 50 bp, Homo sapiens; identical to SEQ ID NO: 268

SEQ ID NO: 803 - DNA, 50 bp, Homo sapiens
gtgggcgtaggaccgtctgagccatgtgtgggatataatctcctggtgcg

SEQ ID NO: 804 - DNA, 50 bp, Homo sapiens
cgccggaggccgccacaacgcaggcgcattcagctaaggaccactccctc

SEQ ID NO: 805 - DNA, 50 bp, Homo sapiens
cttgtggccatctctgtaaagtttctggggaggtgggcacggggtaaacg

SEQ ID NO: 806 - DNA, 50 bp, Homo sapiens
gcatggcaagaagcacccccaacacccagttctcctggagacactgatcg

SEQ ID NO: 807 - DNA, 50 bp, Homo sapiens
caatacaaattaataacaaccagccaattccattgtccttgtatgcatcg

SEQ ID NO: 808 - DNA, 50 bp, Homo sapiens
cgcgggcaggtgctgctggtgggcgagctgtgggagcgcgaacagagccg

SEQ ID NO: 809 - DNA, 50 bp, Homo sapiens
cgggacagcatgtcactccgggaagcctcccgttcaggtgtctggtccct

SEQ ID NO: 810 - DNA, 50 bp, Homo sapiens
agccaggaagcagttggcaaaggggcggggtgacgcggtgacgcaatccg

SEQ ID NO: 811 - DNA, 50 bp, Homo sapiens
cggaacatgtttgggtatagggagccatcagactgcagtgcacacctgac

SEQ ID NO: 812 - DNA, 50 bp, Homo sapiens
cggcccctggatcctaattcagaccagcctgcctctggcaataggaaagt

SEQ ID NO: 813 - DNA, 50 bp, Homo sapiens
tctagtgtctctagggccacctcactttcagcagtctgtcccaggtgtcg

SEQ ID NO: 814 - DNA, 50 bp, Homo sapiens
tacatctgctcccctgggctttctcaattcctaaataatgttgctgttcg

SEQ ID NO: 815 - DNA, 50 bp, Homo sapiens
ggaatgggaggaacgcaatgcgtcccgcgcggcgatctgggatagctgcg

SEQ ID NO: 816 - DNA, 50 bp, Homo sapiens
cgggagctgtcaatcacgaacaccagatccaggggcccagtgtgacacct

SEQ ID NO: 817 - DNA, 50 bp, Homo sapiens
cgctctcctagagagggcttctgatttggagctgttgttttggtctgaga

SEQ ID NO: 818 - DNA, 50 bp, Homo sapiens
cgggcccctggggcctcagagggaccccggctgccactgacatatgaaga

SEQ ID NO: 819 - DNA, 50 bp, Homo sapiens
agtggctgcagcatgggtcaaggtcagagaatccgggagccccacagccg

SEQ ID NO: 820 - DNA, 50 bp, Homo sapiens
aagcgtacgctctgtgccagtccttaggtaaaatgttttactttgtgccg

SEQ ID NO: 821 - DNA, 50 bp, Homo sapiens
cgccagcccgcggagccccagtgactcagagccgtggctcccctggcccg

SEQ ID NO: 822 - DNA, 50 bp, Homo sapiens
cggctcaggaccagcaggcatgcctccttggtctcatgttgaccttagag

SEQ ID NO: 823 - DNA, 50 bp, Homo sapiens
cgcgactcacccacggtggccgggcagagcctgcctcgaacccggcttct

SEQ ID NO: 824 - DNA, 50 bp, Homo sapiens
ggaggaggctttctgctggctagtacttttccaactctgacagtggcacg

SEQ ID NO: 825 - DNA, 50 bp, Homo sapiens
gaagcacttggacaggccatcgggccagaagcacctcgcaagggtgtccg

SEQ ID NO: 826 - DNA, 50 bp, Homo sapiens
tacttgctcagggctttcttcccgcgtgtgcttggcgctgctcacaaacg

SEQ ID NO: 827 - DNA, 50 bp, Homo sapiens
agatgcctaattcagccgggagccgcgggccggagctggctgaaggtccg

SEQ ID NO: 828 - DNA, 50 bp, Homo sapiens
cataaaccagatgtttcttaaaatagcccagttaaatccacccttcctcg

SEQ ID NO: 829 - DNA, 50 bp, Homo sapiens
cgggcggggaaagcgaggtttaactaactccgcagctatattcttccgcc

SEQ ID NO: 830 - DNA, 50 bp, Homo sapiens
cgccaccccacactccctgcctggggaagtcggacgccgccccacacccc

SEQ ID NO: 831 - DNA, 50 bp, Homo sapiens
cagagagcaaatccttcttcctgccctaggctgcagtgctcctaacctcg

SEQ ID NO: 832 - DNA, 50 bp, Homo sapiens
taaaccgagttaatcatcggtccatttgaataatcagtcacgtggtggcg

SEQ ID NO: 833 - DNA, 50 bp, Homo sapiens
caagcagccaagctgctccacgattggctgctttagccttacgtgcgtcg

SEQ ID NO: 834 - DNA, 50 bp, Homo sapiens
gcaaaccgtgaatgtggggcagtgtggggccaagcggtgcgcttggtacg

SEQ ID NO: 835 - DNA, 50 bp, Homo sapiens
tttctcgaggcagggggcacggtagcacagggagcttctctttgtgggcg

SEQ ID NO: 836 - DNA, 50 bp, Homo sapiens
cgacagccaccttatgtggaaaatactcgcgccggccgtcattcataatc

SEQ ID NO: 837 - DNA, 50 bp, Homo sapiens
cggctcagagcacaggctcagaaatcagactgcccacgtttgaaagctgg

SEQ ID NO: 838 - DNA, 50 bp, Homo sapiens
ggcggcgctgggcattctctgccatccttaggggtcctccacagcatgcg

SEQ ID NO: 839 - DNA, 50 bp, Homo sapiens
cgcacgaacccttcatcctgctccaaacagaatgagaatgtcatgtcgca

SEQ ID NO: 840 - DNA, 50 bp, Homo sapiens
cgctgcccaagtctcagatattcgatgcctcacattgccaatatggagtc

SEQ ID NO: 841 - DNA, 50 bp, Homo sapiens; identical to SEQ ID NO: 654

SEQ ID NO: 842 - DNA, 50 bp, Homo sapiens
catggatttaatcagtgtactttttaccttcaggctgtaaattagaaacg

SEQ ID NO: 843 - DNA, 50 bp, Homo sapiens
cgagatttttaacctgaaactgaagaatctggtactgtaagtgtaaagaa

SEQ ID NO: 844 - DNA, 50 bp, Homo sapiens
cgccgagggctccaggcctcgcttccagccatataggtagtaggccgaga

SEQ ID NO: 845 - DNA, 50 bp, Homo sapiens
cgcctgctctgtgccccactacgcttcatgcaggggtccatgtgcagctc

SEQ ID NO: 846 - DNA, 50 bp, Homo sapiens
cagtaactccggccgaatttgttcgggtgtgaggtttaaggccctaatcg

SEQ ID NO: 847 - DNA, 50 bp, Homo sapiens
gtgactggccaaaaaaaaccgcaattttggggtctaattcgattgtgacg

SEQ ID NO: 848 - DNA, 50 bp, Homo sapiens
gggcggtcaggcggggtcaaggccaggcagcggggcgcgtctgcgttgcg

SEQ ID NO: 849 - DNA, 50 bp, Homo sapiens
ctattggttgagttaaccagcattcaaaagcagtcagcgaaccaactacg

SEQ ID NO: 850 - DNA, 50 bp, Homo sapiens
cggacaggttttaaacaacctaccaagagccagcgtgggtgtccacaccc

SEQ ID NO: 851 - DNA, 50 bp, Homo sapiens
cggggtgaacgtctgggtcctcggaaatcctcagtctctggaactgaatt

SEQ ID NO: 852 - DNA, 50 bp, Homo sapiens; identical to SEQ ID NO: 208

SEQ ID NO: 853 - DNA, 50 bp, Homo sapiens
gggaggccttggttggagtgtctgaggcctcagctgggcccctgcctccg

SEQ ID NO: 854 - DNA, 50 bp, Homo sapiens
cgcagcagccagctcatgggccccgcatccaggtgtgtcctgccctcttc

SEQ ID NO: 855 - DNA, 50 bp, Homo sapiens
cgctgggcactgccctgggggctgagcagctgtctccattggtcattcag

SEQ ID NO: 856 - DNA, 50 bp, Homo sapiens
cggaactcgctggaagctgtgatagtcccagctatggtttatcacaggga

SEQ ID NO: 857 - DNA, 50 bp, Homo sapiens
gagctgcagctggggactggtgcgccatgcaattgacatagcgctgctcg

SEQ ID NO: 858 - DNA, 50 bp, Homo sapiens
tacttcagggtaaagaagttcaggtctgccatctccagctctgtctggcg

SEQ ID NO: 859 - DNA, 50 bp, Homo sapiens
cggcggcttccccaggtgctgctccgcccagtgcccccccagatctcaac

SEQ ID NO: 860 - DNA, 50 bp, Homo sapiens
agcatgaccagaatggttcctcagaatcaggaggtgaaacctaacattcg

SEQ ID NO: 861 - DNA, 50 bp, Homo sapiens
agagtggcgcgagcctgcgttttccggccagaggacatggtgcgttttcg

SEQ ID NO: 862 - DNA, 50 bp, Homo sapiens
ggatcccaaataacagatctgggagaatcctgcctcagtacttggatgcg

SEQ ID NO: 863 - DNA, 50 bp, Homo sapiens
cgcccacagggcccagtgggctctgagtaaggtgcacggcacacaccagc

SEQ ID NO: 864 - DNA, 50 bp, Homo sapiens
gcctgggatcatccccaagtgggtctagggtgggcctgtgaggaggagcg

SEQ ID NO: 865 - DNA, 50 bp, Homo sapiens
cgactgcaggcctacggcttcctttgagggctcacgtttacaagataaca

SEQ ID NO: 866 - DNA, 50 bp, Homo sapiens
cggccgccaaccgaccactgactgtctgccaggcatccgggctccctcca

SEQ ID NO: 867 - DNA, 50 bp, Homo sapiens
ggcctccagagctgggtaagtggagggctctgtgtatgttgctgtacacg

SEQ ID NO: 868 - DNA, 50 bp, Homo sapiens
cgcccatgcccgcgctagtgggaggaggctggaaagcgccttcccatttt

SEQ ID NO: 869 - DNA, 50 bp, Homo sapiens
cgggcagatacgagcagattgactcgccaggactgtcattgggccaccgc

SEQ ID NO: 870 - DNA, 50 bp, Homo sapiens
cggcgctgacgccgcaaggccacttctatcgctcccgaccacggtctgtt

SEQ ID NO: 871 - DNA, 50 bp, Homo sapiens
cgcttgtcagagtcaccatctactaccatgtatttcaggcatggggcaaa

SEQ ID NO: 872 - DNA, 50 bp, Homo sapiens
cgaggtgctggtgttcatacagataaggctctcttcattgcaacagccac

SEQ ID NO: 873 - DNA, 50 bp, Homo sapiens
tcatttgcaaatgttactgggggacacaccggctcccagtagggtttccg

SEQ ID NO: 874 - DNA, 50 bp, Homo sapiens
cgcaagagaagagggagcacgcccagaagctgataatgctgcagaacctg

SEQ ID NO: 875 - DNA, 50 bp, Homo sapiens
cgctgtggcagcccaaacgtcagaggcacctcactttgccatggcctggc

SEQ ID NO: 876 - DNA, 50 bp, Homo sapiens
cggcccagtgaacctgagctttggagtttggacgaatagctagtttacct

SEQ ID NO: 877 - DNA, 50 bp, Homo sapiens
cagggactgacgcccctctagcaaagcatctgtggtacatcccagacccg

SEQ ID NO: 878 - DNA, 50 bp, Homo sapiens
gatctcacactttggagacatacccctaagtagaccagaggcatcacacg

SEQ ID NO: 879 - DNA, 50 bp, Homo sapiens
cgcctggagaagcagtgccaagtctattccctaataaagtacttgttgtg

SEQ ID NO: 880 - DNA, 50 bp, Homo sapiens
cggagctgcagaaagaaagggaagaaactaacgttctttctgcgtctggg

SEQ ID NO: 881 - DNA, 50 bp, Homo sapiens
cgctaaaactcacagcctgtggacaaagcctttttggttttcaaatcggc

SEQ ID NO: 882 - DNA, 50 bp, Homo sapiens
cggtcaaaattaaaaacttctgcactgaaaagaaaaaagggagagagaga

SEQ ID NO: 883 - DNA, 50 bp, Homo sapiens
cgcccccaagaagctccccacccccaaaccagaactggtggctggtgact

SEQ ID NO: 884 - DNA, 50 bp, Homo sapiens
attgaatgccaaagttactggtccccaggcagcagctagcacattcaacg

SEQ ID NO: 885 - DNA, 50 bp, Homo sapiens
cgagtggctactgtattgaacaacacagatctagaaacagagtgtggacc

SEQ ID NO: 886 - DNA, 50 bp, Homo sapiens
cgggaacctccacaggctaaggtgagtgccagcactggcaccagccctgg

SEQ ID NO: 887 - DNA, 50 bp, Homo sapiens
caaaggcagacatcgcttcaggtaggttgcagccaaaatggcttccaacg

SEQ ID NO: 888 - DNA, 50 bp, Homo sapiens
ttctttgtggccaagaaattcccaccttaccacttacagggaggccatcg

SEQ ID NO: 889 - DNA, 50 bp, Homo sapiens
cgcagtgtgaaagatgcgagccatagcatctcgtaacatgaaggccatcc

SEQ ID NO: 890 - DNA, 50 bp, Homo sapiens
tattttgtatttggcttgctctggtttctgctggagcttttaaaaaatcg

SEQ ID NO: 891 - DNA, 50 bp, Homo sapiens
cgggattacctagtgccttcacaatcggtcagagctggattcagattcct

SEQ ID NO: 892 - DNA, 50 bp, Homo sapiens
cgggttactactgtacctaccagctgattccagcagccttttctccagcg

SEQ ID NO: 893 - DNA, 50 bp, Homo sapiens
accgttttttcccccgaataaagtgtgcccgtgtagctgtacagatttcg

SEQ ID NO: 894 - DNA, 50 bp, Homo sapiens
tctttcttcacgtcttgttggttactgctacctggcttccaactgttgcg

SEQ ID NO: 895 - DNA, 50 bp, Homo sapiens
agctggccagactgctgcagttttcagtcccaggaactgagaacgtggcg

SEQ ID NO: 896 - DNA, 50 bp, Homo sapiens
ggatttggtctaggctttctggcttggccctggaactctcagcaaggacg

SEQ ID NO: 897 - DNA, 50 bp, Homo sapiens
cgcaggttacttcctataaatggaagaacctgaatcattaaaagataaaa

SEQ ID NO: 898 - DNA, 50 bp, Homo sapiens
taaggcctcggccatgactgtgggtgggtttaactcagctagcagctgcg

SEQ ID NO: 899 - DNA, 50 bp, Homo sapiens
cgagactaagaagtgacccaacctaggtgacccttcagctaggtcactct

SEQ ID NO: 900 - DNA, 50 bp, Homo sapiens
cgcccataaggatataagggagacagagggaaaaaggaggaagctcactc

SEQ ID NO: 901 - DNA, 50 bp, Homo sapiens
gcaggcgggcctcaggggctgtctttcctgcacggctgttgtgtgcttcg

SEQ ID NO: 902 - DNA, 50 bp, Homo sapiens
cggcaccacacagctagtgccggaactttttgtgcagagaaagaagaccc

SEQ ID NO: 903 - DNA, 50 bp, Homo sapiens
ggtagggtggggccaggctgagcctgctctgcgggtcccggaccccagcg

SEQ ID NO: 904 - DNA, 50 bp, Homo sapiens
cgtgcaggccacagtaatggtggtgcaggtggcaggagtaggtgccctcg

SEQ ID NO: 905 - DNA, 50 bp, Homo sapiens
ggacctgaagatctgcatgtatgaccggctctaccaggactctgtgctcg

SEQ ID NO: 906 - DNA, 50 bp, Homo sapiens
tgacactgtcttcggggcacatatggtcatctatgagtgctgtgagcacg

SEQ ID NO: 907 - DNA, 50 bp, Homo sapiens
gaggggctggcccccttcaccctgtgatcgacaggacagagctgggcacg

SEQ ID NO: 908 - DNA, 50 bp, Homo sapiens
atgcatgtttcacaccttctagaactcagagtaaacctagctcttcaacg

SEQ ID NO: 909 - DNA, 50 bp, Homo sapiens
gttaacaactgtcaacagcctatagaagatcaaggctaaattccctaacg

SEQ ID NO: 910 - DNA, 50 bp, Homo sapiens
cgagtttagcttttaaagaatcctttgaagaaggattgcaacgtgactaa

SEQ ID NO: 911 - DNA, 50 bp, Homo sapiens
tagagaaatacattgtaacaatttggtttctttcctgatggcctttatcg

SEQ ID NO: 912 - DNA, 50 bp, Homo sapiens
attacaagcatgagccagggcacccagccagaaatgatatttaaactacg

SEQ ID NO: 913 - DNA, 50 bp, Homo sapiens
ccatgtataagtccccaggactcacaaatctcctctttcaggatatctcg

SEQ ID NO: 914 - DNA, 50 bp, Homo sapiens
cgttaggtcccacggcttgccctgctcagcacctaataaggccattagct

SEQ ID NO: 915 - DNA, 50 bp, Homo sapiens
cgggggctaagccagcgccgtctgcctctccacctagaccagaccccttc

SEQ ID NO: 916 - DNA, 50 bp, Homo sapiens
cggggggtgccaaaaagccctccttctgacggccctcctggagcccaaca

SEQ ID NO: 917 - DNA, 50 bp, Homo sapiens
cgctttacggaaagaacgccccgcgaataaatccggagcggatttgcatc

SEQ ID NO: 918 - DNA, 50 bp, Homo sapiens
cggagccacagagaacacttttgagaccagcagacaaccacagatgtgcg

SEQ ID NO: 919 - DNA, 50 bp, Homo sapiens; identical to SEQ ID NO: 539

SEQ ID NO: 920 - DNA, 50 bp, Homo sapiens
ttcctatcacaatttacaaaaagcctccaaaaaaccttgaaaagcttacg

SEQ ID NO: 921 - DNA, 50 bp, Homo sapiens
caagggaagctcccgcctcagggtctctgtcttcctgtggttaagagacg

SEQ ID NO: 922 - DNA, 50 bp, Homo sapiens
cgctccggctggtaatttccaaacacagagggaagagcatggtggagcaa

SEQ ID NO: 923 - DNA, 50 bp, Homo sapiens
cggaactgtcctcgacccgtgggtacagtattttctcgtatgccaccaag

SEQ ID NO: 924 - DNA, 50 bp, Homo sapiens
ttggtgcagttttgagcgtccgtgtataaggagtggctccgtagtcaccg

SEQ ID NO: 925 - DNA, 50 bp, Homo sapiens
ccatcagtagagcaggagtcgttggctcaatgcagtgctgtctgcaaccg

SEQ ID NO: 926 - DNA, 50 bp, Homo sapiens
cagtgttcggcagaggacaatctgttcgagaataattagattccccctcg

SEQ ID NO: 927 - DNA, 50 bp, Homo sapiens
cggatgcttaattacaaaaggttttgcccctgtagtgaccgggcagcaat

SEQ ID NO: 928 - DNA, 50 bp, Homo sapiens
cagctcccacctgtgcagagtggcggcctgggagctgagcgtggccctcg

SEQ ID NO: 929 - DNA, 50 bp, Homo sapiens
cgcgcagcttccttacaggaggctttgaatgctgcctggagtatatttga

SEQ ID NO: 930 - DNA, 50 bp, Homo sapiens
cgcttaacaatgagcggggctatatgtcttgcgcttgctctgaaggaagg

SEQ ID NO: 931 - DNA, 50 bp, Homo sapiens
cgccgggagctgctcgcccagtgtcagcagagcctggcccaggccatgac

SEQ ID NO: 932 - DNA, 50 bp, Homo sapiens
cgggtgagtgtttaactttcagcccagcttaagatctccgaagacaccca

SEQ ID NO: 933 - DNA, 50 bp, Homo sapiens
gcagcgtgcctacgctttgttgcaggagcaggttggagggacgctggacg

SEQ ID NO: 934 - DNA, 50 bp, Homo sapiens
aggatgttcagtgactgcagccaggcctacctggagagctttttggagcg

SEQ ID NO: 935 - DNA, 50 bp, Homo sapiens
cggcttctgaactttgtactttgcgagtgtactttgtactttggtcctgt

SEQ ID NO: 936 - DNA, 50 bp, Homo sapiens
cggcctcttatctcgacaatcggttgctcttggccagcccgtcctgctgc

SEQ ID NO: 937 - DNA, 50 bp, Homo sapiens
cgcttatgcccagctgctccagctggaaaagacaggactgcaagtgggtt

SEQ ID NO: 938 - DNA, 50 bp, Homo sapiens
cggactttctgcgcagagaaaacactatctcctttggaggctgtgcactt

SEQ ID NO: 939 - DNA, 50 bp, Homo sapiens
cgctgattgcactgtcaagaaggaaaatactgtttgaaaagacaacagga

SEQ ID NO: 940 - DNA, 50 bp, Homo sapiens
aacaaaccattacgtgttccaattagagcggcagcggacctggctggccg

SEQ ID NO: 941 - DNA, 50 bp, Homo sapiens
cggcagtgagtcacttacatagatgtgtgtaacatgccccagagacctca

SEQ ID NO: 942 - DNA, 50 bp, Homo sapiens; identical to SEQ ID NO: 563

SEQ ID NO: 943 - DNA, 50 bp, Homo sapiens
agcgacacttatgtcctcaatttattgctacgttcactcggtcagagtcg

SEQ ID NO: 944 - DNA, 50 bp, Homo sapiens
ttcatgccggacaaatttccctatgaacaatgcagccaagggctgcatcg

SEQ ID NO: 945 - DNA, 50 bp, Homo sapiens
cgtgggcagagtggttggctgccccactgtcagcctgttctgaaagtagg

SEQ ID NO: 946 - DNA, 50 bp, Homo sapiens
tgcagttcttataataaacacttaaacagggccaggcactgcagttcacg

SEQ ID NO: 947 - DNA, 50 bp, Homo sapiens
aggactagatgttcaccttaaatccagccactgctccagactgcaaatcg

SEQ ID NO: 948 - DNA, 50 bp, Homo sapiens
cgcccttttccggtgtcctgggctcaacagggataaaaggaggcagcact

SEQ ID NO: 949 - DNA, 50 bp, Homo sapiens
ggtggggactggcattaggggtgggcgggagagaccctaaaaatccaccg

SEQ ID NO: 950 - DNA, 50 bp, Homo sapiens
cgtgtgtttgtaattgtgtatggtcctgctaatgtttgtgtttatcactg

SEQ ID NO: 951 - DNA, 50 bp, Homo sapiens
cggttcacagcatgggagcatctgtgctataccaagattactcataggat

SEQ ID NO: 952 - DNA, 50 bp, Homo sapiens
cggggctgtcaagtgtctttaaaaggtcccttcagggtggatggaaacca

SEQ ID NO: 953 - DNA, 50 bp, Homo sapiens
gcccccagctctcaaggccggtgcaaggatgggctgtgctcagcgatgcg

SEQ ID NO: 954 - DNA, 50 bp, Homo sapiens
tcctcagactgttgggatgatgagcggacggtgaactcccaccacctgcg

SEQ ID NO: 955 - DNA, 50 bp, Homo sapiens
agcgggagtcacttcggagtggggtgttgacagcgtgaagcccctgagcg

SEQ ID NO: 956 - DNA, 50 bp, Homo sapiens
cgctttcaggggtcaaagacctggcagaaatgacttcccaaccccagatg

SEQ ID NO: 957 - DNA, 50 bp, Homo sapiens
taattgccctcctgtttcctagaaagcttatagggcagcagtaggaaacg

SEQ ID NO: 958 - DNA, 50 bp, Homo sapiens
cgcagaacagcacagtgtcacatggtcttcacggccctgagggcccaact

SEQ ID NO: 959 - DNA, 50 bp, Homo sapiens
cggggaccaccagctgctttgcccatggattggaacgcattgggtggagt

SEQ ID NO: 960 - DNA, 50 bp, Homo sapiens
gctgttcctcatcactcagtacccagtttgctgacgtctacaatattccg

SEQ ID NO: 961 - DNA, 50 bp, Homo sapiens
cgtgattgcacgtatcaagatgtgtgctcttagatttgttttcaatccag

SEQ ID NO: 962 - DNA, 50 bp, Homo sapiens
cggcgctcagcaaatttagatgaatgcaacgcttcaacgtgggctctcga

SEQ ID NO: 963 - DNA, 50 bp, Homo sapiens
gtcttgatgtgaccagagaacaccatgtacaggcaaagcccagcccctcg

SEQ ID NO: 964 - DNA, 50 bp, Homo sapiens
gctgtcgctgcaggtgggaaccgagagccaggaagcccagggagctgtcg

SEQ ID NO: 965 - DNA, 50 bp, Homo sapiens
acaaggggctcctcctttgtcctgggggagttgcatggatcctgtcttcg

SEQ ID NO: 966 - DNA, 50 bp, Homo sapiens
gttccacatagagagcctggctgcatgatcttgaggtcacctaactcccg

SEQ ID NO: 967 - DNA, 50 bp, Homo sapiens
cgcgcctggcctatacaatattttaaataattttgtgtgtgatacaatga

SEQ ID NO: 968 - DNA, 50 bp, Homo sapiens
cgcccgcctcatttgcgccttgcagcactgctggaccaggtatctgcccg

SEQ ID NO: 969 - DNA, 50 bp, Homo sapiens
cgggtcagaccgtcattggccatctcacagtcccggcgctccagagcccc

SEQ ID NO: 970 - DNA, 50 bp, Homo sapiens
cagccattggctgatttgaaagaatacacagggtgtgccagctggtgtcg

SEQ ID NO: 971 - DNA, 50 bp, Homo sapiens
cggtctcagccgtccccatggtctcagccgtccccagggtctcaggagtc

SEQ ID NO: 972 - DNA, 50 bp, Homo sapiens
cggttatatcaacatgatgcccaaacaccaggtctgcttagggtaaaact

SEQ ID NO: 973 - DNA, 50 bp, Homo sapiens
gtacaaatgggtccaggagcttcaacgagcgttccagcttcttcgtaacg

SEQ ID NO: 974 - DNA, 50 bp, Homo sapiens
cgcgcaccgccccgctccacgcccctatcaggattcgggtcctcgtgagg

SEQ ID NO: 975 - DNA, 50 bp, Homo sapiens
cctcgactgctggctgtgatgttctccaacactgctattcacagcgagcg

SEQ ID NO: 976 - DNA, 50 bp, Homo sapiens
gtcccgttcacactgatcgttccatttcctcttgtgtctgagtgggagcg

SEQ ID NO: 977 - DNA, 50 bp, Homo sapiens
cgggggttggtcagcaccacacactgtgcacagggaagatggagcctcgc

SEQ ID NO: 978 - DNA, 50 bp, Homo sapiens; identical to SEQ ID NO: 776

SEQ ID NO: 979 - DNA, 50 bp, Homo sapiens
tcgatgctggtggtctgcgtggtggttcccaacagcagttcaggggctcg

SEQ ID NO: 980 - DNA, 50 bp, Homo sapiens
ggcggctctagcctctcgggggacgttcggctactccagtggggggttcg

SEQ ID NO: 981 - DNA, 50 bp, Homo sapiens
gaacagccacgcatggtccgtgaaatgggatcatgtgcagaacagccacg

SEQ ID NO: 982 - DNA, 50 bp, Homo sapiens
cgccccaccagcccgagagctagaattgaatctgggggacccctgcggcg

SEQ ID NO: 983 - DNA, 50 bp, Homo sapiens
agcactgtagggagtggccgggatcattcgagcccacagatagtgctccg

SEQ ID NO: 984 - DNA, 50 bp, Homo sapiens
cggacagctaatcgttttagtgacaggatgagagagcccttcgtgttctg

SEQ ID NO: 985 - DNA, 50 bp, Homo sapiens
cggcctgacactctcagagatgggattcccaatccatccactctgaaatg

SEQ ID NO: 986 - DNA, 50 bp, Homo sapiens
ccggaggctgatgccttatccattaggccactgggtcaccacaggatccg

SEQ ID NO: 987 - DNA, 50 bp, Homo sapiens; identical to SEQ ID NO: 785

SEQ ID NO: 988 - DNA, 50 bp, Homo sapiens; identical to SEQ ID NO: 601

SEQ ID NO: 989 - DNA, 50 bp, Homo sapiens
cggcagagaggacaggatcctgccacccagcctcatccccatgcttcccc

SEQ ID NO: 990 - DNA, 50 bp, Homo sapiens
tcccaccttgtgaaactttgccaggtgtcgctcacaggggactgggtccg

SEQ ID NO: 991 - DNA, 50 bp, Homo sapiens
agaacatccactctcttccagccagaataccattaatacgtgtgtcagcg

SEQ ID NO: 992 - DNA, 50 bp, Homo sapiens; identical to SEQ ID NO: 189

SEQ ID NO: 993 - DNA, 50 bp, Homo sapiens
cttcctatgtgattttcttccaacattctcccatgaaaaaatgtcagacg

SEQ ID NO: 994 - DNA, 50 bp, Homo sapiens
tgggtgatggaaactgaggcacagtcatccccccagcaggtccttggacg

SEQ ID NO: 995 - DNA, 50 bp, Homo sapiens
cgggccccccagaatggaacacgagggggtggtctcttgctgggctccag

SEQ ID NO: 996 - DNA, 50 bp, Homo sapiens
cgccagcccagggctggatgttttattttggcatgccccaggtgctcttc

SEQ ID NO: 997 - DNA, 50 bp, Homo sapiens
cgcacatgcgcatccaacccgattccacgggttcccaggaggcggattcc

SEQ ID NO: 998 - DNA, 50 bp, Homo sapiens
cgggaagtctgccagttggcagtctctgacgtaaaagatagcagatactg

SEQ ID NO: 999 - DNA, 50 bp, Homo sapiens
agccccactaatagatattctgattctgttggtctggaatgggaaccgcg

SEQ ID NO: 1000 - DNA, 50 bp, Homo sapiens
ggagggcaggtccgggggctctgtgtccccactgggcccccagcactgcg

SEQ ID NO: 1001 - DNA, 50 bp, Homo sapiens
cattaaagctcagactcgggttttccgagcaggtgttgcaagcctagccg

SEQ ID NO: 1002 - DNA, 50 bp, Homo sapiens
cgggtctctaagatgtcttatgaatgcaggtcagagggtcacatgttaac

SEQ ID NO: 1003 - DNA, 50 bp, Homo sapiens
cagtggatagtgaccaactctgtgttggtcgcctcatgtttcccagcacg

SEQ ID NO: 1004 - DNA, 50 bp, Homo sapiens
agcattcgggcatcagaggtttgtggagagggcagcccagtatacaaccg

SEQ ID NO: 1005 - DNA, 50 bp, Homo sapiens
cgtctaaattacttggagttgatctttctaaacaggattgctcagacaca

SEQ ID NO: 1006 - DNA, 50 bp, Homo sapiens
gcctgaggtcaggagtaagcagacaccagcactgctctttctccaagacg

SEQ ID NO: 1007 - DNA, 50 bp, Homo sapiens
cggaaatcagcttgggggccttctagccctgcagctcagaaaagtgtcag

SEQ ID NO: 1008 - DNA, 50 bp, Homo sapiens
accgcgctcagcccgtggaatttactttataatggggaggcagatgatcg

SEQ ID NO: 1009 - DNA, 50 bp, Homo sapiens
cggttgtcagctgccggaagaagagcgtggaattgagaacacagacccct

SEQ ID NO: 1010 - DNA, 50 bp, Homo sapiens
tgggtgccagggggattatcagcgtctgcgcaaggccgatggtggcagcg

SEQ ID NO: 1011 - DNA, 50 bp, Homo sapiens
tgattggatgcaggattggcgggaaggcagcggggtcatcaaaggctgcg

SEQ ID NO: 1012 - DNA, 50 bp, Homo sapiens
cgatgagctcagccccctcagtccaggccgagagccagatgggggtcacg

SEQ ID NO: 1013 - DNA, 50 bp, Homo sapiens
gtccccctcgtttccttttagaagtcattggccgatcgttttaaaagtcg

SEQ ID NO: 1014 - DNA, 50 bp, Homo sapiens
tgggatcaggatcatggtgagatgtatgcctttcccaggaatgctagccg

SEQ ID NO: 1015 - DNA, 50 bp, Homo sapiens
actactctcctggagtggttacagttaaccaaggcaaaaccccttcttcg

SEQ ID NO: 1016 - DNA, 50 bp, Homo sapiens
cggctgttgtcctgcttacaggactccagagcagcaggaaggagctatgt

SEQ ID NO: 1017 - DNA, 50 bp, Homo sapiens
cgcactcggcctgacccacggcgaccctctgtgaccaatcatactaccaa

SEQ ID NO: 1018 - DNA, 50 bp, Homo sapiens
tattccaatttgggcctgcgtgttcggagctcctggtggcggtctccacg

SEQ ID NO: 1019 - DNA, 50 bp, Homo sapiens
cgggctttggtgaccaggtcttctgtcagtgagcacacatagcccggagg

SEQ ID NO: 1020 - DNA, 50 bp, Homo sapiens
cgcctgtgtggacgcttgtgagctgcaccttcatgctattataatggcga

SEQ ID NO: 1021 - DNA, 50 bp, Homo sapiens
gaaaacacacagatgtcaaaccagaggagctgccaagctgggtaccgacg

SEQ ID NO: 1022 - DNA, 50 bp, Homo sapiens
cgccggccccaggtagtatatttttattggcacaactgccggtatttgcc

SEQ ID NO: 1023 - DNA, 50 bp, Homo sapiens
cggaggatgtgtttccagagagctggtctatttcagactgacaggccact

SEQ ID NO: 1024 - DNA, 50 bp, Homo sapiens
cggcgggactagttcctttgggcataatttaggtgttttgtgaaatgtta

SEQ ID NO: 1025 - DNA, 50 bp, Homo sapiens
tgcttagtagctaaaggccaccttctataacacgaaatagtctacaagcg

SEQ ID NO: 1026 - DNA, 50 bp, Homo sapiens
atgccttcttggggcctggcttgctgctgacgggagcactcaggaatgcg

SEQ ID NO: 1027 - DNA, 50 bp, Homo sapiens
cgacaacagggactcggtggacctggccgagctggtgcccgcggtgggcg

SEQ ID NO: 1028 - DNA, 50 bp, Homo sapiens
acagtcgaagcgcactgctctggtgggggatgttggcggtgcaggaggcg

SEQ ID NO: 1029 - DNA, 50 bp, Homo sapiens
cgaggctaaaatgtggggccagaagataaatctaatgagagctgttgcta

SEQ ID NO: 1030 - DNA, 50 bp, Homo sapiens
cgggggtgtgtttctgtttgtgtaactgggtgtgtgtgtatctagccatc

SEQ ID NO: 1031 - DNA, 50 bp, Homo sapiens
tagaaactgatgttattcatagattccacacattgtatagaagaacatcg

SEQ ID NO: 1032 - DNA, 50 bp, Homo sapiens
cacccccagcctcgccacggctcgctccccccggggtcaggccgttaccg

SEQ ID NO: 1033 - DNA, 50 bp, Homo sapiens
cgggactgtggccaaggccctcagaggccatggggagacacgacaagacc

SEQ ID NO: 1034 - DNA, 50 bp, Homo sapiens
cgcaagcacataacaaaagccaagttaagaaggttagcgatttagttgtc

SEQ ID NO: 1035 - DNA, 50 bp, Homo sapiens
tcaccattgccacttaatggtctgaggggggcttttcctgttactgcacg

SEQ ID NO: 1036 - DNA, 50 bp, Homo sapiens
tggatcgcccaggggcgggcagtccctggaagacaggtacggagtggtcg

SEQ ID NO: 1037 - DNA, 50 bp, Homo sapiens
ggaaacgaatctgggaacgagtttgaagcgcggtcaagtttgactcatcg

SEQ ID NO: 1038 - DNA, 50 bp, Homo sapiens
cgagggtcccagtgttccacatccttgccaacgcctgttattgtctgttg

SEQ ID NO: 1039 - DNA, 50 bp, Homo sapiens
cggggtaaccccaggtcccagtgatttccatagacaaaagagatgctggt

SEQ ID NO: 1040 - DNA, 50 bp, Homo sapiens
agtcttgcctggttacaggcctgtgctctccaggcagaacaagcgcagcg

SEQ ID NO: 1041 - DNA, 50 bp, Homo sapiens
gacagttcttgggaagccggcaaactgcaaaagccccatatttccttccg

SEQ ID NO: 1042 - DNA, 50 bp, Homo sapiens
cgggctcctcaggtctgaggcagctctaccactgacttggtgaattttag

SEQ ID NO: 1043 - DNA, 50 bp, Homo sapiens
cgcctctcaagagcacgatgtaagggctccaagatgagtttgggcttccc

SEQ ID NO: 1044 - DNA, 50 bp, Homo sapiens
ggcacttttcagctctaatgctcttgctttccaaaagcaaaacagaaccg

SEQ ID NO: 1045 - DNA, 50 bp, Homo sapiens
cggggcagcacagacacccgggcaaattcatgatctctaccgagtaaata

SEQ ID NO: 1046 - DNA, 50 bp, Homo sapiens
cgtgtcctttaagaactgtaacactcaccgccagggtccacggcttcatt

SEQ ID NO: 1047 - DNA, 50 bp, Homo sapiens
cgtgaactacccagatgcagcatccgctgttggtgtgatctggaacaccc

SEQ ID NO: 1048 - DNA, 50 bp, Homo sapiens
cggaaggctgaagggggagataatgagctaaagaacaactttcaccaccc

SEQ ID NO: 1049 - DNA, 50 bp, Homo sapiens
tttagtttccttcttttattttcctggcaatagcccaccattgtatatcg

SEQ ID NO: 1050 - DNA, 50 bp, Homo sapiens
cggacactgaggccttgtaatgctgttggctgaagagaggtggttggtca

SEQ ID NO: 1051 - DNA, 50 bp, Homo sapiens
tccgcggggcctgagcagagtcggccttgtaaggcagaggtctcggtgcg

SEQ ID NO: 1052 - DNA, 50 bp, Homo sapiens
cggcctcttgtccaccttgccccgaaggcaagtctggatatgaaaagaaa

SEQ ID NO: 1053 - DNA, 50 bp, Homo sapiens
agcttctttgctttgactgttttcctcttgctgcgtctctttggtcttcg

SEQ ID NO: 1054 - DNA, 50 bp, Homo sapiens
cggattgctgcttctaagcctgagaaggtcatttaatgaactcattgagg

SEQ ID NO: 1055 - DNA, 50 bp, Homo sapiens
cgcgccagcccatgtatgccatggaaatatggctgttggaattctggtgt

SEQ ID NO: 1056 - DNA, 50 bp, Homo sapiens
cgtcttggggacgctatggctccagttctgacactcaagaaacgatggat

SEQ ID NO: 1057 - DNA, 50 bp, Homo sapiens
gggcggcttctgatctcttgcggacagctcagattagtggctcgaggccg

SEQ ID NO: 1058 - DNA, 50 bp, Homo sapiens
cttgggtgcccaggtctcttccttcctgaagaatgcggcacttgcttccg

SEQ ID NO: 1059 - DNA, 50 bp, Homo sapiens
cgggcagactgcgatgaaaccccagtttgttgggatcttgctcagctccc

SEQ ID NO: 1060 - DNA, 50 bp, Homo sapiens
ggtttccaggtagcttgatctgttccaggcctggctggggtctgccctcg

SEQ ID NO: 1061 - DNA, 50 bp, Homo sapiens
acccacctgcccgccggatctggaataatgtgactcgctggtttgtcacg

SEQ ID NO: 1062 - DNA, 50 bp, Homo sapiens
tgctgcggttggagcgctccaccaggatgtccagatgggctttgattgcg

SEQ ID NO: 1063 - DNA, 50 bp, Homo sapiens
ggggtttaaggatgattccccactccacagcctgacgcatcgactctgcg

SEQ ID NO: 1064 - DNA, 50 bp, Homo sapiens
ctacgagtcttatttcctgttaacttttttccttttgcttgatcctatcg

SEQ ID NO: 1065 - DNA, 50 bp, Homo sapiens
cggctgggggaggaggcacccacaagactgccttctaggccacaggccca

SEQ ID NO: 1066 - DNA, 50 bp, Homo sapiens
cggagggagggaggaactgctctggaaatcccctaggccagggcctgctc

SEQ ID NO: 1067 - DNA, 50 bp, Homo sapiens
tgttcctgttactcttcgtgaattttctcaccataaaactgttgccaacg

SEQ ID NO: 1068 - DNA, 50 bp, Homo sapiens
gtactttgatgctctacttggcagaatgtattgcttctggtacccacacg

SEQ ID NO: 1069 - DNA, 50 bp, Homo sapiens
gtgctacctagagaggccagcatgtgggctccacatttctgctgaagacg

SEQ ID NO: 1070 - DNA, 50 bp, Homo sapiens
cggtgcttataatacattcctgtgtgtgtgttcactttgatttacggaaa

SEQ ID NO: 1071 - DNA, 50 bp, Homo sapiens
cgtcaggtttatatctttgggatttgaatgtaaagatgttggattttttt

SEQ ID NO: 1072 - DNA, 50 bp, Homo sapiens
cgctccatgtgacagaaacatgagcgtttccaaaatgaaaacactggggg

SEQ ID NO: 1073 - DNA, 50 bp, Homo sapiens
ttcccttgcttttcccagcttccggtagcccaggcattccttgtcttgcg

SEQ ID NO: 1074 - DNA, 50 bp, Homo sapiens; identical to SEQ ID NO: 208

SEQ ID NO: 1075 - DNA, 50 bp, Homo sapiens
cggctagaggatgacccatcttacgcctatggagaacactgcggaaatca

SEQ ID NO: 1076 - DNA, 50 bp, Homo sapiens
ttgcctgtgtgtgatttatgcatgttcccaccagcaggtgtggattctcg

SEQ ID NO: 1077 - DNA, 50 bp, Homo sapiens
cgggctccgatctccggtgtcacagcaaagatccgcgtctttccgatgaa

SEQ ID NO: 1078 - DNA, 50 bp, Homo sapiens
cgcctccccacgcacttccctgtatgggttacaaagcaggtgcacatcca

SEQ ID NO: 1079 - DNA, 50 bp, Homo sapiens
cgtggcacacagagccaggtttccccgaagtagtctaacgctgttgctaa

SEQ ID NO: 1080 - DNA, 50 bp, Homo sapiens
cattggctcttcacttgttgcttggtgttttccttagtcttctgagatcg

SEQ ID NO: 1081 - DNA, 50 bp, Homo sapiens
cgggacatgaagtctaaggggattattttggagctttaaaacgtgatgac

SEQ ID NO: 1082 - DNA, 50 bp, Homo sapiens
cgggaatttggtgagagtcccccacagttctgtctgccaggcactggcga

SEQ ID NO: 1083 - DNA, 50 bp, Homo sapiens
cgcatcctccagccatgctatgcttgcaaagatgtctctggtcattggga

SEQ ID NO: 1084 - DNA, 50 bp, Homo sapiens; identical to SEQ ID NO: 677

SEQ ID NO: 1085 - DNA, 50 bp, Homo sapiens
ggaagtcctctgtgcatgcgtgtctgtgccataggcgtgggaacacctcg

SEQ ID NO: 1086 - DNA, 50 bp, Homo sapiens
cgcctaggtatgggaaatgtaagtatcctcagcagagaccacagttctct

SEQ ID NO: 1087 - DNA, 50 bp, Homo sapiens
cggtcagcactgggtgaatgggttgagggaacccacataaaatccccaag

SEQ ID NO: 1088 - DNA, 50 bp, Homo sapiens
cgatcttccccatggaagacatgcacatgggttaggagaatacaacactc

SEQ ID NO: 1089 - DNA, 50 bp, Homo sapiens
cgcaacaacttctcagctacagtaaatcaggatttggggtgataggaagg

SEQ ID NO: 1090 - DNA, 50 bp, Homo sapiens
caatgtatactatagcaagtggtaccagtgctgcgggtaccagtgctgcg

SEQ ID NO: 1091 - DNA, 50 bp, Homo sapiens
cacagatcagactgctaagtctttacacatccgaaagttagactttgacg

SEQ ID NO: 1092 - DNA, 50 bp, Homo sapiens
gccacgggagatttcatcagaagaatatcagtctattgatatgggtgccg

SEQ ID NO: 1093 - DNA, 50 bp, Homo sapiens
cctccctacaactgtaggttcccctgctgctgggctcagctgggagctcg

SEQ ID NO: 1094 - DNA, 50 bp, Homo sapiens
cgggacagccccgatgccgatgcctgtgttctgtgtccccaggtctaaag

SEQ ID NO: 1095 - DNA, 50 bp, Homo sapiens
gagcccagcggggttagggttcaggtgagggtttagggtcggggcgagcg

SEQ ID NO: 1096 - DNA, 50 bp, Homo sapiens
cgtaccccgggatctggcatttagctggcaatcaaatatgtggtgaaacg

SEQ ID NO: 1097 - DNA, 50 bp, Homo sapiens
gtttaaaattctctcttctgagtaatagtgttcatgtgctcccattctcg

SEQ ID NO: 1098 - DNA, 50 bp, Homo sapiens
actaggtaaacaccacagtaataactgttgcaggtaagaatcatcaggcg

SEQ ID NO: 1099 - DNA, 50 bp, Homo sapiens
cgggaaatgaccaagcgtctccacaatttaggggcaggtcagggtacctt

SEQ ID NO: 1100 - DNA, 50 bp, Homo sapiens
cgccacaaacccaccaaggtgctctcccccgagtacttgtgggaccagca

SEQ ID NO: 1101 - DNA, 50 bp, Homo sapiens
cggcaccactcatgttcataggccagctgaggctcagggggctgacttcc

SEQ ID NO: 1102 - DNA, 50 bp, Homo sapiens
cgctgggcaaccgtaagtatttagggagacagaggtgcctcttctgcaga

SEQ ID NO: 1103 - DNA, 50 bp, Homo sapiens
cgacgcccttgctcacaggctccattgagaggctgccccggtgctcatct

SEQ ID NO: 1104 - DNA, 50 bp, Homo sapiens
cgcagccctgcgggacctgaaaaggcgagactaagtcaccagccaagctg

SEQ ID NO: 1105 - DNA, 50 bp, Homo sapiens
cggtcactgtttgtcttgaaaaaaagagagaaaatgtttgtcattccagg

SEQ ID NO: 1106 - DNA, 50 bp, Homo sapiens
cgtcttctagcaggtatgagggagacaactacaaacgccagaggctcagc

SEQ ID NO: 1107 - DNA, 50 bp, Homo sapiens
agctctcgccgctgaaggggctcagcctggtcgacaaggtgaacaggccg

SEQ ID NO: 1108 - DNA, 50 bp, Homo sapiens
gcagggcagccagctacgaattttgagctctggggcgcgctcacctggcg

SEQ ID NO: 1109 - DNA, 50 bp, Homo sapiens
cgggtcaagagcagtgacctagtggccctcagccctggatggaggacaga

SEQ ID NO: 1110 - DNA, 50 bp, Homo sapiens
cgccattctgttggtactaaggtgttgtggttggtagaagcttaagtgag

SEQ ID NO: 1111 - DNA, 50 bp, Homo sapiens
cgggtcgtggtgactactaatgggtataagatttctttttgagatgatgg

SEQ ID NO: 1112 - DNA, 50 bp, Homo sapiens
tgttgacctcaggtccagtttgtttgcagcctccctcttctgacagatcg

SEQ ID NO: 1113 - DNA, 50 bp, Homo sapiens; identical to SEQ ID NO: 214

(19) United States
(12) Patent Application Publication
(10) Pub. No.: US 2022/0119885 A1
(43) Pub. Date: Apr. 21, 2022

(54) DNA METHYLATION BASED BIOMARKERS FOR LIFE EXPECTANCY AND MORBIDITY

(71) Applicant: The Regents of the University of California, Oakland, CA (US)

(72) Inventors: Stefan Horvath, Los Angeles, CA (US); Ake Tzu-Hui Lu, Los Angeles, CA (US)

(73) Assignee: The Regents of the University of California, Oakland, CA (US)

(21) Appl. No.: 17/282,318

(22) PCT Filed: Oct. 9, 2019
(86) PCT No.: PCT/US19/55444

§ 371 Date: Apr. 1, 2021

Related U.S. Application Data
(60) Provisional application No. 62/744,010, filed on Oct. 10, 2018.

(51) Int. Cl. C12Q 1/6883; C12Q 1/6827

(52) U.S. Cl. CPC C12Q 1/6883; C12Q 2600/154; C12Q 1/6827

(57) ABSTRACT

The disclosure provides seven DNAm-based estimators of plasma protein levels including those of plasminogen activation inhibitor 1 (PAI-1) and growth differentiation factor 15 (GDF15). The predictor of lifespan, DNAm GrimAge (in units of years), is a composite biomarker based on the seven DNAm surrogate markers and a DNAm-based estimator of smoking pack-years. These novel DNAm based biomarkers show the expected relationship with lifestyle factors (including healthy diet or educational attainment) and clinical biomarkers. Overall, these DNAm based biomarkers are expected to find many useful applications including human anti-aging studies.

### CROSS REFERENCE TO RELATED APPLICATIONS

This application claims the benefit under 35 U.S.C. Section 119(e) of co-pending and commonly-assigned U.S. Provisional Patent Application Ser. No. 62/744,010, filed on Oct. 10, 2018, and entitled “DNA METHYLATION BASED BIOMARKERS FOR LIFE EXPECTANCY AND MORBIDITY” which application is incorporated by reference herein.

### STATEMENT OF GOVERNMENT INTEREST

This invention was made with government support under Grant Number AG051425, awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 3, 2019, is named 30435_0361WOU1_SL.txt and is 161,691 bytes in size.

### TECHNICAL FIELD

The invention relates to methods and materials for examining biological aging in individuals.

### BACKGROUND OF THE INVENTION

Studies in invertebrates (yeast, worm, flies) have led to a long list of pharmacological agents that promise to intervene in different aspects of the aging process including stress response mimetics, anti-inflammatory interventions, epigenetic modifiers, neuroprotective agents, hormone treatments. While our arsenal of potential anti-aging interventions is brimming with highly promising candidates that delay aging in model organisms, it remains to be seen whether these interventions delay aging in human cells. To facilitate effective in vitro and ex vivo studies, there is a need for robust biomarkers of aging for human fibroblasts and other widely used cell types. One potential biomarker that has gained significant interest in recent years is DNA methylation (DNAm). Chronological time has been shown to elicit predictable hypo- and hyper-methylation changes at many regions across the genome. Several DNAm based biomarkers of aging have been developed including those using blood-based algorithms and the multi-tissue algorithms. These epigenetic age estimators exhibit statistically significant associations with many age-related diseases and conditions.

Recently developed DNA methylation-based biomarkers allow one to estimate the epigenetic age of an individual. For example, the pan tissue epigenetic clock, which is based on 353 dinucleotide markers, known as CpGs (-C-phosphate-G-), can be used to estimate the age of most human cell types, tissues, and organs (Horvath S. DNA methylation age of human tissues and cell types. Genome Biol. 2013: 14(R115). The estimated age, referred to as “DNA methylation age” (DNAm age), correlates with chronological age when methylation is assessed in certain cell types, tissues, and organs including whole blood, brain, breast, kidney, liver, lung, skin and saliva. Other reports described DNAm-based biomarkers that pertain to a single tissue (e.g. saliva or blood). Recent studies suggested that DNAm-based biomarkers of age capture aspects of biological age. For example, we and others have previously shown that individuals whose DNAm age was greater than their chronological age, i.e. individuals who exhibited epigenetic “age acceleration”, were at an increased risk for death from all causes, even after accounting for known risk factors.

There is a need for improved methods of observing phenomena associated with epigenetic aging, independent of chronological age and traditional risk factors of mortality, as well as improved methods for observing the effects of one or more test agents on the epigenetic aging of human cells.

### SUMMARY OF THE INVENTION

The invention disclosed herein provides methods and materials designed to observe DNA methylation levels at selected sites within the human genome. Using these methods and materials, embodiments of the invention provide a number of different biomarkers useful for predicting human lifespan, i.e. time to death, based on DNA methylation levels in genomic DNA obtained from samples such as blood tissue, blood cells, saliva, or buccal swabs. As discussed in detail below, embodiments of the invention observe methylation levels at a variety sites within the human genome in order to obtain information on a variety of phenomena associated with aging such as life expectancy, mortality, and morbidity. Disclosure that focuses on the prediction of mortality and morbidity in humans show that these DNAm based biomarkers are highly robust and informative for a range of applications. As discussed in detail below, embodiments of the invention include methods of observing genomic methylation in an individual in order to obtain information on one or more physiological factors associated with an epigenetic age of the individual, as well as methods of observing the effects of one or more test agents on genomic methylation that is associated with the epigenetic aging of human cells.

Embodiments of the invention can be used to provide information that complements and enhances conventional biomarker assessments that are widely used in clinical applications. For example, embodiments of the invention can be used to directly predict/prognosticate mortality, as well as provide further information on a host of age-related conditions such as cardiovascular disease, cancer risk, progression in neurodegeneration, and various measures of frailty. Embodiments of the invention can also be used to estimate the plasma levels of a number of different proteins associated with aging. In addition, one embodiment of the invention can be used to provide highly accurate information on the numbers of cigarettes smoked by an individual in their lifetime.

One biomarker embodiment disclosed herein and referred to as “DNAm GrimAge” is based on DNA methylation measurements at 1113 cytosine-phosphate-guanines (CpG) locations within the human genome. Significantly, this DNAm GrimAge methylation measurement at 1113 locations is collectively based on eight individual DNAm based biomarker assays at selected subsets of CpG locations, subsets of biomarkers that provide information on the numbers of cigarettes smoked by an individual (more precisely smoking packyears) as well as the in vivo plasma levels of: (1) adrenomedullin (ADM); (2) beta-2-microglobulin (B2M); (3) cystatin-C; (4) growth differentiation factor 15 (GDF15); (5) leptin; (6) plasminogen activator inhibitor 1 (PAI1); and (7) tissue inhibitor metalloproteinases 1 (TIMP1). Interestingly, plasma levels of ADM, B2M, cystatin C, and leptin relate to many age-related traits including cognitive functioning. For example, ADM levels are observed to be increased in individuals with hypertension and heart failure. Plasma B2M is a clinical biomarker associated with cardiovascular disease, kidney function, and inflammation. Plasma cystatin-C can be used to assess kidney function. Moreover, GDF-15 is involved in age-related mitochondrial dysfunction and PAI-1 plays a central role in a number of age-related subclinical and clinical conditions (and recent genetic studies link PAI-1 to lifespan).

Embodiments of the invention include, for example, methods of obtaining information on one or more physiological factors associated with an age of an individual. These methods comprise obtaining genomic DNA from the individual, observing methylation of the genomic DNA in at least about 42 methylation markers from the group of methylation markers in SEQ ID NO: 1-SEQ ID NO: 1113, and then correlating observed methylation in the methylation markers with the one or more physiological factors associated with the age of an individual such that information on the one or more physiological factors associated with the age of an individual is obtained. Typically, the genomic DNA is obtained from human fibroblasts, keratinocytes, buccal cells, endothelial cells, lymphoblastoid cells, and/or cells obtained from blood, skin, dermis, epidermis or saliva. In typical embodiments of the invention, methylation is observed by a process comprising treatment of genomic DNA from the population of cells from the individual with bisulfite to transform unmethylated cytosines of CpG dinucleotides in the genomic DNA to uracil; and/or genomic DNA is hybridized to a complimentary polynucleotide sequence disposed on a microarray. Optionally in these embodiments, correlating observed methylation in the methylation markers comprises a regression analysis.

In addition, certain embodiments of the invention observe specific constellations of markers in SEQ ID NO: 1-SEQ ID NO: 1113. For example, in one embodiment of the invention, the one or more physiological factors associated with an age of an individual comprises predicted age or lifespan of an individual, or the time to coronary heart disease in the individual, the method comprising observing methylation of the genomic DNA in about 1113 methylation markers from the group of methylation markers in SEQ ID NO: 1-SEQ ID NO: 1113, and then correlating observed methylation in the about 1113 methylation markers with predicted age or lifespan of an individual, or the time to coronary heart disease in the individual, such that information on predicted age or lifespan of an individual, or the time to coronary heart disease in the individual is obtained. In another embodiment of the invention, the one or more physiological factors associated with an age of an individual comprises a number of years in which the individual has smoked in their lifetime, and the method comprises observing methylation of the genomic DNA in about 172 methylation markers from the group of methylation markers in SEQ ID NO: 1-SEQ ID NO: 172, and then correlating observed methylation in the about 172 methylation markers with the number of years in which an individual has smoked in their lifetime, such that information on the number of years in which the individual has smoked in their lifetime is obtained. In another embodiment of the invention, the one or more physiological factors associated with an age of an individual comprises plasma protein levels of tissue inhibitor metalloproteinase 1 in the individual, and the method comprises observing methylation of the genomic DNA in about 42 methylation markers from the group of methylation markers in SEQ ID NO: 173-SEQ ID NO: 214, and then correlating observed methylation in the about 42 methylation markers with plasma protein levels of tissue inhibitor metalloproteinase 1 in an individual, such that information on plasma protein levels of tissue inhibitor metalloproteinase 1 in an individual is obtained. In another embodiment of the invention, the one or more physiological factors associated with an age of an individual comprises plasma protein levels of Cystatin-C in the individual, the method comprising observing methylation of the genomic DNA in about 87 methylation markers from the group of methylation markers in SEQ ID NO: 215-SEQ ID NO: 301, and then correlating observed methylation in the about 87 methylation markers with plasma protein levels of Cystatin-C in an individual, such that information on plasma protein levels of Cystatin-C in an individual is obtained. In another embodiment of the invention, the one or more physiological factors associated with an age of an individual comprises plasma protein levels of Beta-2-microglobulin in the individual, the method comprising observing methylation of the genomic DNA in about 91 methylation markers from the group of methylation markers in SEQ ID NO: 302-SEQ ID NO: 392, and then correlating observed methylation in the about 91 methylation markers with plasma protein levels of Beta-2-microglobulin in an individual, such that information on plasma protein levels of Beta-2-microglobulin in an individual is obtained. In another embodiment of the invention, the one or more physiological factors associated with an age of an individual comprises plasma protein levels of growth differentiation factor 15 in the individual, the method comprising observing methylation of the genomic DNA in about 137 methylation markers from the group of methylation markers in SEQ ID NO: 393-SEQ ID NO: 529, and then correlating observed methylation in the about 137 methylation markers with plasma protein levels of growth differentiation factor 15 in an individual, such that information on plasma protein levels of growth differentiation factor 15 in an individual is obtained. In another embodiment of the invention, the one or more physiological factors associated with an age of an individual comprises plasma protein levels of adrenomedullin in the individual, the method comprising observing methylation of the genomic DNA in about 186 methylation markers from the group of methylation markers in SEQ ID NO: 530-SEQ ID NO: 715, and then correlating observed methylation in the about 186 methylation markers with plasma protein levels of adrenomedullin in an individual, such that information on plasma protein levels of adrenomedullin in an individual is obtained. In another embodiment of the invention, the one or more physiological factors associated with an age of an individual comprises plasma protein levels of Leptin in the individual, the method comprising observing methylation of the genomic DNA in about 187 methylation markers from the group of methylation markers in SEQ ID NO: 716-SEQ ID NO: 902, and then correlating observed methylation in the about 187 methylation markers with Leptin in an individual, such that information on plasma protein levels of Leptin in an individual is obtained. In another embodiment of the invention, the one or more physiological factors associated with an age of an individual comprises plasma protein levels of plasminogen activator inhibitor 1 in the individual, the method comprising observing methylation of the genomic DNA in about 211 methylation markers from the group of methylation markers in SEQ ID NO: 903-SEQ ID NO: 1113, and then correlating observed methylation in the about 211 methylation markers with plasminogen activator inhibitor 1 in an individual, such that information on plasma protein levels of plasminogen activator inhibitor 1 in an individual is obtained.

Yet another embodiment of the invention is a method of observing the effects of a test agent on genomic methylation associated epigenetic aging of human cells. Typically, the method comprise combining the test agent with human cells, observing methylation status in at least 42 of the methylation markers of SEQ ID NO: 1-SEQ ID NO: 1113 in genomic DNA from the human cells, and then comparing the observations from (b) with observations of the methylation status in at least 42 of methylation markers of SEQ ID NO: 1-SEQ ID NO: 1113 in genomic DNA from control human cells not exposed to the test agent such that effects of the test agent on genomic methylation associated epigenetic aging in the human cells is observed. Typically, the method comprises observing methylation of the genomic DNA in about 172 methylation markers from the group of methylation markers in SEQ ID NO: 1-SEQ ID NO: 172; and/or observing methylation of the genomic DNA in about 42 methylation markers from the group of methylation markers in SEQ ID NO: 173-SEQ ID NO: 214; and/or observing methylation of the genomic DNA in about 87 methylation markers from the group of methylation markers in SEQ ID NO: 215-SEQ ID NO: 301; and/or observing methylation of the genomic DNA in about 91 methylation markers from the group of methylation markers in SEQ ID NO: 302-SEQ ID NO: 392; and/or observing methylation of the genomic DNA in about 137 methylation markers from the group of methylation markers in SEQ ID NO: 393-SEQ ID NO: 529; and/or observing methylation of the genomic DNA in about 186 methylation markers from the group of methylation markers in SEQ ID NO: 530-SEQ ID NO: 715; and/or observing methylation of the genomic DNA in about 187 methylation markers from the group of methylation markers in SEQ ID NO: 716-SEQ ID NO: 902; and/or observing methylation of the genomic DNA in about 211 methylation markers from the group of methylation markers in SEQ ID NO: 903-SEQ ID NO: 1113; and/or observing methylation of the genomic DNA in about 1113 methylation markers of SEQ ID NO: 1-SEQ ID NO: 1113. Optionally in these methods, a plurality of test agents are combined with the human cells in vitro (e.g. primary keratinocytes from a single or, alternatively, multiple donors. In illustrative embodiments of the invention, the test agent is a polypeptide, a polynucleotide or a compound having a molecular weight less than 3,000, 2,000, 1,000 or 500 g/mol.

In certain embodiments of the methods disclosed above, the genomic DNA can be obtained from human fibroblasts, keratinocytes, buccal cells, endothelial cells, lymphoblastoid cells, and/or cells obtained from blood, skin, dermis, epidermis or saliva. In typical embodiments of the invention, methylation is observed by a process comprising treatment of genomic DNA from the population of cells from the individual with bisulfite to transform unmethylated cytosines of CpG dinucleotides in the genomic DNA to uracil. Optionally in such methods, genomic DNA is hybridized to a complimentary sequence disposed on a microarray, and/or genomic DNA is amplified by a polymerase chain reaction process. In some embodiments of the invention, methylation in at least 75% of the respective groups of methylation markers in a constellation of markers is observed (e.g. a constellation of 172 markers, a constellation of 42 markers etc.). In certain embodiments of the invention, correlating observed methylation in the methylation markers comprises a regression analysis, for example one that also considers the sex of the individual, and/or the ethnicity/race of the individual. Optionally for example, correlating observed methylation in the methylation markers comprises a regression analysis selected to predict time to coronary heart disease in the individual.

As discussed below, the DNAm GrimAge biomarker disclosed herein can supplement and enhance existing conventional lifespan predictors. In addition, the subsets of DNAm based biomarkers (e.g. for smoking pack-years and protein plasma levels) have interesting applications in their own right. For example, the DNAm based surrogate biomarker of smoking pack-years could complement self-reported assessments of pack-years. The surprising fact that DNAm pack-years outperforms self-reported pack-years when it comes to lifespan prediction could reflect erroneous self-reporting or true biology: maybe DNAm pack-years is a superior measure of long-term exposure to smoke, or of biological response to it. Moreover, the DNAm based surrogate biomarkers of plasma protein levels disclosed herein can be used, for example, by epidemiologists who have access to stored DNA samples but no access to plasma samples.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 provides a schematic of a flowchart for stages of the development and data from the DNAm GrimAge methodology disclosed herein. Surrogate DNAm based biomarkers for smoking pack-years and plasma protein levels were defined and validated using training and test data from the Framingham Heart study (stage 1). Only 12 out of 88 plasma proteins exhibited a correlation r>0.35 with their respective DNAm based surrogate marker in the test data. In stage 2, time-to-death (due to all-cause mortality) was regressed on chronological age, sex, and DNAm based biomarkers of smoking pack-years and the 12 above mentioned plasma protein levels. The ElasticNet regression model automatically selected the following covariates: chronological age (Age), sex (Female), and DNAm based surrogates for smoking pack-years (DNAm PACKYRS), adrenomedullin levels (DNAm ADM), beta-2 microglobulin (DNAm B2M), cystatin C (DNAm Cystatin C), growth differentiation factor 15 (DNAm GDF-15), leptin (DNAm Leptin), plasminogen activation inhibitor 1 (DNAm PAI-1), tissue inhibitor metalloproteinase 1 (DNAm TIMP-1). The linear combination of the covariate values X^{T}β was linearly transformed to be in units of years. Technically speaking DNAm GrimAge is a mortality risk estimator. Metaphorically speaking, it can be interpreted as a DNAm based estimator of biological age or as another epigenetic clock.

FIG. 2 provides a heat map of pairwise correlations of DNAm based biomarkers.

The heat map codes the pairwise Pearson correlations of select variables (surrounding the definition of DNAm GrimAge) in the test data from the Framingham Heart Study (N=625). DNAm GrimAge is defined as a linear combination of chronological age (Age), sex (Female takes on the value 1 for females and 0 otherwise), and eight DNAm based surrogate markers for smoking pack-years (DNAm PACKYRS), adrenomedullin levels (DNAm ADM), beta-2 microglobulin (DNAm B2M), cystatin C (DNAm Cystatin C), growth differentiation factor 15 (DNAm GDF-15), leptin (DNAm Leptin), plasminogen activation inhibitor 1 (DNAm PAI-1), issue inhibitor metalloproteinase 1 (DNAm TIMP-1). The figure also includes an estimator of mortality risk, mortality.res, which can be interpreted as a measure of “excess” mortality risk compared to the baseline risk in the test data. Formally, mortality.res is defined as the deviance residual from a Cox regression model for time-to-death due to all-cause mortality. The rows and columns of the Figure are sorted according to a hierarchical clustering tree. The shades of color (blue, white, and red) visualize correlation values from −1 to 1. Each square reports the Pearson correlation coefficient.

FIGS. 3A-3I show data from meta-analysis forest plots for predicting time-to-death due to all-cause mortality. Each panel reports a meta-analysis forest plot for combining hazard ratios predicting time-to-death based on a DNAm based biomarker (reported in the figure heading) across different strata formed by racial group within cohort. FIG. 3A shows results for AgeAccelGrim. Each row reports a hazard ratio (for time-to-death) and a 95% confidence interval resulting from a Cox regression model in each of 9 strata (defined by cohort and racial groups). Results for (age-adjusted) DNAm based surrogate markers of (FIG. 3B) adrenomedullin (ADM), (FIG. 3C) beta-2 microglobulin (B2M), (FIG. 3D) cystatin C (Cystatin C), (FIG. 3E) growth differentiation factor 15 (GDF-15), (FIG. 3F) leptin, (FIG. 3G) plasminogen activation inhibitor 1 (PAI-1), (FIG. 3H) tissue inhibitor metalloproteinase 1 (TIMP-1) and (FIG. 3I) smoking pack-years (PACKYRS). The sub-title of each panel reports the meta-analysis p-value and a p-value for a test of heterogeneity Cochran Q test (Het.). In FIG. 3A, each hazard ratio (HR) corresponds to a one-year increase in AgeAccelGrim. In FIGS. 3B-3H, each hazard ratio corresponds to an increase in one-standard deviation. In FIG. 3I, hazard ratios correspond to a 1-year increase in pack-years. The most significant meta-analysis P value (here AgeAccelGrim) is marked in red. A non-significant Cochran Q test p-value is desirable because it indicates that the hazard ratios don't differ significantly across the strata. For example, the hazard ratios associated with AgeAccelGrim exhibit insignificant heterogeneity across the strata (Cochran Q test Pₗs=0.16).

FIGS. 4A-4I show data from meta-analysis forest plots for predicting time-to-coronary heart disease. Each panel reports a meta-analysis forest plot for combining hazard ratios predicting time to CHD and the DNAm based biomarker (reported in the figure heading) across different strata formed by racial groups within cohorts. FIG. 4A shows results for AgeAccelGrim. Each row reports a hazard ratio (for time-to-CHD) and a 95% confidence interval resulting from a Cox regression model in each of 9 strata (defined by cohort and racial groups). Results for (age adjusted) DNAm based surrogate markers of (FIG. 4B) adrenomedullin (ADM), (FIG. 4C) beta-2 microglobulin (B2M), (FIG. 4D) cystatin C (Cystatin C), (FIG. 4E) growth differentiation factor 15 (GDF-15), (FIG. 4F) leptin, (FIG. 4G) plasminogen activation inhibitor 1 (PAI-1), (FIG. 4H) tissue inhibitor metalloproteinase 1 (TIMP-1) and (FIG. 4I) smoking pack-years (PACKYRS). The sub-title of each panel reports the meta-analysis p-value and a p-value for a test of heterogeneity Cochran Q test (Het.). In FIG. 4A, each hazard ratio (HR) corresponds to a one-year increase in AgeAccelGrim. In FIGS. 4B-4H, each hazard ratio corresponds to an increase in one-standard deviation. In FIG. 4I, hazard ratios correspond to a one unit increased in DNAm pack-years. The most significant meta-analysis P value (here AgeAccelGrim) is marked.

FIGS. 5A-5I show data from meta-analysis of associations with total number of age-related conditions. Each panel reports a meta-analysis forest plot for combining regression coefficients between the comorbidity index and the DNAm based biomarker (reported in the figure heading) across different strata, which are formed by racial group within cohort. FIG. 5A shows a meta-analysis of the regression slope between AgeAccelGrim and the comorbidity index. Analogous results for (age adjusted) DNAm based surrogate markers of (FIG. 5B) adrenomedullin (ADM), (FIG. 5C) beta-2 microglobulin (B2M), (FIG. 5D) cystatin C (Cystatin C), (FIG. 5E) growth differentiation factor 15 (GDF-15), (FIG. 5F) leptin, (FIG. 5G) plasminogen activation inhibitor 1 (PAI-1), (FIG. 5H) tissue inhibitor metalloproteinase 1 (TIMP-1) and (FIG. 5I) smoking pack-years (PACKYRS). The individual study results were combined using fixed effect meta-analysis (reported in the panel heading). Cochran Q test for heterogeneity across studies (Het.). The effect sizes correspond to one year of age acceleration in panel A, one pack-year in panel I and one standard deviation in other panels for DNAm proteins. The estimate with the most significant meta P value is marked in red.

FIG. 6 provides a grid showing cross sectional correlations between DNAm biomarkers and lifestyle factors. Robust correlation coefficients (biweight midcorrelation [39]) between 1) AgeAccelGrim and its eight age-adjusted underlying DNAm based surrogate biomarkers and 2) 38 variables including self-reported diet, 9 dietary biomarkers, 12 variables related to metabolic traits and central adiposity, and 5 life style factors. The analysis was performed on the WHI cohort in up to 4200 postmenopausal women.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

All publications mentioned herein are incorporated herein by reference to disclose and describe aspects, methods and/or materials in connection with the cited publications. For example, Lu et al., Aging (Albany N.Y.). 2019 Jan. 21; 11(2):303-327. doi: 10.18632/aging 101684; PCT Patent Application No.: PCT/US2019/034829, U.S. Patent Publication 20150259742, U.S. patent application Ser. No. 15/025,185, titled “METHOD TO ESTIMATE THE AGE OF TISSUES AND CELL TYPES BASED ON EPIGENETIC MARKERS”, filed by Stefan Horvath; U.S. patent application Ser. No. 14/119,145, titled “METHOD TO ESTIMATE AGE OF INDIVIDUAL BASED ON EPIGENETIC MARKERS IN BIOLOGICAL SAMPLE”, filed by Eric Villain et al.; and Hannum et al. “Genome-Wide Methylation Profiles Reveal Quantitative Views Of Human Aging Rates.” Molecular Cell. 2013; 49(2):359-367 and patent US2015/0259742, are incorporated by reference in their entirety herein.

The invention disclosed herein provides novel and powerful biomarker predictors of life expectancy, mortality, and morbidity based on DNA methylation levels. Our discoveries surrounding the prediction of mortality and morbidity show that the DNAm based biomarkers disclosed herein are highly robust and informative for a range of applications. Embodiments of the DNAm based biomarkers disclosed herein can provide complementary information that enhances and supplements traditional biomarker assessments that are widely used in clinical applications. For example, embodiments of the invention can be used to directly predict/prognosticate mortality, and further information relating to a host of age-related conditions such as cardiovascular disease, cancer risk, progression in neurodegeneration, and various measures of frailty.

Embodiments of the invention include methods of observing the effects of one or more test agents on genomic methylation associated epigenetic aging of human cells. Typically, these methods comprise combining the test agent(s) with human cells (e.g. for specified period of time such as at least 1-7 days, 1-3 weeks, 1-6 months or the like), and then observing methylation status in at least 42 of the methylation markers of SEQ ID NO: 1-SEQ ID NO: 1113 in genomic DNA from the human cells, and then comparing the observations from (b) with observations of the methylation status in at least 42 of methylation markers of SEQ ID NO: 1-SEQ ID NO: 1113 in genomic DNA from control human cells not exposed to the test agent such that effects of the test agent on genomic methylation associated epigenetic aging in the human cells is observed. Typically, the method comprises observing methylation of the genomic DNA in about 172 methylation markers from the group of methylation markers in SEQ ID NO: 1-SEQ ID NO: 172; and/or observing methylation of the genomic DNA in about 42 methylation markers from the group of methylation markers in SEQ ID NO: 173-SEQ ID NO: 214; and/or observing methylation of the genomic DNA in about 87 methylation markers from the group of methylation markers in SEQ ID NO: 215-SEQ ID NO: 301; and/or observing methylation of the genomic DNA in about 91 methylation markers from the group of methylation markers in SEQ ID NO: 302-SEQ ID NO: 392; and/or observing methylation of the genomic DNA in about 137 methylation markers from the group of methylation markers in SEQ ID NO: 393-SEQ ID NO: 529; and/or observing methylation of the genomic DNA in about 186 methylation markers from the group of methylation markers in SEQ ID NO: 530-SEQ ID NO: 715; and/or observing methylation of the genomic DNA in about 187 methylation markers from the group of methylation markers in SEQ ID NO: 716-SEQ ID NO: 902; and/or observing methylation of the genomic DNA in about 211 methylation markers from the group of methylation markers in SEQ ID NO: 903-SEQ ID NO: 1113; and/or observing methylation of the genomic DNA in about 1113 methylation markers of SEQ ID NO: 1-SEQ ID NO: 1113. Optionally in these methods, a plurality of test agents are combined with the human cells in vitro (e.g. primary keratinocytes from a single or, alternatively, multiple donors. In illustrative embodiments of the invention, the test agent is a polypeptide, a polynucleotide or a compound having a molecular weight less than 3,000, 2,000, 1,000 or 500 g/mol.

Embodiments of the invention include a number of different biomarkers useful for predicting human lifespan, i.e. time to death, based on DNA methylation levels in blood tissue, blood cells, saliva, or buccal swabs. One biomarker embodiment, referred to as “DNAm GrimAge”, can be described on two levels. First, it is based on DNA methylation measurements at 1113 locations, called cytosine-phosphate-guanines (CpGs) in the human genome. Second, at a higher level, these DNA methylation measurements at 1113 locations are further based on eight individual DNAm based biomarkers that measure i) the numbers of cigarettes smoked (more precisely smoking packyears) and ii) the plasma levels of the following seven proteins: (1) adrenomedullin (ADM); (2) beta-2-microglobulin (B2M); (3) cystatin-C; (4) growth differentiation factor 15 (GDF15); (5) leptin; (6) plasminogen activator inhibitor 1 (PAI1); and (7) tissue inhibitor metalloproteinases 1 (TIMP1).

Embodiments of the invention further include, for example, methods of obtaining information on one or more physiological factors associated with an age of an individual. These methods comprise obtaining genomic DNA from the individual, observing methylation of the genomic DNA in at least about 42 methylation markers from the group of methylation markers in SEQ ID NO: 1-SEQ ID NO: 1113, and then correlating observed methylation in the methylation markers with the one or more physiological factors associated with the age of an individual such that information on the one or more physiological factors associated with the age of an individual is obtained. Typically, the genomic DNA is obtained from human fibroblasts, keratinocytes, buccal cells, endothelial cells, lymphoblastoid cells, and/or cells obtained from blood, skin, dermis, epidermis or saliva. In typical embodiments of the invention, methylation is observed by a process comprising treatment of genomic DNA from the population of cells from the individual with bisulfite to transform unmethylated cytosines of CpG dinucleotides in the genomic DNA to uracil, and/or genomic DNA is hybridized to a complimentary polynucleotide sequence disposed on a microarray. Optionally in these embodiments, correlating observed methylation in the methylation markers comprises a regression analysis.

In this context, certain embodiments of the invention observe specific constellations of markers in SEQ ID NO: 1-SEQ ID NO: 1113. For example, in one embodiment of the invention, the one or more physiological factors associated with an age of an individual comprises predicted age or lifespan of an individual, or the time to coronary heart disease in the individual, the method comprising observing methylation of the genomic DNA in about 1113 methylation markers from the group of methylation markers in SEQ ID NO: 1-SEQ ID NO: 1113, and then correlating observed methylation in the about 1113 methylation markers with predicted age or lifespan of an individual, or the time to coronary heart disease in the individual, such that information on predicted age or lifespan of an individual, or the time to coronary heart disease in the individual is obtained. In another embodiment of the invention, the one or more physiological factors associated with an age of an individual comprises a number of years in which the individual has smoked in their lifetime, and the method comprises observing methylation of the genomic DNA in about 172 methylation markers from the group of methylation markers in SEQ ID NO: 1-SEQ ID NO: 172, and then correlating observed methylation in the about 172 methylation markers with the number of years in which an individual has smoked in their lifetime, such that information on the number of years in which the individual has smoked in their lifetime is obtained. In another embodiment of the invention, the one or more physiological factors associated with an age of an individual comprises plasma protein levels of tissue inhibitor metalloproteinase 1 in the individual, and the method comprises observing methylation of the genomic DNA in about 42 methylation markers from the group of methylation markers in SEQ ID NO: 173-SEQ ID NO: 214, and then correlating observed methylation in the about 42 methylation markers with plasma protein levels of tissue inhibitor metalloproteinase 1 in an individual, such that information on plasma protein levels of tissue inhibitor metalloproteinase 1 in an individual is obtained. In another embodiment of the invention, the one or more physiological factors associated with an age of an individual comprises plasma protein levels of Cystatin-C in the individual, the method comprising observing methylation of the genomic DNA in about 87 methylation markers from the group of methylation markers in SEQ ID NO: 215-SEQ ID NO: 301, and then correlating observed methylation in the about 87 methylation markers with plasma protein levels of Cystatin-C in an individual, such that information on plasma protein levels of Cystatin-C in an individual is obtained. In another embodiment of the invention, the one or more physiological factors associated with an age of an individual comprises plasma protein levels of Beta-2-microglobulin in the individual, the method comprising observing methylation of the genomic DNA in about 91 methylation markers from the group of methylation markers in SEQ ID NO: 302-SEQ ID NO: 392, and then correlating observed methylation in the about 91 methylation markers with plasma protein levels of Beta-2-microglobulin in an individual, such that information on plasma protein levels of Beta-2-microglobulin in an individual is obtained. In another embodiment of the invention, the one or more physiological factors associated with an age of an individual comprises plasma protein levels of growth differentiation factor 15 in the individual, the method comprising observing methylation of the genomic DNA in about 137 methylation markers from the group of methylation markers in SEQ ID NO: 393-SEQ ID NO: 529, and then correlating observed methylation in the about 137 methylation markers with plasma protein levels of growth differentiation factor 15 in an individual, such that information on plasma protein levels of growth differentiation factor 15 in an individual is obtained. In another embodiment of the invention, the one or more physiological factors associated with an age of an individual comprises plasma protein levels of adrenomedullin in the individual, the method comprising observing methylation of the genomic DNA in about 186 methylation markers from the group of methylation markers in SEQ ID NO: 530-SEQ ID NO: 715, and then correlating observed methylation in the about 186 methylation markers with plasma protein levels of adrenomedullin in an individual, such that information on plasma protein levels of adrenomedullin in an individual is obtained. In another embodiment of the invention, the one or more physiological factors associated with an age of an individual comprises plasma protein levels of Leptin in the individual, the method comprising observing methylation of the genomic DNA in about 187 methylation markers from the group of methylation markers in SEQ ID NO: 716-SEQ ID NO: 902, and then correlating observed methylation in the about 187 methylation markers with Leptin in an individual, such that information on plasma protein levels of Leptin in an individual is obtained. In another embodiment of the invention, the one or more physiological factors associated with an age of an individual comprises plasma protein levels of plasminogen activator inhibitor 1 in the individual, the method comprising observing methylation of the genomic DNA in about 211 methylation markers from the group of methylation markers in SEQ ID NO: 903-SEQ ID NO: 1113, and then correlating observed methylation in the about 211 methylation markers with plasminogen activator inhibitor 1 in an individual, such that information on plasma protein levels of plasminogen activator inhibitor 1 in an individual is obtained. In this context, the DNAm surrogate marker of PAI-1 level stands out when it comes to associations with type 2 diabetes status, glucose-, insulin-, triglyceride levels and measures of adiposity (body mass index and waist-to-hip ratio).

The DNAm GrimAge biomarker disclosed herein outperforms all considered alternative DNAm based predictors of lifespan and time-to-coronary heart disease. The two-stage construction of DNAm GrimAge (via the use of DNAm based surrogate biomarkers for plasma proteins and smoking) is admittedly involved but well worth the effort for the following reason. First, the resulting lifespan predictor outperforms all of the considered alternative biomarkers including those that were constructed by directly regressing lifespan on DNA methylation (such as our novel biomarker DNAm Mortality). Second, DNAm GrimAge outperforms other DNAm based biomarkers when it comes to predicting time to CHD. Third, DNAm GrimAge can be readily interpreted as a linear combination of age, sex, and surrogate biomarkers for plasma proteins and smoking pack-years. Fourth, the DNAm GrimAge estimate has an intuitive interpretation as physiological age since it is in units of years.

We expect that our other surrogate DNAm based biomarkers (for smoking pack-years and PAI-1 levels) will find interesting applications in their own right. We demonstrate that only a small fraction of plasma proteins (we focused on 7 out of 88) can be imputed based on DNA methylation levels. In the Framingham heart study (FHS) data, the measurement of the plasma proteins (exam 7) preceded the measurement of blood methylation data (exam 8) by 6.6 years, which suggests that long term exposure to elevated/decreased plasma levels are associated with subsequent changes in methylation levels. However, the elucidation of cause-and-effect relationships between certain plasma proteins and DNA methylation changes will require future longitudinal cohort studies and mechanistic studies. Our DNAm based surrogate biomarkers of plasma protein levels could also be interesting to epidemiologists who have access to stored DNA samples but no access to plasma samples. Strong literature support links the selected plasma proteins (used in the construction of GrimAge) to various age-related conditions: ADM levels are increased in individuals with hypertension and heart failure. Plasma B2M is a clinical biomarker associated with cardiovascular disease, kidney function, and inflammation. Plasma cystatin-C can be used to assess kidney function. Overall, ADM, B2M, cystatin C, and leptin relate to many age-related traits including cognitive functioning. GDF-15 is involved in age-related mitochondrial dysfunction. PAI-1 plays a central role in a number of age-related subclinical and clinical conditions[9], and recent genetic studies link PAI-1 to lifespan. The tissue inhibitor of metalloproteinases, TIMP-1, plays an anti-apoptotic function.

Despite their obvious strengths, it is unlikely that DNAm based biomarkers will replace existing clinical biomarkers (such as glucose levels, lipid levels, blood pressure) when it comes to informing patient care. Rather, we expected that these biomarkers will complement existing clinical biomarkers when it comes to evaluating anti-aging interventions in vivo and in vitro. Since DNAm captures important properties of the DNA molecule, these DNAm biomarkers are expected to be proximal to innate aging processes.

The DNAm GrimAge biomarker will enhance existing conventional lifespan predictors. Our DNAm based surrogate biomarker of smoking pack-years could complement self-reported assessments of pack-years. The surprising fact that DNAm pack-years outperforms self-reported pack-years when it comes to lifespan prediction could reflect erroneous self-reporting or true biology: maybe DNAm pack-years is a superior measure of long-term exposure to smoke, or of biological response to it.

Beyond lifespan prediction, AgeAccelGrim (and several of its underlying surrogate biomarkers) relate to most age-related conditions (metabolic syndrome, comorbidity, markers of inflammation such as C-reactive protein levels) in the expected way: increased values are associated with a grim condition. Similarly, higher values of AgeAccelGrim (and several DNAm based surrogate markers) are associated with a blood cell composition that is indicative of older individuals and shorter telomere length. While the reported associations are statistically highly significant, the magnitude of the underlying correlations is relatively small. In light of these data, it is unlikely that AgeAccelGrim is simply a marker of immunosenescence. Future studies will need to evaluate the hypothesis that AgeAccelGrim is associated with several hallmarks of aging including immunosenescence, the decline in hematopoietic stem cells, and cell intrinsic epigenetic changes.

While AgeAccelGrim stands out when it comes to lifespan prediction and prediction of time-to-CHD, our (age-adjusted) DNAm surrogate marker of PAI-1 level stands out when it comes to associations with type 2 diabetes status, glucose-, insulin-, triglyceride levels and measures of adiposity (body mass index and waist-to-hip ratio). Inflammation and metabolic conditions are associated with AgeAccelGrim, age-adjusted DNAm PAI-1 and age-adjusted DNAm TIMP-1. Our dietary analysis reveals that vegetable consumption is associated with slower epigenetic aging which echoes previous results. Using the invention disclosed herein, future longitudinal cohort studies can investigate the influence of various stress factors on AgeAccelGrim. Future genetic studies of AgeAccelGrim promise to identify the underlying genetic variants. Overall, our set of novel DNAm based biomarkers has great potential to monitor and evaluate interventions applied to age-related conditions.

### Overview of the Two-Stage Approach for Defining DNAm GrimAge

We used a novel two-stage approach for defining a novel DNAm based predictor of life span (time to death due to all cause mortality). In stage 1, we defined a collection of surrogate DNAm biomarkers of physiological risk factors and stress factors. In stage 2, we combined these biomarkers into a single composite biomarker of lifespan (DNAm GrimAge). DNAm GrimAge is calibrated to be in units of years, i.e. it can be interpreted as DNA methylation based estimate of physiological age.

We first sought to develop DNA methylation based surrogate biomarkers of plasma protein levels since plasma proteins such as adrenomedullin, C-reactive protein, plasminogen activation inhibitor 1 (PAI-1), and growth differentiation factor 15 (GDF15) accompany important physiological processes and are associated with age-related conditions [12, 13]. Further, we defined a novel DNAm based estimator of smoking pack-years since smoking is a significant risk factor of mortality and morbidity.

The DNAm based biomarkers defined in stage 1 are attractive surrogate biomarkers for epidemiological studies because a) they lend themselves for imputing plasma protein levels and b) they allow checking self reported questionnaire entries surrounding smoking pack-years based on DNA methylation levels. The surrogate biomarkers have limitations, but it is exciting that stored DNA samples (collected decades ago) will allow one to impute plasma levels for a select group of plasma proteins.

In stage 2, we used these surrogate DNAm biomarkers to define a composite biomarker DNAm GrimAge that outperforms existing DNAm based biomarkers of lifespan. To demonstrate that DNAm GrimAge outperforms existing DNAm based predictors of lifespan we carried out a large-scale meta-analysis (involving more than 7000 Illumina array measurements). We also characterized the resulting DNAm GrimAge estimate with respect to lifestyle factors and a host of age-related conditions, e.g. we demonstrate that our DNAm based biomarkers predict time to cardiovascular disease. Finally, we demonstrate that DNAm GrimAge and several underlying surrogate biomarkers (e.g. DNAm PAI-1, DNAm GDF15) are associated with age-related changes in blood cell composition and leukocyte telomere length.

### Practicing the Invention of DNAm GrimAge

To use the epigenetic biomarker, one needs to extract DNA from cells or fluids, e.g. human blood cells, whole blood, peripheral blood mononuclear cells, and saliva (e.g. buccal skin cells). Next, one needs to measure DNA methylation levels in the underlying signature of 1113 CpGs (epigenetic markers) that are being used in the mathematical algorithm. The algorithm leads to an “age” (for each sample or human subject). The higher the value, the higher the risk of death and disease.

### FURTHER ILLUSTRATIVE ASPECTS AND EMBODIMENTS OF THE INVENTION

Novel molecular biomarkers of aging, such as those termed “DNAm age”, “epigenetic age” or “apparent methylomic aging rate” allow one to prognosticate mortality, are interesting to gerontologists (aging researchers), epidemiologists, medical professionals, and medical underwriters for life insurances. Exclusively clinical biomarkers such as lipid levels, body mass index, blood pressures have a long and successful history in the life insurance industry. By contrast, molecular biomarkers of aging have rarely been used.

DNA methylation refers to chemical modifications of the DNA molecule. Technological platforms such as the Illumina Infinium microarray or DNA sequencing-based methods have been found to lead to highly robust and reproducible measurements of the DNA methylation levels of a person. There are more than 28 million CpG loci in the human genome. Consequently, certain loci are given unique identifiers such as those found in the Illumina CpG loci database (see, e.g. Technical Note: Epigenetics, CpG Loci Identification ILLUMINA Inc. 2010). These CG locus designation identifiers are used herein. In this context, one embodiment of the invention is a method of obtaining information useful to observe biomarkers associated with a phenotypic age of an individual by observing the methylation status of one or more of the 1113 methylation marker specific GC loci that are identified in Table 9 below.

The term “epigenetic” as used herein means relating to, being, or involving a chemical modification of the DNA molecule. Epigenetic factors include the addition or removal of a methyl group which results in changes of the DNA methylation levels.

The term “nucleic acids” as used herein may include any polymer or oligomer of pyrimidine and purine bases, preferably cytosine, thymine, and uracil, and adenine and guanine, respectively. The present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “methylation marker” as used herein refers to a CpG position that is potentially methylated. Methylation typically occurs in a CpG containing nucleic acid. The CpG containing nucleic acid may be present in, e.g., in a CpG island, a CpG doublet, a promoter, an intron, or an exon of gene. For instance, in the genetic regions provided herein the potential methylation sites encompass the promoter/enhancer regions of the indicated genes. Thus, the regions can begin upstream of a gene promoter and extend downstream into the transcribed region.

The term “gene” as used herein refers to a region of genomic DNA associated with a given gene. For example, the region can be defined by a particular gene (such as protein coding sequence exons, intervening introns and associated expression control sequences) and its flanking sequence. It is, however, recognized in the art that methylation in a particular region is generally indicative of the methylation status at proximal genomic sites. Accordingly, determining a methylation status of a gene region can comprise determining a methylation status of a methylation marker within or flanking about 10 bp to 50 bp, about 50 to 100 bp, about 100 bp to 200 bp, about 200 bp to 300 bp, about 300 to 400 bp, about 400 bp to 500 bp, about 500 bp to 600 bp, about 600 to 700 bp, about 700 bp to 800 bp, about 800 to 900 bp, 900 bp to 1 kb, about 1 kb to 2 kb, about 2 kb to 5 kb, or more of a named gene, or CpG position.

The phrase “selectively measuring” as used herein refers to methods wherein only a finite number of methylation marker or genes (comprising methylation markers) are measured rather than assaying essentially all potential methylation marker (or genes) in a genome. For example, in some aspects, “selectively measuring” methylation markers or genes comprising such markers can refer to measuring no more than 211, 187, 137, 91, or 42 different methylation markers or genes comprising methylation markers in Table 9.

The invention described herein provides novel and powerful predictors of life expectancy, mortality, and morbidity based on DNA methylation levels. In this context, it is critical to distinguish clinical from molecular biomarkers of aging. Clinical biomarkers such as lipid levels, blood pressure, blood cell counts have a long and successful history in clinical practice. By contrast, molecular biomarkers of aging are rarely used. However, this is likely to change due to recent breakthroughs in DNA methylation-based biomarkers of aging. Since their inception in 2013, DNA methylation (DNAm) based biomarkers of aging promise to greatly enhance biomedical research, clinical applications, patient care, and even medical underwriting when it comes to life insurance policies and other financial products. They will also be more useful for clinical trials and intervention assessment that target aging, since they are more proximal to the biological changes that characterize the aging process compared to upstream clinical read outs of health and disease status.

It is critical to distinguish clinical from molecular biomarkers of aging. Clinical biomarkers such as lipid levels, blood pressure, blood cell counts have a long and successful history in clinical practice. By contrast, molecular biomarkers of aging are rarely used. However, this is likely to change due to recent breakthroughs in DNA methylation-based biomarkers of aging. Since their inception, DNA methylation (DNAm) based biomarkers of aging promise to greatly enhance biomedical research, clinical applications, patient care, and even medical underwriting when it comes to life insurance policies and other financial products. They will also be more useful for clinical trials and intervention assessment that target aging, since they are more proximal to the biological changes that characterize the aging process compared to upstream clinical read outs of health and disease status.

The disclosure presented herein surrounding the prediction of mortality and morbidity show that DNAm based biomarkers are highly robust and informative for a range of applications. DNAm age can not only be used to directly predict/prognosticate mortality but also relate to a host of age-related conditions such as heart disease risk, cancer risk, dementia status, cardiovascular disease and various measures of frailty. Further embodiments and aspects of the invention are discussed below.

DNA methylation of the methylation markers can be measured using various approaches, which range from commercial array platforms (e.g. from Illumina™) to sequencing approaches of individual genes. This includes standard lab techniques or array platforms. A variety of methods for detecting methylation status or patterns have been described in, for example U.S. Pat. Nos. 6,214,556, 5,786,146, 6,017,704, 6,265,171, 6,200,756, 6,251,594, 5,912,147, 6,331,393, 6,605,432, and 6,300,071 and US Patent Application Publication Nos. 20030148327, 20030148326, 20030143606, 20030082609 and 20050009059, each of which are incorporated herein by reference. Other array-based methods of methylation analysis are disclosed in U.S. patent application Ser. No. 11/058,566. For a review of some methylation detection methods, see, Oakeley, E. J., Pharmacology & Therapeutics 84:389-400 (1999). Available methods include, but are not limited to: reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), sodium bisulfite, combined bisulphate-restriction analysis, and methylation sensitive single nucleotide primer extension.

The methylation levels of a subset of the DNA methylation markers disclosed herein are assayed (e.g. using an Illumina™ DNA methylation array or using a PCR protocol involving relevant primers). To quantify the methylation level, one can follow the standard protocol described by Illumina™ to calculate the beta value of methylation, which equals the fraction of methylated cytosines in that location. The invention can also be applied to any other approach for quantifying DNA methylation at locations near the genes as disclosed herein. DNA methylation can be quantified using many currently available assays which include, for example:

a) Molecular break light assay for DNA adenine methyltransferase activity is an assay that is based on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase.

b) Methylation-Specific Polymerase Chain Reaction (PCR) is based on a chemical reaction of sodium bisulfite with DNA that converts unmethylated cytosines of CpG dinucleotides to uracil or UpG, followed by traditional PCR. However, methylated cytosines will not be converted in this process, and thus primers are designed to overlap the CpG site of interest, which allows one to determine methylation status as methylated or unmethylated. The beta value can be calculated as the proportion of methylation.

c) Whole genome bisulfite sequencing, also known as BS-Seq, is a genome-wide analysis of DNA methylation. It is based on the sodium bisulfite conversion of genomic DNA, which is then sequencing on a Next-Generation Sequencing (NGS) platform. The sequences obtained are then re-aligned to the reference genome to determine methylation states of CpG dinucleotides based on mismatches resulting from the conversion of unmethylated cytosines into uracil.

d) The Hpall tiny fragment Enrichment by Ligation-mediated PCR (HELP) assay is based on restriction enzymes' differential ability to recognize and cleave methylated and unmethylated CpG DNA sites.

e) Methyl Sensitive Southern Blotting is similar to the HELP assay but uses Southern blotting techniques to probe gene-specific differences in methylation using restriction digests. This technique is used to evaluate local methylation near the binding site for the probe.

f) ChIP-on-chip assay is based on the ability of commercially prepared antibodies to bind to DNA methylation-associated proteins like MeCP2.

g) Restriction landmark genomic scanning is a complicated and now rarely-used assay is based upon restriction enzymes' differential recognition of methylated and unmethylated CpG sites. This assay is similar in concept to the HELP assay.

h) Methylated DNA immunoprecipitation (MeDIP) is analogous to chromatin immunoprecipitation. Immunoprecipitation is used to isolate methylated DNA fragments for input into DNA detection methods such as DNA microarrays (MeDIP-chip) or DNA sequencing (MeDIP-seq).

i) Pyrosequencing of bisulfite treated DNA is a sequencing of an amplicon made by a normal forward primer but a biotinylated reverse primer to PCR the gene of choice. The Pyrosequencer then analyses the sample by denaturing the DNA and adding one nucleotide at a time to the mix according to a sequence given by the user. If there is a mismatch, it is recorded and the percentage of DNA for which the mismatch is present is noted. This gives the user a percentage methylation per CpG island.

In certain embodiments of the invention, the genomic DNA is hybridized to a complimentary sequence (e.g. a synthetic polynucleotide sequence) that is coupled to a matrix (e.g. one disposed within a microarray). Optionally, the genomic DNA is transformed from its natural state via amplification by a polymerase chain reaction process. For example, prior to or concurrent with hybridization to an array, the sample may be amplified by a variety of mechanisms, some of which may employ PCR See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675. The sample may be amplified on the array. See, for example, U.S. Pat. No. 6,300,070, which is incorporated herein by reference.

In addition to using art accepted modeling techniques on data obtained from embodiments of the invention (e.g. regression analyses), embodiments of the invention can utilize a variety of art accepted technical processes. For example, in certain embodiments of the invention, a bisulfite conversion process is performed so that cytosine residues in the genomic DNA are transformed to uracil, while 5-methylcytosine residues in the genomic DNA are not transformed to uracil. Kits for DNA bisulfite modification are commercially available from, for example, MethylEasy™ (Human Genetic Signatures™) and CpGenome™ Modification Kit (Chemicon™). See also, WO04096825A1, which describes bisulfite modification methods and Olek et al. Nuc. Acids Res. 24:5064-6 (1994), which discloses methods of performing bisulfite treatment and subsequent amplification. Bisulfite treatment allows the methylation status of cytosines to be detected by a variety of methods. For example, any method that may be used to detect a SNP may be used, for examples, see Syvanen, Nature Rev. Gen. 2:930-942 (2001). Methods such as single base extension (SBE) may be used or hybridization of sequence specific probes similar to allele specific hybridization methods. In another aspect the Molecular Inversion Probe (MIP) assay may be used.

The CpG sites discussed herein are found in Table 9 below. The Illumina method takes advantage of sequences flanking a CpG locus to generate a unique CpG locus cluster ID with a similar strategy as NCBI's refSNP IDs (rs#) in dbSNP (see, e.g. Technical Note: Epigenetics, CpG Loci Identification ILLUMINA Inc. 2010).

### Example 1: Illustrative Aspects and Working Embodiments of the Invention

DNAm levels have been used to build accurate composite biomarkers of chronological age [1-6]. DNAm-based age (epigenetic age) estimators, include an estimator developed by Horvath [1], based on 353 CpGs in different somatic tissues, and an estimator developed by Hannum [2], based on 71 CpGs in leukocytes. These estimators predict lifespan after adjusting for chronological age and other risk factors [7-9]. Moreover, they are associated with a host of age-related conditions [10-13]. These DNAm-based age estimators highly correlate with chronological age (r>0.9), but they are less accurate in predicting mortality, healthspan, and various age-related morbidities [10]. Several DNAm-based biomarkers are better mortality predictors because they explicitly use lifespan (time to death) in their construction [3, 4]. For example, Zhang et al (2017) combined mortality associated CpGs [4] into an overall mortality risk score. Levine et al (2018) developed a mortality predictor, DNAm PhenoAge, by regressing a phenotypic measure of mortality risk on CpGs [3].

Many analytical strategies are possible for building mortality predictors from DNAm data. The direct approach involves regressing time-to-death (due to all-cause mortality) on DNAm levels. Here we describe a two-stage procedure: In stage 1, we defined DNAm-based surrogate biomarkers of smoking pack-years and select plasma proteins which have been associated with mortality or morbidity. In stage 2, we regressed time-to-death on these DNAm based surrogate biomarkers. The mortality risk estimate of the regression model is linearly transformed so that it is in units of age. We coin this DNAm-based biomarker of mortality “DNAm GrimAge” because high values are grim news when it comes to lifespan. Using validation data from three ethnic groups, we demonstrate that DNAm GrimAge stands out in terms of its predictive ability for time-to-death due to all-cause mortality and time-to-coronary heart disease. An age-adjusted version of DNAm GrimAge, which can be regarded as a new measure of epigenetic age acceleration (AgeAccelGrim), is associated with a host of age-related conditions, lifestyle factors, biomarkers of immunosenescence, and leukocyte telomere length.

### Overview of the Two-Stage Approach for Defining DNAm GrimAge

We constructed the DNAm GrimAge in two-stages. First, we defined surrogate DNAm biomarkers of physiological risk factors and stress factors. These include the following plasma proteins: adrenomedullin, C-reactive protein, plasminogen activation inhibitor 1 (PAI-1), and growth differentiation factor 15 (GDF15) [14, 15]. In addition, given that smoking is a significant risk factor of mortality and morbidity, we also used DNAm-based estimator of smoking pack-years. Second, we combined these biomarkers into a single composite biomarker of lifespan, DNAm GrimAge, which is expressed in units of years. We then performed a large-scale meta-analysis (involving more than 7000 Illumina array measurements), showing that DNAm GrimAge is a better predictor of lifespan that present DNAm-based predictors.

Our studies reveal a surprising finding: sometimes, the DNAm based surrogate biomarkers (e.g. for smoking pack-years) is a better predictors of mortality than the underlying observed (self-reported) biomarker. We also correlated DNAm GrimAge with lifestyle factors and a host of age-related conditions, e.g. we demonstrate that our DNAm based biomarkers predict time to cardiovascular disease. Finally, we demonstrate that DNAm GrimAge is associated with age-related changes in blood cell composition and leukocyte telomere length.

Training and Test Data from the Framingham Heart Study

We correlated levels of 88 plasma protein variables (measured using an immunoassay) with DNAm array data generated from the same blood samples of n=2,356 individuals from the Framingham heart study (FHS) Offspring Cohort [16] (note 1 below). We randomly split the FHS data into a training set (70% of the FHS pedigrees, N=1731 individuals from 622 pedigrees) and a test data set (30% pedigrees, N=625 individuals from 266 pedigrees, (Table 3). The mean age of individuals donating DNA for the training set was 66 years. The mean age of individuals in the test dataset was 67. These participants had a similar demographic profile, smoking history, and number of years' follow-up as those used for the training set (Table 3).

### Stage 1: DNAm-Based Surrogate Biomarkers of Plasma Proteins and Smoking Pack-Years

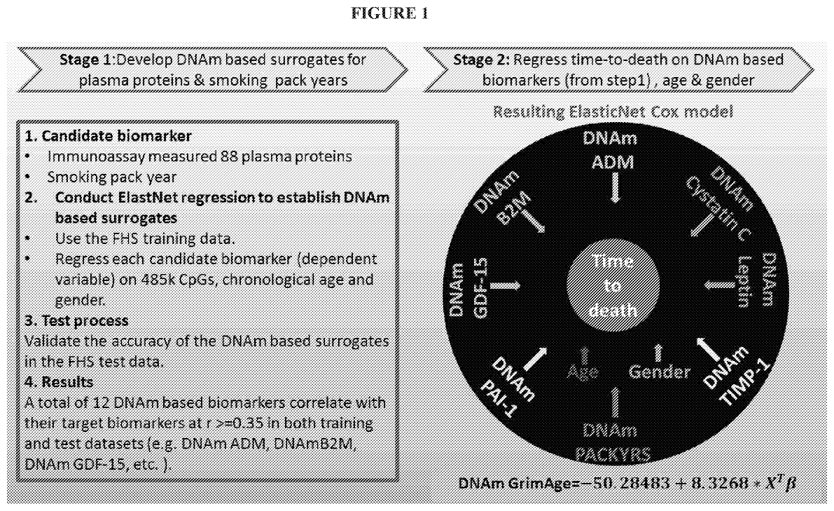

We used the training data to define DNAm-based surrogate markers of 88 plasma protein variables and smoking pack-years. We restricted the analysis to CpGs that are present on both the Illumina Infinium 450K array and the new Illumina EPIC methylation array in order to ensure future compatibility. Each of the 88 plasma protein variables (dependent variable) was regressed on chronological age, sex, and the CpGs levels in the training data using an elastic net regression model, which automatically selected a subset of CpGs (typically fewer than about 200 CpGs) whose linear combination best predicted the corresponding plasma level in the training data (Methods). For example, the DNAm levels of 137 CpGs and 211 CpGs allowed us to estimate the plasma levels of GDF15 and PAI-1, respectively. The predicted DNAm values of GDF15 and PAI-1 can be used as surrogate markers for the observed plasma levels. In general, we denote DNAm-based surrogate markers of plasma proteins and smoking pack-years by adding the prefix “DNAm” to the respective variable name, e.g. DNAm pack-years (FIG. 1 and Table 4).

Not all of the available 88 plasma protein could be successfully imputed based on DNAm data. Only 12 (out of 88) plasma proteins exhibited a correlation coefficient greater than 0.35 between the observed plasma levels and their respective DNAm-based surrogate marker in the test data set, (Table 1). We focused on these 12 DNAm surrogate biomarkers in stage 2. Additionally, we constructed a DNAm-based surrogate of self-reported smoking pack-years, DNAm pack-years, based on a linear combination of 172 CpGs.

### Stage 2: Constructing a Composite Biomarker of Lifespan Based on Surrogate Biomarkers

In stage 2, we developed a predictor of mortality by regressing time-to-death due to all-cause mortality (dependent variable) on the following covariates: the DNAm-based estimator of smoking pack-years, chronological age at the time of the blood draw, sex, and the 12 DNAm-based surrogate biomarkers of plasma protein levels. The ElasticNet Cox regression model automatically selected the following covariates: DNAm pack-years, age, sex, and the following 7 DNAm based surrogate markers of plasma proteins: adrenomedullin (ADM), beta-2-microglobulim (B2M), cystatin C (Cystatin C), GDF-15, leptin (Leptin), PAI-1, and tissue inhibitor metalloproteinases 1 (TIMP-1), (Table 4). DNAm-based biomarkers for smoking pack-years and the 7 plasma proteins are based on typically fewer than about 200 CpGs each, totaling 1,113 unique CpGs (Table 4). Details on the plasma proteins can be found in Note 2 below.

The linear combination of covariates resulting from the ElasticNet Cox regression model can be interpreted as an estimate of the logarithm of the hazard ratio of mortality. We linearly transformed this parameter into an age estimate, i.e., DNAm GrimAge, by performing a linear transformation whose slope and intercept terms were chosen by forcing the mean and variance of DNAm GrimAge to match that of chronological age in the training data (Methods, FIG. 1). In independent test data, DNAm GrimAge is calculated without estimating any parameter because the numeric values of all parameters were chosen in the training data. Following the terminology from previous articles on DNAm-based biomarkers of aging, we defined a novel measure of epigenetic age acceleration, AgeAccelGrim, which, by definition, is not correlated (r=0) with chronological age. Toward this end, we regressed DNAm GrimAge on chronological age using a linear regression model and defined AgeAccelGrim as the corresponding raw residual (i.e. the difference between the observed value of DNAm age minus its expected value). Thus, a positive (or negative) value of AgeAccelGrim indicates that the DNAm GrimAge is higher (or lower) than expected based on chronological age.

Unless indicated otherwise, we used AgeAccelGrim (rather than DNAm GrimAge) in association tests of age-related conditions because age was a confounder in these analyses. For the same reason, we also used age-adjusted versions of our DNA based surrogate markers (for smoking pack-years and the seven plasma protein levels). In general, all association tests were adjusted for chronological age and, when required, other confounders (such as sex, Methods).

### Pairwise Correlations Between DNAm GrimAge and Surrogate Biomarkers

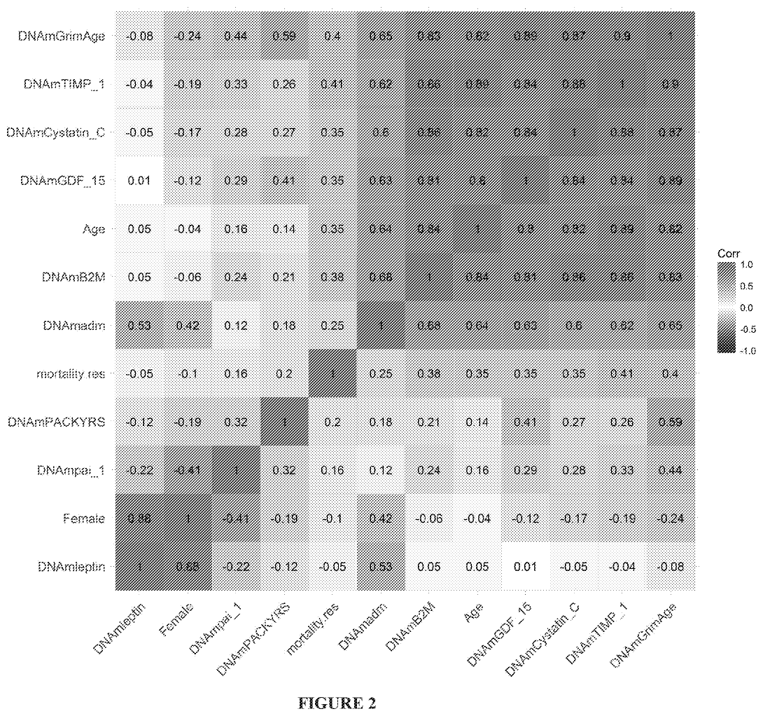

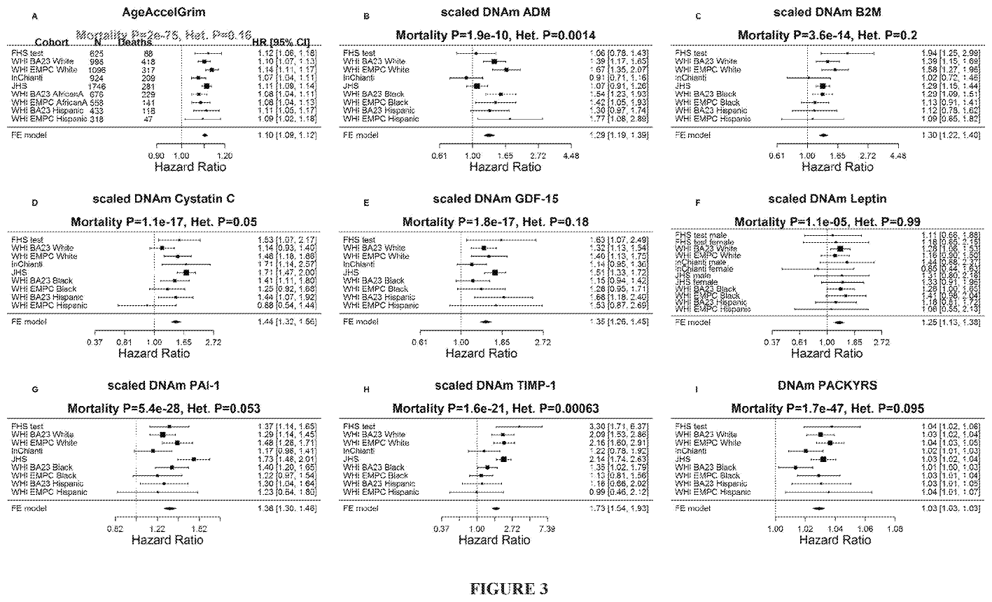

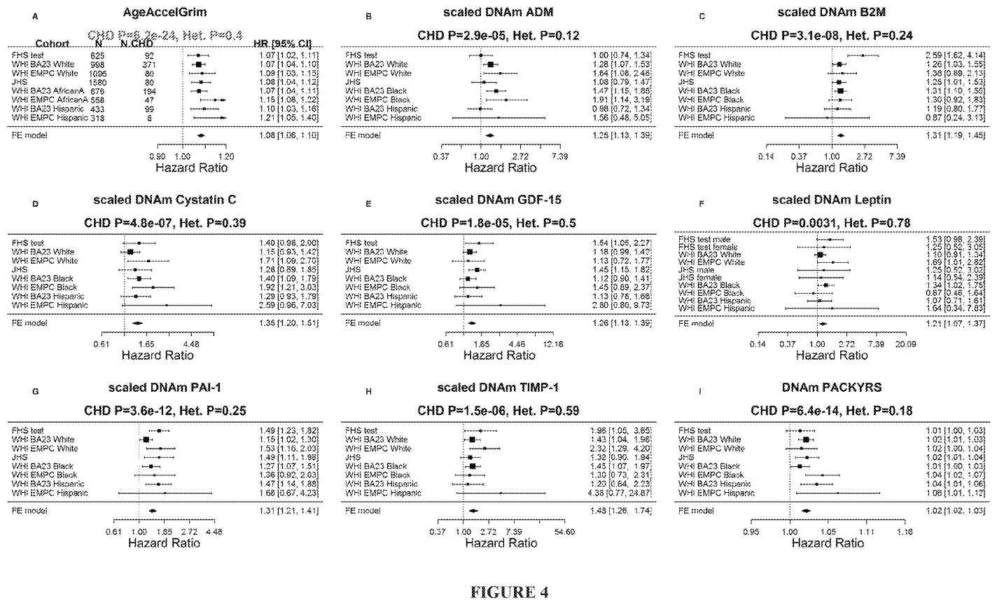

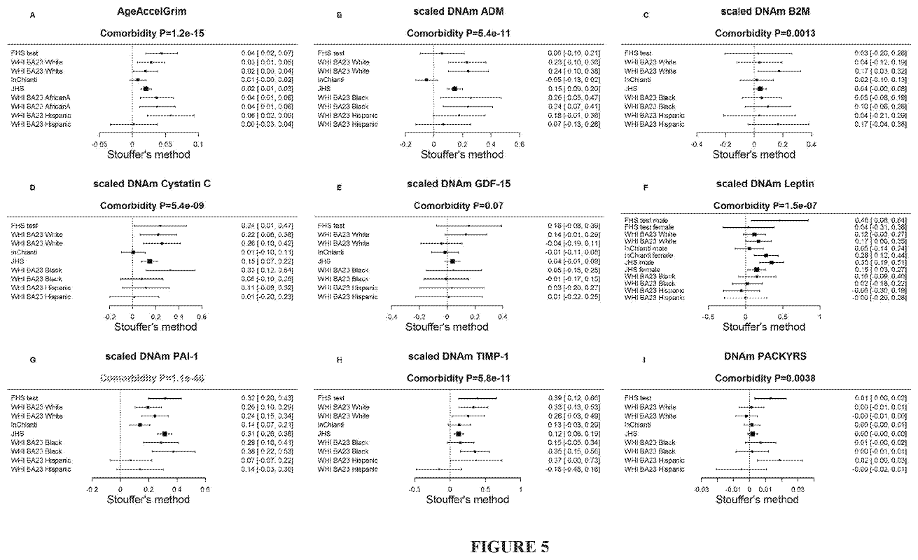

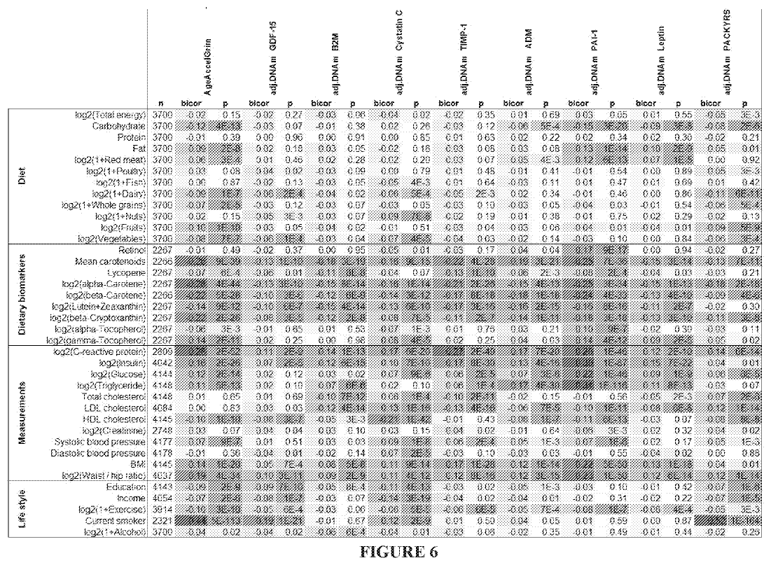

Using the test data from the FHS, we calculated pairwise correlations between DNAm GrimAge and its underlying variables (FIG. 2 and Table 4). DNAm GrimAge is highly correlated with DNAm TIMP-1 (r=0.90) and chronological age (r=0.82). An estimate of excess mortality risk (called mortality residual mortality.res) exhibits higher positive correlations with both DNAm GrimAge and DNAm TIMP_1 (r˜0.40) than with chronological age (r˜0.35, FIG. 2), in keeping with our later finding that these DNAm biomarkers are better predictors of lifespan than chronological age. With the exception of DNAm Leptin, all of the DNAm based biomarkers exhibited positive correlations with the measure of excess mortality risk (0.41≥r≥0.16, FIG. 2). With the exception of DNAm Leptin, all DNAm based surrogate biomarkers exhibited moderate to strong pairwise correlations with each other.

DNAm Leptin is elevated in females consistent with what has been reported in the literature [17, 18]. After stratifying by sex, we find that plasma leptin levels increase weakly with age (r=0.18 and P=2.1E-3 in males; r=0.19, P=4.8E-4 in females).

### Predicting Time-to-Death in Validation Data

To evaluate whether our novel DNAm based biomarkers are better predictors of lifespan than chronological age, we analyzed N=7,375 Illumina methylation arrays generated from blood samples of 6,935 individuals comprising 3 ethnic/racial groups: 50% European ancestry (Caucasians), 40% African Americans, and 10% Hispanic ancestry (Table 2, Methods). The data came from different cohort studies: test data from the FHS, BA23 and EMPC study from the Women's Health Initiative (WHI), the InCHIANTI cohort study, and African Americans from the Jackson Heart Study (JHS). We stratified each cohort by race/ethnicity (resulting in 9 strata) to avoid confounding and to ascertain whether the mortality predictors apply to each group separately.

The mean chronological age at the time of the blood draw was 63.0 years. The mean follow-up time (used for assessing time-to-death due to all-cause mortality) was 13.7 years. Since chronological age is one of the component variables underlying DNAmGrimAge, it is not surprising that the latter is highly correlated with age in each of the study cohorts (r≥0.79).

While each (age-adjusted) component variable underlying DNAm GrimAge is a significant predictor of lifespan (FIG. 3), DNAm pack-years (meta-analysis P=1.7E-47) and DNAm PAI-1(P=5.4E-28) exhibit the most significant meta-analysis P-values. The fixed effects meta-analysis P-values reveal that AgeAccelGrim stands out when it comes to lifespan prediction (meta-analysis P=2.0E-75, FIG. 3A). The same applies when the analysis is restricted to never-smokers or to former/current smokers. AgeAccelGrim remains a highly significant predictor of lifespan after restricting the analysis to never-smokers (N=3,988, meta-analysis P=1.1E-16) or to former/current smokers (P=3.5E-33).

### DNAm Based Surrogates Sometimes Outperform Observed Biomarkers

Our DNAm-based surrogate biomarker for smoking pack-years has to surprising properties. First, it predicts lifespan in never-smokers (P=1.6E-6). Second, the surrogate marker is a more significant predictor of lifespan than self-reported pack-years: P=8.5E-5 for DNAm marker versus P=2.1E-3 for observed pack-years in in the FHS test data; similarly, P=5.3E-4 versus 0.18 in the InChianti Study (Table 5). The superior predictive performance of DNAm based surrogate biomarkers vis-à-vis their observed counter parts also applies to PAI-1 plasma levels (P=8.7E-4 for the DNAm marker versus P=0.074 for the observed levels), TIMP-1 (P=3.8E-4 for the DNAm marker versus P=0.017), and to a lesser extent to cystatin C (P=0.019 for the DNAm estimator versus P=0.054 for the observed level, (Table 6).

### Mortality Prediction Based on Observed Plasma Protein Levels

The AgeAccelGrim is a composite biomarker derived from DNAm-based surrogate biomarkers of plasma protein levels and smoking pack-years. This begs the question whether a predictor of lifespan based on observed plasma protein levels and self-reported smoking pack-years, outperforms its DNAm-based analog. Analogous to our construction of DNAm GrimAge, we used a Cox regression model to regress-time to-death on the observed plasma protein levels and self-reported pack-year in the training data (Methods). The resulting mortality risk estimator (defined as weighted average of the observed biomarkers) was linearly transformed into units of years. The resulting predictor, i.e., observed GrimAge, and its age-adjusted version. i.e., observed AgeAccelGrim, were compared in the FHS, showing similar HRs (observed AgeAccelGrim HR=1.10, P=3.2E-7; observed AgeAccelGrim HR=1.12, P=8.6E-5, Table 7). Overall, this comparison shows that DNAm levels in general and our DNAm-based surrogate biomarkers in particular capture a substantial proportion of the information captured by the 7 selected plasma proteins and self-reported smoking pack-years. Since our study focuses on DNAm based biomarkers, we will only consider DNAm based biomarkers in the following.

### Age-Related Conditions

Our Cox regression analysis of time to coronary heart disease (CHD), reveals that AgeAccelGrim is highly predictive of incident CHD (HR=1.07, P=6.2E-24 and P_{I}z=0.4, FIG. 4A). As expected, several underlying DNAm-based surrogate biomarkers also predict incident CHD notably our age-adjusted versions of DNAm smoking pack-years (HR=1.02, P=6.4E-14) and DNAm PAI-1 (HR=1.31 per SD, P=3.6E-12). Similarly, time-to-congestive heart failure (CHF) is associated with AgeAccelGrim (HR=1.10 and P=4.9E-9), age adjusted DNAm cystatin C (HR=2.02 and P=2.0E-10) and DNAm PAI-1 (HR=1.58 and P=8.9E-10).

Cross sectional studies reveal that AgeAccelGrim is associated with hypertension (odds ratio [OR]=1.04 and P=5.1E-13), type 2 diabetes (OR=1.02 and P=0.01), and physical functioning (Stouffer P=3.8E-13). All of the reported associations are in the expected directions, e.g. higher values of AgeAccelGrim are associated with lower physical functioning levels. In women, early age at menopause is associated with significantly higher values of AgeAccelGrim (P=1.6E-12) and to a lesser extent with all of the age-adjusted versions of the DNAm based surrogate markers, notably DNA cystatin C (P=2.2E-6) and DNAm GDF-15 (P=1.3E-5).

### DNAm Plasminogen Activation Inhibitor I

AgeAccelGrimAge outperforms (age-adjusted versions of) DNAm smoking pack-years and the 7 DNAm-based surrogate markers of plasma protein levels when it comes to predicting time-to-death or time-to-coronary heart disease (FIGS. 3 & 4). However, age-adjusted DNAm PAI-1 outperforms AgeAccelGrim for several age-related traits, notably the comorbidity index (defined as total number of age-related conditions) where Stouffer's meta-analysis P value for DNAm PAI-1 (P=1.1E-46) is more significant than that for AgeAccelGrim (P=1.2E-15, FIG. 5). Similar to AgeAccelGrim, higher levels of age-adjusted DNAm PAI-1 are associated with hypertension status, type 2 diabetes status, time-to-CHD (FIG. 4), time-to-CHF, and early age at menopause and lower levels are associated with disease free status (Stouffer P=2.9E-11) and better physical functioning (Stouffer P=1.2E-12).

### Heritability Analysis

We used pedigree based polygenic models (Methods) to arrive at heritability estimates of AgeAccelGrim and our other biomarkers. Significant heritability estimates can be observed for AgeAccelGrim (h²=0.30, P=0.022) and observed AgeAccelGrim (h²=0.37, P=0.0060, Table 8). Similarly, several of our DNAm based surrogate biomarkers (for PAI1, B2M, ADM, GDF15) and their observed counterparts are highly heritable (Table 8), e.g. DNAm PAI-1 (h²=0.34 and P=7.1E-3), observed PAI-1 levels (h²=0.51, P=6.2E-4), DNAm Beta 2 microglobulin levels (h²=0.45, P=2.4E-3), and observed B2M (h²=0.34, P=3.3E-3). Overall, these results show that many DNAm based biomarkers are under considerable genetic control.

### AgeAccelGrim Versus Other Epigenetic Measures of Age Acceleration

Using the same validation datasets (N=7,375 arrays), we compared DNAm GrimAge with three widely-used DNA-based biomarkers of aging: DNAm age estimator based on different somatic tissues by Horvath (2013) [1],

The DNAm age estimator based on leukocytes by Hannum (2013) [2] and the DNAm PhenoAge estimator by Levine (2018) [3]. The respective age adjusted measures of epigenetic age acceleration will be denoted as AgeAccel (or AgeAccelerationResidual), AgeAccelHannum, and AgeAccelPheno following the notation of previous publications. The four epigenetic measures of age acceleration (including AgeAccelGrim) are in units of year. AgeAccelGrim exhibits moderate positive correlations with each of the three alternative measures of epigenetic age acceleration (0.17≤r≤0.45) and the strongest correlation with AgeAccelPheno. AgeAccelGrim stands out with respect to the meta-analysis P-values for predicting time-to-death: AgeAccelGrim (P=2.0E-75, HR=1.10), AgeAccel (Meta P=8.9E-5, HR=1.02), AgeAccelHannum (Meta P=6.8E-16, HR=1.04), AgeAccelPheno (Meta P=3.5E-36, HR=1.05). The results remain qualitatively the same after restricting the analysis to never-smokers or former/current smokers.

Similarly, AgeAccelGrim stands out when comparing individuals in the top 20% percentile of epigenetic age acceleration to those in the bottom 20% percentile (Stouffer meta-analysis P=2.7E-39), AgeAccelPheno (P=3.0E-20), AgeAccelHannum (P=4.9E-6), and AgeAccel (P=0.14).

When it comes to significant associations with the comorbidity index, age-adjusted DNAm PAI-1(P_{DNAmPAI-1}=1.1E-46, FIG. 5) outperforms all other DNAm-based biomarkers including AgeAccelGrim (P_{AgeAccelGrim}=1.2E-15) and AgeAccelPheno (P_{AgeAccelPheno}==2.7E-18).

AgeAccelGrim is more informative than AgeAccelPheno when it comes to predicting time to CHD (P_{AgeAccelOrtm}=6.2E-24 and HR_{AgeAccelGrim}=1.07 versus P_{AgeAccelPheno}=1.7E-8 and HR_{AgeAccelPheno}=1.03) even after stratifying the analysis by smoking status.

AgeAccelGrim greatly outperforms the other 3 measures of epigenetic age acceleration including predicting time to (any) cancer (AgeAccelGrim P=1.3E-12 versus AgeAccelPheno P=2.7E-3) and as related to an inverse association with early age at menopause in women (AgeAccelGrim P=1.6E-12 versus AgeAccel P=2.2E-3). A sensitivity analysis reveals that the latter finding remains qualitatively the same even after removing the InChianti cohort, which exhibited the strongest negative association between epigenetic age acceleration and age at menopause.

### Multivariate Cox Models Adjusting for Traditional Risk Factors

Our above-mentioned Cox regression models adjusted for age at blood draw (baseline), batch, pedigree, and intra subject correlation as needed. We also fit multivariate Cox regression models that included additional covariates assessed at baseline: body mass index, educational level, alcohol intake, smoking pack-years, prior history of diabetes, prior history of cancer, and hypertension status (Methods). Even after adjusting for these known risk factors for morbidity, AgeAccelGrim remained a highly significant predictor of lifespan (P=5.7E-29) and time-to-CHD (P=3.7E-11) and outperformed previously published measures of epigenetic age acceleration.

### Stratified Analyses

We evaluated AgeAccelGrim and underlying DNAm biomarkers in different strata characterized by age (younger/older than 65 years), body mass index (obese versus non-obese), educational attainment, prevalent condition at baseline such as prior history of cancer, type 2 diabetes, or hypertension. In all of these strata, AgeAccelGrim remains a significant predictor of time-to-death and time-to-CHD. Further, AgeAccelGrim outperforms existing DNAm based biomarkers of aging in all strata except for one (comprised of n=281 individuals with a prior history of cancer).

These subgroup analysis results also confirm that epigenetic age acceleration is an independent predictor of earlier mortality even after adjusting for possible confounders and within major subgroups of the population. With few exceptions, we found that DNAm based PA-1, TIMP-1 and pack-years remained highly significant in each stratum.

### Exceptionally Fast/Slow Agers

The DNAm GrimAge estimate has an intuitive interpretation as physiological age since it is in units of years. However, if someone is 8 years older than expected, that does not mean that this person has on average an 8-year shorter life expectancy. Rather, lifespan calculations need to account for the hazard ratio associated with AgeAccelGim as indicated in the following. It is a statistical co-incidence that the hazard ratio associated with one-year increase in AgeAccelGrim is the same in strata comprised of never-smokers (HR=1.10), former/current smokers (HR=1.10), and among all individuals combined (HR=1.10). This allows us to evaluate the mortality risks in exceptionally fast and slow agers (according to AgeAccelGrim) irrespective of their smoking status. The top 5^{th} percentile and the 95% percentile of AgeAccelGrim corresponds to −7.5 years and +8.3 years. A person in the top 95^{th} percentile of AgeAccelGrim (=8.3 years) faces a hazard of death that is twice that of the average person in their straturm (whose AgeAccelGrim equals 0). Specifically, fast aging status is associated with a hazard ratio of HR=2.2=1·10^{8.3}. Conversely, a slow ager in the bottom 5^{th} percentile (−7.5 years) faces a hazard of death that is half that of the average person in their stratum, HR=0.49=1·10^{7.5}.

### DNAm GrimAge Versus Single Stage Estimators of Lifespan

DNAm GrimAge was built using a novel two-stage approach that critically depended on the development of DNAm-based surrogate biomarkers. To justify the utility of this indirect approach, we compared DNAm GrimAge with several DNAm-based lifespan predictors that were developed by directly regressing lifespan on DNAm data (referred to as single stage mortality predictors). To this end, we developed a new mortality predictor, DNAm Mortality (in year units) by directly regressing time-to-death (due to all-cause mortality) on CpGs in the FHS training data. DNAm Mortality was calculated as linear combination of 59 CpGs. The direct approach entailed the constructions of DNAm Mortality, an ElasticNet Cox regression model, and linear transformation of the mortality risk to ensure that the values of DNAm Mortality are in units of years (Methods). Further, we also evaluated the published mortality predictor from Zhang [4] which, remarkably, is based on only 10 CpGs (Methods). The latter two (single-stage) lifespan predictors are highly correlated with each other (r=0.77 in the FHS test data).

The novel age-adjusted DNAm Mortality estimator (HR=1.07, P=3.0E-44) and both versions of Zhang's mortality risk estimator (continuous HR=1.02, P=4.2E-39) lead to a less significant meta-analysis P-value for lifespan prediction than AgeAccelGrim (HR=1.10, P=2.0E-75). AgeAccelGrim also stands out in terms of its meta-analysis P-value for predicting time-to-CHD (AgeAccelGrim P=1.1E-20, AgeAccelMortality P=1.8E-11, AgeAccelZhang P=1.9E-10).

Because smoking is a major risk factor, it is useful to characterize the different lifespan predictors in terms of their correlation with DNAm pack-years. Age-adjusted DNAm pack-years exhibits positive correlations with both DNAm Mortality and Zhang's mortality predictor (r≥0.55). The connection of single stage mortality predictors to smoking can also be observed at the CpG level. DNAm Mortality, Zhang's mortality predictor, and DNAm pack-years explicitly use CpG cg05575921 (in the AHRR gene on chromosome 5p15.33), which has previously been highlighted by epigenome-wide association studies of cumulative smoking exposure [4, 19]. Overall, these results suggest that the two single-stage lifespan predictors relate more strongly to cumulative smoking exposure than AgeAccelGrim.

Association with Blood Cell Composition

DNAm data allow one to estimate several abundance measures of blood cells as described in (Methods)[20, 21]. We previously showed that DNAm biomarkers of aging, which capture age-related changes in blood cell composition are better predictors of lifespan than those that are independent of blood cell counts [9]. Therefore, we hypothesized that several of our novel DNAm biomarkers would exhibit significant correlations with these imputed measures of blood cell composition. This is indeed the case as can be seen from our large-scale meta-analysis across the validation data. AgeAccelGrim is significantly associated with a decrease in naive CD8+ T cells (r=−0.22, P=9.2E-62), CD4+T cells (r=−0.21, P=1.8E-57), and B cells (r=−0.18, P=9.7E-43) and with an increase in granulocytes/neutrophils (r=0.24, P=1.5E-74) and plasma blasts (r=0.22, P=7.3E-63). While these results demonstrate that AgeAccelGrim is associated with an age-related decline in immune system functioning, our cross-sectional analysis does not allow us to dissect cause-and-effect relationships.

Age-adjusted DNAm TIMP-1 exhibits the most significant correlations with the measures of blood cell composition (e.g. proportion of granulocytes r=0.36, P=2.7E-172) followed by age adjusted DNAm Cystatin C (proportion of CD4+ T cells counts r=−0.33, P=3.4E-142). Although many of our DNAm biomarkers are correlated with blood cell counts, this does not mean that these measures only capture changes in blood cell composition. This point is illustrated by the finding that measures of blood cell composition only correlate weakly with our age-adjusted DNAm surrogate markers of smoking pack-years (strongest correlation r=−0.14) and PAI-1 levels (strongest correlation r=0.17) even though both biomarkers are strongly associated with mortality risk and age-related conditions as shown above.

Association with Leucocyte Telomere Length

Leukocyte telomere length (LTL) has been found to be predictive of mortality and cardiovascular disease.

Our meta-analysis reveals a statistically significant but weak negative correlation between LTL and AgeAccelGrim (r=−0.12 and meta P=3.3E-10) across data from the FHS, WHI (BA23 sub-study) and JHS (total N=2,702, 27% White and 73% African American). Similarly, LTL exhibits (weak) negative correlations with our DNAm based surrogate biomarkers for GDF-15 (r=−0.10, meta P=3.4E-7), DNAm PAI-1 (r=−0.10, meta P=5.1E-8) and smoking pack-years (r=−0.09 and meta P=2.9E-6).

### Diet, Education, and Life Style Factors

Several previous measures of epigenetic age acceleration in blood have been shown to exhibit statistically significant but weak correlations with lifestyle factors and biomarkers of metabolic syndrome [3, 22]. Here we revisited these cross-sectional studies in the WHI (comprising approximately 4000 postmenopausal women, Methods) with our novel measures of AgeAccelGrim and its underlying DNAm-based surrogate biomarkers (FIG. 6).

All of our (age-adjusted) DNAm-based biomarkers correlate with plasma biomarkers measuring vegetable consumption, but AgeAccelGrim (robust correlation coefficient r=−0.26, P=9E-39, FIG. 6) and DNAm PAI-1 (r=−0.25, P=7E-36) stand out in terms of their strong relationship with mean carotenoid levels (FIG. 6). Similar, but far less significant negative associations, could be observed for self-reported measures of fruit-, vegetable-, and dairy intake. The following results were unexpected because they had not been observed with previous DNAm-based biomarkers of aging: (self-reported) proportion of carbohydrate consumption was associated with lower AgeAccelGrim (robust correlation r=−0.12, P=4E-13) and DNAm PAI-1 (r=−0.15, P=3E-20). Conversely, an increased proportion of fat intake (but not protein intake) was associated with increased AgeAccelGrim (r=0.09, P=2E-8) and DNAm PAI-1 (r=0.13, P=1E-14). Measures of lipid metabolism, triglyceride levels and HDL cholesterol levels, were significantly correlated with AgeAccelGrim (r=0.11 and r=−0.10, respectively) and even more so with (age adjusted) DNAm PAI-1 levels (r=0.34 and r=−0.11). Similarly, measures of glucose metabolism, insulin- and glucose levels, exhibited positive correlations with AgeAccelGrim (r=0.16 and r=0.12, respectively) and with (age adjusted) DNAm PAI levels (r=0.30 and r=0.22).

Similar to what we observed with previous DNAm based biomarkers of aging, plasma C-reactive protein levels exhibited comparatively strong positive correlations with DNAm-based biomarkers, particularly AgeAccelGrim (r=0.28, P=2E-52), DNAm TIMP-1 (r=0.27, P=2E-49), and DNAm PAI-1 (r=0.26, P=1E-46).

Measures of adiposity, BMI and waist-to-hip ratio, are associated with increased AgeAccelGrim, age-adjusted DNAm PAI-1, and other DNAm based surrogate biomarkers. Higher education and income are associated with lower AgeAccelGrim (P=2E-9 and P=2E-6). AgeAccelGrim stands out when it comes to detecting a beneficial effect of physical exercise (r=−0.10, P=3E-10).

Several of our results in the WHI could be replicated in a smaller data (N<625 individuals) from the FHS test dataset that included lipid and metabolic biomarker data. In the FHS, hemoglobin A1C and albumin levels (in urine) exhibited significant positive correlations with AgeAccelGrim, age-adjusted DNAm PAI-1 (0.10≤r≤50.12 and 1.4E-07≤P≤2.3E-03), and to a lesser extent with our other DNAm based surrogate biomarkers.

DNAm-based biomarkers are strong predictors of aging [10, 23]. Several recent articles describe DNAm-based biomarkers for measuring tissue age and for predicting lifespan. This work shows that DNAm GrimAge, which in essence is as a linear combination of age, sex, and surrogate biomarkers for plasma proteins and smoking pack-years, outperforms all other DNAm-based predictors of lifespan and CHD. Moreover, the DNAm-based biomarkers of smoking and PAI-1 might be used independently of DNAm GrimAge to test a host of hypotheses linking smoking and PAI-1 to aging and its related diseases. The DNAm based surrogate biomarker of smoking might complement self-reported assessments of pack-years. The surprising fact that DNAm pack-years outperforms self-reported pack-years in predicting lifespan could reflect erroneous self-reporting. Alternatively, DNAm pack-years may capture intrinsic variation across individuals in the lasting biological damage that results from smoking, i.e., inter-individual sensitivities to smoking.

Notably, only a small fraction of plasma proteins (7 out of 88) could be imputed in this work based on DNAm. In the FHS data, the measurement of the plasma proteins (exam 7) preceded the measurement of blood DNAm data (exam 8) by 6.6 years, suggesting that DNAm is not just a snapshot of the status of these proteins at the time of blood collection. That said, the elucidation of cause-and-effect relationships between plasma proteins and DNAm will require future longitudinal cohort studies and mechanistic evaluations. Our DNAm-based surrogate biomarkers of plasma protein levels may be leveraged by researchers who rely on bio-banked DNA samples without the availability of plasma samples.

Strong evidence supports links between plasma proteins used in the construction of GrimAge and various age-related conditions: ADM levels are increased in individuals with hypertension and heart failure[24]. Plasma B2M is a clinical biomarker associated with cardiovascular disease, kidney function, and inflammation [25]. Plasma cystatin-C is used to assess kidney function [26]. Overall, ADM, B2M, cystatin C, and leptin relate to many age-related traits including cognitive functioning [27-29]. GDF-15 is involved in age-related mitochondrial dysfunction [29]. PAI-1 plays a central role in a number of age-related subclinical and clinical conditions [30], and recent genetic studies link PAI1 to lifespan [31]. The tissue inhibitor of metalloproteinases, TIMP-1, plays an anti-apoptotic function [32].

While AgeAccelGrim stands out when it comes to lifespan prediction and prediction of time-to-CHD, our (age-adjusted), DNAm surrogate marker of PAI-1 level stands out when it comes to associations with type 2 diabetes status, glucose-, insulin-, triglyceride levels and measures of adiposity (body mass index and waist-to-hip ratio).

Inflammation and metabolic conditions are associated with AgeAccelGrim, age-adjusted DNAm PAI1 and age-adjusted DNAm TIMP-1.

Despite their obvious strengths, DNAm-based biomarkers are unlikely to replace existing clinical indices, e.g., blood glucose, blood lipids, blood pressure, etc., in medical practice. Rather, these biomarkers might complement existing clinical biomarkers when evaluating the individual's overall ‘aging’ status. Since DNAm captures important properties of the DNA molecule, these DNAm biomarkers are proximal to innate aging processes [10].

Finally, beyond lifespan prediction, AgeAccelGrim (and several of its underlying surrogate biomarkers) relate to most age-related conditions (metabolic syndrome, comorbidity, markers of inflammation such as C-reactive protein levels) in the expected way, i.e., increased values are associated with deleterious effect. Similarly, higher values of AgeAccelGrim (and several DNAm-based surrogate markers) are associated with a blood cell composition that is indicative of older individuals and shorter telomere length. While the reported associations are statistically significant, the magnitude of the underlying correlations is relatively small in keeping with the concept that aging is multi-factorial and perhaps the most complex of all human traits.

### Methods

### Study Cohort

To establish DNAm based estimators and DNAm GrimAge, we used 2,356 individuals composed of 888 pedigrees from the FHS cohort [16], a large-scale longitudinal study started in 1948, initially investigating risk factors for cardiovascular disease (CVD). The FHS cohort contains medical history and measurements, immunoassays at exam 7, and blood DNA methylation profiling at exam 8. The technology of immunoassay was based on Luminex xMAP assay, an extension of the enzyme-linked immunosorbent assay (ELISA) performed with multiple analyte-specific capture antibodies bound to a set of fluorescent beads. The DNA methylation profiling was based on the Illumina Infinium HumanMethylation450K BeadChip.

We assigned 70% pedigrees (1731 individuals/622 pedigrees) to the training process and the remaining 30% of pedigrees (625 individuals/266 pedigrees) to the FHS test data (Table 3). The training dataset was used to build the DNAm based surrogate markers for plasma proteins, smoking pack-years, and the composite biomarker DNAm GrimAge.

Validation Data from 5 Cohorts

Our validation analyses involved 7,375 Illumina arrays measuring blood methylation levels in N=6,935 individuals from five independent cohorts: the FHS test dataset (N=625), WHI BA23 (N=2107), WHI EMPC study (N=1972), JHS (N=1747), and InChianti (N=924 from 1 to 2 longitudinal measures on 484 individuals, Table 2). All the statistical analyses were adjusted for the correlation structure due to pedigree effects or repeated measurements as described below.

Estimation of Surrogate DNAm Based Biomarkers

We developed estimators for plasma proteins based on blood methylation data. We leveraged immunoassay measurements in the FHS which profiled 88 plasma protein biomarkers (in units of pg/mL), including cardiovascular disease related plasma proteins such as C-reactive protein[33] and growth differentiation factor 15 (GDF-15)[34]. For each protein marker, missing values were imputed by the respective median value. The median missing rate was <0.3%. Next the resulting observed plasma levels were regressed on DNAm data in the FHS training data.

Each plasma protein was regressed on the CpGs using the elastic net regression model implemented in the R package glmnet. Ten-fold cross validation was performed in the FHS training data to specify the underlying tuning parameter A.

We required the predicted variable associated with the target variable with ≥0.35 correlation in both training and test datasets. Only 12 out of 88 proteins exhibited a correlation greater than 0.35 between observed plasma levels and their respective DNAm based estimators in the FHS test data (Table 1). The missing rates of the 12 ImmunoAssay proteins were less than 0.7%. The correlation estimates have a distribution of 0.64±0.12 [0.43, 0.86] (mean±SD [range]) in the training dataset and a distribution of 0.43±0.09 [0.35, 0.66] in the test dataset.

### DNA Methylation Data

The Illumina 450K Infinium array profiled almost 486k CpG markers across the whole genome. Methylation beta values were generated using the Bioconductor minfi package with background correction. Our analysis focused on the subset of 450,161 CpGs that are also available on the Illumina EPIC array.

### Smoking Pack-Years

The variable “smoking pack-years” attempts to measure the cumulative amount of cigarettes consumed by the smoker. It is calculated by the number of packs of cigarettes smoked per day multiplied by the number of years the person smoked. We computed smoking pack-years using the information up to exam 8 in the FHS cohort.

### Definition of DNAm GrimAge

We again used an elastic net Cox regression model[35] to regress time-to-death (due to all-cause mortality) since exam 7 on the 12 DNAm based surrogate markers for plasma proteins and on DNAm PACKYR, chronological age, and sex. In the training dataset, we performed 10-fold cross validation to specify the value of the tuning parameter λ.

Linearly Transform DNAm GrimAge into Units of Years

The final ElasticNet Cox model listed in Table 4 results in an uncalibrated DNAm GrimAge estimate, which can be interpreted as the linear combination of the covariates, X^{T}β, or alternatively as the logarithm of the hazard ratio, log[h(t)/h₀(t)]=X^{T}β, where h₀(t) is the baseline hazard at time. The linear combination, X^{T}β, can be interpreted as an uncalibrated version of DNAm GrimAge. To facilitate an intuitive interpretation as a physiological age estimator, we linearly transformed it so that the resulting estimate would be in units of years. Toward this end, we imposed the following requirement:

the mean and variance of the resulting value, DNAm GrimAge, should be the same as the mean and variance of the age variable in the FHS training data (exam 7). This resulted in the following transformation

DNAm GrimAge=−50.28483+8.3268*X^{T}β,

### Observed DNAm GrimAge Model

Our finalized DNAm GrimAge is composed of 7 DNAm proteins, DNAm pack-years, age and gender, yielding DNAm based age acceleration measure: AgeAccelGrim (in units of year). We fitted a Cox regression model based on the observed proteins, self-report pack-years, age, and gender, using the FHS training dataset. This yielded the observed value of GrimAge. We computed age acceleration measures based on the observed and DNAm based GrimAge and compared their performances as risk factors for mortality, using the FHS training and test datasets, respectively.

### Statistical Models Used in Validation Analysis

Validation analysis was performed on 7,735 observations across 6,395 individuals (Table 2) coming from five datasets: the FHS test dataset (N=625), WHI BA23 (N=2107), WHI EMPC (N=1972), Jackson Heart Study (JHS, N=1747), and InChianti study (N=924 from 1 to 2 longitudinal measures on 484 individuals, Table 2). Our validation analysis involved i) Cox regression for time to death, for time-to-CHD, and for time to coronary heart failure, ii) linear regression for our DNAm based measures (independent variable) associated with and number of age-related conditions (dependent variable) and physical function score, respectively, iii) linear regression for age at menopause (independent variable) associated with our DNAm measure, iv) logistic regression analysis for estimating the odds ratios of our DNAm based measure associated with any cancer, hypertension, type 2 diabetes, emphysema, and disease free status. The variable of “number of age-related conditions” includes arthritis, cataract, cancer, CHD, CHF, emphysema, glaucoma, lipid condition, osteoporosis, type 2 diabetes, etc. In our validation analysis, we used AgeAccelGrim (the age-adjusted measure of DNAm GrimAge), and used the scaled measures of seven DNAm surrogates for plasma proteins based on the mean and standard deviation (SD) of the FHS training dataset such that the effect size was approximately corresponding to one SD. All the models were adjusted for age, and adjusted for batch effect as needed. To avoid the bias due to familial correlations from pedigrees in the FHS cohort or the intra subject correlations from the repeated measures, we accounted for the correlations accordingly in all the analyses in the following. In Cox regression analysis, we used robust standard errors, the Huber sandwich estimator, implemented in R coxph function. We used linear mixed models with a random intercept term, implemented in lme R function. We used generalized estimation equation models (GEE), implemented in R gee function, for our logistic regression models. Additional covariates related to demographic characteristics, psychosocial behaviors and clinical covariates were adjusted in multivariate Cox models analysis. The additional covariates include BMI (category), education attainment (category), alcohol consumption (gram/day), self-report smoking pack-years, three medical covariates: status of cancer, hypertension and type 2 diabetes at baseline. The categories associated with BMI ranges are a) 18.5-25 (normal), b) 25 to 30 (over), and c) >30 (obese). The categories associated with education attainment are a) less than high school, b) high school degree, c) some college, and d) college degree and above. Both smoking pack-years and education variables were not available in the JHS cohort. Smoking category (never, former and current) was used in the analysis using the JHS cohort.

### Meta-Analysis

Unless specified, we used fixed effect models weighted by inverse variance to combine the results across validation study sets into a single estimate by using the metafor R function. A few statistics were combined via Stouffer's method due to different scaling of effect sizes across study sets, such as number of age-related conditions, disease free status and physical function scores.

### Heritability Analysis

We conducted heritability analysis using the polygenic models defined in SOLAR [36] to estimate narrow sense h². The robust polygenic model (with t-dist option) was used to estimate heritability of AgeAccelGrim and DNAm based proteins. Heritability is defined as the total proportion of phenotypic variance attributable to genetic variation in the polygenic model. All the analysis were adjusted for age (except AgeAccelGrim) and gender and were performed in R solarius [37].

### Two Stage Estimate of Mortality Versus a Single Stage Estimate of Mortality

To develop our single stage mortality estimator, DNAm Mortality, we used ElasticNet Cox regression to regress time-to-death on the CpG markers, chronological age and sex in the FHS training data. We used the same options in the training process (i.e., 10-fold cross validation for choosing the lambda tuning parameter). The final estimator for DNA Mortality is a linear combination of 59 CpGs and chronological age. We used the same method for calibration as we did for DNAm GrimAge and to arrive at an estimate in units of years (DNAm Mortality). A secondary analysis was to compare with the two estimators of mortality developed by Zhang (on the basis of 10 CpGs)[4]. One estimator is a composite score of these 10 CpGs with weights determined by Cox regression with lasso penalty. Using the same method, we calibrated this estimate in units of year (arriving at DNAmZhang) in order to ensure a fair comparison with our other predictors of lifespan. The other Zhang estimator was defined as the total scores of the 10 CpGs with aberrant methylation[4], referred as DNAmZhangScore (range from 0 to 10). Of the 10 CpGs, cg06126421 and cg23665802 were absent in the JHS cohort and were replaced by the medians based on the FHS training data.

### AgeAccelGrim Versus Blood Cell Composition

The imputed blood cell abundance measures were related to DNAm Grim Age models using the validation study sets: FHS test, WHI BA23, JHS, and InChianti, involving n=6,003 individuals. The following imputed blood cell counts were analyzed: B cell, naïve CD4+ T, CD4+ T, naïve CD8+ T, CD8+ T, exhausted cytotoxic CD8+ T cells (defined as CD8 positive CD28 negative CD45R negative), plasma blasts, natural killer cells, monocytes, and granulocytes. The blood cell composition imputation of the naive T cells, exhausted T cells, and plasma blasts was based on the Horvath method [38]. The remaining cell types were imputed using the Houseman method [21]. To avoid confounding by age, we used AgeAccelGrim and adjusted all DNAm based surrogate biomarkers by chronological age (by forming residuals). The correlation results were combined across studies via the same fixed effect models.

### Diet, Exercise, Education, and Lifestyle Factors

We performed a robust correlation analysis (biweight midcorrelation, bicor [39]) between 1) AgeAccelGrim and its eight age-adjusted components and 2) 38 variables including 12 self-reported dietary variables, behavioral variables, 9 dietary biomarkers, 12 variables related to metabolic related traits and central adiposity, and 5 life style factors. We combined the individuals from the WHI BA23 and WHI EMPC, up to ˜4000 postmenopausal women. The bicor analysis was conducted in the whole sample and in the three groups stratified by ethnicity. Blood biomarkers were measured from fasting plasma collected at baseline. Food groups and nutrients are inclusive, including all types and all preparation methods, e.g. folic acid includes synthetic and natural, dairy includes cheese and all types of milk. The individual variables are explained in [22].

### EXAMPLE 1 REFERENCES

1. Horvath S: DNA methylation age of human tissues and cell types. Genome Biol 2013, 14.
2. Hannum G, Guinney J, Zhao L, Zhang L, Hughes G, Sadda S, Klotzle B, Bibikova M, Fan J B, Gao Y, et al: Genome-wide methylation profiles reveal quantitative views of human aging rates. Mol Cell 2013, 49:359-367.
3. Levine M E, Lu A T, Quach A, Chen B H, Assimes T L, Bandinelli S, Hou L, Baccarelli A A, Stewart J D, Li Y, et al: An epigenetic biomarker of aging for lifespan and healthspan. Aging (Albany N.Y.) 2018.
4. Zhang Y, Wilson R, Heiss J, Breitling L P, Saum K U, Schottker B, Holleczek B, Waldenberger M, Peters A, Brenner H: DNA methylation signatures in peripheral blood strongly predict all-cause mortality. Nat Commun 2017, 8:14617.
5. Durso D F, Bacalini M G, Sala C, Pirazzini C, Marasco E, Bonafe M, do Valle I F, Gentilini D, Castellani G, Faria AMC, et al: Acceleration of leukocytes' epigenetic age as an early tumor and sex-specific marker of breast and colorectal cancer. Oncotarget 2017, 8:23237-23245.
6. Lin Q, Weidner C I, Costa I G, Marioni R E, Ferreira M R, Deary I J, Wagner W: DNA methylation levels at individual age-associated CpG sites can be indicative for life expectancy. Aging (Albany N.Y.) 2016, 8:394-401.
7. Marioni R, Shah S, McRae A, Chen B, Colicino E, Harris S, Gibson J, Henders A, Redmond P, Cox S, et al: DNA methylation age of blood predicts all-cause mortality in later life. Genome Biol 2015, 16:25.
8. Perna L, Zhang Y, Mons U, Holleczek B, Saum K U, Brenner H: Epigenetic age acceleration predicts cancer, cardiovascular, and all-cause mortality in a German case cohort. Clin Epigenetics 2016, 8:64.
9. Chen B H, Marioni R E, Colicino E, Peters M J, Ward-Caviness C K, Tsai P C, Roetker N S, Just A C, Demerath E W, Guan W, et al: DNA methylation-based measures of biological age: meta-analysis predicting time to death. Aging (Albany N.Y.) 2016, 8:1844-1865.
10. Horvath S, Raj K: DNA methylation-based biomarkers and the epigenetic clock theory of ageing. Nat Rev Genet 2018, 19:371-384.
11. Zheng S C, Widschwendter M, Teschendorff A E: Epigenetic drift, epigenetic clocks and cancer risk. Epigenomics 2016, 8:705-719.
12. Jung M, Pfeifer G P: Aging and DNA methylation. BMC Biology 2015, 13:1-8.
13. Nwanaji-Enwerem J C, Weisskopf M G, Baccarelli A A: Multi-tissue DNA methylation age: Molecular relationships and perspectives for advancing biomarker utility. Ageing Res Rev 2018, 45:15-23.
14. Ignjatovic V, Lai C, Summerhaves R, Mathesius U, Tawfilis S, Perugini M A, Monagle P: Age-related differences in plasma proteins: how plasma proteins change from neonates to adults. PLoS One 2011, 6:e17213.
15. Ridker P M, Buring J E, Cook N R, Rifai N: C-reactive protein, the metabolic syndrome, and risk of incident cardiovascular events an 8-year follow-up of 14 719 initially healthy American women. Circulation 2003, 107:391-397.
16. Dawber T R, Meadors G F, Moore F E, Jr.: Epidemiological approaches to heart disease: the Framingham Study. Am J Public Health Nations Health 1951, 41:279-281.
17. Considine R V, Sinha M K, Heiman M L, Kriauciunas A, Stephens T W, Nyce M R, Ohannesian J P, Marco C C, McKee L J, Bauer T L, et al.: Serum immunoreactive-leptin concentrations in normal-weight and obese humans. N Engl J Med 1996, 334:292-295.
18. Rosenbaum M, Nicolson M, Hirsch J, Heymsfield S B, Gallagher D, Chu F, Leibel R L: Effects of gender, body composition, and menopause on plasma concentrations of leptin. J Clin Endocrinol Metab 1996, 81:3424-3427.
19. Gao X, Jia M, Zhang Y, Breitling L P, Brenner H: DNA methylation changes of whole blood cells in response to active smoking exposure in adults: a systematic review of DNA methylation studies. Clin Epigenetics 2015, 7:113.
20. Horvath S, Gurven M, Levine M E, Trumble B C, Kaplan H, Allayee H, Ritz B R, Chen B, Lu A T, Rickabaugh T M, et al: An epigenetic clock analysis of race/ethnicity, sex, and coronary heart disease. Genome Biol 2016, 17:171.
21. Houseman E, Accomando W, Koestler D, Christensen B, Marsit C, Nelson H, Wiencke J, Kelsey K: DNA methylation arrays as surrogate measures of cell mixture distribution. BMC Bioinformatics 2012, 13:86.
22. Quach A, Levine M E, Tanaka T, Lu A T, Chen B H, Ferrucci L, Ritz B, Bandinelli S, Neuhouser M L, Beasley J M, et al: Epigenetic clock analysis of diet, exercise, education, and lifestyle factors. Aging (Albany N.Y.) 2017.
23. Jylhava J, Pedersen N L, Hagg S: Biological Age Predictors. EBioMedicine 2017, 21:29-36.
24. Wong H K, Cheung T T, Cheung B M: Adrenomedullin and cardiovascular diseases. JRSM Cardiovasc Dis 2012, 1.
25. Liabeuf S, Lenglet A, Desjardins L, Neirynck N, Glorieux G, Lemke H D, Vanholder R, Diouf M, Choukroun G, Massy Z A, European Uremic Toxin Work G: Plasma beta-2 microglobulin is associated with cardiovascular disease in uremic patients. Kidney Int 2012, 82:1297-1303.
26. Ferguson T W, Komenda P, Tangri N: Cystatin C as a biomarker for estimating glomerular filtration rate. Curr Opin Nephrol Hypertens 2015, 24:295-300.
27. Larrayoz I M, Ferrero H, Martisova E, Gil-Bea F J, Ramirez M J, Martinez A: Adrenomedullin Contributes to Age-Related Memory Loss in Mice and Is Elevated in Aging Human Brains. Front Mol Neurosci 2017, 10:384.
28. Smith L K, He Y, Park J S, Bieri G, Snethlage C E, Lin K, Gontier G, Wabl R, Plambeck K E, Udeochu J, et al: beta2-microglobulin is a systemic pro-aging factor that impairs cognitive function and neurogenesis. Nat Med 2015, 21:932-937.
29. Fujita Y, Taniguchi Y, Shinkai S, Tanaka M, Ito M: Secreted growth differentiation factor 15 as a potential biomarker for mitochondrial dysfunctions in aging and age-related disorders. Geriatr Gerontol Int 2016, 16 Suppl 1:17-29.
30. Cesari M, Pahor M, Incalzi R A: Plasminogen activator inhibitor-1 (PAI-1): a key factor linking fibrinolysis and age-related subclinical and clinical conditions. Cardiovasc Ther 2010, 28:e72-91.
31. Khan S S, Shah S J, Klyachko E, Baldridge A S, Eren M, Place A T, Aviv A, Puterman E, Lloyd-Jones D M, Heiman M, et al: A null mutation in &lt;em&gt;SERPINE1&lt;/em&gt; protects against biological aging in humans. Science Advances 2017, 3.
32. Ashutosh, Chao C, Borgmann K, Brew K, Ghorpade A: Tissue inhibitor of metalloproteinases-1 protects human neurons from staurosporine and HIV-1-induced apoptosis: mechanisms and relevance to HIV-1-associated dementia. Cell Death Dis 2012, 3:e332.
33. Ridker P M: High-sensitivity C-reactive protein: potential adjunct for global risk assessment in the primary prevention of cardiovascular disease. Circulation 2001, 103:1813-1818.
34. Andersson C, Enserro D, Sullivan L, Wang T J, Januzzi J L, Jr., Benjamin E J, Vita J A, Hamburg N M, Larson M G, Mitchell G F, Vasan R S: Relations of circulating GDF-15, soluble ST2, and troponin-I concentrations with vascular function in the community: The Framingham Heart Study. Atherosclerosis 2016, 248:245-251.
35. Zou H, Hastie T: Regularization and variable selection via the elastic net (vol B 67, pg 301, 2005). Journal of the Royal Statistical Society Series B-Statistical Methodology 2005, 67:768-768.
36. Almasy L, Blangero J: Multipoint quantitative-trait linkage analysis in general pedigrees. Am J Hum Genet 1998, 62:1198-1211.
37. Ziyatdinov A, Brunel H, Martinez-Perez A, Buil A, Perera A, Soria J M: solarius: an R interface to SOLAR for variance component analysis in pedigrees. Bioinformatics 2016, 32:1901-1902.
38. Horvath S, Levine A J: HIV-1 infection accelerates age according to the epigenetic clock. J Infect Dis 2015.
39. Langfelder P, Horvath S: WGCNA: an R package for weighted correlation network analysis. BMC Bioinformatics 2008, 9:559.

### EXAMPLE 2: DATASETS AND MATERIALS OF THE INVENTION

### Note 1: Description of Datasets

Our study involved four cohorts: the Framingham Heart Study (FHS) offspring cohort, the Women's Health Initiative (WHI), Jackson Heart Study (JHS), and Invecchiare in Chianti, aging in the Chianti area (InChianti). We established our DNAm GrimAge model using the individuals from the FHS cohort. Of the FHS cohort, 1731 individuals were used for the training process in establishing the DNAm GrimAge model and 625 individuals were used for the test process at stage 1. Validation analysis was performed on 7,735 observations across 6,395 individuals coming from five independent datasets: the FHS test dataset (N=625), WHI BA23 (N=2107), WHI EMPC (N=1972), JHS (N=1747), and InChianti study (N=924 from 1 to 2 longitudinal measures on 484 individuals). Below we describe each study cohort/datasets in more detail.

### Framingham Heart Study Cohort

The FHS cohort¹ is a large-scale longitudinal study started in 1948, initially investigating the common factors of characteristics that contribute to cardiovascular disease (CVD), https://www.framinghamheartstudy.org/index.php. The study at first enrolled participants living in the town of Framingham, Mass., who were free of overt symptoms of CVD, heart attack or stroke at enrollment. In 1971, the study started FHS Offspring Cohort to enroll a second generation of the original participants' adult children and their spouses (n=5124) for conducting similar examinations². Participants from the FHS Offspring Cohort were eligible for our study if they attended both the seventh and eighth examination cycles and consented to having their molecular data used for study. We used the 2,356 participants from the group of Health/Medical/Biomedical (IRB, MDS) consent and available for both Immunoassay array DNA methylation array data. The FHS data are available in dbGaP (accession number: phs000363.v16.p10 and phs000724.v2.p9).

We computed the total of number age-related conditions based on dyslipidemia, hypertension, cardiovascular disease (including coronary heart disease [CHD] or congestive heart failure [CHF]), type 2 diabetes, cancer and arthritis. Time to CHD or time to CHF was truncated at zero if it occurred before exam 8. Deaths among the FHS participants that occurred prior to Jan. 1, 2013 were ascertained using multiple strategies, including routine contact with participants for health history updates, surveillance at the local hospital and in obituaries of the local newspaper, and queries to the National Death Index. Death certificates, hospital and nursing home records prior to death, and autopsy reports were requested. When cause of death was undeterminable, the next of kin were interviewed. The date and cause of death were reviewed by an endpoint panel of 3 investigators.

DNA methylation quantification Peripheral blood samples were collected at the 8^{th} examination. Genomic DNA was extracted from buffy coat using the Gentra Puregene DNA extraction kit (Qiagen) and bisulfite converted using EZ DNA Methylation kit (Zymo Research Corporation). DNA methylation quantification was conducted in two laboratory batches using the Illumina Infinium HumanMethylation450 array (Illumina). Methylation beta values were generated using the Bioconductor minfi package with Noob background correction³.

### Women's Health Initiative

The WHI is a national study that enrolled postmenopausal women aged 50-79 years into the clinical trials (CT) or observational study (OS) cohorts between 1993 and 1998^{4.5}. We included 4,079 WHI participants with available phenotype and DNA methylation array data: 2,107 women from “Broad Agency Award 23” (WHI BA23) and 1,972 women from “Epigenetic Mechanisms of PM-Mediated CVD Risk” (WHI EMPC). WHI BA23 focuses on identifying miRNA and genomic biomarkers of coronary heart disease (CHD), integrating the biomarkers into diagnostic and prognostic predictors of CHD and other related phenotypes, and other objectives can be found in https://www.whi.org/researchers/data/WHIStudies/StudySites/BA23/Pages/home.aspx. WHI EMPC is a study of epigenetic mechanisms underlying associations between ambient particulate matter (PM) air pollution and cardiovascular disease⁶. WHI EMPC and BA23 span three WHI sub-cohorts including GARNET, WHIMS and SHARe. 936 EMPC participants were not in any of the WHI GWAS (either GARNET, WHIMS, SHARe, MOPMAP, HIPFX, or GECCO). The largest overlap was with SHARE & GARNET. There was almost no overlap with WHIMS & MOPMAP.

The total number of age-related conditions was based on Alzheimer's disease, amyotrophic lateral sclerosis, arthritis, cancer, cataract, CVD, glaucoma, emphysema, hypertension, and osteoporosis.

DNA methylation quantification for BA23 In brief, bisulfite conversion using the Zymo EZ DNA Methylation Kit (Zymo Research, Orange, Calif., USA) as well as subsequent hybridization of the HumanMethylation450k Bead Chip (Illumina, San Diego, Calif.), and scanning (iScan, Illumina) were performed according to the manufacturers protocols by applying standard settings. DNA methylation levels (β values) were determined by calculating the ratio of intensities between methylated (signal A) and un-methylated (signal B) sites. Specifically, the β value was calculated from the intensity of the methylated (M corresponding to signal A) and un-methylated (U corresponding to signal B) sites, as the ratio of fluorescent signals β=Max(M,0)/[Max(M,0)+Max(U,0)+100]. Thus, β values range from 0 (completely un-methylated) to 1 (completely methylated).

DNA methylation quantification for WHI EMPC Illumina Infinium HumanMethylation450 BeadChip data from the Northwestern University Genomics Core Facility for WHI EMPC participants sampled in stages 1a, 1b, and 2 were quality controlled, normalized and batch adjusted. Beta-mixture quantile normalization was implemented using BMIQ⁷ and empirical Bayes methods of batch adjustment for stage and plate were implemented in ComBat⁸.

### Lifestyle Factors and Dietary Assessment in the Women's Health Initiative (WHI)

WHI participants completed self-administered questionnaires at baseline which provided personal information on a wide range of topics, including sociodemographic information (age, education, race, income), and current health behaviors (recreational physical activity, tobacco and alcohol exposure, and diet). Participants also visited clinics at baseline where certified Clinical Center staff collected blood specimens and measured anthropometrics (weight, height, hip and waist circumferences) and blood pressures (systolic, diastolic). Body mass index and waist to hip ratio were calculated from these measurements.

Dietary intake was assessed at baseline using the WHI Food Frequency Questionnaire 9. Briefly, participants were asked to report on dietary habits in the past three months, including intake, frequency, and portion sizes of foods or food groups, along with questions concerning topics such as food preparation practices and types of added fats. Nutrient intake levels were then estimated from these responses. For current drinker, we use the threshold of more than one serving equivalent (14 g) within the last 28 days.

### Jackson Heart Study

The JHS is a large, population-based observational study evaluating the etiology of cardiovascular, renal, and respiratory diseases among African Americans residing in the three counties (Hinds, Madison, and Rankin) that make up the Jackson, Miss. metropolitan area¹⁰. Data and biologic materials have been collected from 5306 participants, including a nested family cohort of 1,498 members of 264 families. The age at enrollment for the unrelated cohort was 35-84 years; the family cohort included related individuals >21 years old. Participants provided extensive medical and social history, had an array of physical and biochemical measurements and diagnostic procedures, and provided genomic DNA during a baseline examination (2000-2004) and two follow-up examinations (2005-2008 and 2009-2012). The study population is characterized by a high prevalence of diabetes, hypertension, obesity, and related disorders. Annual follow-up interviews and cohort surveillance are ongoing. In our analysis, we used the visits at baseline from 1747 individuals as part of project JHS ancillary study ASN0104, available with both phenotype and DNA methylation array data. Total numbers of age-related conditions were based on hypertension, type 2 diabetes, kidney dysfunction based on ever dialysis, and CVD.

DNA methylation quantification Peripheral blood samples were collected at the baseline. Methylation beta values were generated using the Bioconductor minfi package with Noob background correction³.

### Invecchiare in Chianti, Aging in the Chianti Area (InChianti)

The InChianti (Invecchiare in Chianti, aging in the Chianti area) cohort is a representative population-based study of older persons enrolling individuals aged 20 years and older from two areas in the Chianti region of Tuscany, Italy, http://inchiantistudy.net/wp/. One major goal of the study is to translate epidemiological research into geriatric clinical tools, ultimately advancing clinical applications in older persons. Of the cohort, 924 observations from 484 individuals with both phenotype information and DNA methylation data were including in our studies. The observations were collected from baseline in 1998 and the third follow-up visit in 2007. All participants provided written informed consent to participate in this study. The study complied with the Declaration of Helsinki. The Italian National Institute of Research and Care on Aging Institutional Review Board approved the study protocol. We computed the total number of age-related conditions based on cancer, hypertension, myocardial infarction, Parkinson's disease, stroke and type 2 diabetes.

DNA methylation quantification. Genomic DNA was extracted from buffy coat samples using an AutoGen Flex and quantified on a Nanodrop1000 spectrophotometer prior to bisulfite conversion. Genomic DNA was bisulfite converted using Zymo EZ-96 DNA Methylation Kit (Zymo Research Corp., Irvine, Calif.) as per the manufacturer's protocol. CpG methylation status of 485,577 CpG sites was determined using the Illumina Infinium HumanMethylation450 BeadChip (Illumina Inc., San Diego, Calif.) as per the manufacturer's protocol and as previously described¹¹. Initial data analysis was performed using GenomeStudio 2011.1 (Model M Version 1.9.0, Illumina Inc.). Threshold call rate for inclusion of samples was 95%. Quality control of sample handling included comparison of clinically reported sex versus sex of the same samples determined by analysis of methylation levels of CpG sites on the X chromosome¹¹. Methylation beta values were generated using the Bioconductor minfi package with Noob background correction³.

### Note 2. DNAm Based Surrogates for Plasma Proteins

The model of DNAm GrimAge is composed of seven DNAm based plasma proteins, DNAm based pack years, age and gender. Below we briefly describe the seven plasma proteins. ADM (adrenomedullin) is a vasodilator peptide hormone. Plasma ADM, initially isolated from adrenal gland, is increased in individuals with hypertension and heart failure¹². A recent study showed that ADM was involved in age-related memory loss in mice and aging human brains¹³.

B2M (Beta-2 microglobulin) is a component of major histocompatibility complex class 1 (MHC I) molecular. Plasma B2M is a clinical biomarker associated with cardiovascular disease, kidney function, inflammation severity¹⁴. B2M is a pro-aging factor associated with cognitive and regenerative function in aging process and suggests B2M may be targeted therapeutically in old age¹⁵. A previous study showed that systemic B2M accumulation in aging blood promoted age-related cognitive dysfunction and impairs mouse models¹⁵.

Cystatin C or cystatin 3 (formerly gamma trace, post-gamma-globulin, or neuroendocrine basic polypeptide) is mainly used as a biomarker of kidney function. Plasma cystatin-C is a clinical relevant biomarker indicating kidney function¹⁶. Cystatin-C seems plays a role in cardiovascular disease¹⁷ or amyloid deposition associated with Alzheimer's disease¹⁸.

GDF-15 (growth differentiation factor 15) is one of transforming growth factor beta subfamily. GDF-15 has been implicated in aging and age-related disorders. It also plays a role in age-related mitochondria dysfunction¹⁹.

Leptin is a hormone predominantly in adipose cells. Leptin plays a role in regulating energy balance by inhibiting hunger and is implicated in Alzheimer's disease²⁰.

Plasminogen activator inhibitor antigen type 1(PAI-1) is the major inhibitor of tissue-type plasminogen activator and unokinase plasminogen activator. PAI-1, released in response to inflammation process, plays a central role in a number of age-related subclinical (i.e., inflammation, atherosclerosis, insulin resistance) and clinical conditions (i.e., obesity, comorbidities)²¹.

TIMP-1 or TIMP metallopeptidase inhibitor 1 is a tissue inhibitor of metalloproteinases. It is also involves chromatin structures, promoting cell proliferation in a wide range of cell types, and may also have an anti-apoptotic function²².

### Estimation of Blood Cell Counts Based on DNAm Levels

We estimated blood cell counts using two different software tools. First, Houseman's estimation method²³ was used to estimate the proportions of CD8+ T cells, CD4+ T, natural killer, B cells, and granulocytes (mainly neutrophils). Second, the Horvath blood cell estimation method, implemented in the advanced analysis option of the epigenetic clock software^{24,25}, was used to estimate the percentage of exhausted CD8+ T cells (defined as CD28−CD45RA−), the number (count) of naïve CD8+ T cells (defined as CD45RA+CCR7+) and plasma blasts cells. We and others have shown that the estimated blood cell counts have moderately high correlations with corresponding flow cytometric measures^{23,26}.

### TABLES

TABLE 1
Reproducibility and age correlations of DNAm
based surrogate biomarkers

| | Training | | Test | |
|---|---|---|---|---|
| | (N = 1731) | | (N = 625) | |
| | Observed | | Observed | |
| Correlation (r) | biomarker | Age | biomarker | Age |
| DNAm based surrogate | | | | |
| adrenomedullin | 0.65 | 0.63 | 0.38 | 0.64 |
| beta-2-microglobulin | 0.62 | 0.83 | 0.43 | 0.85 |
| CD56 | 0.86 | 0.17 | 0.36 | 0.17 |
| ceruloplasmin | 0.56 | 0.04 | 0.49 | −0.02 |
| cystatin-C | 0.58 | 0.81 | 0.39 | 0.83 |
| EGF fibulin-like ECM protein1 | 0.59 | 0.72 | 0.41 | 0.87 |
| growth differentiation factor 15 | 0.74 | 0.71 | 0.53 | 0.81 |
| leptin | 0.68 | 0.06 | 0.35 | 0.05 |
| myoglobin | 0.50 | −0.04 | 0.38 | 0.03 |
| plasminogen activator | 0.69 | 0.19 | 0.36 | 0.16 |
| inhibitor 1 | | | | |
| serum paraoxonase/ | 0.57 | −0.22 | 0.51 | −0.22 |
| arylesterase 1 | | | | |
| tissue Inhibitor | 0.43 | 0.92 | 0.35 | 0.90 |
| Metalloproteinases 1 | | | | |
| smoking pack-years | 0.79 | 0.17 | 0.66 | 0.13 |

### Legend

This table reports the correlation coefficients between the observed marker (i.e. observed plasma protein level or self-reported smoking pack-years) and its respective DNAm based surrogate marker in 1) the FHS training data and 2) the FHS test data. Each of the DNA based surrogate biomarkers (rows) leads to a correlation r≥0.35 in both training and test datasets (columns 2 and 4). DNAm based pack-years is highly correlated with the self-report pack-years in both training and test datasets (r≥0.66). The table also reports the correlation coefficients between the DNAm based surrogate biomarkers (rows) and chronological age in the FHS training and test data (columns 3 and 5).

TABLE 2
Overview of the cohorts used in the validation analysis

| Years of | | | | Smoking status | | | | |
|---|---|---|---|---|---|---|---|---|
| Study | N | Female | Age | Never | Former | Current | Pack-years | Follow-up |
| FHS* | 625 | 53% | 66.9 ± 8.64 | 37% | 52% | 10% | 14.7 ± 19.91 | 7.7 ± 1.78 |
| test | | | [61, 73] | | | | [0, 23] | [7.3, 8.8] |
| WHI BA23 | 2107 | 100% | 65.3:17.1 | 52% | 36% | 10% | 9.5 ± 18.55 | 16.97 ± 463 |
| | | | [60, 70.9] | | | | [0, 12.5] | [15.8, 19.9] |
| WHI EMPC | 1972 | 100% | 63.3 ± 7.03 | 52% | 38% | 9% | 9 ± 17.27 | 18 ± 4.02 |
| | | | [57.9, 68.7] | | | | [0, 12.5] | [17.9, 20.1] |
| JHS | 1747 | 63% | 56.2 ± 12.31 | 65% | 21% | 14% | NA | 11.7 ± 2.55 |
| | | | [46.5, 65.4] | | | | | [11.2, 13.1] |
| InChianti** | 924 | 54% | 67 ± 16.64 | 57% | 29% | 14% | 10.3 ± 17.33 | 5.4 ± 4.84 |
| | (484) | | [60, 78] | | | | [0, 16.8] | [0.1, 9.3] |

NA = not available.
Quantitative variables are presented in the format of mean ± SD [25^{th}, 75^{th}].
*The distribution of age is based on exam 8.
**The statistics are based on the number of 924 observations across 484 individuals.

### Legend

This table summarizes the characteristics of 6,935 individuals (corresponding to 7,375 Illumina arrays) from five independent cohorts that were used in our validation analysis. For example, up to two longitudinal measurements were available for each of 484 individuals in the InChianti cohort.

TABLE 3
Characteristics of the Framingham Heart Study
Description of the Framingham Heart Study (FHS) Offspring Cohort
that was used to train and test DNAm GrimAge.
We assigned 70% of pedigrees to the training dataset and the
remaining 30% pedigrees to the test dataset.

| | Training | | Test | |
|---|---|---|---|---|
| | N = 1731 | | N = 625 | |
| N_{pedigree} | 622 | | 266 | |
| Female | 54% | | 53% | |
| Smoking | | | | |
| Never | 41% | | 37% | |
| Former | 51% | | 52% | |
| Current | 8% | | 10% | |
| Age | | | | |
| exam 7 | 59.6 ± 9.05 | [53;67] | 60.3 ± 8.59 | [54;66] |
| exam 8 | 66.2 ± 9.08 | [59;73] | 66.9 ± 8.64 | [61;73] |
| PACK years | 13.8 ± 20.17 | [0;22] | 14.7 ± 19.91 | [0;23] |
| Follow-up (years) | | | | |
| since exam 7 | 14.5 ± 1.69 | [14;15.6] | 14.4 ± 1.82 | [13.9;15.6] |
| since exam 8 | 7.9 ± 1.65 | [7.5;8.9] | 7.7 ± 1.78 | [7.3;8.8] |

Quantitative variables are presented in the format of mean±one standard deviation, SD and the interval: [25^{th}, 75th] percentile.
Immunoassay array was profiled at exam 7;
DNA methylation array was profiled at exam 8.

TABLE 4
Multivariate regression model for estimating DNA GrimAge
Coefficient values for computing the uncalibrated version of DNAm GrimAge based on
the underlying covariates (rows): chronological age, sex (an indicator of female), and eight DNA
methylation (DNAm) based variables (rows). The columns report the name of the covariate (e.g.
DNAm based biomarker), its abbreviation, regression coefficient, and the number of underlying
CpGs that underlie the surrogate biomarker (defined in stage 1). As noted, this model yields
uncalibrated DNAm GrimAge. The finalized DNAm GrimAge is based on transforming the raw
variable into a distribution in units of year (see Methods).

| | | | Number |
|---|---|---|---|
| Covariate | Abbreviation | Coefficients | of CpCs |
| DNAm adrenomedullin | DNAm ADM | 0.007903 | 186 |
| DNAm beta-2-microglobulin | DNAm B2M | 4.59E−7 | 91 |
| DNAm cystatin-C | DNAm Cystatin C | 3.5E−6 | 87 |
| DNAm growth differentiation factor 15 | DNAm GDF-15 | 0.000349 | 137 |
| DNAm leptin | DNAm Leptin | −7.3E−6 | 187 |
| DNAm plasminogen activator inhibitor 1 | DNAm PAI-1 | 2.56E−5 | 211 |
| DNAm tissue inhibitor metalloproteinases 1 | DNAm TIMP-1 | 0.000144 | 42 |
| DNAm pack years | DNAm PACKYRS | 0.030398 | 172 |
| Chronological age | Age | 0.030082 | — |
| Female | Female | −0.22847 | — |

TABLE 5
Comparing self-reported versus DNAm based estimates of smoking pack years
predicting time-to-death.
Two Cox regression models for time-to-death (due to all-cause mortality) were used to
evaluate the predictive power of self-reported smoking pack years and its DNAm based
surrogate biomarker, respectively. The survival analysis was conducted across five datasets from
four independent study cohorts as detailed in the first column). The column report the variables,
the sample size, the tota number of deaths during the follow-up period, the hazard ratio
associated with a 1 unit increase in the variable, and the corresponding Cox regression p-value.
To compare the performance of the two variables, one should focus on the p-value as opposed to
the hazard ratio (because the latter depends on the distribution of the underlying variable).
Surprisingly, this comparative analysis indicates that the DNAm based surrogate biomarker of
smoking pack years leads to a more significant (smaller) Cox regression p-value than that of the
self-reported variable.

| Data | Pack years | N | # death | HR | P |
|---|---|---|---|---|---|
| FHS train | DNAm surrogate | 1729 | 219 | 1.04 | 8.10E−23 |
| | Self-reported | 1729 | 219 | 1.02 | 1.12E−11 |
| FHS test | DNAm surrogate | 625 | 88 | 1.04 | 8.51E−5 |
| | Self-reported | 625 | 88 | 1.01 | 2.13E−3 |
| WHI BA23 | DNAm surrogate | 2107 | 765 | 1.02 | 7.17E−12 |
| | Self-reported | 2029 | 734 | 1.01 | 7.03E−12 |
| WHI EMPC | DNAm surrogate | 1972 | 505 | 1.03 | 2.1E−19 |
| | Self-reported | 1904 | 484 | 1.02 | 6.4E−19 |
| InChianti | DNAm surrogate | 924 | 209 | 1.02 | 5.33E−4 |
| | Self-reported | 924 | 209 | 1.01 | 1.78E−1 |

HR = hazard ratio

TABLE 6
Comparing ImmunoAssay versus DNAm based estimates of plasma proteins wrt.
predicting time-to-death.
Two Cox regression models for time-to-death (due to all-cause mortality) were used to
evaluate the predictive power of our study plasma proteins measured by ImmunoAssay versus its
DNAm based surrogate biomarker, respectively. The survival analysis was adjusted for
chronological age, center, and family structure, conducted in the FHS training (N/number of
death = 1729/219) and test(N/number of death = 625/88) datasets, respectively. For each plasma
protein, we list the hazard ratios (HR) and the corresponding Cox regression p-values of the
protein levels based on ImmunoAssay measure and DNA methylation estimate, respectively. All
the test variables were standardized based on the FHS training dataset such that each HR
corresponds to an increase in one-standard deviation. To compare the performance of the two
variables, one should focus on the p-value as opposed to the hazard ratio (because the latter
depends on the distribution of the underlying variable).
The analysis for leptin levels was stratified by gender due to its highly correlation with gender.

| | | HR* | | P-value | |
|---|---|---|---|---|---|
| Protein | data | ImmunoAssay | DNAm | ImmunoAssay | DNAm |
| Adrenomedullin | training | 1.23 | 1.36 | 4.53E−4 | 3.85E−7 |
| | test | 1.33 | 1.06 | 2.92E−2 | 7.23E−1 |
| Beta-2 | training | 1.31 | 1.51 | 7.42E−8 | 9.87E−8 |
| Microglobulin | test | 1.22 | 1.94 | 133E−2 | 2.92E−3 |
| Cystatin C | training | 1.25 | 1.53 | 1.32E−4 | 1.44E−8 |
| | test | 1.13 | 1.53 | 5.40E−2 | 1.92E−2 |
| GDF-15 | training | 1.24 | 1.20 | 3.82E−5 | 2.58E−3 |
| | test | 1.83 | 1.63 | 6.86E−13 | 2.32E−2 |
| Leptin | training male | 1.28 | 2.23 | 1.03E−1 | 9.12E−6 |
| | test male | 0.99 | 1.11 | 9.74E−1 | 6.83E−1 |
| | training female | 0.93 | 0.97 | 4.81E−1 | 8.38E−1 |
| | test female | 1.15 | 1.18 | 3.25E−3 | 5.91E−1 |
| PAI-1 | training | 1.14 | 1.31 | 3.95E−2 | 3.63E−6 |
| | test | 1.19 | 1.37 | 7.42E−2 | 8.69E−4 |
| TIMP-1 | training | 1.33 | 2.61 | 1.38E−5 | 1.06E−8 |
| | test | 1.29 | 3.30 | 1.71E−2 | 3.79E−4 |

*in units of one SD.
The p value based on a DNAm variable was marked in bold if it was more significant than the one based on the corresponding ImmunoAssay variable.
GDF-15 = growth differentiation factor 15; PAI-1 = plasminogen activation inhibitor 1; TIMP-1 = tissue inhibitor metalloproteinase 1.

TABLE 7
Mortality prediction in the FELS based on DNAm- and observed versions of
AgeAccelGrim
The fact that AgeAccelGrim is a composite biomarker based on DNAm based surrogate
biomarkers of plasma protein levels and smoking pack-years begs the question whether a
predictor of lifespan based on observed values, i.e. observed plasma protein levels and self-
reported smoking pack-years, outperforms its DNAm based analog? To our surprise, relatively
little is gained by using observed values as will be shown in the following. Analogous to our
construction of DNAm GrimAge, we used a Cox regression model to regress-time to-death on
the observed plasma protein levels and self-reported pack-year in the training data (Methods). As
before, the resulting mortality risk estimator (defined as linear combination of the observed
biomarkers) was linearly transformed into units of years. The resulting predictor will be denoted
as observed GrimAge and its age-adjusted version as observed AgeAccelGrim. The table reports
the predictive accuracy for the two age acceleration measures based on observed values versus
DNAm based surrogates when it comes to predicting time to death (due to all-cause mortality).
The table reports the sample size (N), the number of deaths during the follow up period, the
hazard ratio associated with a 1 unit increase in the variable, the Z statistic, and the Cox
regression p value. To compare the performance of the two variables, one should focus on the p-
value as opposed to the hazard ratio (because the latter depends on the distribution of the
underlying variable).
Rows correspond to the results in the training data and the test data of the FIB. An unbiased
comparison in the test from the HIS reveals that the hazard ratio associated with observed
AgeAccelGrim (HR = 1.10, P = 3.2E−7) is similar to that of its DNAm based analog
AgeAccelGrim (HR = 1.12, P = 8.6E−5).
The results in the training data of the FHS are biased and should be ignored.

| FHS | AgeAccelGrim | N | # death | HR | Z | P |
|---|---|---|---|---|---|---|
| Training | Observed | 1729 | 219 | 1.12 | 10.54 | 5.65E−26 |
| (N = 1731) | DNAm based | 1729 | 219 | 1.14 | 11.85 | 2.15E−32 |
| Test | Observed | 625 | 88 | 1.10 | 5.11 | 3.18E−7 |
| (N = 625) | DNAm based | 625 | 88 | 1.12 | 3.93 | 8.55E−5 |

HR = hazard ratio.

TABLE 8
Heritability analysis of observed and DNAm based variables
Robust polygenic model was performed to estimate heritability using the FHS test
pedigree datasets. We estimated the heritability of observed and DNAm based AgeAccelGrim
and it associated proteins. The observed AgeAccelGrim was defined in manuscript and the
observed protein levels were based on ImmunoAssay measures. Additional analysis was applied
to other epigenetic measures of age acceleration: age-adjusted DNAm PhenoAge
(AgeAccelPheno)²⁸, age-adjusted DNAm age based on Hannum et al. (AgeAccelHannum) ²⁷ and
age adjusted DNAm age based on Horvath (AgeAccelResidual)²⁴.

| | Heritability | | P value | |
|---|---|---|---|---|
| Trait | ImmunoAssay | DNAm | ImmunoAssay | DNAm |
| AgeAccelGrim | 0.37 | 0.30 | 6.0E−3 | 2.2E−2 |
| AgeAccelPheno | NA | 0.11 | NA | 2.5E−1 |
| AgeAccelHannum | NA | 0.19 | NA | 7.7E−2 |
| AgeAccelerationResidual | NA | 0.44 | NA | 1.8E−3 |
| Adrenomedullin | 0.42 | 0.38 | 3.7E−3 | 2.9E−3 |
| Beta 2 microglobulin | 0.34 | 0.45 | 3.3E−3 | 2.4E−3 |
| Cystatin C | 0.32 | 0.09 | 2.9E−2 | 3.1E−1 |
| GDF-15 | 0.24 | 0.21 | 6.0E−2 | 9.1E−2 |
| Leptin male | 0.58 | 0.31 | 4.8E−2 | 1.8E−1 |
| Leptin females | 0.34 | 0.38 | 1.1E−1 | 1.3E−1 |
| PAI-1 | 0.34 | 0.51 | 7.1E−3 | 6.2E−4 |
| TIMP-1 | 0.31 | 0.24 | 2.0E−2 | 9.7E−2 |

GDF-15 = growth differentiation factor 15; PAI-1 = plasminogen activation inhibitor; TIMP-1 = tissue inhibitor metalloproteinase 1; NA = not applicable.

TABLE 9
CpG Methylation Sites Associated with one or more physiological factors
associated with an age of an individual

| cg | | Sequence |
|---|---|---|
| identifier | DNA Sequence | ID Number |
| cg20462449 | CGGGACCGTTTGGCCCTCTGTGACCCAAGTCAGCCTCGGCGAAGTGCTTT | 1 |
| cg19802390 | CGCGGCCAGGCAGCGAACCCCACTAGGAGGCACCCCAGGCCCCTGGGCTT | 2 |
| cg14084907 | CGCGCCTGGGCCCCCAGGACCTCTTGCAGACGGGAGTCCCTCAGACAGTG | 3 |
| cg23251761 | GGTTGCTTCCTTTTGTGCCCCCCGGGGGTCAGGAATCCCAGGTTTCTCCG | 4 |
| cg14825555 | CTGAGGCCACCTGGGGCTGGGGATCCCACTCTTCTTGCAGCTGTTGAGCG | 5 |
| cg04967775 | AGGGCCCCCTGGGCCTGTACCAAGGTCTGCGACAGATGGGTGGGGAGCCG | 6 |
| cg05817517 | GCCTGGCAGAATCAGGAGCCGGGGGAGTAGGGCTCCCGGAAAGTTTATCG | 7 |
| cg00500789 | GAGGCAGTGGGCGGGGCTGAAGCTGCAGGTCTGGCAGAGGGCGGGCTGCG | 8 |
| cg23800435 | GTGGGCGGGGCTGAAGCTGCAGGTCTGGCAGAGGGCGGGCTGCGGCAGCG | 9 |
| cg08871545 | CGCAAGCATGAAGCGTTTAACTGTGCAACACAAATCAACAACCTGGGCTC | 10 |
| cg04135242 | CGCACCCACTGAATCAGAAGCTGTAGGAGCGGGAGCCAGTGGTTTGCATT | 11 |
| cg25189904 | CGCTGCCAAACCCCTACACGAAAGTTTGGTTCTGTTCCAGAAACTATGGA | 12 |
| cg16511983 | GGACAAAAATCCTCGGTGGCGGGGGCAAAAAGTCGCGGTGACAAAAAGCG | 13 |
| cg12067764 | TTTGAAGCCCTTATGTCTATGGTAATCTGTTACAGCCCCCGCAGGAAACG | 14 |
| cg09396704 | CGGGCACCTTGCTGCTGGCCCTCGGCCCCACGCACCTGCCCTTGGCTGCC | 15 |
| cg17440248 | CGTGAATTGAGATGTCATGCCATTGTAAGATTTTTAAAAAGGGAAGGACA | 16 |
| cg01491219 | CGGCGCGCACTGCACAATGAACATGCAAAACACCTTCAACTGCACTTGCT | 17 |
| cg02356786 | CCATAGCGGTGGGCCTTTGGCGACGTCAGAGGCGCGGGTGTTCGCCTACG | 18 |
| cg19012696 | AGCTCCTGCCGTGGTGGTGAAATATTGTGGACTGCTTGGCTGACTACACG | 19 |
| cg17886420 | CGTGCTCAGCTGATAAAATGCTAAATAGCATCAATGCTGACCATGCTTCT | 20 |
| cg12465010 | CGTGTGTACACATGTATGTCTTTCAAGCAAGGCTATGTATTACTCAGAAC | 21 |
| cg15777335 | GTCAGGGAGATAAGTCCCTTTGCGGAATGTCAAGACGGTGAGCAGGCGCG | 22 |
| cg03634479 | CGCTTCTTAAGGGAAAGGCATGAGGTGGGACAGGCAAACCTTGGCAAGTG | 23 |
| cg13458005 | CGGCAGGCATCCCCTCTACCTGACCGGCTTCTTCGGAGGATACATCTTGG | 24 |
| cg14371731 | ATTAGCGGAGCCTCCGCCTATGATTGGCTTCGCCCGGGAAGCTGGAGACG | 25 |
| cg23322172 | CGGGGGCTCTTCCTGCTCAAGCTGGGACTTAGCCTGCTCATGTTGTTGGC | 26 |
| cg02089135 | CTTTTTTTGGCATTGGAATTGGCAAGTGGCTGCCCTTGTCTGTTACAACG | 27 |
| cg05747812 | CGGGGTGCAGGTCAGGGCTCCAGGTGTCACCTGCCTGTCCACACGGGGCT | 28 |
| cg11019008 | CGCCGTGTCCCTCACGCTGGCTTTCAGAAGGCTTCACGCAAGTGGGCTCA | 29 |
| cg02580250 | CGGGTGCTGTTATCACCCCAATTTCACCAATGAGGAAAGAGGTGCAGAGA | 30 |
| cg24869272 | GTGGTTTTCTACCTGGACCTCTCCTTCATCTTCCTCCTAGAACTGAAGCG | 31 |
| cg23938637 | GCTGTGGCGTCTCCAACTACACTGACTGGTTCGAGGTGTACAACGCCACG | 32 |
| cg09690118 | CGTGGACTTCCCATCCCCTGGACCCCACGGCGGGGACAAGGAAACCTACA | 33 |
| cg01518025 | AGCCTCACTGAGGAAGTGACTGTCCCAGGCTCTAGAGGTGCCTGAGGGCG | 34 |
| cg06809342 | AGACGGGGAGGCCGGGCAGAGACGCTCCTCACCTCCCAGACAGGGTTGCG | 35 |
| cg00161556 | CGACCCTTTGTGGAGGGCACTGGAGGAGCTGGCATTTAAGCCCTGTCCAG | 36 |
| cg06911753 | AATGCGTCGCAGTCAAGACAGCGCAGGGGCCTGGGCTGCCGGGCGCTGCG | 37 |
| cg04265051 | CAAGGGCACACAGCGAGGCAGTTTCAGGGCGGGCAGCCTGGGGCCCCACG | 38 |
| cg16385335 | CGGGGCAGTGCTTTCCCAGAAGGATTGCTCGGCATCCTGCCCTTCCCAGA | 39 |
| cg11660018 | CGCACAGCACTGAGTGCTGTAAGGAAATGCAAAGCATTGGTGGCCCTGCC | 40 |
| cg23771366 | CGGTGCCTCCAAAGAACAGAAACCTGTATTCCCATGGCAGGGCCACCAAT | 41 |
| cg12051762 | TCTCAAACTCGGGGCCAGAGAACAGTGAAGTAGGAGCAGCCGTAAGTCCG | 42 |
| cg10130564 | CGCCCCGGCCCGCTCCATCTCCAAAGCATGCAGAGAATGTCTCGGCAGCC | 43 |
| cg19174059 | GCACACACAGCAGCAGCACTGCCACCCAGAGCAGGGCTAAGATGAACACG | 44 |
| cg04832325 | CGGGTATTACTGCCATCGTCGTACCAGCAGGGAAACAAACTGAGACTCCG | 45 |
| cg08161922 | CGCGGCTCCATACTATTGCCCAGTGTTTTCCGAGCATCTCCATCCTTGGC | 46 |
| cg00417288 | CAGTCCGTCATCTCATCTGGGGAAACTGTTTGCCTTTTTCTGATACTACG | 47 |
| cg21368094 | CGTGTGCACATGCGCACTCTCATATGCCACCCCATGTGCCTTTAGTCCAC | 48 |
| cg05661164 | CAGGAGATGCCGCGGTAGGGGGCGGCGGTGATGCAGCAGCGGCTTGGCCG | 49 |
| cg09244872 | CGGGTGCCCAGGAGCCCACAAAACGGCCAGAGGAGAAATGCTTTCAAAGG | 50 |
| cg22277972 | CGGCCTTCTGGCTGGCAAGCCCCTGTGGGCCAGGAAGCTCAGACCTAACC | 51 |
| cg13406893 | AGTCACTGAATCATGAGTGACGGTAACATAAATGCCCATCGGGGGAAACG | 52 |
| cg12384004 | ACTCGCCTTGATGTTCATTGGGGTTCAAAATAATCAAGTTCTGCAGAACG | 53 |
| cg17116694 | GGCCAAGGCAGGCGGCTGGGAGGCGTAGGTTGTAGCGAGCCGAGATCACG | 54 |
| cg25939203 | ACGATGTTGGCTCACTGCATCCTCCACCTTCTGGGTTCAAGCGATTCTCG | 55 |
| cg02922734 | GTCATCCCCGGGGAGAGGCTCTCAATGGGGAGTCCCATCTGCAGGCTGCG | 56 |
| cg01873886 | CGGGTTCCTTTTAAAGTGCTGCTAGCGCGCACTCGCCCTCTCAGCGTTGC | 57 |
| cg01637125 | CAAGGCCACTGCAATTTTTGGGATCTAGTGAGTCTGTAGGAAATGTAACG | 58 |
| cg10920224 | TGAGGTCAGGCGTGCCCTTGCGTAGCACAATCGCGGGGAGGGGTGGCTCG | 59 |
| cg18425651 | GCATGTGGCATGGGTGGGGTGGCCAGCACGCTACAGGGGTTTCCTATGCG | 60 |
| cg13316619 | CGGAAGAGGCTCCAGTGTCCTCAGCAGACATGCTTTATTTGGTTAAAGCA | 61 |
| cg23047825 | AAGCTTCAGTGAATGTCCCCCGCTTCTAAGATCCTCTTCTTTTTGTCACG | 62 |
| cg08923669 | CGCCAGGCTCGCGATGCCTCTACACAAGTATCCCGTGTGGCTCTGGAAGC | 63 |
| cg06259664 | CGGGAGAACCTTCTGTGCAGGCCCGGCGCTGCTCCACTCCAGGAAAGAAA | 64 |
| cg05090127 | GAGGCAGGGCAGGCAGACCTGCAGTCACTCCTCTGCAGAGTCGCACACCG | 65 |
| cg01960979 | CGCAGAGAAGGAAATAACTTGTTCAAAAGAACCTACTGGGGTGTGTGTAC | 66 |
| cg06213463 | GGGAGTGGGCTGGCAGTGCGCGCACAGCGGCGGCGAGTGGGTCGTGCACG | 67 |
| cg01773760 | TCGTTCCTCATGAAAACATCTTGAGTCCAGGATCTCCAATTGCACTCACG | 68 |
| cg01130056 | TTCTCTCAAAACTCATCACCCACTTTTCTTTAACTGTTTGTTATAAGACG | 69 |
| cg03974717 | CGGACCTAGACTCCAACAGTGACAGCTCTGAGTAAAGAAGTCGCCGAAGC | 70 |
| cg26573797 | GAAGGAGAAGCACCCGGAGCCGGGGGGCTCCTGTGTCTACTGCAAAAGCG | 71 |
| cg06127256 | TGGCCGGGATCCCGTGCTTGGCATACAGCCGGTCTTGTTCTCATTGTCCG | 72 |
| cg03574306 | TTCATGGGTGTGGTAGCTTGTGAGACCGGCCGGCTGCGCCCATGCCCCCG | 73 |
| cg08439518 | TTTAACCTTTCCAGGCCTCAGTTTCTTCATCTGTTCAGTGGAAATGATCG | 74 |
| cg11235787 | TAGTTTTCACCACCCTGCCTGGAGCAGCACAGCCAATATTGGCAGACTCG | 75 |
| cg20361594 | CGCAAGCCGGCCCTCACCCAGTATATTGATAGGGCCATTGAGGGGTTCCG | 76 |
| cg13029847 | CGGCCTGCTAAGGAACCTCAATTAATAGCTCACTGTAGCCTTCTGATTCT | 77 |
| cg00126959 | GACCCAGAGGCACTGGGGTAGGGCTCGGCAGGGCGGGTCCGGGCGGAGCG | 78 |
| cg24093538 | CTGGGCTCCATGTGTAATTTCAGGGCTCATGGCCACACCCAGCATTGACG | 79 |
| cg16067628 | CGGGTAAGCCCCGCTGTGTTCTCATACCCGAACGGCAGGAACACAGGCAG | 80 |
| cg15258649 | CGCACAGAGCCGCACGGGACTCAGTTTCCACACACACAGAGCCACACGGG | 81 |
| cg03294557 | TAAGAATAACTTTTGCCTCCTACTGTGATTCTGAGGCCTCCCCAATCACG | 82 |
| cg23777956 | GTGGTGACAGAGCCACAGAGGGCTGTGAGCTTGCCCGGCCCCAGGTAACG | 83 |
| cg03636183 | TGGTGGTGGGGCTGCCGGCCAATGGGCTGGCGCTGTGGGTGCTGGCCACG | 84 |
| cg06418475 | CGGGGCGCTGAGCTCAGGCCCAGAACTGGCTGATTTCAGGGATACCCAGG | 85 |
| cg27152890 | CGGCGCAGACACCCCGTTCGGACGATCAGAGAGTGCCCCAACCCTACACC | 86 |
| cg09438320 | GATGACCGGGATACAGTTTGCTGCATTGCAAGGGCTCACCCAGTGTTCCG | 87 |
| cg12157673 | GTCAAAGGACAAGCCCCTGGGACCTGCCCAATGCTGGTTCAGGGGAAGCG | 88 |
| cg22455450 | CGGGGCGGAATATACGATTTCATGCTGATGGCCCACAGAACATAACAGAA | 89 |
| cg08980304 | CGTGTGGACGCGGGAGGAAACCAGAGTGCCCAGAGACAACCCACGCAGAC | 90 |
| cg26881591 | CCATGCAGGGCTCTAGACAGCGGCGCTGCAGAGCTTCCTGAGGTCGGCCG | 91 |
| cg14528537 | CTAGCTTTAGCTGCACATTAGAGTCACCTGCAGCTCTTTTCAAACTGTCG | 92 |
| cg04053045 | GGGGGGTCAATACTCACCTGGTCGTAGACAGGAGCCCTGGAGTCGGAGCG | 93 |
| cg05515143 | CGTGTCTCAGTGACGCTGATTCTGCTTCACAACAAAAGCAAAACAAGAAC | 94 |
| cg06625640 | CGGAAAAGGGAGTGGACAGGAGGGGTACAGAACCAGGCTAATGAGACAAT | 95 |
| cg00102512 | GCTGGCTTTCAGGATCACTATCCAGCAGCAGCTCCGACTTCCAGATTCCG | 96 |
| cg06372850 | CGCTCTCTGGCAAATGTAGAGGCTATTTCTGCTTTTTACAAGAGGTGCTG | 97 |
| cg16978914 | CGGCCCTCCAAAATTGCCCCCACATTGGCTGCCTTATAAATATGTCTGTG | 98 |
| cg00706683 | CGGGTTCAGTCATTGAAAGCCTTCTTGTAATTGCTGAAGGGTATTTGTTT | 99 |
| cg06644428 | CCTCTTTCTGCTCGCCGAAGGCCGCGATGGTGCCATAGGTGAGCTTTTCG | 100 |
| cg21566642 | CGCCTCCTGAGCCTGACTCTGCTCTTCCCGCAGCTTTGGCCCCCCGGGGC | 101 |
| cg21120063 | CGGGCACGATGTCCCAGGAGGACAGAGTTTGCATTGATGAAAACCCTTCC | 102 |
| cg00356999 | CGCTGGGTCTGCTCCCATTGGCTCAGGTCATCTGAGCAGCTGTGCTCTGT | 103 |
| cg02931526 | GGTGCTCTGGACCGGCAGCCATGGGAATGTCCTAGTCGGCAGCAGTGTCG | 104 |
| cg19393314 | CTCACGTACAGTCTCCTCCCTGTACACGAAGGAGACTCTGCTCTAACACG | 105 |
| cg05303999 | CGCTGGGATCGTGGCTTATGGCGCAGAGACTTCCCCATGACCAATCATTC | 106 |
| cg16416603 | CGGGCACCCTGAGGTTGCCCCCATTCTTTGAACAGCTATGGACGGGGACC | 107 |
| cg05276137 | CGGGCTTCCCGGCAGCAGGTTACTCTTTAACGCTCGACTGCCCACCTCCA | 108 |
| cg16983817 | CGCCCTGATTGGATAGTAGTGCAGGCACCGCGCCCTCAGGCCTTGCGTGT | 109 |
| cg00744433 | CGCAACTGCAGTTTAGCCTGGTGAGTTGCAATTCTGGCTGCACATGAAAA | 110 |
| cg08706141 | AGTTTTACAAGCCCTCACATAAATCTACACAGCTAACATGCACAAGTCCG | 111 |
| cg03860256 | CGCCTTGCGCGCTCGGCATGACGTCACTCCCACCAGGCACCTCGCCTTGT | 112 |
| cg15876825 | AGGCGATCCTTGCGCTTGGGCAGACAGCACGTCACGGCTTTTATCTGCCG | 113 |
| cg21770393 | AGTCAGACGTCAGGACCTGCTGTGGGGATGGACCCACAGCTCATCCCCCG | 114 |
| cg01206872 | CCCAGTACTTTGGGACGCCAAGGTGGGTGGGTCACAAGGTCAGGAGATCG | 115 |
| cg02019988 | TACCCCAGCTAGGCCCCTTCCCCGCAGAGGATGTGTTTCTGTTGTCCTCG | 116 |
| cg04785972 | GAGGTGGGCGAGGCAGGGCCCACAGACATTGGTGCAGGCTCAGCCGTGCG | 117 |
| cg19859270 | CGACTGGCCACACAATGGCCAGGTAGCGGTCAACACTCATGCAAGTGAGC | 118 |
| cg16665506 | CTTCCCCTCAGCCTTGGTTTCCATATCTGGAAGATGGGTTACCAATAGCG | 119 |
| cg01328703 | ATGTCACTTGCACAAGAGCCAGTCATGCCACAGGGCTAGCCTTAATTTCG | 120 |
| cg17894318 | AAATAACTTGTCCTTTACTTACCCTGCACATCAAATCTTCTCGCCCTGCG | 121 |
| cg17500754 | ATTTTTATTTTATTCCTGCCATTGTTTCTCCTTCGAGTCAATATCTTCCG | 122 |
| cg21924273 | GGGCGCCAGCCGGCGAGGAGCGACGGGAGCCTGGGGTGGGTGGCGAGGCG | 123 |
| cg22518157 | CGGCTGTGGTTTCCTGTGCGTCCCAGGTGAGATCTTGCTCTTTACAGGGT | 124 |
| cg22659049 | AGGACGACAAATGGCAAGATGTGAGTTGTGGCCAAGGCGCGCGGGCAGCG | 125 |
| cg25806704 | CGGGCTTAGTAAGCCCCTGGAGAAAGGAATCAAAGATGTTTCTGAGGCCT | 126 |
| cg07973162 | CGCTGGCCACCCTCCTAAAGGATCACTGTGGTGCCAGGCAGGAATGAGCT | 127 |
| cg10144483 | CGGTGTGCAAACCTGCAATGAATTGCCCAGTGCCTTCTTGTTTGTAACGT | 128 |
| cg11554391 | ATTATAGAGAATATTAAGATGCTTTTGGATCCTGAGCTTCCCAGGAAGCG | 129 |
| cg05575921 | CGGCTGGGTCTCATCTGACACGCAGCCTTCCAGCCTCTCATTGCCCGAGG | 130 |
| cg03604011 | AGGGAAGTTGGGAGTATTGTGCTCCCCTGAGAGCCGTTGCCACGGTTACG | 131 |
| cg12035880 | CCAGCAGCAGCGGCAGCAAGGTTTACAATCCTAAACCATGCAGGCTTCCG | 132 |
| cg19601991 | TAGCCATTTGGCCTCGGCATCACGGTGCGGGAGCGTGGGGCGCAGACTCG | 133 |
| cg26921969 | CAAGCAAGACTCTCCACCCACAAACTGCATATTCTTTAAAGTCACTGTCG | 134 |
| cg04836987 | CGCTTAAGGACCTTATAGCTAGCAAATGGCAGAGACAAGCTTGGGGCAGA | 135 |
| cg15658426 | AGATGTTATCAGCTCAGCGAGACTTTCCATGATCTCCCTAAAATGAATCG | 136 |
| cg21777188 | CGCCCAGAACTGGGACTTCCCAGGTTCTCATAGTGATTGTAGATATCAAT | 137 |
| cg05845376 | GCTAGTGAGCCGCGCGCTGGGCCGGCAGCAGGGGGTTATTTTAGTGCGCG | 138 |
| cg07039560 | GGCTGGAGGCGGCTGGGGGCCCGGCCAGTGCGCCAGGAGCCGCAGGAACG | 139 |
| cg25587069 | GGCAGGCAGGCGGCAGGCAGAGCGCGCTCTCCGGGCAGTCTGAAGGACCG | 140 |
| cg13667243 | CGCTCCGCCGGTGCTCGCTCTATTTGAAAACGCTGACTGTTGGTCCCCCA | 141 |
| cg12363682 | ACAGAACCCCAGAAAAACTGTCCCACAACCCCCAGTGAATGACCAAATCG | 142 |
| cg07412232 | CGGAAGCTGCACCCTAAATTCCCCGGGACCTGTGGCAGGCCTTCCTGGTG | 143 |
| cg15174951 | AGCCACGAAGAAAACAGTTCAACTCAGACCAAAAGATAAAGCTGATCTCG | 144 |
| cg18892128 | GCCTGGTCCCCGAAAGCGCTCAGTCAGAATCCGAGAGCACTGTGGCGGCG | 145 |
| cg14924781 | CGCCTCTCACAGACTGCTTCCCCAGCAGGGTGCAGCTTCTTACAGACTGG | 146 |
| cg25955180 | TGATTCAGTCCAGTGATTGGGTTTGTGGCTCCAGGCCTCGCCCACAGACG | 147 |
| cg27067781 | CGTCTCGCCTTGCGAGCAAGCTCGGAATCCAGTTCCTCAGGAACCCCTCC | 148 |
| cg21091547 | CAGCAAAGAAATGACTATAGTTGGAACACAGGACTTTTGCCTCCTGCCCG | 149 |
| cg26470115 | CCCATCCATGACTTCTTATGTGTGCTGTCGGCAGCTGACCAGCGGCCACG | 150 |
| cg04344923 | AGGCAGCAGCCCAGTGGCCTCTTGCTGTGGAGCGCGGGGGCAGCCTCCCG | 151 |
| cg06413272 | CGGGTGTTGTGTCTCTGCCTATCTTCACACATGCACCCTTGTGAACTTGG | 152 |
| cg02817030 | CGGACTCATTGCCATGCATGCCTAGACATTAATATGTGTTCATGGAGGGC | 153 |
| cg26403206 | TGTGCTCAGATTCACAAAGTGAAAGTGCAAAACCCCGTGTTAGCTCATCG | 154 |
| cg15777014 | CGGGCTATAGCGAGACCCCCCATAGTGAGATCTGCTGCGGGACCTAGAGT | 155 |
| cg01704252 | CGGTGGCCTGTTGAGGCCCAACTCATGCCTCTGGCACCCTTTCAAGAGGC | 156 |
| cg15600935 | GCAAGGTGAACCCACCTTGATCCCGGGGCTGCAGAGCCAGTGCGCCTGCG | 157 |
| cg25949550 | CGGTTGGCAAGAGCTTGGCTAGAGAGACTGATGTAGATGGCAGCTTCTTA | 158 |
| cg11207515 | CGAGAGGCATTTCAGCAGAATTTTGAAGGTGGAGTGAAGGAAGTCACTAT | 159 |
| cg09159795 | AAACACTCTCCCGGCTCAGAAACGGTGCAGGTGCATTTACATGTCAATCG | 160 |
| cg07338119 | CGCGGGCTCTATCTGCAGCATCCACCCCCTCAGCAGACGCTGGCATAAGC | 161 |
| cg09608412 | GGGGCCGCAGTGTCCTTGGCTCAGGCCTCCTACGCAGGGAAGCAGGGTCG | 162 |
| cg14700531 | CGGGAAAGTATCACTTGTGACTGTTTGCACTAGGTGCAAAAGACAGATAG | 163 |
| cg03900851 | TCTGTAGAGAAGTCGACCGAGAAGTGAGATGCGGTGGCGACCTCGGAGCG | 164 |
| cg27272363 | CGCTGCCCATGCCCAGACCAAAAATGGGGTGTCTTTTTAGTCTAAAAGGA | 165 |
| cg16821867 | CGGCCAGACCCGTCACTCAGATCCCCGCACCAGGCCAGTGAAGAGGAGCC | 166 |
| cg15410835 | CCAGCCCTTCTGGCACAGATGCTGTGCATGTGAGGTGCTTCTCAGAAGCG | 167 |
| cg00844308 | AGGAGATGAGCTATGTCTTCCAACCTCTCCTTTTGGAATAAAACCTTTCG | 168 |
| cg13728106 | CGACGACAAAGAAGATTTTGATGAAGTTCAAATTAGAGTCAGCGTGCTCA | 169 |
| cg27087885 | GTAAAGTGACACCCATCCCACTCATCCCATTGCTGCCAATGACTTCAACG | 170 |
| cg20448001 | CACCTTCCACTTTAAAGAACACCAAGGTCGGCTGGGTGTGGTGGCTCACG | 171 |
| cg15480287 | CGAGCCCAGGAAATAAGTGCGGGTGACCGAATTCAATCCTGCATCCAACA | 172 |
| cg19414383 | CGGCCCCACTTAGCCTGAAACGGGGCCCCATGCAGGGTAGAGGCCTGCCA | 173 |
| cg25536676 | CGCACACCGAGTGGCACGACCTGCAGAGGTTACCGCCAGGTTTCCATCCT | 174 |
| cg22820188 | TGGGGTGAGCCACTTTCATTTTCCCAGCGGGGCCAGGCAGTCTTTGCTCG | 175 |
| cg08410533 | CGCAGGTCTGAGAGACATCACAGCATCACAGCGTTTAGTAGTCTTAAATG | 176 |
| cg12483947 | CGACTACCACTGCTGGTGCAAGGACCAATCAGCGACCTATTGAAGTGCCT | 177 |
| cg23371436 | CGGGATGGTGTGACAGGCAAGCCAAAGTTGTATCTGAAAACTCTGCAACA | 178 |
| cg24894584 | GGTCCGCTGGGGCAGAGTGCGGAGTGAAGGGGTGCACTGGGCACTCAGCG | 179 |
| cg26266429 | CTTTAAGATGTCTGACAGTGCCCTAAGGGCACGTGCTCCACAAACAAACG | 180 |
| cg10068417 | CTCAAAGAAAATAAGCCAGGACCAGGTGACCCAGTGGGATCCGCGAAGCG | 181 |
| cg25572904 | TGCGGCCACTGCAAAGCGATGTAAATGTCCCAGAGTGGGGCAGCTGGTCG | 182 |
| cg13543355 | CGTGCCACCTGGCCAACCTCGAAAGGAGGCCCTGCTGGGTTCTTCATCTT | 183 |
| cg26481784 | CGGCGGACCCAGCTGACCAAAGGAACTACAGAAGAGGACGAAGAAAGCGA | 184 |
| cg13898384 | TCGCTTTCCAGCCTTTTGTTTCTGGATCCTCAAAACTCAGAACGTGGCCG | 185 |
| cg19459791 | TGGAGCAAATATAGGTCTAGACACGTAGGGCCTGTGAGGTTTTCCTGACG | 186 |
| cg03172657 | GCGTGTGCTCAGCGTGTGGTTACCGTGTGCACAGCACGAGCTTGAACTCG | 187 |
| cg18181703 | CGTGGCCACTCTTCAGCATCTCTGTCGGAAGACCGTCAACGGCCACCTGG | 188 |
| cg07573872 | CGGGAATGGTGACTCAGCCTTCCAGGAACCTGCGTGGCGTCTGTTTTTTT | 189 |
| cg09051966 | CGGGCGCAGCCCCAGGATGCAACAGCTCTCATACAATTGGCCAGGAAGGC | 190 |
| cg25841091 | CGGGTTTCCCAAGTGCTGGACGCGCAACAGGTTTCCATAAACCCAAAGYT | 191 |
| cg12516875 | CGGCGAGTGAGTGAAAAATTACATTTTCACTTGGGTTCTGAACATTCAGT | 192 |
| cg10753966 | CGGGGAAACTCTGAATTGGTGGTTGCTTAGAAAACACATAGAAGTGTGTA | 193 |
| cg23944298 | TCAGGGYTGAAAACGCAACAGCCAAAATTAACTCAGTGGATGGCTCAACG | 194 |
| cg07553761 | CGCCGGTGGCCGACGGCTTCTGAGGAATTATCTTTTACTTGGCGCCACAC | 195 |
| cg10202557 | CGAGATTAAAGATTCAGAAAGTGAGTAAAGTTAACACTGAGCCCTTGGGC | 196 |
| cg05604079 | AGTTGGTGAGCCAGGGAAGTCTTCCCTCGCCCTGAGGGTGCGGGGAAGCG | 197 |
| cg11444009 | CGGGGCCAAAGAGGAGCGCAGGTTTAAAAAGCAAGTGTATTAGCAAGTTT | 198 |
| cg25149516 | CGCCCACGCGGACCCCTCTCCGTGTGCTTGTCTGTACTTCCCTGATGGGG | 199 |
| cg18584747 | CGGCCGGAGGGCCCTGAACCCTAGGCCAAGACTCATCCACAACCTAGGGG | 200 |
| cg20405584 | CGGGAGATCCTCCCGCGGCCGGAGGGCCCTGAACCCTAGGCCAAGACTCA | 201 |
| cg11299854 | CGTGAGCGGGATCCTGAGTCTGAGTTGCCGCTTAACCCGGCATTTTATAA | 202 |
| cg24708145 | CGGAGGCAAGCAAGCTCCATGTGGTGGGGACAGCGCATTGCGACCTCCAC | 203 |
| cg25663874 | CCATGCTATAAGTGAGGTAAATAAATATGCTGAGCACCATCGCACTTACG | 204 |
| cg25325512 | CGCGGCATTCAGCAGAACTCATGATGAAGGAAACAGTTCCATGGCAGGGT | 205 |
| cg13001142 | CGACAAAATCTGACCAGGCTAGATCTGAATCTGTTAATTTCTGAGACGGT | 206 |
| cg06649410 | CGCAGAGGAGAATGTCAGATGCTCAGCTCGGTCCCCTCCGCCTGACGCTC | 207 |
| cg17501210 | CGCCCGATTCAGACAGCTGGACTCAGAGGGATTCTGCTCCACAGAGAAAC | 208 |
| cg03473532 | CGTATGTGTTTGAGATAGCAGTTGTTTACTATCACTTGAAAATTCTGAAT | 209 |
| cg20468415 | TCATGAACTGGAGCCTGAAGTGGCTCTGAGGCCTCCCCTGGAAAGGAACG | 210 |
| cg11782409 | CGGTGGATTCCTCTCGGGACACATCTGCCACCCGCTGGACTGGCAAAACA | 211 |
| cg07205627 | CGACCATTTGTATGTGTATCTATGTCAGAAAGAATCTTTATTAAAATATT | 212 |
| cg07274490 | TACCATGTAATATACCACTAAACACACACTATGTAATACACTACTAAACG | 213 |
| cg07929447 | TTGTCTCAGATTACAGGAACCAAATGCATGTCTGGCCTTGTGCTGCTTCG | 214 |
| cg00695391 | CGAGCCCGCCCAGCCTCTGAACCTCCGTGTCTTCATCTGTAAATTGAGGG | 215 |
| cg16305333 | GTGCTCTCAGTCCAATTCACACTGAATGAATGAATCAGTCCCAGCCATCG | 216 |
| cg00970752 | CGCTGAGGTCCCGTTTCTGTAGCTGAGTAGCCCGGTGACACTGCCCTCCG | 217 |
| cg04967775 | AGGGCCCCCTGGGCCTGTACCAAGGTCTGCGACAGATGGGTGGGGAGCCG | 218 |
| cg06901711 | CGCCGCAGTCCCAGTCGAGCCGCGACCCTTCCGGCTGCCCCCACCCCACC | 219 |
| cg01431830 | TGTGCCTTGACGGGCTGTCCCAGGCGCCCCTGGAATTGATGCAGTGTCCG | 220 |
| cg10578779 | CGCAGAAGAAAGCTGCTCAAGTGCTGGAGTGGCCAACCCCATGCCGTTCC | 221 |
| cg26359730 | CGCACATCCTCAGCTAGCAGCATGGAGCTGAGTTCAGAGGAGGCACTTAG | 222 |
| cg10585941 | CGGGAGGGAAAGGTGTTAGATGGTGGTTTTAACTCACCAGGGTTTGAATT | 223 |
| cg01939962 | CGGCCTGATTCGAAGACTGGAATAGAGGATTTCTAGAATTCTGTGACTCC | 224 |
| cg06809285 | CATGTCCCAGCTCAGCTGGTCCTGAGAAGCTGCCTGGTAGACAGTCACCG | 225 |
| cg11336382 | TAGAACCAACTGGATACTGCACCCCTCAGTATTGGGGCGTCACAGCACCG | 226 |
| cg08410533 | CGCAGGTCTGAGAGACATCACAGCATCACAGCGTTTAGTAGTCTTAAATG | 227 |
| cg04196119 | CAAGTCCTTTCTGGTTCCCGTGGTTTTTCAGGTACTGCGGTTCTTATTCG | 228 |
| cg12329405 | CTCCCTACGTGGGGGACGTGCAGGATGATGCGGGGTCGGGGGGGATTTCG | 229 |
| cg12379046 | TCCGCAAAAAGCAGGATCCATTCACTTTAGAGAAGTGCCCTCAACAGGCG | 230 |
| cg03950121 | TGTGGTCTCCCCAGACCACCAGGTGCTTCACACACGCACAGGGGCACTCG | 231 |
| cg03096347 | TGCAGCCTCGTAAGCCTCATGGAAGAGCTTGGTTTGTATCCTTTGGAACG | 232 |
| cg22620356 | CAGTGCTGAGCCGGATCCAGAAAATTGAACAGGTCCTGAAGGAGCAGCCG | 233 |
| cg17310258 | CGTCCCCGAAATGAATAATGCAGGCAGGAGCCAAGATTTCTGCATTAGCG | 234 |
| cg18886347 | CGGTGTGGAGGCTTTTTTTGTCAGAAGCTCTGGGGTTTGGCTCTTCTCTG | 235 |
| cg14592365 | CCTCTTTGCTGATCTGGACTCAGCCTGGTATTCTTGTCACAAGGTCATCG | 236 |
| cg07631435 | TTTTTAACCAGTTTGAACATTTTACTACTTCCTAACATCAGAAGCACCCG | 237 |
| cg03112631 | GTGTGGCAAGGCTGAGCTAGTTCGTCTGCCAGGTACCAGAGCAAGCCTCG | 238 |
| cg01635346 | CACTATGTTTGCTGCTTGCTATTCACTTATGGTACATACACAGTCAGTCG | 239 |
| cg25966649 | GGGACGGTGGTAGTGCTTCTCAACATTGGCACTGGCTCCATTGTCATTCG | 240 |
| cg23510415 | TGGGGAGCCAGCTGACTTAACAGGCCTGGATTACATTCTTGCTTCCAACG | 241 |
| cg18159533 | TGCCATTTAATCTAACGCCACCTTCTACGGACCAGAGCAGGTGCTCTGCG | 242 |
| cg16982073 | CGGTTAAATTCGTGAGGCTGTTTGCTGTGTACACAATGCTAAGCAGAAAA | 243 |
| cg25572904 | TGCGGCCACTGCAAAGCGATGTAAATGTCCCAGAGTGGGGCAGCTGGTCG | 244 |
| cg02150207 | TCTGGTCCTGGCCTGGGGGGGTCGTGGTCTCGCTGGTGCAGCCTCCTCCG | 245 |
| cg13649020 | CGCCACCATCGAGTGTGAGCAGCCCCAGCCCGACCTCTACAAGTAAGCGG | 246 |
| cg06903325 | CGCTGTGAAACCTTTGCCTTTGGGTGTCATGGTGGAAGCAAATCTTAGAA | 247 |
| cg05078817 | CGCCAGGGCAAACATTCCTTGGGCGCCTCGGGCTTTTCCCTAGGGAAGCA | 248 |
| cg13898384 | TCGCTTTCCAGCCTTTTGTTTCTGGATCCTCAAAACTCAGAACGTGGCCG | 249 |
| cg13634681 | CGGAGAGACAGCCGCTGTGGTTAGGGGTGTGCATCCTTCTACCATGCCCA | 250 |
| cg01624571 | CGTGGATCTAGCCACAGAAAGGCAACAGCGCTTCAAAGAGGCTCTCCGTA | 251 |
| cg14153919 | CGCTTTTGCAAAACAGGCCGCACCTAGCCCAGATTCTGGAGCTGATTCAG | 252 |
| cg05575505 | CGACTCTGAGTTCTGACAGGGAAATACAATGAGTTTTATGTGGACAGAGT | 253 |
| cg19987111 | CTCTCTCCCAGACCTCAGGTAAAATTAATGTTCTAGGAACGTATGCCACG | 254 |
| cg05638439 | TGGGGTGGCCGTCTGCTCCGTCTTTGGTGGTGAGAACCCGCACCGGTCCG | 255 |
| cg26470314 | CGGTGTCTCCGCTCCTTGCCCGTGGCCAGGTGGTGGTGCTCCCAATGCCC | 256 |
| cg24429836 | CGGGCTTTAAGGTGGCCCTTGCAGATCTTGCAGACATTTTCGTGGGCTCC | 257 |
| cg04120520 | AGACTCCCTCTGAACACCTGTGGAACATGCTGACCTCCCAGTGTCTTTCG | 258 |
| cg09319676 | CGCACATGTGAGGCCACCATTCATACCACACCCTGGGTGGGTCTTGAGTT | 259 |
| cg20433952 | TCTGCTCATTAACACCCTGTTAATAGGAACCCCAGAAACCTGAGCTTCCG | 260 |
| cg02220125 | CGGCCTTGCACACCATCTTAAGTGTGACGCCACACTGCCCTTCACAGCCT | 261 |
| cg18181703 | CGTGGCCACTCTTCAGCATCTCTGTCGGAAGACCGTCAACGGCCACCTGG | 262 |
| cg10701033 | CGCGCCTGGTGCAGCCCTCACCTCACCAGGTETTGATCATCTTAGTGGTT | 263 |
| cg07529654 | CGCACTTAATATGATTATTTTTTGATGCCAGGTCCACCAAGTGAAGATTG | 264 |
| cg27145096 | CGTGTAAAGGTCAGAGGTCCATTCACCAATGCAATGATCCTCACTGCCCC | 265 |
| cg07573872 | CGGGAATGGTGACTCAGCCTTCCAGGAACCTGCGTGGCGTCTGTTTTTTT | 266 |
| cg14528319 | TATTGCAGTATCCTAAAGCGTGCTGTATCTCTCTGTCCGGCTTTTGCACG | 267 |
| cg09920725 | CGGTCTTGTAACAATTGGATGGATGCCTTTGAAGAGCCCCTGTCCCTATT | 268 |
| cg24000528 | TCTCCTCCCCTGCACTGGCTCTGGGTGCTTATCTCTGCAGAAGCTTCTCG | 269 |
| cg13772414 | CGGGGTCTAGGGAAGAGAGATTTCCTAACCTAGACTACAGGCATCAGGAG | 270 |
| cg08007899 | TGATTCCTCCCCCTTTGAAAGAGCAGGCAGCATTTTTCCAAGGCAACCCG | 271 |
| cg15808795 | CGCTGGTCTCGGGGGGGAACCCAACAGACTCACATTTCATGTGAAAGGAA | 272 |
| cg09845806 | AAAGATGTCATTACACAAATACACAAATACTAGCATGCAGACACACAACG | 273 |
| cg03706056 | GATGTATCTCCCACCCAGGAGTGTTTCTTGGCCAGGTGTGGTGGCTCACG | 274 |
| cg24110177 | CGACAACTCTGTAGTGATTTAGAAGAGAATTGCAAGACATTAACACAGAG | 275 |
| cg10753966 | CGGGGAAACTCTGAATTGGTGGTTGCTTAGAAAACACATAGAAGTGTGTA | 276 |
| cg00481951 | GTTTCAGCACCTGGGTCAGCGCTTCCCAGGGTCAGCACCAGGGATAGACG | 277 |
| cg04748223 | CGCAGCTGTCGTCTTTGTACCCGGCTTTGCGCACTCCATTGTGGACGCGA | 278 |
| cg22518157 | CGGCTGTGGTTTCCTGTGCGTCCCAGGTGAGATCTTGCTCTTTACAGGGT | 279 |
| cg22714094 | GCACCTTTGTGAGGTGTTTGTGGGGTTGGGGGAGCTTCAGGCGCTACTCG | 280 |
| cg10177080 | CGGTACTTCCCTGATGGGGCCCACGCGGACCCCTCTCCAGTGTGCTTGTC | 281 |
| cg15845365 | ATCTTCCCAGTACAGAGCTTAGCATACATTGAATAACTGGCATCTGCTCG | 282 |
| cg27628536 | ACTCTGTCAGATATCCTCGATATTTGGGGATAGTAGTTCAAAAACAGCCG | 283 |
| cg24765579 | AGCTGTTTCCCTCAGGCGGCCTGTTCCACGCCAGGATTAGTTTTGCTTCG | 284 |
| cg15988937 | CGGTTTGCAGGTTCAAAGATGTCTAAAAGAGAATGGCCAGAATGTATATA | 285 |
| cg02081065 | ATCCCTCATCTGGACTTTGAGTGATTGTGTTGCTGGCATGGACCCTTTCG | 286 |
| cg12612118 | CAGAGCCCACTTAGTGCGCGCTAGCTGGGCAGGGATAGGGGTCCTATTCG | 287 |
| cg12298697 | ACAGTATTTTTGTCACTCCAAGTATCCCTTATCTTGCCCTTTTATGGTCG | 288 |
| cg20660989 | CTGAAAGGCGCATTGTCAGCCTGGGGAGGCGACGCGGCGCGAGGACAGCG | 289 |
| cg03546163 | CGGAGGGCTTATTCTATGTAAATAGTTGAAAGGAACTGGATAAGACTGTA | 290 |
| cg25325512 | CGCGGCATTCAGCAGAACTCATGATGAAGGAAACAGTTCCATGGCAGGGT | 291 |
| cg00684824 | CGCCCAGCTGGAGGCTGAGATGTCAGGATTTGTCTTGCAAAGTTACTTCA | 292 |
| cg02713068 | CGCAAAACTTAAGGCTATAGAAGATGGGGAGAGTATCTTTTGATGCAGAC | 293 |
| cg06649410 | CGCAGAGGAGAATGTCAGATGCTCAGCTCGGTCCCCTCCGCCTGACGCTC | 294 |
| cg17501210 | CGCCCGATTCAGACAGCTGGACTCAGAGGGATTCTGCTCCACAGAGAAAC | 295 |
| cg03807873 | CGCTGCACTCAGCCACGTTACGCGGAGTTCATCTGGGGAAGGAGACAAAT | 296 |
| cg21899558 | GAAGGGTCACACATGTCCCAGAATCCCCCAGGAGTGATGCAAATGGAGCG | 297 |
| cg07025011 | AAACCCTAAACACTGAAGTCTCCTCTGGTCAGGTGACCCCTATCAGTCCG | 298 |
| cg16847637 | GCCAGGAGTCCTCGGTCCTGGGAGGGTTTAAAAGCCAGGGGGCCGTCTCG | 299 |
| cg06563451 | GACCCTGGGCTTGGGGGGATTTTTTTGGCCTGGAGGGCTCCTCTTCTCCG | 300 |
| cg02716826 | GGCCAGACACCCAGGACCCAAATAACCTAATAATGGCTCATTTGCCTCCG | 301 |
| cg13947317 | CGGCATTGGTCCTTTATTGAACATCCTCCCAAAGCTGGGGCTAGGGTTCA | 302 |
| cg22510139 | CCAGATGTTTTCACTCGATTTTGCAAATCTGCTCCAGGTCTAAGACATCG | 303 |
| cg10116490 | CGGGTCTTCACTCTGGTTGGCTGCCAGATCCTCCAGGGGCGGGGTCTCAC | 304 |
| cg27396830 | TACAGAACCCCTGAAACATTCCTGCCAAATACAGTCGCACGAATTTGTCG | 305 |
| cg19414383 | CGGCCCCACTTAGCCTGAAACGGGGCCCCATGCAGGGTAGAGGCCTGCCA | 306 |
| cg03345668 | CGCACAGCAACTGGGGTGTCCTGACCAGTGAGCTAGAGTTCCACCTTACT | 307 |
| cg17719473 | TTATAATCCAAAAATATTTATTAACCATCTGCTCTGTGACAAGCACTGCG | 308 |
| cg20569940 | CTTTGGCTCGAGCTCCAGGTGCAGATTTACGTTCATTTTCTTTTGTTCCG | 309 |
| cg26156167 | AAAAACTTTTCTGAGCCGGAGCCTCCAGCGCGCTGTGTATTCGTTTTACG | 310 |
| cg10578779 | CGCAGAAGAAAGCTGCTCAAGTGCTGGAGTGGCCAACCCCATGCCGTTCC | 311 |
| cg15631106 | CGCAGATTCCCCATTTGTGAGCAGAGTTAAGCTGAAGGAGCGTGGGCATG | 312 |
| cg22704788 | CGGCCCCAGAAGGCAAGGCGATTGTTAGGAGGAATTAAACAGGCTAGTGC | 313 |
| cg20240347 | CGCCCGGCGACCACTGGTGGTTCTATGCTGCTGGGGTGAAAAGTCTGAAG | 314 |
| cg17803430 | AGGACACTGGCCAGCCTCCAGTTGGTTTCTGAAGCTAGCAGTCTTAGTCG | 315 |
| cg20800892 | CGCCCTGCCCTGGGATTCTCAGACCTTGAAAGCCACAAGTCTTGAAAGTC | 316 |
| cg08410533 | CGCAGGTCTGAGAGACATCACAGCATCACAGCGTTTAGTAGTCTTAAATG | 317 |
| cg12329405 | CTCCCTACGTGGGGGACGTGCAGGATGATGCGGGGTCGGGGGGGATTTCG | 318 |
| cg14984434 | CTGAATTCTGGTACCTGTTAATGACAGCTTCTGAGAGGTTTCTTTTATCG | 319 |
| cg07690734 | TAACCTTGAACAAAGATCGTCGACGGGCGGCCCTCACTGGAAACCTTTCG | 320 |
| cg17310258 | CGTCCCCGAAATGAATAATGCAGGCAGGAGCCAAGATTTCTGCATTAGCG | 321 |
| cg14955976 | CGAGTTGTAAGTCAACCTGCTGGGTTGTGCTGAAGAGAAATTGGAGACTG | 322 |
| cg06975311 | TTAGGGCCTGGAGATGGGCAGACCCAGGCTCACATCCTAGCTCTGACACG | 323 |
| cg16246188 | CGGCTCTAAGCGGGGCAAGAGCTGCTTTTAATTTGAGACGTACACAGTTT | 324 |
| cg07631435 | TTTTTAACCAGTTTGAACATTTTACTACTTCCTAACATCAGAAGCACCCG | 325 |
| cg12211040 | ACACATACCCCACTAAAGAAGATTCACAAGGTTGGGTGCAGTGGTTCACG | 326 |
| cg11764442 | ACTATGTACCTGGAAATAGCAGGGGCTCAGGGTTTGATCCTATGAGACCG | 327 |
| cg10535933 | CGGCTACGAGCACAGAGCATCATCATCATCAGCCTGCCAGCCGGCCCCGC | 328 |
| cg01624571 | CGTGGATCTAGCCACAGAAAGGCAACAGCGCTTCAAAGAGGCTCTCCGTA | 329 |
| cg24521756 | CCTCTGGACAGTACTTTTGAGACACACTTATTTCTACCACGCACCTTGCG | 330 |
| cg07842062 | CGAGAAGTTCAAGTTCAAGCTGCAGAACACCAGTGTGCAGAAGGAGCACT | 331 |
| cg08072101 | CGCGCAGGAAAAGGGAAACAAACAGCAGCTTCGCAGCCTCCCTGGGCCTG | 332 |
| cg03955530 | CTATGGTCAACTGAGTCAGGGCTGGATTTCTCACCCACCCTGGCCAATCG | 333 |
| cg07839457 | CGCTCCTATTCACCGAACCCCTGGCTGGCTGACCCGGGAAAAACAGTCTA | 334 |
| cg04976151 | TCCACCAGTTCTCTTATCAATGGACAGTACAAACAGTACTACCACGAACG | 335 |
| cg03400403 | CGCGTGTTTCTCTGCGCTGTGGTCGTCATGGACCGGAAGCGTGTGCAGCG | 336 |
| cg04745805 | TTTTCTTCTTTGGCTTTTCCATCACTGCTGGCTACAAGTGTTTGCAACCG | 337 |
| cg14545159 | GGGGTTGCCGGCACCTTCTCTTTCTCCCCAGGTCCTTTCATCTGATTCCG | 338 |
| cg18181703 | CGTGGCCACTCTTCAGCATCTCTGTCGGAAGACCGTCAACGGCCACCTGG | 339 |
| cg17100770 | CGCACGGCCTCCAGCAGCAAGCTGATGCTGTCCTACAAGAAGCCAAAAGG | 340 |
| cg27145096 | CGTGTAAAGGTCAGAGGTCCATTCACCAATGCAATGATCCTCACTGCCCC | 341 |
| cg13077366 | GTCCTAAAGCACCAGGATAGATGACGAGCAGGGCCAAGTCCACACAAGCG | 342 |
| cg21656736 | TTTAAGCTATCAATGTGGATGTGCTGGTGCTGAGACCCACCTCCAACACG | 343 |
| cg07573872 | CGGGAATGGTGACTCAGCCTTCCAGGAACCTGCGTGGCGTCTGTTTTTTT | 344 |
| cg21736089 | CTCCTCCGAGTAGCCGGCGCCGTGGATGATGCGCATCTGCTTGATGAACG | 345 |
| cg07543883 | ACTGGACCCTCGGCTCTTCCTTGGACTTCTTGTGTGTTCTGTGAGCTTCG | 346 |
| cg10224107 | TGGGGCTTTAAAAACCACAGCCCTTGGGCAGGAGGGACCTTCGATCCTCG | 347 |
| cg01909777 | CGCTCCCTCTTAGGTCACCTGCCATTTCAGACAGGGAAGAGAACAGTGAA | 348 |
| cg03760035 | AGCAGCTCCACCGGGGATGCGGCTCGTCAGGGGGTGTACGAGAACTTCCG | 349 |
| cg03706056 | GATGTATCTCCCACCCAGGAGTGTTTCTTGGCCAGGTGTGGTGGCTCACG | 350 |
| cg13092901 | CGCCACAGAGGGCTTCCCCAGGTCCAGGCCGGGCCAGCCTTGACCTCTAA | 351 |
| cg15849439 | CGAACCTGATGTGGAGACAAAGTATCCTTCATGGACTTTGAATTTGATAT | 352 |
| cg12605080 | AAGGGTAGCTGCTGCCACTGCTACATTCTACAGAAAGCTCAATGAGTCCG | 353 |
| cg01163330 | CGGCTCCCTCACACCCACTGAGAGCATCATGTCCCTGGGCACGCACTCCC | 354 |
| cg10753966 | CGGGGAAACTCTGAATTGGTGGTTGCTTAGAAAACACATAGAAGTGTGTA | 355 |
| cg19501902 | CGCTGCTTCAACTGGAGTTAACCAAGTGAACACAGAGCTTTACAAAGAAC | 356 |
| cg13287247 | CTCGGTCTCGTGTGTCTGTGTGTGCCAGTCCCTTGTGCTGACACAGGACG | 357 |
| cg22930808 | TATGCAGTTGTAATTCTGACAGTATTGCCAAATTGTTCACAGTAAAGACG | 358 |
| cg08122652 | CGGCAGAGAGGCCTGAGATAAGGTTTTTGCACATGGCTGGTAAGACTGGA | 359 |
| cg00959259 | CGGTTGTCAGCACCATATTTCTAAAATGCAAATCTGGCAAGTCATTTTCT | 360 |
| cg20576510 | CGCGGCTGTACCACATAGAGAAAGATATGCACTAGTTCAAAGAGAATGCC | 361 |
| cg10202557 | CGAGATTAAAGATTCAGAAAGTGAGTAAAGTTAACACTGAGCCCTTGGGC | 362 |
| cg27134386 | AGGCCCCAAGGTTCCTGACAGAGCTGCTGTTGGGTGTTGGTGTAAAGCCG | 363 |
| cg10584300 | AAAAAGACTGCAGCTCCCATCTTAATTGCTCTCTCATGCTTGTGTGTGCG | 364 |
| cg03274876 | GAATTCTGCTGGCGCTGCAGCTGCAGAATGGTCGGCGGTGGCGGGAAGCG | 365 |
| cg17287155 | AGCTTCAGACACAGTCAACGTGCTGCCAACGTGGGGAGGGCCGGAGGACG | 366 |
| cg04764584 | CGGAGATGAAATTAATGTGAACCACTGAGGCAAAGGCATGAGGCCCCCTC | 367 |
| cg15679283 | CGGCTGCCCCCTCACCAGCACCCTCCGATTCCAGCGACAGCCTCTTGGCT | 368 |
| cg17533522 | GTCCCTGGAGCCAGGCGTTAATCATTGACCTGATTAGCAAGAACGCTCCG | 369 |
| cg14868212 | CGACATGACAAGATTTAAGGGAGGCACATCTCACAGACAAGCATGAAAAC | 370 |
| cg16274199 | CTGGCCCGGCCGCCCCTCAGCTATTTGTTCACGTAATGCGATTGGAAACG | 371 |
| c213309828 | TAAATGGACTTACTTTGAGGCCATGATCAACATTTAAATCTTGCTCTTCG | 372 |
| cg12612118 | CAGAGCCCACTTAGTGCGCGCTAGCTGGGCAGGGATAGGGGTCCTATTCG | 373 |
| cg03415429 | CGGACACTTCCGGTGGAAGGACTGAGCGGCGCTACACTTCAAGAATTCCG | 374 |
| cg15947697 | TTATTGTTTTCCTGTCTTCTACAGCAGAATTGGATATTCCCAAACAATCG | 375 |
| cg11867651 | CGGGGTCTGGGTTTTCTTGTCCCACTGGGAGTTTCAAGCCCCAGGTAGAA | 376 |
| cg17096289 | TCAAGGGAGGGCGATATTCCAGTGCTGATCCCATTTTCCTCCCCTCCTCG | 377 |
| cg01517384 | TCAGGAAAACTCATGCCATTCTCCATTCAACGGAGGGCGACATTCTAGCG | 378 |
| cg16928551 | CGTCCAGCAGACAAAGACTATGGTTTAATGAAAATAGATGAACCAAGCAC | 379 |
| cg26987613 | ACTGATGATTTCCAGGAACTACCAAAGCCACGGATCAGCTGAGTTACCCG | 380 |
| cg20660989 | CTGAAAGGCGCATTGTCAGCCTGGGGAGGCGACGCGGCGCGAGGACAGCG | 381 |
| cg21288889 | AACAACAAAAAACTTAAATTTCCCTTGACTTTTAAGTTCACTCTTGTTCG | 382 |
| cg06649410 | CGCAGAGGAGAATGTCAGATGCTCAGCTCGGTCCCCTCCGCCTGACGCTC | 383 |
| cg17501210 | CGCCCGATTCAGACAGCTGGACTCAGAGGGATTCTGCTCCACAGAGAAAC | 384 |
| cg02515033 | TCCCACCCCTTCCATTCTGTTGAGCTGAGACGCGTGTTTTGGTCGTTTCG | 385 |
| cg00907204 | CCTGATTTACACTTGGCTCCACCCATAAAGTTCCCACTGATTATCACACG | 386 |
| cg16847637 | GCCAGGAGTCCTCGGTCCTGGGAGGGTTTAAAAGCCAGGGGGCCGTCTCG | 387 |
| cg03473532 | CGTATGTGTTTGAGATAGCAGTTGTTTACTATCACTTGAAAATTCTGAAT | 388 |
| cg07205627 | CGACCATTTGTATGTGTATCTATGTCAGAAAGAATCTTTATTAAAATATT | 389 |
| cg14508358 | CGGTCACGTGATACCCCCCAGTGTACCTTTCCTGCAGCGTGTTACGTGGC | 390 |
| cg04661709 | CTGGGGCTGAGACCCCATTTCTGGGAGGCTCTTCCCAGTGCTGGAGACCG | 391 |
| cg13428009 | TCTTTGAGCCCCAGCTTCCTCATGTGTAAAACAGGATAATAGCAGTGTCG | 392 |
| cg21789941 | CTGCTGGGGAGCAGTGGACGCCCTCCAGGGTCTAGGGAGCAGATAAGACG | 393 |
| cg22866430 | GCACCCCCCCATCCACTTCATGTTCAGAAAACTCAAAGAGTCAGAAAACG | 394 |
| cg18234973 | AGCTCTGGAATCCAACTGCCTGAGTGGCAAACCTATCTGTAAGACTTACG | 395 |
| cg26110733 | GGGCCTTTAGTGCTGGCTCTTGAGTACCCAGACCCCAGTTAAGAAATACG | 396 |
| cg10636246 | CGACACCCTCAAGGGAGGAGTGCAGGCACTCAAAGATTTGAGTCACAGGC | 397 |
| cg10578779 | CGCAGAAGAAAGCTGCTCAAGTGCTGGAGTGGCCAACCCCATGCCGTTCC | 398 |
| cg17591574 | TGGCCTTAGGTTCTAGTGGCTCTGGTGTGGGCCTGTGCCCAGCCTTTACG | 399 |
| cg13371627 | TTTATAAACCTGCTATTCCTTTCCTTCGGATCCCTGGTTTTCCATCTCCG | 400 |
| cg12458003 | CTGGGCTTGACAGCTCAGAGCAGTGTCCCCAGCTGTGGCCCTAGAAGGCG | 401 |
| cg13180152 | TTTGCCTCTTTTTCAGTCTTGTAATCCCAAACAAAATAGAAGAGCCATCG | 402 |
| cg21459583 | AGAAGCACGTGGCACCTGTCATAGGCCCCTCTGGCTGCTACAAGTTCCCG | 403 |
| cg18278247 | CGGCACCTATCCCAGCAAGATCCCGGGAACAAAATCCTGGAAGACCCTAT | 404 |
| cg11295183 | CGCGTGAGTGTCCTAAGGAGTCCAAGCACAACAGATGGCTGAGTCACACT | 405 |
| cg18388639 | CGAGCCTGTTGTCAACATTAGCCCCGGGTTTCCCAGCACCAACTCCAGCG | 406 |
| cg06752157 | CGGAGCGTGAGCCTAGATTCTTTGGAAGGCTGTACAGTTTAAAATCTAAT | 407 |
| cg18148156 | CGGCAGGTTGGAAATCCAGTTTGTGGCTGATGCAAGCAAACCATGCTGCA | 408 |
| cg19935065 | CGGTTGTGAGGTGCTCACGTGTTTTGGAGATAGCAAAAGTCTCAAATAAT | 409 |
| cg10549018 | TGTCTCCTCATCTCCTGGATCTTTGCCCAGCAAAACCTCCAAAGAGACCG | 410 |
| cg07927953 | CGCACAGCCAGCAGGCGCCTGGGCTCCACTCTCTACCTGGAATCACCAGG | 411 |
| cg01057893 | CGGCAGTCTTCTCCACTGAGGGACTGGGCTGGGAAGTCCTGCGTTTCAGT | 412 |
| cg04431596 | CGGCCCCACCCGGTTCTTGTGTCAAGACAAAAAGAAAACCCAGGTGGCCT | 413 |
| cg06806711 | CGCTGATAGACATCAGGTGACAGGAAATCAGTAGCTTCTGCTACCTTGGG | 414 |
| cg13620770 | CGGCAAGCATCATCGCCAGGCCCCAGGCCTCCTGTGGGACGCCAGTCACC | 415 |
| cg03764506 | CACTTCCTGACTAAGGAGCTGCAGCGATACATCGAAGGGCTCAAGAAGCG | 416 |
| cg07582047 | TCCAGGAAGCAGCAGAGGTCGCGCAGCTCACGGTTCTCTGCCTGCAGACG | 417 |
| cg16649728 | CCAGCTTGAAGCCCGGTGCCAGATGCATAAGCCACTTGCCCGAGAGCACG | 418 |
| cg06854487 | CGTAGCCAGTGCGAGGTTGGCTCTACTTTGGGTTTATGGGTTGGGCATAA | 419 |
| cg25371036 | CGGGCAGCTCTACTGCTTGGAGAAGAGAAAGCTAGGTAACTGTACAGTTG | 420 |
| cg20941739 | CACAGCCAGAGCACTTACAGTGAGCCGAGCCACAGTCCAGCAAGCTGCCG | 421 |
| cg05021075 | CGACACTGTGATAATCATGAAGTTTTGTGTGAGAAAGATAAGGGAGAAGA | 422 |
| cg01975858 | ATAGCTATTCATCCTTAGACCCTGTCCAAACAGAGCAACAGTCATATACG | 423 |
| cg20923047 | TATTTTTGAAGGAGCTGGTCAGCATTTGCCTAGACTCTCGGGACAACACG | 424 |
| cg19783150 | CCCTCTAGTGAGACCCATTAGTGTATAAGCTCTGCCTACACCGAGTTTCG | 425 |
| cg01641177 | CGGCCATGCCCTGGCCCCAGAAGTCGTTGGTGAAGATGCCCCAGCAGAGC | 426 |
| cg07677157 | CGGTGATCCATAGCAAAAGAGCCTATGAGTCAGGTGATGATCCATTCATC | 427 |
| cg21579274 | ACTACTTTGGGCCTCGGTTTCCCTGCTCCTTGTAGATCAGAGAAGGGACG | 428 |
| cg01687878 | CGGCCATGTCTGGTCACTGCGTATGAATGACATCTGTCTCTGCAACGCTT | 429 |
| cg18159533 | TGCCATTTAATCTAACGCCACCTTCTACGGACCAGAGCAGGTGCTCTGCG | 430 |
| cg04323814 | CGCAGTGACAGGAATAATGCCACTGTCCTGCTGAGTGGGAAAGGAGAGGG | 431 |
| cg04836038 | AGAGGTCTCAGGAAAGTAGCCTTTATTTATGTGGCACCGATCGGAACCCG | 432 |
| cg16188984 | CGCACAGCACGGAAAGGGAAGGCAGCAGCCACATGGACCTGGGCCCACGG | 433 |
| cg11605750 | CGGCAACCCCGGCCAGACAGGGGGTATCCAGAGGGCCCAGGACACCCACC | 434 |
| cg18607491 | AAGAACGACTCTCCGCAATGCGCATGACAGAAAAGCGGCTGCTTCTACCG | 435 |
| cg14870958 | TCGAACGCTGGACCCTTGAGACATGTGACGGGACCTGAGGGCAGCCACCG | 436 |
| cg19491388 | CGGAGTTTCTGTTACCTGCAGGGAATGCTCTCTTAACTAATCCAGGGGTG | 437 |
| cg02135821 | CGGAGGCTGGGGAGATGACATCAGAAAACCTGGTCCAAACTGCTCCAAAA | 438 |
| cg09279803 | CTTTACTCAATGCCAAGATCCACATTAACACAATTCCACTGAATCCTCCG | 439 |
| cg26481784 | CGGCGGACCCAGCTGACCAAAGGAACTACAGAAGAGGACGAAGAAAGCGA | 440 |
| cg19459791 | TGGAGCAAATATAGGTCTAGACACGTAGGGCCTGTGAGGTTTTCCTGACG | 441 |
| cg16730765 | CTCTTGATGACCTCGTTGTCTGTGCTGGCACCATGGGTCTCATCCTGACG | 442 |
| cg19987111 | CTCTCTCCCAGACCTCAGGTAAAATTAATGTTCTAGGAACGTATGCCACG | 443 |
| cg02880119 | AAGTGTCAACGAGATGACTTGCGCTACAGCGGCACACTGGTGAGGAACCG | 444 |
| cg22616007 | CGCATCTGCTTCCCATGAGTCTCTTGGGGAAGACAAAGCTCACATTCTTC | 445 |
| cg06946797 | CTCAGGGATCTGTGAGCAGAGAGGCCTGGCTGCAAAGGGTCTGGAGGACG | 446 |
| cg26837962 | TGTGACAGCCCTGGGCTGAAAGACTCCCGGTGGCCGTAGTGTAATAGCCG | 447 |
| cg08659340 | CAACTGCTGGTCTGTTTTCTGCCCCTGTAGTTTTGTATGAATTCTTTACG | 448 |
| cg07984286 | CGCAATGTAAAATTATGTCCAGTTGTAGATGTGTGTGTTGGGGTTTAACT | 449 |
| cg00417823 | CGGGCTGGAATTGAGCTCAGCTAAACAAAGTCCTTCAGCTTTGAAGGGAG | 450 |
| cg05180206 | CGGACAAGATAATTATGATATAACAAAAGCTTGTGTTATTTAAGGTGGGG | 451 |
| cg09899215 | CGTCCCTGGAGCCCACGAAAATGTCTGCAAGATCTGCAAGGGCCACCTTA | 452 |
| cg04379563 | CGTCCCGACTTCACGCGAGGGTTTATACCTGAACAGACACAGCAGGGAAA | 453 |
| cg09834822 | CGCCCTCTCTGTGTAGCTGGAAGAGCCTGACCACTCACGTCTCTCCAGGC | 454 |
| cg18951537 | AACAGCCATGATTCCTAAATATTAAACATTTTCCTCATCTGACAGGATCG | 455 |
| cg23842572 | ACAGAGACAGAGCCCAAGAATAGAGGCACACGGGGAAGTAGACAACATCG | 456 |
| cg25613227 | CGAGGGTAGTTGGTCAGAGTTGTGCGAGCTGGTTGCAGGGAAGGCTACCG | 457 |
| cg07660627 | CAAGCTCTGTGCAAAATAATCTGTAGTGCTTGCTATAACGAGCTATAACG | 458 |
| cg22156456 | CGGGGATAGGCAGGTAAGTAGGATCAAGGAGTGCAAACAGCCAAGTGTGA | 459 |
| cg18181703 | CGTGGCCACTCTTCAGCATCTCTGTCGGAAGACCGTCAACGGCCACCTGG | 460 |
| cg25876186 | TCGGGGAAGCTCAGGGTCTTGGGGTCTCAGGGCGGGGAGGCAGGACGACG | 461 |
| cg23691771 | CGGGCAGATCCCAAGGTTTCCTGATATTCCCTACTGCTGCAGTCTCTGCT | 462 |
| cg00870265 | GTCTTCATTGTTACTCAGCGGGGACACCCTCCTGGCGCTCCGGGGGCTCG | 463 |
| cg03739265 | CGCTGGAGACCAAAATCTTGGGCTATGACCACCCTGGGCAACATAGTGAG | 464 |
| cg03636183 | TGGTGGTGGGGCTGCCGGCCAATGGGCTGGCGCTGTGGGTGCTGGCCACG | 465 |
| cg02291424 | CGCGGCTTGACTAGTAAAGTGCTCAACTATTCTCGACCACCATTGGGTAT | 466 |
| cg26853093 | ATGTGGCGGGGGCTTTGGACCCTGGCGGCCCAAGCGGCACGTGGGCCTCG | 467 |
| cg03110633 | CGGCCGCCGCCCCCACCCGACCCAACCGCCCGCACAAAAGGCCGGAGCCG | 468 |
| cg07127225 | CGGGCTCGGTCCAGACTGACAACAATGCCCTCCTCGTACCTGAAGAACTT | 469 |
| cg06022832 | ACGAAGGACAGTGATCCCTAACAGTTCAGCAGTCCCTACTGTGGCAACCG | 470 |
| cg21566642 | CGCCTCCTGAGCCTGACTCTGCTCTTCCCGCAGCTTTGGCCCCCCGGGGC | 471 |
| cg01940273 | GCACGGGGCCTGGCACGTAGGGGGCCTTCATTGCATCTCTCTTCCCTTCG | 472 |
| cg08110610 | CGGGTGGGGACTAAACAAAAAGGTCAAGGAGAAATGTCAACCTCAAGATG | 473 |
| cg00684178 | CGCTGGGCTCTAGCTCTGTATCTGTGCTCCTGGCAGGCCAGCACCCCATC | 474 |
| cg24580066 | TCAAACTGCCGCGGGCGTGCGGGTTGACGCCGAGTGTGTGTGTCGGGGCG | 475 |
| cg04588969 | TTCCAGTTCGTGCCATCAAGGAGGGCGACCTGAGCACCAAGTACGATGCG | 476 |
| cg21727276 | TGAGATCTCTGTCTCTATCCTATCCTGTCCCTGGCCTTCTGAGGCAAGCG | 477 |
| cg09051966 | CGGGCGCAGCCCCAGGATGCAACAGCTCTCATACAATTGGCCAGGAAGGC | 478 |
| cg18345924 | TGCATACTGATTTCTGAACATTAAATCTGCATCCAATCGTGTTTCATCCG | 479 |
| cg18638931 | AAAACACCAACAAAAAACGACTCCTAACATCACTCTGGGTGGTGAAAGCG | 480 |
| cg27380813 | CGATCCGGAATTCAGGATGAGCCATGAAGTTATGGATGGAGGTTCGAGAT | 481 |
| cg03827386 | CGTTGTTGTTGTTTTAGTTACGTTTAGCTTTGTCAGCAACACTTTGGCCC | 482 |
| cg15849439 | CGAACCTGATGTGGAGACAAAGTATCCTTCATGGACTTTGAATTTGATAT | 483 |
| cg06349851 | CGGCCAGAGTCTTATCCCCTGGCTCTGGCTGATGAGGAACACGTAGAATG | 484 |
| cg25598319 | CGCGGGGGGTCCCCCTGGCACCTCCAAGCCCATCCCATTGACGAAGTAAC | 485 |
| cg25845889 | CGAGAGGGCAGCTTTAAGACATTTTAGAATTGTTTCTTACCATTGTCTTT | 486 |
| cg15228492 | CAAGTCCAGTTTTCCACCTAAATATCACTTGAGGCCAACATTCTCTTTCG | 487 |
| cg03900798 | CACCTGACAGACAAAGCCCTGGGTCCAAAATGGCTCATCGGGGTCAGGCG | 488 |
| cg20576510 | CGCGGCTGTACCACATAGAGAAAGATATGCACTAGTTCAAAGAGAATGCC | 489 |
| cg12988996 | TGTGCCTCCCTGGCAGTCAAAGTTCTAAGGGGGACTCCTGAAGCCAGGCG | 490 |
| cg11569478 | CGGACTCGTTTTATTGACTGACAGTCAGTGCTAGCAGTGACTTCAGCTGC | 491 |
| cg01603456 | GTCATAGAAGGCTGTGTTGCCCCCAGCAAGAGCAAAGCCACCGCTCCACG | 492 |
| cg18584747 | CGGCCGGAGGGCCCTGAACCCTAGGCCAAGACTCATCCACAACCTAGGGG | 493 |
| cg17543884 | GAAAACCCAGCAGAACATAACCGTGCAGGTCTCCGACGTCAATGACAACG | 494 |
| cg22090064 | CGCAAGAGAAGAAAAAAGGTAAGAGTTTAGTTCTCAAAAAGACAAAGAGT | 495 |
| cg11923914 | CGAAGGAAACCTTCGCTGGCTCCCTAGTGCCCTGGAATAACACTGAAACC | 496 |
| cg18394552 | TTTAGGCAGGTGGCCAATTTTGGTCCAGCTGGTGGCAAGGGCAGGTGTCG | 497 |
| cg24801535 | CGGCAGGTTTTGGGACTCATTCCTGCAAGCCCAAGCCAGAACTTGTTCTG | 498 |
| cg12325588 | CGCCATTCCCCAGCACCCGCAGCGGGCACTGTGGCTGCACGCCGCCCTCG | 499 |
| cg23367341 | CTGTCAGTAGATCTCAGCGGGAGAGCAGTGCTGGTATGTGAATGTCCTCG | 500 |
| cg04987004 | CAGTCCCAGCTTTCACTGCCAGGGTCCCAGTCAGATTCCAGGAATTTGCG | 501 |
| cg24859433 | CGCCCCTGGAGAGGCCTCTTGGGGAATGAACTGTCCCTCCCAACCTTGGA | 502 |
| cg10940833 | CGGCGGCAGGAACTATCAGTAGACAGCTGCTGCTTCCATGAAACGGAAAA | 503 |
| cg11539695 | CGGGCCCTGCAGTCCTTAATAAAAAATTGCAGATTTCATTCTAGGTGTCA | 504 |
| cg26987613 | ACTGATGATTTCCAGGAACTACCAAAGCCACGGATCAGCTGAGTTACCCG | 505 |
| cg17598713 | AGATCCTAGGTGAGGGCAGGTTGACTGGCAGGGCTGGTTTTTCGGGATCG | 506 |
| cg26433444 | AAGGCCTGGCTGGCTGCGAGCTCAGGAGGCCGCCTGAGGACTGCACACCG | 507 |
| cg25325512 | CGCGGCATTCAGCAGAACTCATGATGAAGGAAACAGTTCCATGGCAGGGT | 508 |
| cg02272457 | CGGCCCCACAAATTACGTGTGTGCAGGATTAAGGGAAACCAGCAAGGGGT | 509 |
| cg10169261 | CGCGCCTTTTATTGACGCAAACCTCCCGTTCTCGCGGGAACTTGGGAACG | 510 |
| cg03098356 | CGCCATCTTCACACCCCGCTCCGTCGCGTACGCACCTGGGTCACGTGATA | 511 |
| cg06649410 | CGCAGAGGAGAATGTCAGATGCTCAGCTCGGTCCCCTCCGCCTGACGCTC | 512 |
| cg15822010 | CGGCCCTGCACACGTGTCCACTGTGACGCGTCTGTCATGGTCCCCTCGGC | 513 |
| cg15089567 | CGGCTGGGGACCGTAGCAAGAGCAAGAGGGCTCCTCAGCAGTCCCTGTGG | 514 |
| cg00332048 | CGCCAGTATGGAAACACAGTGTGTAAAGCAAGCTTCGAGAGAGGAAAGAG | 515 |
| cg01391867 | CGCCTACAACTCTCTCGGTCAAACAACCGAAAACATCCCGTGCCGGAAGC | 516 |
| cg07113802 | GGTGCGCCCTGGGCAGCTGGGTGGCCTCAAGCCCTACTGGCGAGACTCCG | 517 |
| cg03473532 | CGTATGTGTTTGAGATAGCAGTTGTTTACTATCACTTGAAAATTCTGAAT | 518 |
| cg15573979 | GGATGCACTGCGAGCCTGCCAGGAATTCCAGGCCTCGCAGTGTTCTGGCG | 519 |
| cg08202754 | CGCTTCCACATTTGGACGCCCCCACCCAGCGGCCTCCGTGGCACACACCG | 520 |
| cg14260002 | GGAGGACTTGGGGTGTGGCAGGGGAGCAGCCAAGCCGGCAGATGACTACG | 521 |
| cg06563451 | GACCCTGGGCTTGGGGGGATTTTTTTGGCCTGGAGGGCTCCTCTTCTCCG | 522 |
| cg03460603 | ACACAGAAATGGGGGTTCTCACACTACAGATTAAGGAGTGTCCCAGGCCG | 523 |
| cg25448355 | CGGGACAAGAAACAACATGTGAGCAGCACAGCCATTCAGTTGGGATGCTG | 524 |
| cg24493971 | GGCGGGCCTTGCCTCCAGCAGGGGTGTGAAGAGCCCAGGCTTATTTTCCG | 525 |
| cg02716826 | GGCCAGACACCCAGGACCCAAATAACCTAATAATGGCTCATTTGCCTCCG | 526 |
| cg14355192 | TCCTGCAGGCCTCACCATAGGGCACCTCTTTGGCCCAATTCAGGGCTCCG | 527 |
| cg13649864 | CTCTGAAGCCTGCACCTCTCCATTAAACACAGAACAGCTGCCAGAGGACG | 528 |
| cg23549061 | CGGTGTGAGTGCTCACTCAATGCAATGTGTTGGTGATGGTAACTGTCCTT | 529 |
| cg13947317 | CGGCATTGGTCCTTTATTGAACATCCTCCCAAAGCTGGGGCTAGGGTTCA | 530 |
| cg21272576 | CGCTGCTGCCTGGGACTCAGTGATTATCCCATTTTGCAGACCAGAAAGAG | 531 |
| cg03522107 | AGCCGGACAAGTGAGACCAGGCGGCCCCTTGGGTGGGCAGGGTCTGTCCG | 532 |
| cg03259703 | CGGCTCAGCTTGCTCACCCACCCCAGAAGGAGCGATTTGCATTTATGAAG | 533 |
| cg05461666 | CGGGCAAGGAAAGGATACAGGTATCTGCCTTTCGCATGGCACTGAGCAAA | 534 |
| cg15860924 | CGGGACAAGCTTGACAAGGTGGGTGTCTTCCTGGACTATGACCAAGGCTT | 535 |
| cg10327168 | CGGTAGAGAATATTTTGACTCCTTGAGTAATGGGAGTTCTTTTGTGGTCT | 536 |
| cg24393673 | CGCGCTGCAGTCACGCCTCCCGCTGCCAGCCCGGCACCGGGATCTTAATC | 537 |
| cg23923856 | GGAGGAGGATCCCCGAGAGGGGCGCAAGTTGCGATGTCCGGAAGCCGACG | 538 |
| cg14476101 | CGCCAGCAGATACAAAGGCAGACAAAGTGAGCGAGGCAGTTTCCAGGACC | 539 |
| cg20569940 | CTTTGGCTCGAGCTCCAGGTGCAGATTTACGTTCATTTTCTTTTGTTCCG | 540 |
| cg10578779 | CGCAGAAGAAAGCTGCTCAAGTGCTGGAGTGGCCAACCCCATGCCGTTCC | 541 |
| cg05996213 | CGGGTGGAAGGAGAATGAGGAGGCAACCAAAATAAGATCAAAAAGTCAGG | 542 |
| cg03424844 | CGATGAGCAAAATCACACATCCTCATGTCCATCTGGTGCTCTGTATTGTG | 543 |
| cg19570545 | GCGGAGCCAGCGCTCTTGGGGGCAGGGCTGCGAGTCCCCTGTGTGGGACG | 544 |
| cg20248954 | CGTGACCTGTCTGTACCCTAGCGCGGGGCTTCTGTACGCTGCTAGGAAGC | 545 |
| cg08138571 | GAAGCAGTTAAGACAACAATACCCAGGACTAGCCAGATCCCGAATTACCG | 546 |
| cg23814988 | CGTCGGGCTCACAGAAGGCTCAGGTTTCCTCCAGTATAGTTCAGGGGATG | 547 |
| cg20801751 | CGCCTCATACAAGAACACAGAACAGGGAGCTTGTCTCCTTGGGGATTAGA | 548 |
| cg11333189 | CGCTGCGGCAAACCTAGGAGGGACCCAAAAGCAGAAAATCCTAGAAAAGA | 549 |
| cg08055490 | CGGAAACTGATCTAGTCAGGGAGGTCAGAGAATATCTCCCTGAGGAAAAG | 550 |
| cg02613108 | CGCACTGACTGGTGAGCGCTTTGCACCAATTAGTAGCAGGTGCTACAGCC | 551 |
| cg12329405 | CTCCCTACGTGGGGGACGTGCAGGATGATGCGGGGTCGGGGGGGATTTCG | 552 |
| cg20245361 | CGGGGTGCATTAGCTGTGACTTTTGCTGCATCCAGTCATGCACATGTGCC | 553 |
| cg07793207 | GCTTTCTGCTTTCACCGGGAGGCCCTTTAATTGTTACCAAAAATAGGACG | 554 |
| cg07733920 | AGGTACTGGATGCACGGCAGGCCCTGCCACTGCTTCTCAGAACCCTCTCG | 555 |
| cg02415341 | AATTCTTTCACAGTTCTGGAGTCAAGAAGTCCAAAATGAAGGTGTTCACG | 556 |
| cg12893697 | CGGCCCTACACGGGCACCGGGCCTCAGTCCTGCCAGGCCCACGCCATCCT | 557 |
| cg18886347 | CGGTGTGGAGGCTTTTTTTGTCAGAAGCTCTGGGGTTTGGCTCTTCTCTG | 558 |
| cg19083871 | GAAGCTGGCGACGAACCCGTGTCTCAAGAATTTCTACGGCTTGTTTCCCG | 559 |
| cg13875901 | CGGCTGCAGGCCCCAGAGTCCACGAGGGTGGGTACAAATCCCCTGGGCAC | 560 |
| cg02970861 | TGATTGGCCCAAAGCATCGTGAATATTCATGACAGCAGCCACAAAGGCCG | 561 |
| cg26967875 | TTCTCGGGTCTTCGTGAGCATTAAGTCAATGAACCCAGGGCACCAAAGCG | 562 |
| cg00574958 | AGGCCAGCGAACATCCAGCTGTCAGTTGGTCTGGGGACTATCAGCATTCG | 563 |
| cg22355889 | GAAGATGGTGAACCTGAGATGACCTGTCAATGGGATTCCCAGACAGTCCG | 564 |
| cg05839709 | TGGATAAAGGGGTTTCTAATACCCAGCTCACATGGCTGTGGTGACAAACG | 565 |
| cg19061798 | GTGAACACCTGCCGAAGCCTTTGCCTTGGGAGAGGCTCCAGCAAAATCCG | 566 |
| cg11932091 | CGGGCACTGACTGGGGACCCAGGTGAGTCTAGCAAATGACAAGGCCCAAC | 567 |
| cg26527903 | CGGCAAGGTTTATAACGAGAATAAATGAAATAAATACAAAATCCAGAATA | 568 |
| cg24420742 | CGGGGACTTGATATGGAAAGAATTAATGTACTGGCTTTTTTGTATAGATG | 569 |
| cg10948795 | CGGGTAGGGTTAGGCTCAGTTCAGGCAGAACCTGCCATTCGCTCATTCAC | 570 |
| cg17117459 | ATTCATCTCTAGTCTCTGTAAAATGCCAGCCCTCTTCTGGGTTAACAGCG | 571 |
| cg18331061 | CGCACCGTCTCACCTGAGTTTTCTGCCACTGGATCATGTGGGTCTGGTTG | 572 |
| cg21879240 | GAGTGTTCCCCATGCTGACTTCTGCCAGGCTGCAGGGAATAAAGCTAACG | 573 |
| cg20656525 | CGGAAAGGTTCATTGGCTAATTTGCCACAGTTGGTTTTCAATATGTCCAT | 574 |
| cg14543641 | CTGTGCAGACGCCATTTGCCCCCAGCTTAATTGAGCATATCTCTCCCTCG | 575 |
| cg04634427 | CGGTGGGAACGGGAAGCTTCCCCAATCAGACTGCATCCTGAGGGACAGGA | 576 |
| cg01624571 | CGTGGATCTAGCCACAGAAAGGCAACAGCGCTTCAAAGAGGCTCTCCGTA | 577 |
| cg05575505 | CGACTCTGAGTTCTGACAGGGAAATACAATGAGTTTTATGTGGACAGAGT | 578 |
| cg06051159 | CGGCCTTTGTGGATGCCAACCTTTGTCACAGCCAGGAGGGGGCCTCCCTC | 579 |
| cg00994306 | CGGCCTCAGGCCAAGCATGAGAACAACGTCAGACAAATCCCCATTGATTG | 580 |
| cg02295856 | CGCCGACCCACAGGGCACAATGGTCTACAAAGTTGGAAATGAAGGTCTGC | 581 |
| cg12991522 | GCACGGGCAGCTGAGCTGGGGGTACAGGGCACCCCCGTGTCCCAGGGTCG | 582 |
| cg00036119 | CAGGAAACTCCTACCTCATCACGGCAGCTGCATGTGTGGGTAAGCCCACG | 583 |
| cg05995267 | CGGCAGTTCTTAGTGAGGGCAATTCATTCAATCCAGAGCCAGAAACAGGA | 584 |
| cg06722193 | CGGCCACCTCAGACTTCTGTATAGGAGGAGGCCTGACTGGGAGATCCCTG | 585 |
| cg05782444 | TGTCCCAGGATGACATCAAGGGCATTCAGGAGCTCTATGGTAAACCTCCG | 586 |
| cg26943759 | TATGGACAGTCTTTTGCCTGGCGTGGGGGCCAAGTGGACAATACCACACG | 587 |
| cg05737638 | CTCCCTCCACGGGACCCAGCCTGCAGAAGGGTCCTGCAGGAAGTGGCACG | 588 |
| cg21649005 | GAGTTTCTACTCTGCCTACAGGTCTCGGGTGAATTCACACAAACTCTTCG | 589 |
| cg06419850 | CAAGATCCAGGGACCTAGAGGCCTCGGCGATGACACTGCGCTGAACGACG | 590 |
| cg19761014 | CGCCAATGGTTCCAGCAAAGCCCCAGGACTGCTCTGCTCCTGGTCTTGTA | 591 |
| cg05174890 | TCAGAGCTGTTTTTTTTCGTGGGTAACTTCCAGTTGCTCAAGGGTCCACG | 592 |
| cg10431713 | CGGCTCCACACTTCCCCAGCGTGGCCTGCATTTCTATTGGGCAGCTGGCG | 593 |
| cg02483931 | TATGAGAGGCCAGAATATTGCTTCCGTTCCCTTTTCATCTCTGCACACCG | 594 |
| cg07477034 | CGCCCTGATAGCACCGCAGAGGTTCTGATCTAATTGATCTGGGATGCGGC | 595 |
| cg15815084 | ATATACAGTGGCCATGAAGCAGGCACCACTTAGAAACCTCTGTATTAACG | 596 |
| cg12054453 | CGGCAGAAGCTTCACCACAAAAGCGAAATGGGCACACCACAGGTAAGACT | 597 |
| cg03568017 | CGGCTAGAGACTCACTGACTCATGCGTGTGTGGTAGGAATCTTCCAGGAA | 598 |
| cg02633924 | CGGGACGGGGACTTCCTGGAAGATTCCTACCACACACGCATGAGTCAGTG | 599 |
| cg18181703 | CGTGGCCACTCTTCAGCATCTCTGTCGGAAGACCGTCAACGGCCACCTGG | 600 |
| cg27637521 | GGAAACTTGCTGTGGGTGACCATGGCGCACGGAGCCAGCGTGGATCTGCG | 601 |
| cg05420896 | GGGTGGTACAAGGGGCGCCCCTTCGCTGTTTTAAAATCTGATGCTTCACG | 602 |
| cg19283806 | GATTTCTCCTTGAACAATCCCCGCAAAGATAGCAGCCAAAAAAGGATGCG | 603 |
| cg27569863 | GAAAGCACTAAATTATCCTGCAAACTCCCGGCTGATGCCTAAGGAATTCG | 604 |
| cg13611456 | CGGAAGCACAGTTTGCAACAGTTTGGGGTTGTCTTATTTTCACTGCTGGC | 605 |
| cg10677351 | CGCACGGATGGGACCTGCTTCTCACTCCTGCACAGCTGAACCTCCAGGAC | 606 |
| cg15011409 | CCAGGAGTGCGCTGCTCTCGGGAAGGCATCCCATGGCCTGAGCAGCAGCG | 607 |
| cg17676631 | GCTGGGTCCCCGTGTAGCACTTAGCTAAAGATGATCAGCAGCTGAGAACG | 608 |
| cg11594160 | ACAGTATACAGTCGGTGCTCAGGATGCATTTGTTTTCCTGTCCCCCAGCG | 609 |
| cg26950531 | TGCCGGCCACGTGGTTGCTGGGCAGGTGTGGGGTCAGGCTGTGGATCCCG | 610 |
| cg22538557 | CGGCTCAGCCAACCCCCTAGATAATAGCGGTTTCTCAAACTGTGGTCCCG | 611 |
| cg09920725 | CGGTCTTGTAACAATTGGATGGATGCCTTTGAAGAGCCCCTGTCCCTATT | 612 |
| cg10354495 | CCTATTCTTCCAGAACCTTCCTGGAGTATTCCGTTTTCTCTAGGACAACG | 613 |
| cg04659537 | CGGGGGAATTCATTGATAAGGAGAGCAAGCATCCCAAGAACATCCCCATC | 614 |
| cg14088844 | CGGACACAGCACCAGGTCCAGATTGGCAAGAAAGGACCCTGGGCTTGCTC | 615 |
| cg07258300 | ATCTTCAAGTCTCACTTTGGTCCTCAGTTTGTAGTATCTATTGCAGACCG | 616 |
| cg26191447 | CGGTACCAGTAACTGCCAGGAAAAGGAAGGGAATATGTCCAGAAAAAGCA | 617 |
| cg02831419 | TGGTTATTCCCCAGGAGCCGCGCGAAGCATGAGCTAATTTTCAGTGAGCG | 618 |
| cg20550050 | CGCACATGAGGCACAGAGAAAGGACAAAACGCAGCCACACCTGAGCTGCG | 619 |
| cg06266189 | GTGCCTCCCTTCTGCACTGTGGGTGTGTGGAGCTGTCTTTCTTGGAGACG | 620 |
| cg12791136 | TGAGCAGGAGCGAGACAGAGCACCACGGGGCACGTGAGGCCAGGGCACCG | 621 |
| cg23278885 | TCCCACAGTGAACCCCTCTAGGTGCAATTACAGGATGATTTTGTGTCTCG | 622 |
| cg08074820 | GCCCCAGGACCCCGGGATGTGCAGCCTAGGGGTGGGGTTGAGGTTCTGCG | 623 |
| cg11086127 | ACCTCTTGTGGTATTCCTCTATCAGGCAATCTGGGTGGGCAGAGACTTCG | 624 |
| cg00412842 | AGTCTTAGCTTAAGGAAATGTGCCACCTGCGTGCAGGGATGAGGTCATCG | 625 |
| cg27294156 | CCATGCTGCGGATCCTGTGCCTGGCACTCTGCAGCCTGCTGACTGGCACG | 626 |
| cg22163406 | TTCATCGCAATGGGCTGTATAGCTTGGCCCGTCTCATTGTAACAAATTCG | 627 |
| cg08334432 | TGGGCTAGCCCAGCGCCACCTTCCTGGAGACTGTCTGGTGGAAGACACCG | 628 |
| cg06133392 | CGCTGCTGAATGCACAGCACTGTGAATGCACTTCCATCACTGAATATACA | 629 |
| cg23573129 | CGGGGCTGAGTACTTGGCTGACCACCCTAGGGCCACACCAGCCCAGGGGA | 630 |
| cg20585830 | CGCACAATACGTGGAAGGCCATGGAAGGCATCTTCATCAAGCCTAGTGTG | 631 |
| cg19253577 | AAGCACTTGATGTTTGCCCTCACACTGTTCCTCTCCCATTCCAGTTGTCG | 632 |
| cg09219182 | GGCCTTACACCAGTGCAGAGGGCAGGCTTGAGGCTGATCTGGTACACACG | 633 |
| cg01708387 | TAGAATAGTGACTGGCACATAATAGGTGTTCAATAAATGTCAACCCACCG | 634 |
| cg12918464 | CGCCTCAGCTGGTGCTCCAAGACCTGCTGGCCAGACTGGTGAGGCCAGGT | 635 |
| cg14508777 | CGGGGACAGGGCTCCTAATGTGTTTGTCAAGTAAATAATGAGTAATAATC | 636 |
| cg05993589 | CGGTGAGGCCAGTGCACTCAAGTTTGGGAACTACCAATATAGAGTAAAAG | 637 |
| cg23431721 | ACATACGTCACATTGGCAGTGTTTGTAGGATATCGGGCTGTAGGGATCCG | 638 |
| cg07105440 | GCTAGCTGCCGGGCATTGGGAGGGCTTGACGGGCCAGAGGTGACTGAGCG | 639 |
| cg03929796 | CGGCACCTCGCCTCGACCTTGCCATTTTATACTCAATTGGGGCGTAGGGT | 640 |
| cg23511118 | GCAGGTCATTACTATGGGTAACTGGGGCTCCATCTCTCTGAAATACCACG | 641 |
| cg13155421 | CCCTGGCTGGTGTTTCATGTCCACCAGCTCTGCGTTCAGCACAGCACTCG | 642 |
| cg03840504 | CGTCTGTCCCAAAGGGCGACACAATCCTGAGGCAGCTGTTCGCAAACTCG | 643 |
| cg19389001 | CGGCGCACCTCTCCAGTGGGACCCGCAGGACGCCGTCTCTCAGCCCCACG | 644 |
| cg15709989 | TAAGGCCCTTCAGAAATAGGATCGACAGGGCTGGAGTGGAGGGGCCCCCG | 645 |
| cg13296371 | CTAGCATGGTGCCTAACAGCTCCTACCAAATAGTAGATGCTCAGTAAACG | 646 |
| cg21761813 | CGGTCAGTCTGCCTACAGAGTGCTTGCCAGGCAATGAAGGAAGAGCATGC | 647 |
| cg00398048 | CGTGCGAACATGAACTAATAATCCCTTTGGGTGTCTGGAATGCCTGCCCA | 648 |
| cg25149516 | CGCCCACGCGGACCCCTCTCCGTGTGCTTGTCTGTACTTCCCTGATGGGG | 649 |
| cg10177080 | CGGTACTTCCCTGATGGGGCCCACGCGGACCCCTCTCCAGTGTGCTTGTC | 650 |
| cg09578605 | CAACACTGTGAAACCCTGTCTCTACTAAAATACAAAATATTAGTCAGTCG | 651 |
| cg01817885 | CGGTAGGGTTTGGAGGTATTATTCAGTGTAGAGGAAACAGTATGCATAAA | 652 |
| cg12980795 | CGCTGGCAAGATGAATCACAATAACAACTCTGAAGGCAAGAGGATTGGTG | 653 |
| cg01256539 | CGGCAGAATTGTGCTTAATAGTTGGTGAGGTTTCTATGGGCGATGTGGAA | 654 |
| cg19076659 | CTTCCTTCAGGGAGTCGTATATATGAGAGGGCTTTAGCTTGCTCTCCTCG | 655 |
| cg05182912 | CGGGACACGTACTATCTGCCTCAGCCTAGTGCTAAGCACTTTACACATTT | 656 |
| cg00543335 | CGGGGCTCATGGCTAAAGTTAAGGTACTGATCAAAGTTTTGGATGTAAAT | 657 |
| cg21584251 | CGCTCCAGGCAGCGCTCCACTTCCGCGGCTTCGCCCTTGACAGCGCCCTG | 658 |
| cg00558975 | GGAGGGCATCTTATTTTACCAGGTAAACAAGGGTCACTAAGAGGCATGCG | 659 |
| cg01187498 | TTCACACTTAGGGCTCAGGACGCCCCTGTAGGCAGGGTGCTGGTGGGACG | 660 |
| cg22983827 | CGCAAAAGCAAGATTCGCTCCAGGAGTTTCATGCCACAGGATGGTCGACG | 661 |
| cg02782857 | GCTCATCACCTGGGGGCAGCTTTTGTACCTGCACCCTGTATGAGGCTCCG | 662 |
| cg17286012 | GGTCTCTGGCATCTGTCCCGTCTCCAGCACAAACAACATCTGGGCAATCG | 663 |
| cg02066343 | GCTGAGGGTGCCAGGTTGTCCTCCAAGGCCATCAGAAAGAAGGCACCACG | 664 |
| cg25325512 | CGCGGCATTCAGCAGAACTCATGATGAAGGAAACAGTTCCATGGCAGGGT | 665 |
| cg08373528 | CAGCCTCATGAACTCCATGGGTGTCGTCACGCTCCTCATTTGGCTCTTCG | 666 |
| cg15290848 | CGTTCAGTGCCACTCAGGCCTGGCTGCGAAGATACAGATGGTATATGTGA | 667 |
| cg08877853 | GGCAGTGTTGACTGCGTTCCATACCGGGACATCCAACACAACATTTGTCG | 668 |
| cg01883195 | TGATTCTGGACCTCAGCTCTTCAACAAAGCTGAGGTCCAGAATCAAACCG | 669 |
| cg00936722 | AGCTTTCTGGGTCTCCTTAATGAGCCTGAGGCCTGCTGCCTGCAAGTGCG | 670 |
| cg00554421 | CCTGAGGCTCTGGGAGCAGATGGGAGCACAGGCTCTGTTTTGGCCCTGCG | 671 |
| cg11287647 | TGCAATGATAAGTTTGCTGAACTGCATCTCTGCCCAATGCCACTTCCTCG | 672 |
| cg13917964 | CCCATGCAGGAGAAACCCGTTTGGTGTTTACTACTAAGTCATGATCTCCG | 673 |
| cg00277397 | CGCTGTGTGTGTTGAGACACACGCAACATTCGTGCACATAGAACTCATGA | 674 |
| cg20352798 | CGGTGCCCCACAGGCCACTGTGCAGGATCTCCACTCGGCCCTGACACAGG | 675 |
| cg10119082 | CGGGTCCCCAGTCCTCAAGGTCCCAGCACAATGCCCCAGGAGGAAGACTC | 676 |
| cg17981101 | CGCCCCAAGTCATGAGGGAAGCTTCCAGACTGACTCAGGCTCAGGCTGGC | 677 |
| cg03494277 | CGGTGACTGCAGGCTCCTGGATCCCATGGAGCAGAAACCTGAACTTGTCT | 678 |
| cg09315887 | TGGCTACCGTGCTGTGGGGTGCCTCGAAAGTGTGAATCACCAAATATTCG | 679 |
| cg04599158 | TGTTCAATGTAATAAACAACTGGCTCTTTGCTGCTTCAGATCTGTGCTCG | 680 |
| cg02627240 | TTATCAAAGACTTTATATTTTCTGTAAAATACTACACAGTAAATCTTCCG | 681 |
| cg01026009 | CTACCCTACTGGAAAATAGTGTTAATTATTATTCAGTTCTATTTCCATCG | 682 |
| cg03276920 | TCTTCTTTCCACAGGTTCTCAGGCAAGAGCCACCTGCTATTGCCGAACCG | 683 |
| cg13398470 | CGTGGCTATGCTGTGGTCTCCCTGAGCCTACTCTAGCTGGAAAGGTAATT | 684 |
| cg07205627 | CGACCATTTGTATGTGTATCTATGTCAGAAAGAATCTTTATTAAAATATT | 685 |
| cg05065162 | CGACACTGCACATCACCGAGACCAGGCACAGTGCCGGTTATGCCTAAGCA | 686 |
| cg12396523 | CTCCCGAGTTCGCCCAACTTACTACTCTGCATGCTGGTGCCTAGTCTGCG | 687 |
| cg12831076 | CAACGGAACGAAGCTGCATCTAAGTCTTATTCTAGAATTCTTTGTGACCG | 688 |
| cg22902266 | CGGACAATTGGGGCCAAACGAGGAAGGACACAGACCCAAAAGCCAGACCC | 689 |
| cg04661709 | CTGGGGCTGAGACCCCATTTCTGGGAGGCTCTTCCCAGTGCTGGAGACCG | 690 |
| cg01355108 | AACAATTATGGGCTTAAAGAATTGATCATTACAGCCCCTGGGATTTAGCG | 691 |
| cg26581729 | CGCCATAAGTGCCCCCAGAACTTCAGCGCCCACCATGGCGCACAAGGCCG | 692 |
| cg18476633 | CGGTGAGTCATGCTTCAGGTTTGCCAGGGCCTGTGTCCCGCAAAAAGGCG | 693 |
| cg26341773 | CGCTGGGAGGCACCACTTGACATGACCTGGGACAGGTGGCTCAGTCTCTC | 694 |
| cg25034591 | CTCTGCATCCTCCCCAAGCAAAACCTCACCAAGGTGGTGAGATCCTGACG | 695 |
| cg03102848 | CGGGGGCGCCGCCTAGAAACACACAATGGCCCCTCCTGGCCAACCCCCCG | 696 |
| cg18154457 | CGCGTCATAAAGGACAAGCAGACCCAACTGAACCGCGGCTTTGCLTTCAT | 697 |
| cg11924796 | CAACCTCAGGTTCCTTTTAATGGAGTAGTCACTTATTCATTCAACATACG | 698 |
| cg08871010 | ACAGTCATACTGTCCTGAGTAAGGACAGTCAACAAAGGATCATTTCTCCG | 699 |
| cg06780601 | CGCGTAACACTGTGCCTGGCTTTGCAAATATATTTTACAGATAGTGTAGT | 700 |
| cg27395754 | CTGACCAAGGCATCACAATAAATACAGTTAGCAGCACACAGAAGCCTGCG | 701 |
| cg02587153 | CGACATTAAAAGACCAATGGAGAATGTATATTAGCAATCTGTAAGGCAGT | 702 |
| cg15192905 | CGCCGCCATCTCAGGTCTCTTGGCTTTGCCAGGGCCCACCGGAGAAAACT | 703 |
| cg08352336 | GTCAGTTTTCTCCGGTGGGCCCTGGCAAAGCCAAGAGACCTGAGATGGCG | 704 |
| cg01997410 | GAGAGGCGCAGGCGAGCTGGAGGGGAGCGGCGCGCGGCGGCCAGTGGGCG | 705 |
| cg16730484 | GAGAGTTCAAAGTGAGCTTACTTCCAGTCTCACGTACAGATGATTCTACG | 706 |
| cg22686892 | CGGGATGGAAGTTAGCGAATTTGGGCAACTTGCCTCTACTGCCCAGTCGA | 707 |
| cg14073590 | CGTGGGCGAGTTGGTGGCAAAATCTGAACTGGATACTCACGGAGGGCCCG | 708 |
| cg07989867 | CTTTGGTGCAGAACCACCGCATCCACTTTCTCAGGCCCAGGGGAGGGGCG | 709 |
| cg21080294 | AAGCAGGCATGGTTGATTTCCCGCTAGCGGGAACGATCTGGCCTCAGACG | 710 |
| cg14680768 | CGGCAGAGAGTTGCCACTGCAACGCCTGGCTCCCAGCCCTCCCACCCAGC | 711 |
| cg10142252 | CGCCATCTCAGCCCTGGTGAGCCCCAAATCTGAGAGGGAGGGAAGGTCCT | 712 |
| cg18717447 | CGCCCCCAGCTGCCAGTCCCCAGCAGCTCAGTCCTGCAGTGAGAGTCTTG | 713 |
| cg03960747 | CGGAGGCCAGGCCACGCAGAACCTCACAGACCAAAGCTGCTTCGCAGTCG | 714 |
| cg16832801 | CCTGCGGGACTACCAGAAGGTGCTAGTGCAGCTGGAGAGCCTGGAGACCG | 715 |
| cg11781622 | CGGCCGGGACTCTCCATGTCCTATGGACTCTCGCTGAAGCCACTCGGCCC | 716 |
| cg13947317 | CGGCATTGGTCCTTTATTGAACATCCTCCCAAAGCTGGGGCTAGGGTTCA | 717 |
| cg24145109 | GCGGTGTGCACTGTGACCCGCCTTCCCGGGAAAGTCAGAGCACAGAAGCG | 718 |
| cg19694465 | CGCCCCTGCCTGCTCGCTGCACTCCAGGTCACAGTTGGCACAGCGCACCG | 719 |
| cg15211499 | GAGGAGTGGGTGGGAGACACGCGTGTCCATGCCGCGAGGGTGTGTCGACG | 720 |
| cg10511249 | CGCCCTCGGAGCCCCTGCAGTCCCCTCACCGGGCGCTCCCAAACAGCCCG | 721 |
| cg16383389 | GCCGAATTTCTGAGGTATCCGGGCTCTGGTGGACTGAGGGGGGCGACGCG | 722 |
| cg04607246 | CGGGGAGGGGGTAGTTGTACTCTGCTTGTACAGTCCTTGAGCCCAGTTTA | 723 |
| cg08926056 | TCCCACTTTTGTGAACCTTGAGTTTTTGAAACTTTCACTGAAAAAAACCG | 724 |
| cg14652203 | CGCAGGAGGAGTTGGGAGGACAAAAGGAGAAAATGAACTAGTTGGGTTAC | 725 |
| cg20248954 | CGTGACCTGTCTGTACCCTAGCGCGGGGCTTCTGTACGCTGCTAGGAAGC | 726 |
| cg23814988 | CGTCGGGCTCACAGAAGGCTCAGGTTTCCTCCAGTATAGTTCAGGGGATG | 727 |
| cg14626196 | TCTTTTAACCCAGCTAACACTTCTGTTAATAGTGTCTTCAGCCAGATGCG | 728 |
| cg16704703 | GGGGCAGGACATCTCCCAGACAGCTGGGGACACCCTTTCCAGCGGGAGCG | 729 |
| cg19328485 | CGGTGGGGACACAGAACCAAACCATATCGTTTGGTATTCCAGAATTTCTG | 730 |
| cg04254609 | TCTTTGCAACGTCCCTCAAGCACTTAAGGAGAGGCGGCAGGGCCCAAGCG | 731 |
| cg02974659 | AGCAGGACAGGCACCCGCTGGCCGGGAGGTCATTGACTGGCAGAACGACG | 732 |
| cg19795482 | GGAGGAGGACTTGAATACACGCGTCGCTGACTCAAAGATGGTCCCCTCCG | 733 |
| cg17133967 | CCCGGAGCAGTGGGTTCCTCTGCCGGGCCAGGAGGGTGATGGAGGGACCG | 734 |
| cg17639894 | CGCGCCGGCTGACGTCACCCTGTGTCGACCAGCCCCCTGAGCCCGGAAAG | 735 |
| cg13657511 | GAAGCACAAAGGCGACGTGACTCCTAGAATCTGTCCAGAACCCATGGTCG | 736 |
| cg26411299 | GTGACAGCTGAGCTGAGACCTCAAGACACAGTCACCTGGGCCACCTTCCG | 737 |
| cg05425050 | CTTATGCAAATCACTTAGGTACATGCAAAAGTATCCCTTCTCCCGGAGCG | 738 |
| cg11058730 | CGCGCCGCATCTCCGGGAACAACAGTCTCCCTCCCGAGCATCACAGCCAG | 739 |
| cg05590616 | CGCTCCTCCTAGCTAGCGGCCGCCGCCCGCCGCCGCCTGCGCCTCCAGCT | 740 |
| cg07268726 | CGGCCCCAGCCTGCCCAGGGCCCAGCTGCTGGAGACCCGCAGCTCGTCCC | 741 |
| cg02780017 | CGGAGGGCTCAGGTTACACCCCATAGGAGGGTTCAGGTGTCCCCACATCT | 742 |
| cg05710301 | CTCCAGCTGGCGCACTACAAGGTAAGAACTGACCGCCTAGGGTTTTGTCG | 743 |
| cg00574958 | AGGCCAGCGAACATCCAGCTGTCAGTTGGTCTGGGGACTATCAGCATTCG | 744 |
| cg02580722 | CGCGCAGTTAAAAAAGAACTCTTALTTTGGTTGGTTGGAAAAGAGCTGAT | 745 |
| cg11507178 | GTCAGAGTTCGGGTGTCTGTGGGTCTCTGAGCCTCTGCTGGCAGCACCCG | 746 |
| cg08832227 | CGCAGCTCAAGACCCTGGCGCAGTTCCCCAACACGCTGCTGGGCAACCCT | 747 |
| cg22047262 | CGGCTGGCAGGGGCCCTGTGCCCCCAGGTACCCCGTACCCCTGCCAAAGT | 748 |
| cg11043909 | CGCAAGACTAGGCAACCTCCAGCCAGTCCCTGGGTCGGGCGGATCCTCCC | 749 |
| cg06705834 | AACATGAGACAACTGAAGTGCCCTGGTTTATGTGCCCTGCTCCTCCTCCG | 750 |
| cg17177219 | ATTTCACAGATAAGGAAACTGAGGCCCAAGATGGCACAGTGGCCTCGACG | 751 |
| cg24805559 | GAGACATGGCGACGGCGAAGGTGGCAGCCTGGAGCCTTGGGCTGAGGTCG | 752 |
| cg10323552 | CGCCCAGCCCAGATTTTGGCAGTGCCGAAGCTCCACACTTTCCTAAGCAG | 753 |
| cg13649020 | CGCCACCATCGAGTGTGAGCAGCCCCAGCCCGACCTCTACAAGTAAGCGG | 754 |
| cg01936220 | ATTGTCTCCCAATTTCTTCGTCTGTAAAATGGGGCTGGCAGCAGTGCCCG | 755 |
| cg09225388 | CAGCCACTACTCCAGACTTTGTTCCTACAACTGACGATTGCCAGAATCCG | 756 |
| cg05241355 | CGGATCCGTTTGAAACCTGTCCGGTCCCTGCTTTCTATGCCAGGCAAAAA | 757 |
| cg21112391 | CGTGGGGTAGGGACTTTCATCAGGACATAGAGTGGCAGTCATGTGCTTCG | 758 |
| cg12155356 | AGTGTCCGAGTAAGCCTAATGACACACATTCACATAACTAAGAAGACACG | 759 |
| cg08140104 | AGTCCACGTGTCAGAGGTGTTTGAACCAGAGCAACCCTATCTTAAATACG | 760 |
| cg01823956 | ACTTTCTCCTTCTTGTTTTTATTCGGCATGGCTGGAGCGGGCCGCCGCCG | 761 |
| cg10439456 | CGCAGAGAGATGTACCAACACTGTGCTCCTGAGTGACTTATAAAGAGCCA | 762 |
| cg21791657 | TTTAAGTTAAAATTGCCCAGACGTGGTATTCTGAATCTGGGTGATTGGCG | 763 |
| cg19510604 | CGCTCCTGGTTGTCACATTCTGGCTCTTTTATGGGGTGGGAAATCTTTCA | 764 |
| cg07120314 | CGCTGCCAGGAGGAGCCGAGGCGAGTGGGCGTGGCTCTGGCGAGTGCCCG | 765 |
| cg10530344 | CGCCAGCCCCAAGCAAGCCAGGCAGTGTCCTGGTGTGGCAGCTCACAGGC | 766 |
| cg21658515 | TCATCCAGGCTGCAGTGCAATGGAGCGATCTTGGCTTACCATAACTTCCG | 767 |
| cg16708495 | GTGGTGGAGATGCCTGACATGGCTTGGGGACCTGCTGCCGGGGGAGCTCG | 768 |
| cg03997502 | GTGCAGGGGGAGCTGGCGCAGCTCAAGGCCTGGGTGAGGAAGCTGCAGCG | 769 |
| cg08405844 | CGGGGCTCTCCCAGATGCCCACGCTGCCTGCTCCCACTGGAGACGGGGAC | 770 |
| cg05144928 | AATTGGAATGTGGCAGGGAAACGCTGGGAGACCCCTCCCCATCCTTCTCG | 771 |
| cg09351263 | CGGCAACATCACTCCAGAGAGCCCAGCAGCTTCCTGGGAGAGGCAGGCTG | 772 |
| cg22510362 | GAGGGTCAGGTCTGTGCTGGGGGTGAGGTCCATGCTGGGGGTCAGGTCCG | 773 |
| cg03072681 | GTCTGGTCAAGAGAGATCCAGACAAAATCAACCTTCTGCGTCAACAGCCG | 774 |
| cg06959205 | CGCAGGGCACTCGGAGCAGGTCTCTGGCTGCCTGGCACAGGAAGGGCCTC | 775 |
| cg00892703 | CGCTGATACTGACATTTACAGATGAAACCGTCTGAGACCCGCTTCCCAGC | 776 |
| cg06912355 | AGGGTGTCGGCAGCAAGATGGCTCCGGGGGTTTAGACACTGCTGGCTTCG | 777 |
| cg13606988 | CGCACCCCAGCCAGAGAGGGAATACTGCACCTCCCAGAATGTGGCCTGGG | 778 |
| cg16569347 | AAGTACCCCTGCCGCTATTGTGAGAAAGTGTTTGCTCTGGCGGAGTACCG | 779 |
| cg12165215 | TCAGGAGAAATTTCTAACTGTAACCAAAGGGAGCAGGCAGGACCCAGACG | 780 |
| cg08667899 | CGCGGCTCAACCAAGCCTAGAAGTGGGGGCGGGGAATCTTCCGCCCTCCT | 781 |
| cg01130991 | CGGTATTGTCATGTTGCATCAAGGTTCCTCATTGCAGACAGAAATCTGCC | 782 |
| cg12054453 | CGGCAGAAGCTTCACCACAAAAGCGAAATGGGCACACCACAGGTAAGACT | 783 |
| cg18181703 | CGTGGCCACTCTTCAGCATCTCTGTCGGAAGACCGTCAACGGCCACCTGG | 784 |
| cg10508317 | AGCTGAAGGTCTTGAGGCGCAGGCTGGTGTCCAGGGGGCGGCTCATCCCG | 785 |
| cg27637521 | GGAAACTTGCTGTGGGTGACCATGGCGCACGGAGCCAGCGTGGATCTGCG | 786 |
| cg04243822 | CGCACCCAGCCCTTTACAAGCAGCACAAACGTATTTGGTTGTCTGGGGCC | 787 |
| cg12486944 | CAGGAGGCATGTGGGGGCCTCTGCCAGGCACTGCACAGGAGGGCGCGTCG | 788 |
| cg24391240 | GCCTCGCGGGATTAGGGGCCGGTAGAGGTGGGCGGGTCGGGCGGAAGTCG | 789 |
| cg19617213 | CGCCGAGGCCCGCTGCCCGCCCCCGCCCGCCCCCGCCCGCCCGCCTCACC | 790 |
| cg12846567 | CGCCCGGCTGGCTAGACCATTGCTAATGGCTCAAGAGYTCAGTTCTAGCA | 791 |
| cg03358468 | CGCCTGCAATTAAAGGCCAGATTGGAGGCATCTGATCCCCAGCCCTGATT | 792 |
| cg18569704 | TGTTGGACCTCTAAACGGTAATTACGGGCGACAACGCAGATCCAGGATCG | 793 |
| cg15001547 | GAGCAGCAGCAGCATGTACCTGGCCATGCTGGACGGAGATGCTTTGAACG | 794 |
| cg02991799 | GCAGCCGCAGAGACCCTTGGGAAGTGTGGTCTTCGGGTGCCGGCGGTTCG | 795 |
| cg25450266 | CGGAAGGGATGTTGTTTTGCTTCCGCAGACACCTGACCTCCAACTTCCTA | 796 |
| cg22030032 | CGGGAATGGTGCTGTCTGCCCAGCCCCACCCGGCLTTGTAATGAACACTT | 797 |
| cg09915601 | TGCCTGCAAACTAAGGCAGCATTATTCAGACCTGGGCTACCCTGGTCCCG | 798 |
| cg05971102 | CGGTCTGGYTAGAAAATATCCAGCCCCACAGCAAGGGGAAAGAGTAACAT | 799 |
| cg07520608 | CGGATTTCTGAAAATGTGGTTATTCCAGAGAATTCCTTCACGAGATTTAA | 800 |
| cg01412762 | CGGGTGCTTCCAACCAGTCTGCTGCTCCTGCCCCACCCGTGCACGACAGC | 801 |
| cg09920725 | CGGTCTTGTAACAATTGGATGGATGCCTTTGAAGAGCCCCTGTCCCTATT | 802 |
| cg12977946 | GTGGGCGTAGGACCGTCTGAGCCATGTGTGGGATATAATCTCCTGGTGCG | 803 |
| cg20150163 | CGCCGGAGGCCGCCACAACGCAGGCGCATTCAGCTAAGGACCACTCCCTC | 804 |
| cg04946715 | CTTGTGGCCATCTCTGTAAAGTTTCTGGGGAGGTGGGCACGGGGTAAACG | 805 |
| cg08645207 | GCATGGCAAGAAGCACCCCCAACACCCAGTTCTCCTGGAGACACTGATCG | 806 |
| cg14716323 | CAATACAAATTAATAACAACCAGCCAATTCCATTGTCCTTGTATGCATCG | 807 |
| cg01956624 | CGCGGGCAGGTGCTGCTGGTGGGCGAGCTGTGGGAGCGCGAACAGAGCCG | 808 |
| cg00497251 | CGGGACAGCATGTCACTCCGGGAAGCCTCCCGTTCAGGTGTCTGGTCCCT | 809 |
| cg27114661 | AGCCAGGAAGCAGTTGGCAAAGGGGCGGGGTGACGCGGTGACGCAATCCG | 810 |
| cg03443360 | CGGAACATGTTTGGGTATAGGGAGCCATCAGACTGCAGTGCACACCTGAC | 811 |
| cg17758363 | CGGCCCCTGGATCCTAATTCAGACCAGCCTGCCTCTGGCAATAGGAAAGT | 812 |
| cg14928764 | TCTAGTGTCTCTAGGGCCACCTCACTTTCAGCAGTCTGTCCCAGGTGTCG | 813 |
| cg11850311 | TACATCTGCTCCCCTGGGCTTTCTCAATTCCTAAATAATGTTGCTGTTCG | 814 |
| cg14234805 | GGAATGGGAGGAACGCAATGCGTCCCGCGCGGCGATCTGGGATAGCTGCG | 815 |
| cg20747455 | CGGGAGCTGTCAATCACGAACACCAGATCCAGGGGCCCAGTGTGACACCT | 816 |
| cg12112556 | CGCTCTCCTAGAGAGGGCTTCTGATTTGGAGCTGTTGTTTTGGTCTGAGA | 817 |
| cg17262492 | CGGGCCCCTGGGGCCTCAGAGGGACCCCGGCTGCCACTGACATATGAAGA | 818 |
| cg07871532 | AGTGGCTGCAGCATGGGTCAAGGTCAGAGAATCCGGGAGCCCCACAGCCG | 819 |
| cg08548559 | AAGCGTACGCTCTGTGCCAGTCCTTAGGTAAAATGTTTTACTTTGTGCCG | 820 |
| cg27115863 | CGCCAGCCCGCGGAGCCCCAGTGACTCAGAGCCGTGGCTCCCCTGGCCCG | 821 |
| cg22650271 | CGGCTCAGGACCAGCAGGCATGCCTCCTTGGTCTCATGTTGACCTTAGAG | 822 |
| cg19778944 | CGCGACTCACCCACGGTGGCCGGGCAGAGCCTGCCTCGAACCCGGCTTCT | 823 |
| cg17025841 | GGAGGAGGCTTTCTGCTGGCTAGTACTTTTCCAACTCTGACAGTGGCACG | 824 |
| cg06841024 | GAAGCACTTGGACAGGCCATCGGGCCAGAAGCACCTCGCAAGGGTGTCCG | 825 |
| cg27326062 | TACTTGCTCAGGGCTTTCTTCCCGCGTGTGCTTGGCGCTGCTCACAAACG | 826 |
| cg10747042 | AGATGCCTAATTCAGCCGGGAGCCGCGGGCCGGAGCTGGCTGAAGGTCCG | 827 |
| cg18098839 | CATAAACCAGATGTTTCTTAAAATAGCCCAGTTAAATCCACCCTTCCTCG | 828 |
| cg13457961 | CGGGCGGGGAAAGCGAGGTTTAACTAACTCCGCAGCTATATTCTTCCGCC | 829 |
| cg10407935 | CGCCACCCCACACTCCCTGCCTGGGGAAGTCGGACGCCGCCCCACACCCC | 830 |
| cg01096266 | CAGAGAGCAAATCCTTCTTCCTGCCCTAGGCTGCAGTGCTCCTAACCTCG | 831 |
| cg05613002 | TAAACCGAGTTAATCATCGGTCCATTTGAATAATCAGTCACGTGGTGGCG | 832 |
| cg05890887 | CAAGCAGCCAAGCTGCTCCACGATTGGCTGCTTTAGCCTTACGTGCGTCG | 833 |
| cg06440348 | GCAAACCGTGAATGTGGGGCAGTGTGGGGCCAAGCGGTGCGCTTGGTACG | 834 |
| cg21833476 | TTTCTCGAGGCAGGGGGCACGGTAGCACAGGGAGCTTCTCTTTGTGGGCG | 835 |
| cg26036479 | CGACAGCCACCTTATGTGGAAAATACTCGCGCCGGCCGTCATTCATAATC | 836 |
| cg25629712 | CGGCTCAGAGCACAGGCTCAGAAATCAGACTGCCCACGTTTGAAAGCTGG | 837 |
| cg12716346 | GGCGGCGCTGGGCATTCTCTGCCATCCTTAGGGGTCCTCCACAGCATGCG | 838 |
| cg02514143 | CGCACGAACCCTTCATCCTGCTCCAAACAGAATGAGAATGTCATGTCGCA | 839 |
| cg15016740 | CGCTGCCCAAGTCTCAGATATTCGATGCCTCACATTGCCAATATGGAGTC | 840 |
| cg01256539 | CGGCAGAATTGTGCTTAATAGTTGGTGAGGTTTCTATGGGCGATGTGGAA | 841 |
| cg08260658 | CATGGATTTAATCAGTGTACTTTTTACCTTCAGGCTGTAAATTAGAAACG | 842 |
| cg25024993 | CGAGATTTTTAACCTGAAACTGAAGAATCTGGTACTGTAAGTGTAAAGAA | 843 |
| cg22157525 | CGCCGAGGGCTCCAGGCCTCGCTTCCAGCCATATAGGTAGTAGGCCGAGA | 844 |
| cg21227060 | CGCCTGCTCTGTGCCCCACTACGCTTCATGCAGGGGTCCATGTGCAGCTC | 845 |
| cg17255947 | CAGTAACTCCGGCCGAATTTGTTCGGGTGTGAGGTTTAAGGCCCTAATCG | 846 |
| cg26372792 | GTGACTGGCCAAAAAAAACCGCAATTTTGGGGTCTAATTCGATTGTGACG | 847 |
| cg26867866 | GGGCGGTCAGGCGGGGTCAAGGCCAGGCAGCGGGGCGCGTCTGCGTTGCG | 848 |
| cg09144073 | CTATTGGTTGAGTTAACCAGCATTCAAAAGCAGTCAGCGAACCAACTACG | 849 |
| cg11074353 | CGGACAGGTTTTAAACAACCTACCAAGAGCCAGCGTGGGTGTCCACACCC | 850 |
| cg10000096 | CGGGGTGAACGTCTGGGTCCTCGGAAATCCTCAGTCTCTGGAACTGAATT | 851 |
| cg17501210 | CGCCCGATTCAGACAGCTGGACTCAGAGGGATTCTGCTCCACAGAGAAAC | 852 |
| cg26960221 | GGGAGGCCTTGGTTGGAGTGTCTGAGGCCTCAGCTGGGCCCCTGCCTCCG | 853 |
| cg24172278 | CGCAGCAGCCAGCTCATGGGCCCCGCATCCAGGTGTGTCCTGCCCTCTTC | 854 |
| cg01753176 | CGCTGGGCACTGCCCTGGGGGCTGAGCAGCTGTCTCCATTGGTCATTCAG | 855 |
| cg21843627 | CGGAACTCGCTGGAAGCTGTGATAGTCCCAGCTATGGTTTATCACAGGGA | 856 |
| cg19080320 | GAGCTGCAGCTGGGGACTGGTGCGCCATGCAATTGACATAGCGCTGCTCG | 857 |
| cg20915897 | TACTTCAGGGTAAAGAAGTTCAGGTCTGCCATCTCCAGCTCTGTCTGGCG | 858 |
| cg26116499 | CGGCGGCTTCCCCAGGTGCTGCTCCGCCCAGTGCCCCCCCAGATCTCAAC | 859 |
| cg07665217 | AGCATGACCAGAATGGTTCCTCAGAATCAGGAGGTGAAACCTAACATTCG | 860 |
| cg02505126 | AGAGTGGCGCGAGCCTGCGTTTTCCGGCCAGAGGACATGGTGCGTTTTCG | 861 |
| cg13565994 | GGATCCCAAATAACAGATCTGGGAGAATCCTGCCTCAGTACTTGGATGCG | 862 |
| cg17561365 | CGCCCACAGGGCCCAGTGGGCTCTGAGTAAGGTGCACGGCACACACCAGC | 863 |
| cg09194449 | GCCTGGGATCATCCCCAAGTGGGTCTAGGGTGGGCCTGTGAGGAGGAGCG | 864 |
| cg26206456 | CGACTGCAGGCCTACGGCTTCCTTTGAGGGCTCACGTTTACAAGATAACA | 865 |
| cg21462914 | CGGCCGCCAACCGACCACTGACTGTCTGCCAGGCATCCGGGCTCCCTCCA | 866 |
| cg17560136 | GGCCTCCAGAGCTGGGTAAGTGGAGGGCTCTGTGTATGTTGCTGTACACG | 867 |
| cg19033875 | CGCCCATGCCCGCGCTAGTGGGAGGAGGCTGGAAAGCGCCTTCCCATTTT | 868 |
| cg18995788 | CGGGCAGATACGAGCAGATTGACTCGCCAGGACTGTCATTGGGCCACCGC | 869 |
| cg14387545 | CGGCGCTGACGCCGCAAGGCCACTTCTATCGCTCCCGACCACGGTTTGTT | 870 |
| cg23083672 | CGCTTGTCAGAGTCACCATCTACTACCATGTATTTCAGGCATGGGGCAAA | 871 |
| cg02758183 | CGAGGTGCTGGTGTTCATACAGATAAGGCTCTCTTCATTGCAACAGCCAC | 872 |
| cg20308511 | TCATTTGCAAATGTTACTGGGGGACACACCGGCTCCCAGTAGGGTTTCCG | 873 |
| cg21412053 | CGCAAGAGAAGAGGGAGCACGCCCAGAAGCTGATAATGCTGCAGAACCTG | 874 |
| cg26071410 | CGCTGTGGCAGCCCAAACGTCAGAGGCACCTCACTTTGCCATGGCCTGGC | 875 |
| cg09625066 | CGGCCCAGTGAACCTGAGCTTTGGAGTTTGGACGAATAGCTAGTTTACCT | 876 |
| cg11235848 | CAGGGACTGACGCCCCTCTAGCAAAGCATCTGTGGTACATCCCAGACCCG | 877 |
| cg02311013 | GATCTCACACTTTGGAGACATACCCCTAAGTAGACCAGAGGCATCACACG | 878 |
| cg21664443 | CGCCTGGAGAAGCAGTGCCAAGTCTATTCCCTAATAAAGTACTTGTTGTG | 879 |
| cg05127178 | CGGAGCTGCAGAAAGAAAGGGAAGAAACTAACGTTCTTTCTGCGTCTGGG | 880 |
| cg19110434 | CGCTAAAACTCACAGCCTGTGGACAAAGCCTTTTTGGTTTTCAAATCGGC | 881 |
| cg25697726 | CGGTCAAAATTAAAAACTTCTGCACTGAAAAGAAAAAAGGGAGAGAGAGA | 882 |
| cg03204600 | CGCCCCCAAGAAGCTCCCCACCCCCAAACCAGAACTGGTGGCTGGTGACT | 883 |
| cg11049305 | ATTGAATGCCAAAGTTACTGGTCCCCAGGCAGCAGCTAGCACATTCAACG | 884 |
| cg13501581 | CGAGTGGCTACTGTATTGAACAACACAGATCTAGAAACAGAGTGTGGACC | 885 |
| cg01564693 | CGGGAACCTCCACAGGCTAAGGTGAGTGCCAGCACTGGCACCAGCCCTGG | 886 |
| cg10509982 | CAAAGGCAGACATCGCTTCAGGTAGGTTGCAGCCAAAATGGCTTCCAACG | 887 |
| cg20557017 | TTCTTTGTGGCCAAGAAATTCCCACCTTACCACTTACAGGGAGGCCATCG | 888 |
| cg05373692 | CGCAGTGTGAAAGATGCGAGCCATAGCATCTCGTAACATGAAGGCCATCC | 889 |
| cg05470074 | TATTTTGTATTTGGCTTGCTCTGGTTTCTGCTGGAGCTTTTAAAAAATCG | 890 |
| cg08262933 | CGGGATTACCTAGTGCCTTCACAATCGGTCAGAGCTGGATTCAGATTCCT | 891 |
| cg15062055 | CGGGTTACTACTGTACCTACCAGCTGATTCCAGCAGCCTTTTCTCCAGCG | 892 |
| cg12889449 | ACCGTTTTTTCCCCCGAATAAAGTGTGCCCGTGTAGCTGTACAGATTTCG | 893 |
| cg23955970 | TCTTTCTTCACGTCTTGTTGGTTACTGCTACCTGGCTTCCAACTGTTGCG | 894 |
| cg05530348 | AGCTGGCCAGACTGCTGCAGTTTTCAGTCCCAGGAACTGAGAACGTGGCG | 895 |
| cg11595135 | GGATTTGGTCTAGGCTTTCTGGCTTGGCCCTGGAACTCTCAGCAAGGACG | 896 |
| cg06298190 | CGCAGGTTACTTCCTATAAATGGAAGAACCTGAATCATTAAAAGATAAAA | 897 |
| cg20686207 | TAAGGCCTCGGCCATGACTGTGGGTGGGTTTAACTCAGCTAGCAGCTGCG | 898 |
| cg14686949 | CGAGACTAAGAAGTGACCCAACCTAGGTGACCCTTCAGCTAGGTCACTCT | 899 |
| cg18003791 | CGCCCATAAGGATATAAGGGAGACAGAGGGAAAAAGGAGGAAGCTCACTC | 900 |
| cg05295197 | GCAGGCGGGCCTCAGGGGCTGTCTTTCCTGCACGGCTGTTGTGTGCTTCG | 901 |
| cg23106779 | CGGCACCACACAGCTAGTGCCGGAACTTTTTGTGCAGAGAAAGAAGACCC | 902 |
| cg10143960 | GGTAGGGTGGGGCCAGGCTGAGCCTGCTCTGCGGGTCCCGGACCCCAGCG | 903 |
| cg07284273 | CGTGCAGGCCACAGTAATGGTGGTGCAGGTGGCAGGAGTAGGTGCCCTCG | 904 |
| cg17747265 | GGACCTGAAGATCTGCATGTATGACCGGCTCTACCAGGACTCTGTGCTCG | 905 |
| cg09971184 | TGACACTGTCTTCGGGGCACATATGGTCATCTATGAGTGCTGTGAGCACG | 906 |
| cg00935435 | GAGGGGCTGGCCCCCTTCACCCTGTGATCGACAGGACAGAGCTGGGCACG | 907 |
| cg15651727 | ATGCATGTTTCACACCTTCTAGAACTCAGAGTAAACCTAGCTCTTCAACG | 908 |
| cg13990487 | GTTAACAACTGTCAACAGCCTATAGAAGATCAAGGCTAAATTCCCTAACG | 909 |
| cg00456299 | CGAGTTTAGCTTTTAAAGAATCCTTTGAAGAAGGATTGCAACGTGACTAA | 910 |
| cg04974804 | TAGAGAAATACATTGTAACAATTTGGTTTCTTTCCTGATGGCCTTTATCG | 911 |
| cg13574848 | ATTACAAGCATGAGCCAGGGCACCCAGCCAGAAATGATATTTAAACTACG | 912 |
| cg25616777 | CCATGTATAAGTCCCCAGGACTCACAAATCTCCTCTTTCAGGATATCTCG | 913 |
| cg12386614 | CGTTAGGTCCCACGGCTTGCCCTGCTCAGCACCTAATAAGGCCATTAGCT | 914 |
| cg15355146 | CGGGGGCTAAGCCAGCGCCGTCTGCCTCTCCACCTAGACCAGACCCCTTC | 915 |
| cg19471466 | CGGGGGGTGCCAAAAAGCCCTCCTTCTGACGGCCCTCCTGGAGCCCAACA | 916 |
| cg14709691 | CGCTTTACGGAAAGAACGCCCCGCGAATAAATCCGGAGCGGATTTGCATC | 917 |
| cg21280384 | CGGAGCCACAGAGAACACTTTTGAGACCAGCAGACAACCACAGATGTGCG | 918 |
| cg14476101 | CGCCAGCAGATACAAAGGCAGACAAAGTGAGCGAGGCAGTTTCCAGGACC | 919 |
| cg19693031 | TTCCTATCACAATTTACAAAAAGCCTCCAAAAAACCTTGAAAAGCTTACG | 920 |
| cg16700924 | CAAGGGAAGCTCCCGCCTCAGGGTCTCTGTCTTCCTGTGGTTAAGAGACG | 921 |
| cg20246707 | CGCTCCGGCTGGTAATTTCCAAACACAGAGGGAAGAGCATGGTGGAGCAA | 922 |
| cg01365152 | CGGAACTGTCCTCGACCCGTGGGTACAGTATTTTCTCGTATGCCACCAAG | 923 |
| cg20327105 | TTGGTGCAGTTTTGAGCGTCCGTGTATAAGGAGTGGCTCCGTAGTCACCG | 924 |
| cg25741192 | CCATCAGTAGAGCAGGAGTCGTTGGCTCAATGCAGTGCTGTCTGCAACCG | 925 |
| cg13678787 | CAGTGTTCGGCAGAGGACAATCTGTTCGAGAATAATTAGATTCCCCCTCG | 926 |
| cg08614871 | CGGATGCTTAATTACAAAAGGTTTTGCCCCTGTAGTGACCGGGCAGCAAT | 927 |
| cg22660341 | CAGCTCCCACCTGTGCAGAGTGGCGGCCTGGGAGCTGAGCGTGGCCCTCG | 928 |
| cg19647685 | CGCGCAGCTTCCTTACAGGAGGCTTTGAATGCTGCCTGGAGTATATTTGA | 929 |
| cg03153178 | CGCTTAACAATGAGCGGGGCTATATGTCTTGCGCTTGCTCTGAAGGAAGG | 930 |
| cg10531748 | CGCCGGGAGCTGCTCGCCCAGTGTCAGCAGAGCCTGGCCCAGGCCATGAC | 931 |
| cg01508796 | CGGGTGAGTGTTTAACTTTCAGCCCAGCTTAAGATCTCCGAAGACACCCA | 932 |
| cg06439736 | GCAGCGTGCCTACGCTTTGTTGCAGGAGCAGGTTGGAGGGACGCTGGACG | 933 |
| cg18853287 | AGGATGTTCAGTGACTGCAGCCAGGCCTACCTGGAGAGCTTTTTGGAGCG | 934 |
| cg13446658 | CGGCTTCTGAACTTTGTACTTTGCGAGTGTACTTTGTACTTTGGTCCTGT | 935 |
| cg03322338 | CGGCCTCTTATCTCGACAATCGGTTGCTCTTGGCCAGCCCGTCCTGCTGC | 936 |
| cg09566331 | CGCTTATGCCCAGCTGCTCCAGCTGGAAAAGACAGGACTGCAAGTGGGTT | 937 |
| cg21992507 | CGGACTTTCTGCGCAGAGAAAACACTATCTCCTTTGGAGGCTGTGCACTT | 938 |
| cg01438090 | CGCTGATTGCACTGTCAAGAAGGAAAATACTGTTTGAAAAGACAACAGGA | 939 |
| cg01562537 | AACAAACCATTACGTGTTCCAATTAGAGCGGCAGCGGACCTGGCTGGCCG | 940 |
| cg17044529 | CGGCAGTGAGTCACTTACATAGATGTGTGTAACATGCCCCAGAGACCTCA | 941 |
| cg00574958 | AGGCCAGCGAACATCCAGCTGTCAGTTGGTCTGGGGACTATCAGCATTCG | 942 |
| cg17058475 | AGCGACACTTATGTCCTCAATTTATTGCTACGTTCACTCGGTCAGAGTCG | 943 |
| cg21960184 | TTCATGCCGGACAAATTTCCCTATGAACAATGCAGCCAAGGGCTGCATCG | 944 |
| cg02395454 | CGTGGGCAGAGTGGTTGGCTGCCCCACTGTCAGCCTGTTCTGAAAGTAGG | 945 |
| cg24675983 | TGCAGTTCTTATAATAAACACTTAAACAGGGCCAGGCACTGCAGTTCACG | 946 |
| cg21574681 | AGGACTAGATGTTCACTTTAAATCCAGCCACTGCTCCAGACTGCAAATCG | 947 |
| cg17850642 | CGCCCTTTTCCGGTGTCCTGGGCTCAACAGGGATAAAAGGAGGCAGCACT | 948 |
| cg01031441 | GGTGGGGACTGGCATTAGGGGTGGGCGGGAGAGACCCTAAAAATCCACCG | 949 |
| cg01638369 | CGTGTGTTTGTAATTGTGTATGGTCCTGCTAATGTTTGTGTTTATCACTG | 950 |
| cg07539443 | CGGTTCACAGCATGGGAGCATCTGTGCTATACCAAGATTACTCATAGGAT | 951 |
| cg15997315 | CGGGGCTGTCAAGTGTCTTTAAAAGGTCCCTTCAGGGTGGATGGAAACCA | 952 |
| cg13842421 | GCCCCCAGCTCTCAAGGCCGGTGCAAGGATGGGCTGTGCTCAGCGATGCG | 953 |
| cg02925268 | TCCTCAGACTGTTGGGATGATGAGCGGACGGTGAACTCCCACCACCTGCG | 954 |
| cg26975476 | AGCGGGAGTCACTTCGGAGTGGGGTGTTGACAGCGTGAAGCCCCTGAGCG | 955 |
| cg13589330 | CGCTTTCAGGGGTCAAAGACCTGGCAGAAATGACTTCCCAACCCCAGATG | 956 |
| cg02853801 | TAATTGCCCTCCTGTTTCCTAGAAAGCTTATAGGGCAGCAGTAGGAAACG | 957 |
| cg06147196 | CGCAGAACAGCACAGTGTCACATGGTCTTCACGGCCCTGAGGGCCCAACT | 958 |
| cg24126612 | CGGGGACCACCAGCTGCTTTGCCCATGGATTGGAACGCATTGGGTGGAGT | 959 |
| cg26407029 | GCTGTTCCTCATCACTCAGTACCCAGTTTGCTGACGTCTACAATATTCCG | 960 |
| cg01802397 | CGTGATTGCACGTATCAAGATGTGTGCTCTTAGATTTGTTTTCAATCCAG | 961 |
| cg01885071 | CGGCGCTCAGCAAATTTAGATGAATGCAACGCTTCAACGTGGGCTCTCGA | 962 |
| cg01308343 | GTCTTGATGTGACCAGAGAACACCATGTACAGGCAAAGCCCAGCCCCTCG | 963 |
| cg15471661 | GCTGTCGCTGCAGGTGGGAACCGAGAGCCAGGAAGCCCAGGGAGCTGTCG | 964 |
| cg16558846 | ACAAGGGGCTCCTCCTTTGTCCTGGGGGAGTTGCATGGATCCTGTCTTCG | 965 |
| cg17382302 | GTTCCACATAGAGAGCCTGGCTGCATGATCTTGAGGTCACCTAACTCCCG | 966 |
| cg25291250 | CGCGCCTGGCCTATACAATATTTTAAATAATTTTGTGTGTGATACAATGA | 967 |
| cg01021485 | CGCCCGCCTCATTTGCGCCTTGCAGCACTGCTGGACCAGGTATCTGCCCG | 968 |
| cg27505538 | CGGGTCAGACCGTCATTGGCCATCTCACAGTCCCGGCGCTCCAGAGCCCC | 969 |
| cg24924577 | CAGCCATTGGCTGATTTGAAAGAATACACAGGGTGTGCCAGCTGGTGTCG | 970 |
| cg00564555 | CGGTCTCAGCCGTCCCCATGGTCTCAGCCGTCCCCAGGGTCTCAGGAGTC | 971 |
| cg02193806 | CGGTTATATCAACATGATGCCCAAACACCAGGTCTGCTTAGGGTAAAACT | 972 |
| cg05732130 | GTACAAATGGGTCCAGGAGCTTCAACGAGCGTTCCAGCTTCTTCGTAACG | 973 |
| cg02189786 | CGCGCACCGCCCCGCTCCACGCCCCTATCAGGATTCGGGTCCTCGTGAGG | 974 |
| cg00534468 | CCTCGACTGCTGGCTGTGATGTTCTCCAACACTGCTATTCACAGCGAGCG | 975 |
| cg03699074 | GTCCCGTTCACACTGATCGTTCCATTTCCTCTTGTGTCTGAGTGGGAGCG | 976 |
| cg01829657 | CGGGGGTTGGTCAGCACCACACACTGTGCACAGGGAAGATGGAGCCTCGC | 977 |
| cg00892703 | CGCTGATACTGACATTTACAGATGAAACCGTCTGAGACCCGCTTCCCAGC | 978 |
| cg27113347 | TCGATGCTGGTGGTCTGCGTGGTGGTTCCCAACAGCAGTTCAGGGGCTCG | 979 |
| cg09826306 | GGCGGCTCTAGCCTCTCGGGGGACGTTCGGCTACTCCAGTGGGGGGTTCG | 980 |
| cg25567048 | GAACAGCCACGCATGGTCCGTGAAATGGGATCATGTGCAGAACAGCCACG | 981 |
| cg06513864 | CGCCCCACCAGCCCGAGAGCTAGAATTGAATCTGGGGGACCCCTGCGGCG | 982 |
| cg11020638 | AGCACTGTAGGGAGTGGCCGGGATCATTCGAGCCCACAGATAGTGCTCCG | 983 |
| cg05815247 | CGGACAGCTAATCGTTTTAGTGACAGGATGAGAGAGCCCTTCGTGTTCTG | 984 |
| cg21348173 | CGGCCTGACACTCTCAGAGATGGGATTCCCAATCCATCCACTCTGAAATG | 985 |
| cg22850234 | CCGGAGGCTGATGCCTTATCCATTAGGCCACTGGGTCACCACAGGATCCG | 986 |
| cg10508317 | AGCTGAAGGTCTTGAGGCGCAGGCTGGTGTCCAGGGGGCGGCTCATCCCG | 987 |
| cg27637521 | GGAAACTTGCTGTGGGTGACCATGGCGCACGGAGCCAGCGTGGATCTGCG | 988 |
| cg16252995 | CGGCAGAGAGGACAGGATCCTGCCACCCAGCCTCATCCCCATGCTTCCCC | 989 |
| cg03524223 | TCCCACCTTGTGAAACTTTGCCAGGTGTCGCTCACAGGGGACTGGGTCCG | 990 |
| cg05495454 | AGAACATCCACTCTCTTCCAGCCAGAATACCATTAATACGTGTGTCAGCG | 991 |
| cg07573872 | CGGGAATGGTGACTCAGCCTTCCAGGAACCTGCGTGGCGTCTGTTTTTTT | 992 |
| cg22519313 | CTTCCTATGTGATTTTCTTCCAACATTCTCCCATGAAAAAATGTCAGACG | 993 |
| cg05313771 | TGGGTGATGGAAACTGAGGCACAGTCATCCCCCCAGCAGGTCCTTGGACG | 994 |
| cg09522532 | CGGGCCCCCCAGAATGGAACACGAGGGGGTGGTCTCTTGCTGGGCTCCAG | 995 |
| cg15783941 | CGCCAGCCCAGGGCTGGATGTTTTATTTTGGCATGCCCCAGGTGCTCTTC | 996 |
| cg24115922 | CGCACATGCGCATCCAACCCGATTCCACGGGTTCCCAGGAGGCGGATTCC | 997 |
| cg18593194 | CGGGAAGTCTGCCAGTTGGCAGTCTCTGACGTAAAAGATAGCAGATACTG | 998 |
| cg25654926 | AGCCCCACTAATAGATATTCTGATTCTGTTGGTCTGGAATGGGAACCGCG | 999 |
| cg10853231 | GGAGGGCAGGTCCGGGGGCTCTGTGTCCCCACTGGGCCCCCAGCACTGCG | 1000 |
| cg19717326 | CATTAAAGCTCAGACTCGGGTTTTCCGAGCAGGTGTTGCAAGCCTAGCCG | 1001 |
| cg13650654 | CGGGTCTCTAAGATGTCTTATGAATGCAGGTCAGAGGGTCACATGTTAAC | 1002 |
| cg02875404 | CAGTGGATAGTGACCAACTCTGTGTTGGTCGCCTCATGTTTCCCAGCACG | 1003 |
| cg14889658 | AGCATTCGGGCATCAGAGGTTTGTGGAGAGGGCAGCCCAGTATACAACCG | 1004 |
| cg02591356 | CGTCTAAATTACTTGGAGTTGATCTTTCTAAACAGGATTGCTCAGACACA | 1005 |
| cg26471058 | GCCTGAGGTCAGGAGTAAGCAGACACCAGCACTGCTCTTTCTCCAAGACG | 1006 |
| cg25939861 | CGGAAATCAGCTTGGGGGCCTTCTAGCCCTGCAGCTCAGAAAAGTGTCAG | 1007 |
| cg03913456 | ACCGCGCTCAGCCCGTGGAATTTACTTTATAATGGGGAGGCAGATGATCG | 1008 |
| cg24506130 | CGGTTGTCAGCTGCCGGAAGAAGAGCGTGGAATTGAGAACACAGACCCCT | 1009 |
| cg26009035 | TGGGTGCCAGGGGGATTATCAGCGTCTGCGCAAGGCCGATGGTGGCAGCG | 1010 |
| cg05308744 | TGATTGGATGCAGGATTGGCGGGAAGGCAGCGGGGTCATCAAAGGCTGCG | 1011 |
| cg22663545 | CGATGAGCTCAGCCCCCTCAGTCCAGGCCGAGAGCCAGATGGGGGTCACG | 1012 |
| cg02767242 | GTCCCCCTCGTTTCCTTTTAGAAGTCATTGGCCGATCGTTTTAAAAGTCG | 1013 |
| cg13603203 | TGGGATCAGGATCATGGTGAGATGTATGCCTTTCCCAGGAATGCTAGCCG | 1014 |
| cg20951650 | ACTACTCTCCTGGAGTGGTTACAGTTAACCAAGGCAAAACCCCTTCTTCG | 1015 |
| cg12683410 | CGGCTGTTGTCCTGCTTACAGGACTCCAGAGCAGCAGGAAGGAGCTATGT | 1016 |
| cg16887334 | CGCACTCGGCCTGACCCACGGCGACCCTCTGTGACCAATCATACTACCAA | 1017 |
| cg26126740 | TATTCCAATTTGGGCCTGCGTGTTCGGAGCTCCTGGTGGCGGTCTCCACG | 1018 |
| cg10120040 | CGGGCTTTGGTGACCAGGTCTTCTGTCAGTGAGCACACATAGCCCGGAGG | 1019 |
| cg25761955 | CGCCTGTGTGGACGCTTGTGAGCTGCACCTTCATGCTATTATAATGGCGA | 1020 |
| cg22871721 | GAAAACACACAGATGTCAAACCAGAGGAGCTGCCAAGCTGGGTACCGACG | 1021 |
| cg22778797 | CGCCGGCCCCAGGTAGTATATTTTTATTGGCACAACTGCCGGTATTTGCC | 1022 |
| cg27243685 | CGGAGGATGTGTTTCCAGAGAGCTGGTCTATTTCAGACTGACAGGCCACT | 1023 |
| cg06500161 | CGGCGGGACTAGTTCCTTTGGGCATAATTTAGGTGTTTTGTGAAATGTTA | 1024 |
| cg17215151 | TGCTTAGTAGCTAAAGGCCACCTTCTATAACACGAAATAGTCTACAAGCG | 1025 |
| cg01127300 | ATGCCTTCTTGGGGCCTGGCTTGCTGCTGACGGGAGCACTCAGGAATGCG | 1026 |
| cg12511214 | CGACAACAGGGACTCGGTGGACCTGGCCGAGCTGGTGCCCGCGGTGGGCG | 1027 |
| cg07031797 | ACAGTCGAAGCGCACTGCTCTGGTGGGGGATGTTGGCGGTGCAGGAGGCG | 1028 |
| cg18405719 | CGAGGCTAAAATGTGGGGCCAGAAGATAAATCTAATGAGAGCTGTTGCTA | 1029 |
| cg21614211 | CGGGGGTGTGTTTCTGTTTGTGTAACTGGGTGTGTGTGTATCTAGCCATC | 1030 |
| cg06813250 | TAGAAACTGATGTTATTCATAGATTCCACACATTGTATAGAAGAACATCG | 1031 |
| cg15705999 | CACCCCCAGCCTCGCCACGGCTCGCTCCCCCCGGGGTCAGGCCGTTACCG | 1032 |
| cg09000779 | CGGGACTGTGGCCAAGGCCCTCAGAGGCCATGGGGAGACACGACAAGACC | 1033 |
| cg12473849 | CGCAAGCACATAACAAAAGCCAAGTTAAGAAGGTTAGCGATTTAGTTGTC | 1034 |
| cg27336439 | TCACCATTGCCACTTAATGGTCTGAGGGGGGCTTTTCCTGTTACTGCACG | 1035 |
| cg00480331 | TGGATCGCCCAGGGGCGGGCAGTCCCTGGAAGACAGGTACGGAGTGGTCG | 1036 |
| cg13724379 | GGAAACGAATCTGGGAACGAGTTTGAAGCGCGGTCAAGTTTGACTCATCG | 1037 |
| cg15012625 | CGAGGGTCCCAGTGTTCCACATCCTTGCCAACGCCTGTTATTGTCTGTTG | 1038 |
| cg01658265 | CGGGGTAACCCCAGGTCCCAGTGATTTCCATAGACAAAAGAGATGCTGGT | 1039 |
| cg00252095 | AGTCTTGCCTGGTTACAGGCCTGTGCTCTCCAGGCAGAACAAGCGCAGCG | 1040 |
| cg26805150 | GACAGTTCTTGGGAAGCCGGCAAACTGCAAAAGCCCCATATTTCCTTCCG | 1041 |
| cg02373784 | CGGGCTCCTCAGGTCTGAGGCAGCTCTACCACTGACTTGGTGAATTTTAG | 1042 |
| cg07951602 | CGCCTCTCAAGAGCACGATGTAAGGGCTCCAAGATGAGTTTGGGCTTCCC | 1043 |
| cg19719391 | GGCACTTTTCAGCTCTAATGCTCTTGCTTTCCAAAAGCAAAACAGAACCG | 1044 |
| cg08284371 | CGGGGCAGCACAGACACCCGGGCAAATTCATGATCTCTACCGAGTAAATA | 1045 |
| cg26816491 | CGTGTCCTTTAAGAACTGTAACACTCACCGCCAGGGTCCACGGCTTCATT | 1046 |
| cg06690548 | CGTGAACTACCCAGATGCAGCATCCGCTGTTGGTGTGATCTGGAACACCC | 1047 |
| cg18376497 | CGGAAGGCTGAAGGGGGAGATAATGAGCTAAAGAACAACTTTCACCACCC | 1048 |
| cg06176987 | TTTAGTTTCCTTCTTTTATTTTCCTGGCAATAGCCCACCATTGTATATCG | 1049 |
| cg22744079 | CGGACACTGAGGCCTTGTAATGCTGTTGGCTGAAGAGAGGTGGTTGGTCA | 1050 |
| cg16400903 | TCCGCGGGGCCTGAGCAGAGTCGGCCTTGTAAGGCAGAGGTCTCGGTGCG | 1051 |
| cg09884076 | CGGCCTCTTGTCCACCTTGCCCCGAAGGCAAGTCTGGATATGAAAAGAAA | 1052 |
| cg12599971 | AGCTTCTTTGCTTTGACTGTTTTCCTCTTGCTGCGTCTCTTTGGTCTTCG | 1053 |
| cg26403843 | CGGATTGCTGCTTCTAAGCCTGAGAAGGTCATTTAATGAACTCATTGAGG | 1054 |
| cg07017437 | CGCGCCAGCCCATGTATGCCATGGAAATATGGCTGTTGGAATTCTGGTGT | 1055 |
| cg21036336 | CGTCTTGGGGACGCTATGGCTCCAGTTCTGACACTCAAGAAACGATGGAT | 1056 |
| cg03714531 | GGGCGGCTTCTGATCTCTTGCGGACAGCTCAGATTAGTGGCTCGAGGCCG | 1057 |
| cg23036136 | CTTGGGTGCCCAGGTCTCTTCCTTCCTGAAGAATGCGGCACTTGCTTCCG | 1058 |
| cg10189661 | CGGGCAGACTGCGATGAAACCCCAGTTTGTTGGGATCTTGCTCAGCTCCC | 1059 |
| cg05218245 | GGTTTCCAGGTAGCTTGATCTGTTCCAGGCCTGGCTGGGGTCTGCCCTCG | 1060 |
| cg04018738 | ACCCACCTGCCCGCCGGATCTGGAATAATGTGACTCGCTGGTTTGTCACG | 1061 |
| cg02115397 | TGCTGCGGTTGGAGCGCTCCACCAGGATGTCCAGATGGGCTTTGATTGCG | 1062 |
| cg12927358 | GGGGTTTAAGGATGATTCCCCACTCCACAGCCTGACGCATCGACTCTGCG | 1063 |
| cg05466385 | CTACGAGTCTTATTTCCTGTTAACTTTTTTCCTTTTGCTTGATCCTATCG | 1064 |
| cg01702009 | CGGCTGGGGGAGGAGGCACCCACAAGACTGCCTTCTAGGCCACAGGCCCA | 1065 |
| cg06560379 | CGGAGGGAGGGAGGAACTGCTCTGGAAATCCCCTAGGCCAGGGCCTGCTC | 1066 |
| cg21710255 | TGTTCCTGTTACTCTTCGTGAATTTTCTCACCATAAAACTGTTGCCAACG | 1067 |
| cg20459712 | GTACTTTGATGCTCTACTTGGCAGAATGTATTGCTTCTGGTACCCACACG | 1068 |
| cg18718984 | GTGCTACCTAGAGAGGCCAGCATGTGGGCTCCACATTTCTGCTGAAGACG | 1069 |
| cg03707967 | CGGTGCTTATAATACATTCCTGTGTGTGTGTTCACTTTGATTTACGGAAA | 1070 |
| cg02027561 | CGTCAGGTTTATATCTTTGGGATTTGAATGTAAAGATGTTGGATTTTTTT | 1071 |
| cg00695799 | CGCTCCATGTGACAGAAACATGAGCGTTTCCAAAATGAAAACACTGGGGG | 1072 |
| cg18116267 | TTCCCTTGCTTTTCCCAGCTTCCGGTAGCCCAGGCATTCCTTGTCTTGCG | 1073 |
| cg17501210 | CGCCCGATTCAGACAGCTGGACTCAGAGGGATTCTGCTCCACAGAGAAAC | 1074 |
| cg26905845 | CGGCTAGAGGATGACCCATCTTACGCCTATGGAGAACACTGCGGAAATCA | 1075 |
| cg03730314 | TTGCCTGTGTGTGATTTATGCATGTTCCCACCAGCAGGTGTGGATTCTCG | 1076 |
| cg03394401 | CGGGCTCCGATCTCCGGTGTCACAGCAAAGATCCGCGTCTTTCCGATGAA | 1077 |
| cg19196326 | CGCCTCCCCACGCACTTCCCTGTATGGGTTACAAAGCAGGTGCACATCCA | 1078 |
| cg13550401 | CGTGGCACACAGAGCCAGGTTTCCCCGAAGTAGTCTAACGCTGTTGCTAA | 1079 |
| cg08549335 | CATTGGCTCTTCACTTGTTGCTTGGTGTTTTCCTTAGTCTTCTGAGATCG | 1080 |
| cg17220237 | CGGGACATGAAGTCTAAGGGGATTATTTTGGAGCTTTAAAACGTGATGAC | 1081 |
| cg22322818 | CGGGAATTTGGTGAGAGTCCCCCACAGTTCTGTCTGCCAGGCACTGGCGA | 1082 |
| cg08695830 | CGCATCCTCCAGCCATGCTATGCTTGCAAAGATGTCTCTGGTCATTGGGA | 1083 |
| cg17981101 | CGCCCCAAGTCATGAGGGAAGCTTCCAGACTGACTCAGGCTCAGGCTGGC | 1084 |
| cg01337813 | GGAAGTCCTCTGTGCATGCGTGTCTGTGCCATAGGCGTGGGAACACCTCG | 1085 |
| cg25977769 | CGCCTAGGTATGGGAAATGTAAGTATCCTCAGCAGAGACCACAGTTCTCT | 1086 |
| cg07592681 | CGGTCAGCACTGGGTGAATGGGTTGAGGGAACCCACATAAAATCCCCAAG | 1087 |
| cg09584650 | CGATCTTCCCCATGGAAGACATGCACATGGGTTAGGAGAATACAACACTC | 1088 |
| cg01070197 | CGCAACAACTTCTCAGCTACAGTAAATCAGGATTTGGGGTGATAGGAAGG | 1089 |
| cg17744279 | CAATGTATACTATAGCAAGTGGTACCAGTGCTGCGGGTACCAGTGCTGCG | 1090 |
| cg13518625 | CACAGATCAGACTGCTAAGTCTTTACACATCCGAAAGTTAGACTTTGACG | 1091 |
| cg03393241 | GCCACGGGAGATTTCATCAGAAGAATATCAGTCTATTGATATGGGTGCCG | 1092 |
| cg22660904 | CCTCCCTACAACTGTAGGTTCCCCTGCTGCTGGGCTCAGCTGGGAGCTCG | 1093 |
| cg26074100 | CGGGACAGCCCCGATGCCGATGCCTGTGTTCTGTGTCCCCAGGTCTAAAG | 1094 |
| cg01029867 | GAGCCCAGCGGGGTTAGGGTTCAGGTGAGGGTTTAGGGTCGGGGCGAGCG | 1095 |
| cg14268714 | CGTACCCCGGGATCTGGCATTTAGCTGGCAATCAAATATGTGGTGAAACG | 1096 |
| cg13673536 | GTTTAAAATTCTCTCTTCTGAGTAATAGTGTTCATGTGCTCCCATTCTCG | 1097 |
| cg14457452 | ACTAGGTAAACACCACAGTAATAACTGTTGCAGGTAAGAATCATCAGGCG | 1098 |
| cg21208062 | CGGGAAATGACCAAGCGTCTCCACAATTTAGGGGCAGGTCAGGGTACCTT | 1099 |
| cg12020464 | CGCCACAAACCCACCAAGGTGCTCTCCCCCGAGTACTTGTGGGACCAGCA | 1100 |
| cg13873209 | CGGCACCACTCATGTTCATAGGCCAGCTGAGGCTCAGGGGGCTGACTTCC | 1101 |
| cg13499318 | CGCTGGGCAACCGTAAGTATTTAGGGAGACAGAGGTGCCTCTTCTGCAGA | 1102 |
| cg13915886 | CGACGCCCTTGCTCACAGGCTCCATTGAGAGGCTGCCCCGGTGCTCATCT | 1103 |
| cg22249566 | CGCAGCCCTGCGGGACCTGAAAAGGCGAGACTAAGTCACCAGCCAAGCTG | 1104 |
| cg17511128 | CGGTCACTGTTTGTCTTGAAAAAAAGAGAGAAAATGTTTGTCATTCCAGG | 1105 |
| cg02495445 | CGTCTTCTAGCAGGTATGAGGGAGACAACTACAAACGCCAGAGGCTCAGC | 1106 |
| cg16088894 | AGCTCTCGCCGCTGAAGGGGCTCAGCCTGGTCGACAAGGTGAACAGGCCG | 1107 |
| cg13573587 | GCAGGGCAGCCAGCTACGAATTTTGAGCTCTGGGGCGCGCTCACCTGGCG | 1108 |
| cg03788610 | CGGGTCAAGAGCAGTGACCTAGTGGCCCTCAGCCCTGGATGGAGGACAGA | 1109 |
| cg03990195 | CGCCATTCTGTTGGTACTAAGGTGTTGTGGTTGGTAGAAGCTTAAGTGAG | 1110 |
| cg08147391 | CGGGTCGTGGTGACTACTAATGGGTATAAGATTTCTTTTTGAGATGATGG | 1111 |
| cg05548952 | TGTTGACCTCAGGTCCAGTTTGTTTGCAGCCTCCCTCTTCTGACAGATCG | 1112 |
| cg07929447 | TTGTCTCAGATTACAGGAACCAAATGCATGTCTGGCCTTGTGCTGCTTCG | 1113 |

### EXAMPLE 2 REFERENCES

1. Dawber, T. R., Meadors, G. F. & Moore, F. E., Jr. Epidemiological approaches to heart disease: the Framingham Study. Am J Public Health Nations Health 41, 279-81 (1951).
2. Kannel, W. B., Feinleib, M., McNamara, P. M., Garrison, R. J. & Castelli, W. P. An investigation of coronary heart disease in families. The Framingham offspring study. Am J Epidemiol 110, 281-90 (1979).
3. Aryee, M. J. et al. Minfi: a flexible and comprehensive Bioconductor package for the analysis of Infinium DNA methylation microarrays. Bioinformatics 30, 1363-9 (2014).
4. Anonymous, A. Design of the Women's Health Initiative clinical trial and observational study. The Women's Health Initiative Study Group. Control Clin Trials 19, 61-109 (1998).
5. Anderson, G. et al. Implementation of the women's health initiative study design. Ann Epidemiol 13, S5-17 (2003).
6. Whitsel, E. Epigenetic Mechanisms of PM-Mediated CVD Risk (WHI-EMPC; R01-ES020836). in National Institutes of Health. U.S. Department of Health and Human Services. Research Portfolio Online Reporting Tools (2016).
7. Teschendorff, A. E. et al. A beta-mixture quantile normalization method for correcting probe design bias in Illumina Infinium 450 k DNA methylation data. Bioinformatics 29, 189-196 (2013).
8. Johnson, W. E., Rabinovic, A. & Li, C. Adjusting batch effects in microarray expression data using Empirical Bayes methods. Biostatistics 8, 118-127 (2007).
9. Patterson, R. E. et al. Measurement characteristics of the Women's Health Initiative food frequency questionnaire. Annals of epidemiology 9, 178-187 (1999).
10. Taylor, H. A., Jr. et al. Toward resolution of cardiovascular health disparities in African Americans: design and methods of the Jackson Heart Study. Ethn Dis 15, S6-4-17 (2005).
11. Moore, A. Z. et al. Change in Epigenome-Wide DNA Methylation Over 9 Years and Subsequent Mortality: Results From the InCHIANTI Study. J Gerontol A Biol Sci Med Sci (2015).
12. Wong, H. K., Cheung, T. T. & Cheung, B. M. Adrenomedullin and cardiovascular diseases. JRSM Cardiovasc Dis 1(2012).
13. Larrayoz, I. M. et al. Adrenomedullin Contributes to Age-Related Memory Loss in Mice and Is Elevated in Aging Human Brains. Front Mol Neurosci 10, 384 (2017).
14. Liabeuf, S. et al. Plasma beta-2 microglobulin is associated with cardiovascular disease in uremic patients. Kidney Int 82, 1297-303 (2012).
15. Smith, L. K. et al. beta2-microglobulin is a systemic pro-aging factor that impairs cognitive function and neurogenesis. Nat Med 21, 932-7 (2015).
16. Ferguson, T. W., Komenda, P. & Tangri, N. Cystatin C as a biomarker for estimating glomerular filtration rate. Curr Opin Nephrol Hypertens 24, 295-300 (2015).
17. van der Laan, S. W. et al. Cystatin C and Cardiovascular Disease: A Mendelian Randomization Study. J Am Coll Cardiol 68, 934-45 (2016).
18. Paterson, R. W. et al. Cerebrospinal fluid markers including trefoil factor 3 are associated with neurodegeneration in amyloid-positive individuals. Transl Psychiatry 4, e419 (2014).
19. Fujita, Y., Taniguchi, Y., Shinkai, S., Tanaka, M. & Ito, M. Secreted growth differentiation factor 15 as a potential biomarker for mitochondrial dysfunctions in aging and age-related disorders. Geriatr Gerontol Int 16 Suppl 1, 17-29 (2016).
20. Maioli, S. et al. Alterations in brain leptin signalling in spite of unchanged CSF leptin levels in Alzheimer's disease. Aging Cell 14, 122-9 (2015).
21. Cesari, M., Pahor, M. & Incalzi, R. A. Plasminogen activator inhibitor-1 (PAI-1): a key factor linking fibrinolysis and age-related subclinical and clinical conditions. Cardiovasc Ther 28, e72-91(2010).
22. Ashutosh, Chao, C., Borgmann, K., Brew, K. & Ghorpade, A. Tissue inhibitor of metalloproteinases-1 protects human neurons from staurosporine and HIV-1-induced apoptosis: mechanisms and relevance to HIV-1-associated dementia. Cell Death Dis 3, e332 (2012).
23. Houseman, E. et al. DNA methylation arrays as surrogate measures of cell mixture distribution. BMC Bioinformatics 13, 86 (2012).
24. Horvath, S. DNA methylation age of human tissues and cell types. Genome Biol 14, R115 (2013).
25. Horvath, S. & Levine, A. J. HIV-1 Infection Accelerates Age According to the Epigenetic Clock. J Infect Dis 212, 1563-73 (2015).
26. Horvath, S. et al. An epigenetic clock analysis of race/ethnicity, sex, and coronary heart disease. Genone Biol 17, 171 (2016).
27. Hannum, G. et al. Genome-wide methylation profiles reveal quantitative views of human aging rates. Mol Cell 49, 359-67 (2013).
28. Levine, M. E. et al. An epigenetic biomarker of aging for lifespan and healthspan. Aging (Albany N. Y.) 10, 573-591 (2018).
29. Zhang, Y. et al. DNA methylation signatures in peripheral blood strongly predict all-cause mortality. Nat Comnun 8, 14617 (2017).
30. Horvath, S. & Levine, A. J. HIV-1 infection accelerates age according to the epigenetic clock. J Infect Dis (2015).
31. Langfelder, P. & Horvath, S. WGCNA: an R package for weighted correlation network analysis. BMC Bioinformatics 9, 559 (2008).

All publications mentioned herein are incorporated herein by reference to disclose and describe the methods and/or materials in connection with which the publications are cited (e.g. Lu et al., Aging (Albany N.Y.). 2019 Jan. 21; 11(2):303-327. doi: 10.18632/aging 101684). Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A method of obtaining information on one or more physiological factors associated with an age of an individual, the method comprising:
obtaining genomic DNA from the individual;
observing methylation of the genomic DNA in at least about 42 methylation markers from the group of methylation markers in SEQ ID NO: 1-SEQ ID NO: 1113;
correlating observed methylation in the methylation markers with the one or more physiological factors associated with the age of an individual such that information on the one or more physiological factors associated with the age of an individual is obtained.

2. The method of claim 1, wherein the one or more physiological factors associated with an age of an individual comprises a number of years in which the individual has smoked in their lifetime, the method comprising:
observing methylation of the genomic DNA in about 172 methylation markers from the group of methylation markers in SEQ ID NO: 1-SEQ ID NO: 172;
correlating observed methylation in the about 172 methylation markers with the number of years in which an individual has smoked in their lifetime, such that information on the number of years in which the individual has smoked in their lifetime is obtained.

3. The method of claim 1, wherein the one or more physiological factors associated with an age of an individual comprises plasma protein levels of tissue inhibitor metalloproteinase 1 in the individual, the method comprising:
observing methylation of the genomic DNA in about 42 methylation markers from the group of methylation markers in SEQ ID NO: 173-SEQ ID NO: 214;
correlating observed methylation in the about 42 methylation markers with plasma protein levels of tissue inhibitor metalloproteinase 1 in an individual, such that information on plasma protein levels of tissue inhibitor metalloproteinase 1 in an individual is obtained.

4. The method of claim 1, wherein the one or more physiological factors associated with an age of an individual comprises plasma protein levels of Cystatin-C in the individual, the method comprising:
observing methylation of the genomic DNA in about 87 methylation markers from the group of methylation markers in SEQ ID NO: 215-SEQ ID NO: 301;
correlating observed methylation in the about 87 methylation markers with plasma protein levels of Cystatin-C in an individual, such that information on plasma protein levels of Cystatin-C in an individual is obtained.

5. The method of claim 1, wherein the one or more physiological factors associated with an age of an individual comprises plasma protein levels of Beta-2-microglobulin in the individual, the method comprising:
observing methylation of the genomic DNA in about 91 methylation markers from the group of methylation markers in SEQ ID NO: 302-SEQ ID NO: 392;
correlating observed methylation in the about 91 methylation markers with plasma protein levels of Beta-2-microglobulin in an individual, such that information on plasma protein levels of Beta-2-microglobulin in an individual is obtained.

6. The method of claim 1, wherein the one or more physiological factors associated with an age of an individual comprises plasma protein levels of growth differentiation factor 15 in the individual, the method comprising:
observing methylation of the genomic DNA in about 137 methylation markers from the group of methylation markers in SEQ ID NO: 393-SEQ ID NO: 529;
correlating observed methylation in the about 137 methylation markers with plasma protein levels of growth differentiation factor 15 in an individual, such that information on plasma protein levels of growth differentiation factor 15 in an individual is obtained.

7. The method of claim 1, wherein the one or more physiological factors associated with an age of an individual comprises plasma protein levels of adrenomedullin in the individual, the method comprising:
observing methylation of the genomic DNA in about 186 methylation markers from the group of methylation markers in SEQ ID NO: 530-SEQ ID NO: 715;
correlating observed methylation in the about 186 methylation markers with plasma protein levels of adrenomedullin in an individual, such that information on plasma protein levels of adrenomedullin in an individual is obtained.

8. The method of claim 1, wherein the one or more physiological factors associated with an age of an individual comprises plasma protein levels of Leptin in the individual, the method comprising:
observing methylation of the genomic DNA in about 187 methylation markers from the group of methylation markers in SEQ ID NO: 716-SEQ ID NO: 902;
correlating observed methylation in the about 187 methylation markers with Leptin in an individual, such that information on plasma protein levels of Leptin in an individual is obtained.

9. The method of claim 1, wherein the one or more physiological factors associated with an age of an individual comprises plasma protein levels of plasminogen activator inhibitor 1 in the individual, the method comprising:
observing methylation of the genomic DNA in about 211 methylation markers from the group of methylation markers in SEQ ID NO: 903-SEQ ID NO: 1113;
correlating observed methylation in the about 211 methylation markers with plasminogen activator inhibitor 1 in an individual, such that information on plasma protein levels of plasminogen activator inhibitor 1 in an individual is obtained.

10. The method of claim 1, wherein the one or more physiological factors associated with an age of an individual comprises predicted age or lifespan of an individual, the method comprising:
observing methylation of the genomic DNA in about 1113 methylation markers from the group of methylation markers in SEQ ID NO: 1-SEQ ID NO: 1113;
correlating observed methylation in the about 1113 methylation markers with predicted age or lifespan of an individual, such that information on predicted age or lifespan of an individual an individual is obtained.

11. The method of claim 1, wherein genomic DNA is obtained from human fibroblasts, keratinocytes, buccal cells, endothelial cells, lymphoblastoid cells, and/or cells obtained from blood, skin, dermis, epidermis or saliva.

12. The method of claim 1, wherein:
methylation is observed by a process comprising treatment of genomic DNA from the population of cells from the individual with bisulfite to transform unmethylated cytosines of CpG dinucleotides in the genomic DNA to uracil; and/or
genomic DNA is hybridized to a complimentary sequence disposed on a microarray.

13. The method of claim 1, wherein correlating observed methylation in the methylation markers comprises a regression analysis.

14. The method of claim 1, wherein genomic DNA is amplified by a polymerase chain reaction process.

15. A method of observing the effects of a test agent on genomic methylation associated epigenetic aging of human cells, the method comprising:
(a) combining the test agent with human cells;
(b) observing methylation status in at least 42 of the methylation markers of SEQ ID NO: 1-SEQ ID NO: 1113 in genomic DNA from the human cells;
(c) comparing the observations from (b) with observations of the methylation status in at least 42 of methylation markers of SEQ ID NO: 1-SEQ ID NO: 1113 in genomic DNA from control human cells not exposed to the test agent such that effects of the test agent on genomic methylation associated epigenetic aging in the human cells is observed.

16. The method of claim 1, wherein the method comprises:
observing methylation of the genomic DNA in about 172 methylation markers from the group of methylation markers in SEQ ID NO: 1-SEQ ID NO: 172;
observing methylation of the genomic DNA in about 42 methylation markers from the group of methylation markers in SEQ ID NO: 173-SEQ ID NO: 214;
observing methylation of the genomic DNA in about 87 methylation markers from the group of methylation markers in SEQ ID NO: 215-SEQ ID NO: 301;
observing methylation of the genomic DNA in about 91 methylation markers from the group of methylation markers in SEQ ID NO: 302-SEQ ID NO: 392;
observing methylation of the genomic DNA in about 137 methylation markers from the group of methylation markers in SEQ ID NO: 393-SEQ ID NO: 529;
observing methylation of the genomic DNA in about 186 methylation markers from the group of methylation markers in SEQ ID NO: 530-SEQ ID NO: 715;
observing methylation of the genomic DNA in about 187 methylation markers from the group of methylation markers in SEQ ID NO: 716-SEQ ID NO: 902;
observing methylation of the genomic DNA in about 211 methylation markers from the group of methylation markers in SEQ ID NO: 903-SEQ ID NO: 1113; and/or
observing methylation of the genomic DNA in about 1113 methylation markers of SEQ ID NO: 1-SEQ ID NO: 1113.

17. The method of claim 15, wherein a plurality of test agents are combined with the human cells.

18. The method of claim 15, wherein the cells are primary keratinocytes from multiple donors.

19. The method of claim 15, wherein the method observes human cells in vitro.

20. The method of claim 15, wherein the test agent is a compound having a molecular weight less than 3,000, 2,000, 1,000 or 500 g/mol.
